(19) United States
(12) Patent Application Publication
(10) Pub. No.: US 2009/0082466 A1
(43) Pub. Date: Mar. 26, 2009

(54) Abuse Resistant and Extended Release Formulations and Method of Use Thereof

(76) Inventor: Najib Babul, Blue Bell, PA (US)

Correspondence Address: TheraQuest Biosciences, Inc., 146 Medinah Drive, Blue Bell, PA, 19422-3212, US

(21) Appl. No.: 12/223,327

(22) PCT Filed: Jan. 29, 2007
(86) PCT No.: PCT/US2007/002378

§ 371 Date: Jul. 29, 2008

Related U.S. Application Data
(60) Provisional application No. 60/762,489, filed on Jan. 27, 2006.

(51) Int. Cl. A61K 31/135; A61K 47/00

(52) U.S. Cl. 514/646; 514/783; 514/785

(57) ABSTRACT

The present invention is in the field of oral, abuse resistant pharmaceutical compositions of opioids, extended release pharmaceutical compositions of opioids and extended release abuse resistant pharmaceutical compositions of opioids and the use thereof for the treatment of pain. The present invention is also directed to extended release pharmaceutical compositions and the use thereof for preventing or minimizing the risk of opioid abuse and/or opioid toxicity from either intentional or unintentional tampering. The present invention is further directed at a method of preventing or minimizing the risk of opioid abuse and/or opioid toxicity from either intentional or unintentional tampering.

The application claims the benefit of U.S. Provisional Application No. 60/762,489, filed Jan. 27, 2006, which is herein incorporated by reference in its entirety.

### FIELD OF THE INVENTION

The present invention is in the field of oral, abuse resistant pharmaceutical compositions of opioids, extended release pharmaceutical compositions of opioids and extended release abuse resistant pharmaceutical compositions of opioids and the use thereof for the treatment of pain.

### BACKGROUND ART

Currently, medical practitioners may choose from several well-accepted classes of pharmaceutical agents in their attempts to alleviate and prevent pain. Nonlimiting examples of agents used include nonsteroidal anti-inflammatory agents (NSAIDs), e.g., aspirin, ibuprofen, ketoprofen, diclofenac; opioids, e.g., morphine, hydromorphone, hydrocodone, levorphanol, oxycodone, tramadol, and codeine; cyclooxygenase-2 (COX-2) selective NSAIDs, e.g., celecoxib, valdecoxib, etoricoxib, lumiracoxib, and rofecoxib; acetaminophen; tricyclic antidepressants, e.g., amitriptyline, desipramine, nortriptyline; non-tricyclic antidepressants, e.g., doxepin, duloxetine, paroxetine, venlafaxine; antiepileptics, e.g., gabapentin, pregabalin, carbamazepine, oxcarbazepine, lamotrigine; voltage sensitive N-type calcium channel blockers, e.g., ziconotide and alpha adrenergic agonists, e.g., clonidine.

An important goal of analgesic therapy is to achieve continuous relief of pain. Regular administration of an analgesic is generally required to ensure that the next dose is given before the effects of the previous dose have worn off. Continuous suppression of pain through the use of around the clock opioid analgesics is now recommended in treatment guidelines (Principles of Analgesic Use in the Treatment of Acute Pain and Cancer Pain, Fifth Ed., American, Pain Society (2003); Evidence Based Report of the U.S. Agency for Healthcare Research and Quality (AHRQ) on the Management of Cancer Pain, Report No. 35, AHRQ Publication No. 02-E002, October 2001; Carr et al. J Nat Cancer Inst Monograph 2004; 32:23-31; Agency for Health Care Policy and Research Clinical Practice Guidelines for Cancer Pain Management, Guideline No. 9, AHCPR Publication No. 94-0592, March 1994; Agency for Health Care Policy and Research Clinical Practice Guideline for Acute Pain Management, Guideline No. 1, AHCPR Publication No. 92-0032, February, 1992; Guideline for the Management of Cancer Pain in Adults, American Pain Society, 2005; Guideline for the Management of Pain in Osteoarthritis, Rheumatoid Arthritis, and Juvenile Chronic Arthritis, 2^{nd} Ed., American Pain Society, 2002).

Conventional (so called “immediate-release”, “rapid release” or “short acting”) opioid analgesics have been demonstrated to provide short-lived plasma levels, thereby requiring dosing every 4-6 hours in chronic pain. In contrast, extended release oral opioids are designed to maintain effective plasma levels throughout a 12 or 24-hour dosing interval. Extended release opioid formulations have now become the standard of care for the management of chronic pain. Use of extended release opioids can result in fewer interruptions in sleep, reduced dependence on caregivers, improved compliance, enhanced quality of life outcomes, and increased control over the management of their pain. In addition, such formulations can provide more constant plasma concentrations and clinical effects, less frequent peak to trough fluctuations and fewer side effects, compared with short acting opioids (Babul et al. Journal of Pain and Symptom Management 2004; 28:59-71; Matsumoto et al., Pain Medicine 2005; 6:357-66; Dhaliwal et al., Journal of Pain Symptom Management 1995; 10:612-23; Hays et al., Cancer 1994; 74:1808-16; Arkinstall et al., Pain 1995; 62:169-78; Hagen et al., Journal of Clinical Pharmacology 1995; 35:38-45; Peloso et al., Journal of Rheumatology 2000; 27:764-71).

Several studies have suggested the benefits of extended release over immediate release opioids. Ferrell et al (Oncol Nur Forum 1989; 4:521-6) compared 12-hourly controlled release morphine and short-acting analgesics in cancer pain and demonstrated that compliance increased as the required dosing frequency decreased, and noncompliance resulted in suboptimal pain control and poor quality-of-life outcomes. Arkinstall et al. (Pain 1995; 62:169-78) demonstrated that around that twice daily administration of controlled release codeine provided superior to pain control than a PRN regimen of acetaminophen plus codeine.

An important drawback with the use of opioid is the risk of drug addiction, drug diversion and drug abuse. Although the use of opioids for non-medical purposes has existed throughout recorded human history, their abuse has increased significantly in the past two decades (Drug Abuse Warning Network, http://dawninfo.samhsa.gov/; Drug Enforcement Administration, http://www.deadiversion.usdoj.gov/; National Survey on Drug Use & Health, http://www.oas.samhsa.gov/nhsda.htm; American Association of Poison Control Centers Toxic Exposure Surveillance System, http://www.aapcc.org/annual.htm).

Our increased understanding of the clinical pharmacology of opioids and data from well controlled clinical trials in chronic non-cancer pain (Peloso et al., Journal of Rheumatology 2000; 27:764-71; Caldwell, et al., Journal of Pain and Symptom Management 2002; 23:278-91; Matsumoto et al., Pain Medicine 2005; 6:357-66; Arkinstall et al., Pain 1995; 62:169-78) and neuropathic pain (Watson and Babul, Neurology 1998; 50:1837-41) have resulted in more widespread use in patients with non-malignant pain (for a review, see Sloan and Babul, Expert Opinion on Drug Delivery 2006; 3:489-97). This in turn has led to concerns about the increased non-medical use of opioids through both licit and illicit channels. For instance, unsuspecting clinicians may prescribe opioids for pain to individuals with an addiction disorder or individuals with pain who divert a portion of their prescribed dose to other individuals. There have also been documented cases of inappropriate prescribing or dispensing of opioids by physicians and pharmacists, with its eventual diversion into the non-medical marketplace. Additionally, non-medical supplies of pharmaceutical grade opioids are often obtained through prescription forgeries and break-ins into pharmacies.

Pharmaceutical dosage forms containing opioids have been used for non-medical purposes in a variety of settings: i) by patients with pain who have developed an addiction disorder following initiation of opioid therapy; ii) by patients with pain who had a pre-existing addiction disorder; iii) by patients with an addiction disorder seeking opioids for their euphoriant properties.

Non-medical users of opioid analgesics are either recreational drug users who may use such agents episodically, or individuals with an addiction disorder who may require frequent maintenance doses. Opioid analgesics may be ingested whole, crushed and ingested, crushed or vaporized and snorted or injected intravenously after attempted extraction of the active pharmaceutical ingredient. The manipulation of pharmaceutical dosage forms of opioids has been documented for many decades. For instance, pentazocine (Talwin®), a synthetic opioid was crushed, extracted and injected intravenously by drug addicts.

The introduction of extended release morphine (MS Contin®) revolutionized the management of cancer pain. MS Contin® gained wide-spread acceptance due to its global availability, significant pharmacokinetic and pharmacodynamic data, and the convenience of an extended-release formulation. However, the incidence and severity of side effects limits the use of morphine in some patients (Hagen and Babul, Cancer 1997; 79:1428-37). In patients with renal impairment, morphine's principal metabolites, morphine-3-glucuronide and morphine-6-glucuronide can accumulate. Morphine-3-glucuronide accumulation has been implicated in hyperalgesia, respiratory stimulation, and behavioral excitatory properties through nonopioid receptor mechanisms. Morphine-6-glucuronide accumulation has been implicated in increasing levels of nausea and sedation in patients with renal impairment (Babul and Darke, Clin Pharm Ther, 1993; 54:286-92).

Clinicians treating cancer pain with opioids have reported significant variability among patients in efficacy and side effects with available opioid analgesics. Patients with poor analgesic efficacy or safety outcomes on one opioid frequently tolerate another opioid well. This clinical observation led to the development of oxycodone ER (OxyContin®). Due to the limitations associated with extended release morphine noted above and the “stigma” associated with its use (i.e., association with addiction, advanced cancer, dying and death), extended release oxycodone gained rapid acceptance by patients with chronic non-cancer pain. However, its widespread use for the treatment of chronic non-malignant pain was also associated with its diversion into the non-medical supply for use both by addicts and recreational drug users.

The popularity of extended release oxycodone among addicts and recreational drug users was due to a large amount of drug per tablet (a 12 or 24 hour supply). Commercially available immediate release opioid tablets and capsules are usually administered every 4 to 6 hours and they release their dose into the systemic circulation over one to two hours. New, extended release formulations are designed to gradually release their much larger opioid content over a 12 or 24-hour period. Most recreational drug users and addicts have a unit of use which is one tablet or capsule. The 12 or 24-hour supply of opioid contained in one tablet or capsule, instead of 4 to 6 tablets or capsules means that there is a greater risk that such formulations may be highly sought by drug addicts and recreational drug users alike, for non-medical use. Intentional or inadvertent tampering from extended release formulations will rapidly deliver a massive dose and produce profound a variety of serious and life threatening side effects, including respiratory depression and failure, sedation, cardiovascular collapse, coma and death.

Addicts and recreational drug users commonly use extended release opioids by a variety of routes of administration. Commonly used methods include 1) parenteral (e.g., intravenous injection), 2) intranasal (e.g., snorting), and 3) episodic or repeated oral ingestion of intact or crushed tablets or capsules.

One mode of abuse involves the extraction of the opioid component from the dosage form by first mixing the table or capsule with a suitable solvent (e.g., water or alcohol), and then filtering and/or extracting the opioid component from the mixture for intravenous injection. Another mode of abuse of extended release opioids involves dissolving the drug in water, alcohol or another “recreational solvent” to hasten its release and to ingest the contents orally, in order to provide high peak concentrations and maximum euphoriant effects.

A number of strategies have been introduced to minimize the abuse of mood altering drugs. Primary among these schemes is a legal infrastructure that controls the manufacture, distribution and sale of such drugs. In the United States, the vast majority of opioid drugs having clinically useful and approved effects are restricted to dispensing on a prescription-only basis. Most of these drugs are “scheduled” as “controlled drugs”, such that distribution of the drug is subject to strict controls and overview. The idea behind scheduling opioid drugs as “controlled” is to ensure that the drugs are dispensed only for the amelioration of legitimate therapeutic maladies, and not for any mood-altering effect “high” or euphoria that may be produced by the drug when used in supra-therapeutic doses or administered by non-approved routes of administration.

While the scheduling of opioids as “controlled drugs” has reduced abuse of the drugs, it has not been entirely successful. For example, some persons who are legitimately prescribed the drugs sometimes divert the drugs to persons seeking their procurement for “recreational uses.” These “recreational drug users” are frequently willing to pay significant sums of money for the drugs. In other cases, certain health professionals, unfortunately, have been found to be culprits in the non-approved distribution of opioid drugs.

It is believed that the most widely used diversion techniques at the “street level” are “doctor shopping” and prescription forgeries. In the case of the former, individuals who may or may not have a legitimate ailment requiring a doctor's prescription for controlled substances, visit numerous doctors, sometimes in several states, to acquire large amounts of controlled substances they abuse or sell to others.

Scheduling of opioid drugs has also had the unintentional side-effect of causing physicians, fearful of being accused of permitting “opioid overuse”, to prescribe suboptimal doses of opioids to patients in need of them, and to prescribe less effective drugs to patients that are not similarly scheduled. This phenomenon is described in the literature as “opiophobia” or “narcophobia”.

There is a growing recognition in the medical community that a large number of patients suffer from the undertreatment of pain. Among the reasons frequently cited as causative of undertreatment are: (1) the failure to prescribe enough drug at the right dosage interval to reach a steady-state threshold commensurate with the pain relief needed; (2) failure of patients to comply with a given dosage regimen; and (3) the reluctance of many physicians to prescribe analgesics categorized as controlled drugs based on often unfounded concerns of future addiction and fear of regulatory sanctions. For example, it has been reported that with respect to cancer pain, a large percentage of cancer patients suffer debilitating pain despite treatment with analgesics (Cleeland et al., New England Journal of Medicine 1994; 330:592-596).

Attempts have been made to deter or minimize the abuse of orally administered opioids. These attempts have generally focused on the inclusion in the oral dosage form of an opioid antagonist, which is not orally active, but which will substantially block the analgesic effects of the opioid if one attempts to dissolve the opioid and administer it parenterally. A further evolution of this strategy has involved the inclusion in the oral dosage form of a sequestered, orally bioavailable opioid antagonist, which is released only upon product tampering (e.g., crushing, extraction). In this circumstance, the opioid antagonist is not expected to be orally active under normal conditions of use but would nullify the euphoriant effects of either oral or intravenous administration upon product tampering.

For example, commercially available Talwin®Nx tablets from Sanofi-Winthrop contain a combination of pentazocine and naloxone. Pentazocine is a partial agonist at the μ opioid receptors and also has affinity at κ opioid receptors, whereas, naloxone is an antagonist of μ receptors. Talwin Nx contains pentazocine hydrochloride equivalent to 50 mg base and naloxone hydrochloride equivalent to 0.5 mg base. Talwin Nx is indicated for the relief of moderate to severe pain. The amount of naloxone present in this combination has no action when taken orally, and will not interfere with the pharmacologic action of pentazocine. However, this amount of naloxone given by injection has profound antagonistic action to opioid analgesics. Thus, the inclusion of naloxone is intended to curb a form of misuse of oral pentazocine, which occurs when the dosage form is solubilized and injected. Therefore, this dosage has lower potential for parenteral misuse than previous oral pentazocine formulations. Similarly, a drug known as Valoron®N (Goedecke), that comprises tilidine (50 mg) and naloxone (4 mg), has been available in Germany for the management of severe pain.

A fixed combination of buprenorphine and naloxone was introduced in 1991 in New Zealand (Temgesic®Nx, Reckitt & Colman) for the treatment of pain.

U.S. Pat. No. 4,457,933 to Gordon et al. teaches the reduction in the oral abuse potential of the analgesics oxycodone, propoxyphene and pentazocine by combining the analgesic with naloxone in a specific range. Naloxone is combined with the selected analgesic a ratio of 2.5-5:1 part.

U.S. Pat. No. 6,228,863 to Palermo et al. teaches the reduction of the abuse potential of oral dosage forms of opioid analgesics by selecting the particular opioid agonist and antagonist pair, and the concentrations of the same such that the antagonist cannot be easily extracted from the agonist (at least a two-step extraction process being needed to separate the drugs—see also, WO 99/32120). The antagonist is in such a concentration that the combination will cause an aversive effect in a physically dependent human subject but not in a naive individual (See also, WO 99/32119).

U.S. Pat. No. 3,773,955 to Pachter et al. describes orally effective analgesic compositions which contain from about 0.1 mg to about 10 mg naloxone with the opioid analgesic. Upon extraction of the composition, parenteral administration is dissuaded, as the dose of naloxone is high enough to prevent the production of analgesia, euphoria or physical dependence from the opioid analgesic. WO 01/58447 describes a controlled-release composition which contains an opioid agonist and opioid antagonist that provides an analgesic amount of the opioid agonist over 8 hours along with an amount of opioid antagonist to attenuate a side effect of the opioid agonist. WO 01/58451 discloses an oral dosage form comprising an opioid agonist in releasable form and a sequestered opioid antagonist which is substantially not released when the dosage form is administered intact but is released upon tampering. As indicated above WO 99/32120 further describes selecting the opioid agonist and antagonist with respect to physical properties so as to require at least a two-step extraction process to separate the opioid agonist from the antagonist, the amount of opioid antagonist being otherwise sufficient to counteract opioid agonist effect if administered parenterally.

U.S. Pat. No. 3,493,657 to Lewenstein, et al. describes compositions comprising naloxone and morphine or oxymorphone, which compositions were said to provide a strong analgesic effect without the occurrence of undesired side effects such as hallucinations.

U.S. Pat. No. 4,582,835 to Lewis describes a method of treating pain by administering a sublingually effective dose of buprenorphine with naloxone. Lewis describes dosage ratios of naloxone to buprenorphine from 1:3 to 1:1 for parenteral administration, and from 1:2 to 2:1 for sublingual administration.

U.S. Pat. No. 6,559,159 to Carroll et al. describes the use of kappa receptors antagonist for the treatment of opioid related addictions. One such compound is naltrexone, which is commercially available in the tablet form Revia® for the treatment of alcohol dependence and for the blockade of exogenously administered opioids.

U.S. Pat. Nos. 6,277,384, 6,375,957 and 6,475,494 describe oral dosage forms including a combination of an orally active opioid agonist and an orally active opioid antagonist in a ratio that, when delivered orally, is analgesically effective but that is aversive in a physically dependent subject.

U.S. Pat. Nos. 3,980,766, 4,070,494 and 6,309,668 describe formulations designed to prevent the injection of compositions meant for oral administration.

U.S. Pat. No. 3,980,766 describes the incorporation of an ingestible solid which causes a rapid increase in viscosity upon concentration of an aqueous solution thereof.

U.S. Pat. No. 4,070,494 describes the incorporation of a non-toxic, water gelable material in an amount sufficient to render the drug resistant to aqueous extraction.

U.S. Pat. No. 6,309,668 describes a tablet for oral administration containing two or more layers comprising one or more drugs and one or more gelling agents within separate layers of the tablet. The resulting tablet forms a gel when combined with the volume of water necessary to dissolve the drug; this formulation thus reduces the extractability of the drug from the tablet. It should be noted that although these compositions preclude abuse by injection, this approach fails to prevent abuse by crushing and swallowing or snorting the formulation, which are commonly reported methods of abuse associated with OxyContin®.

U.S. Pat. Nos. 3,773,955 and 3,966,940 describe formulations containing a combination of opioid agonists and antagonists, in which the antagonist does not block the therapeutic effect when the admixture is administered orally, but which does not produce analgesia, euphoria or physical dependence when administered parenterally by an abuser.

U.S. Pat. No. 4,457,933 describes a method for decreasing both the oral and parenteral abuse potential of strong analgesic agents by combining an analgesic dose of the analgesic agent with an antagonist in specific, relatively narrow ratios.

The problem with all of the above schemes that incorporate opioid antagonists into the opioid preparation to deter abuse is that opioid antagonists themselves have side effects that may be disadvantageous. For example, nalorphine causes unpleasant reactions that range from anxiety, to “crazy feelings,” to hallucinations, respiratory depression and miosis. Seizures have been reported with naloxone, albeit infrequently, and in postoperative patients, pulmonary edema and ventricular fibrillation have been seen with high dosages. Naltrexone has been reported to have the capacity to cause hepatocellular injury when given in doses as low as fivefold or less of therapeutic doses. Nalmefene, although usually well tolerated, has been reported to cause nausea, vomiting and tachycardia in some individuals. Small doses of any of these opioid antagonists can also precipitate an abstinence syndrome in opioid tolerant patients, resulting in drug withdrawal. Symptoms of opioid withdrawal include body aches, diarrhea, gooseflesh, loss of appetite, nervousness or restlessness, runny nose, sneezing, tremors or shivering, stomach cramps, nausea, trouble with sleeping, increased sweating, increased yawning, weakness, increased heart rate or fever. These symptoms can be severe, requiring hospitalization and reinstitution of the opioid agonist (Sloan and Babul, Expert Opinion on Drug Delivery 2006; 3:489-97).

Purdue Pharma (Euro-Celtique SA) have reported that one opioid tolerant volunteer among a 24-subject group receiving their extended release opioid agonist with a sequestered opioid antagonist developed severe opioid withdrawal, requiring hospitalization (Sloan and Babul, Expert Opinion on Drug Delivery 2006; 3:489-97).

There is a need, therefore, for novel methods of deterring or preventing opioid abuse which do not require the incorporation of opioid antagonists into the formulation.

A number of reported cases of opioid toxicity are a result of inadvertent or unintentional medical use of opioids. It is not uncommon for patients who have difficulty swallowing, to crush the contents of tablets or open a capsule, and swallow the contents with liquids or on soft food. In the case of most immediate release formulations, this generally produces no significant harm, with marginally higher peak concentrations (Cₘₐₓ) and time to peak concentrations (tₘₐₓ). However, in the case of extended release opioid formulations, crushing the oral solid dosage form destroys the controlled-release mechanism and results in a rapid surge of drug into the bloodstream, with the entire 12 or 24-hour drug supply released immediately with toxic effects. For this reason, all extended release formulations available for sale in the United States carry a warning to the prescriber and patient not to crush or tamper with the oral solid dosage form (see Prescribing Information for MS Contin®, OxyContin®, Avinza® and Kadian®, Physician's Desk Reference, 2005, Thompson P D R, Montvale, N.J.).

The above abuse deterrent formulations can cause serious harm to patients by precipitating an opioid abstinence syndrome through the liberation of the opioid antagonist. There is therefore a need for a “passive” abuse deterrent system to protect both medical and non-medical users of opioids from intentional or unintentional opioid toxicity, without unnecessary harm to either group from the abuse deterrent technology.

Similarly, abuse deterrent pharmaceutical compositions containing aversive substances can cause serious harm to subjects if injected intravenously and the long terms safety of small amounts of such aversive substances which would be inevitably released in the gastrointestinal tract is unknown.

There is also need, therefore, for novel methods of preventing opioid abuse which do not require the incorporation of aversive and potentially unsafe agents into the formulation.

In 2005, a serious new clinical problem arose with the therapeutic use of extended release opioids, particularly extended release formulations in capsule dosage forms, when co-ingested with alcohol. In this setting, the opioid analgesic was being used for legitimate medical purposes (e.g., to treat pain) and was being ingested as an untampered or intact formulation. Although subjects with chronic pain are discouraged from using opioids with alcohol, the co-ingestion of opioids with alcohol, especially in the setting of intractable pain is widespread. The problem was discovered with a once-a-day extended release formulation of the opioid hydromorphone HCL (Palladone® capsules). Palladone® capsules were introduced in the United States and Canada in 2004. In 2005, Palladone® capsules were withdrawn from the market in both countries due to dose-dumping when co-ingested with alcohol. In a 24-subject study, patients consuming 240 mL of 40% ethanol had a 6-fold mean increase in peak plasma hydromorphone concentration compared with co-ingestion of Palladone® capsules with water. One subject experienced a 16-fold increase when the drug was ingested with 40% alcohol compared with water. Patients consuming 240 mL of 20% ethanol had a 2-fold mean increase in peak plasma hydromorphone concentration. One subject in this group experienced a 6-fold increase when the drug was ingested with 20% alcohol compared with water. In some subjects, 8 ounces of 4% alcohol (equivalent to ⅔ of a typical serving of beer) resulted in almost twice the peak plasma hydromorphone concentration than when the drug was ingested with water. In requesting the withdrawal of Palladone® capsules, FDA noted that the manufacturer of “Palladone® provided FDA data that showed that drinking alcohol while taking Palladone® capsules may cause rapid release of hydromorphone, leading to high drug levels in the body, with potentially fatal effects. High drug levels of hydromorphone may depress or stop breathing, cause coma, and even cause death. The Agency has concluded that the overall risk versus benefit profile of Palladone® is unfavorable due to a potentially fatal interaction with alcohol. Pharmacokinetic data indicate that the co-ingestion of Palladone® and alcohol results in dangerous increases in the peak plasma concentrations of hydromorphone. These elevated levels may be lethal, even in opioid tolerant patients.” (Sloan and Babul, Expert Opinion on Drug Delivery 2006; 3:489-97; http://www.fda.gov/cder/drug/infopage/palladone/default.htm)

FDA has since noted that a number of other capsule formulations of extended release opioids may be similarly vulnerable to dose dumping when co-ingested with alcohol. In vitro studies performed by the FDA have demonstrated that when Avinza® (once-daily extended release morphine) 30 mg was mixed with 900 mL of buffer solutions containing ethanol, the dose of morphine that was released was alcohol concentration-dependent, leading to a more rapid release of morphine. While the relevance of in vitro lab tests regarding Avinza® to the clinical setting remains to be determined, this acceleration of release may correlate with in vivo rapid release of the total morphine dose, which could result in the absorption of a potentially fatal dose of morphine. (http://www.fda.gov/medwatch/SAFETY/2005/AVINZA_DHCP_Letter_Oct. 2005.pdf; Sloan and Babul, Expert Opinion on Drug Delivery 2006; 3:489-97)

There is therefore also need, therefore, for novel methods of preventing excessive peak concentrations (dose dumping) of opioids when they are co-ingested for medical purposes at prescribed doses with alcohol.

To date, no extended release formulations of opioids with abuse deterrent technology of any kind have been submitted for Marketing Application (New Drug Application) or been commercialized anywhere in the world. Indeed if prior drug development history is any guide, most such strategies are unlikely to be developed or commercialized and the optimal formulation(s) will likely be apparent only through postmarketing surveillance of several formulations with competing technologies. In addition, regional differences in patterns of abuse mean that different abuse deterrence strategies may be useful in different part of the world. Finally, experience with substance abuses indicates that those who are habitual abusers, particularly those who inject drugs intravenously, have a remarkable ability to defeat abuse deterrence strategies through physical and chemical manipulation of opioids and other drugs of abuse. Such addicts are frequently only one step behind strategies to deter abuse. With the ready access to information from their well knit network and more recently, from websites on how to optimally extract the active agent from pharmaceutical dosage forms and maximize euphoriant effects, the development of abuse deterrent formulations has become a major pharmaceutical, clinical, regulatory and law enforcement challenge.

In view of this, it is not surprising that the Food and Drug Administration's Division of Anesthetic, Analgesic and Rheumatology Drug Products and the U.S. Drug Enforcement Administration have encouraged companies to develop wide ranging abuse deterrent strategies for opioids, particularly extended release opioids and as “inducement”, offered that such products may include in their prescribing information data about their products abuse deterrent properties (FDA Perspectives on Opioid Risk Management. Opioid Risk Management Meeting, Tufts Healthcare Institute, Boston, Mar. 29, 2005; DEA Perspectives on Opioid Risk Management. Opioid Risk Management Meeting, Tufts Healthcare Institute, Boston, Mar. 29, 2005).

In summary, various attempts have been made and are described in prior art to develop abuse-deterrent dosage forms. Clearly there is a need for a delivery system for commonly used oral dosage formulations of drugs, and in particular analgesics such as opioid analgesics, for patients seeking drug therapy and which deters abuse and minimizes or reduces the potential for psychological dependence. In particular, there is a need for formulations that simultaneously provide robust abuse deterrence properties and an extended release pharmacokinetic profile suitable for every 12 or 24 hour oral administration. There is also a need for extended release formulations of opioids that are stable (i.e., do not dose dump) when used at therapeutic doses for medical purposes in conjunction with alcohol. An ideal formulation will provide a extended release pharmacokinetic profile suitable for every 12 or 24 hour release and will be resistant to crushing at room temperature and upon freezing, melting to allow for filtration and/or aspiration into a syringe and extraction with recreational solvents, all without doing harm to pain patients or patients with a substance abuse disorder, through the use of aversive agents or opioid antagonists.

### BRIEF SUMMARY OF THE INVENTION

The present invention is directed at pharmaceutical compositions of opioids to provide abuse deterrence properties.

The present invention is directed at pharmaceutical compositions of opioids to provide extended release properties.

The present invention is directed at pharmaceutical compositions of opioids that provide simultaneous abuse deterrence properties and extended release properties.

The present invention is directed at pharmaceutical compositions of opioids that provide simultaneous abuse deterrence properties and extended release properties using substantially the same ingredients to achieve abuse deterrence properties and extended release.

The present invention is directed at liquid pharmaceutical compositions of opioids that solidify at room temperature to provide abuse deterrence properties.

The present invention is directed at liquid pharmaceutical compositions of opioids that solidify at room temperature to provide extended release properties.

The present invention is directed at liquid pharmaceutical compositions of opioids that solidify at room temperature to provide simultaneous abuse deterrence properties and extended release properties.

The present invention is directed at liquid pharmaceutical compositions of opioids that solidify at room temperature to provide simultaneous abuse deterrence properties and extended release properties using substantially the same ingredients to achieve abuse deterrence properties and extended release.

The present invention is directed at oral opioid pharmaceutical compositions and methods for preventing or minimizing the risk of opioid toxicity from either intentional or unintentional tampering.

The present invention is directed at oral opioid pharmaceutical compositions and methods for deterring opioid abuse by drug addicts and/or recreational drug users.

The present invention is directed at oral opioid pharmaceutical compositions and methods for deterring surreptitious adulteration of liquid beverages.

The present invention is directed at oral opioid pharmaceutical compositions of and methods to resist, deter, discourage, diminish, delay and/or frustrate the intentional, unintentional or accidental physical manipulation or tampering of the dosage form (e.g., crushing, shearing, grinding, chewing, dissolving, melting, needle aspiration, inhalation, insufflation, extraction by mechanical, thermal and chemical means, and/or filtration).

The present invention is directed at oral opioid pharmaceutical compositions of and methods to resist, deter, discourage, diminish, delay and/or frustrate the intentional, unintentional or accidental use or misuse of the dosage form (a) outside the scope of specific instructions for use provided by a qualified medical professional; (b) outside the supervision of a qualified medical professional; and (c) outside the approved instructions on proper use provided by the drug's legal manufacturer (e.g., intravenous use, intranasal use, inhalational use and oral ingestion to provide high peak concentrations)

The present invention is directed at oral opioid pharmaceutical compositions of and methods to resist, deter, discourage, diminish, delay and/or frustrate the intentional, unintentional or accidental conversion of an extended release dosage form of the invention into a more immediate release form.

The present invention is directed at oral opioid pharmaceutical compositions of and methods to resist, deter, discourage, diminish, delay and/or frustrate the intentional and iatrogenic increase in physical and psychic effects sought by recreational drug users, addicts, and patients with pain who have an addiction disorder.

The present invention is directed at oral opioid pharmaceutical compositions of and methods to resist, deter, discourage, diminish, delay and/or frustrate the attempts at surreptitious administration of the dosage form to a third party (e.g., in a beverage).

The present invention is directed at oral opioid pharmaceutical compositions of and methods to resist, deter, discourage, diminish, delay and/or frustrate attempts to divert the opioid dosage form into the non-medical supply chain.

The present invention is directed at oral opioid pharmaceutical compositions of and methods to resist, deter, discourage, diminish, delay and/or frustrate the use of the dosage form for medically unapproved or unintended mood altering purposes.

The present invention is directed at oral opioid pharmaceutical compositions of and methods to resist, deter, discourage, diminish, delay and/or frustrate intentional, unintentional or accidental attempts directed at changing the physical, pharmaceutical, pharmacological and/or medical properties of the dosage form from what was intended by the manufacturer.

The present invention is directed at oral opioid pharmaceutical compositions that provide extended release delivery of the drug and the use thereof for the treatment of pain and other medical maladies.

Opioid analgesics of the present invention can be formulated with the substantially the same ingredients to deter abuse and minimize opioid toxicity on tampering while simultaneously providing an extended release pharmacokinetic profile suitable for every 4, 6, 8, 12 or 24 hour dosing, without the need to include an aversive agent or an opioid antagonist in the formulation.

Compositions and methods of the present invention provide (i) abuse deterrence; (ii) extended release; and (iii) simultaneous abuse deterrence and extended release, prepared using compounds selected from the group consisting of: (a) hydrogenated Type I or Type II vegetable oils; (b) polyoxyethylene stearates and distearates; (c) glycerol monostearate; (d) poorly water soluble, high melting point (mp=40 to 100° C.) waxes, and mixtures thereof, said group of compounds hereinafter referred to as “abuse deterrent, extended release” or “ADER”.

A first aspect of the present invention is directed to a novel method for reducing the peak concentration (Cₘₐₓ) of the opioid analgesic, said method comprising administering the opioid analgesic and a suitable amount of ADER.

A second aspect of the present invention is directed to a novel method for reducing the early post-dose partial area under the plasma opioid concentration time curve (AUC₀₋ₜ, e.g., AUC₀₋₂, AUC₀₋₄ and AUC₀₋₆) of the opioid analgesic, said method comprising administering the opioid analgesic and a suitable amount of ADER.

A third aspect of the present invention is directed to a novel method for reducing the average plasma opioid concentration (Cave) early post-dose (e.g., Cave₀₋₁, Cave₀₋₂), said method comprising administering the opioid analgesic and a suitable amount of ADER.

A fourth aspect of the present invention is directed to a novel method for reducing the incidence of opioid toxicity upon tampering of the opioid analgesic, said method comprising administering the opioid analgesic and a suitable amount of ADER.

A fifth aspect of the present invention is directed to a novel method for reducing the intensity of opioid toxicity upon tampering of the opioid analgesic, said method comprising administering the opioid analgesic and a suitable amount of ADER.

A sixth aspect of the present invention is directed to a novel method for reducing the intensity or frequency of one or more signs and symptoms of opioid toxicity, including nausea, vomiting, somnolence, stupor, coma, respiratory depression, apnea, respiratory arrest, circulatory depression, bradycardia, hypotension, shock and skeletal muscle flaccidity, said method comprising administering the opioid analgesic and a suitable amount of ADER.

A seventh aspect of the present invention is directed to novel pharmaceutical compositions for use in reducing the peak concentration (Cₘₐₓ) of the opioid analgesic, said method comprising administering the opioid analgesic and a suitable amount of ADER.

An eighth aspect of the present invention is directed to novel pharmaceutical compositions for reducing the early post-dose partial area under the plasma opioid concentration time curve (AUC₀₋ₜ, e.g., AUC₀₋₂, AUC₀₋₄ and AUC₀₋₆) of the opioid analgesic, said method comprising administering the opioid analgesic and a suitable amount of ADER.

A ninth aspect of the present invention is directed to novel pharmaceutical compositions for reducing the average opioid plasma concentration (Cave) early post-dose (e.g., Cave₀₋₁, Cave₀₋₂), said method comprising administering the opioid analgesic and a suitable amount of ADER.

A tenth aspect of the present invention is directed to novel pharmaceutical compositions for reducing the incidence of opioid toxicity, said method comprising administering the opioid analgesic and a suitable amount of ADER.

An eleventh aspect of the present invention is directed to novel pharmaceutical compositions for reducing the intensity of opioid toxicity, said method comprising administering the opioid analgesic and a suitable amount of ADER.

An twelfth aspect of the present invention is directed to novel pharmaceutical compositions for reducing the intensity or frequency of one or more symptoms, including nausea, vomiting, somnolence, stupor, coma, respiratory depression, apnea, respiratory arrest, circulatory depression, bradycardia, hypotension, shock and skeletal muscle flaccidity, said method comprising administering the opioid analgesic and a suitable amount of ADER.

A thirteenth aspect of the present invention is directed to a novel method and pharmaceutical compositions for preventing or minimizing excessive peak concentrations (dose dumping) of therapeutic doses of extended release opioids used for medical purposes, when they are co-ingested with alcohol.

A fourteenth aspect of the present invention is directed to a novel method and pharmaceutical compositions for reducing the solvent extraction efficiency of the dosage form upon tampering.

A fifteenth aspect of the present invention is directed to a novel method and pharmaceutical compositions for reducing the filtration efficiency of the dosage form upon tampering.

These and many other objects and embodiments of the invention are further described herein.

### DETAILED DESCRIPTION OF THE INVENTION

In some preferred embodiments, the present invention is directed at pharmaceutical compositions of opioids to provide abuse deterrence properties.

In some preferred embodiments, the present invention is directed at pharmaceutical compositions of opioids to provide extended release properties.

In some preferred embodiments, the present invention is directed at pharmaceutical compositions of opioids that provide simultaneous abuse deterrence properties and extended release properties.

In some preferred embodiments, the present invention is directed at pharmaceutical compositions of opioids that provide simultaneous abuse deterrence properties and extended release properties using substantially the same ingredients to achieve abuse deterrence properties and extended release.

In some preferred embodiments, the present invention is directed at liquid pharmaceutical compositions of opioids that solidify at room temperature to provide abuse deterrence properties.

In some preferred embodiments, the present invention is directed at liquid pharmaceutical compositions of opioids that solidify at room temperature to provide extended release properties.

In some preferred embodiments, the present invention is directed at liquid pharmaceutical compositions of opioids that solidify at room temperature to provide simultaneous abuse deterrence properties and extended release properties.

In some preferred embodiments, the present invention is directed at liquid pharmaceutical compositions of opioids that solidify at room temperature to provide simultaneous abuse deterrence properties and extended release properties using substantially the same ingredients to achieve abuse deterrence properties and extended release.

In some preferred embodiments, the present invention is directed at oral opioid pharmaceutical compositions and the use thereof for preventing or minimizing the risk of opioid toxicity from either intentional or unintentional tampering.

In some preferred embodiments, the present, invention is directed at oral opioid pharmaceutical compositions and the use thereof for deterring opioid abuse by drug addicts and/or recreational drug users.

In some preferred embodiments, the present invention is directed at oral opioid pharmaceutical compositions that provide extended release delivery of the drug and the use thereof for the treatment of pain and other medical maladies.

In some preferred embodiments, opioid analgesics of the present invention can be formulated with the substantially the same ingredients to deter abuse and minimize opioid toxicity on tampering while simultaneously providing an extended release pharmacokinetic profile suitable for every 4, 6, 8, 12 or 24 hour dosing, without the need to include an aversive agent or an opioid antagonist in the formulation.

In some preferred embodiments, opioid pharmaceutical compositions and methods of the present invention provide (i) abuse deterrence; (ii) extended release; and (iii) simultaneous abuse deterrence and extended release, prepared using compounds selected from the group consisting of: (a) hydrogenated Type I or Type II vegetable oils (e.g., Hydrokote® 112); (b) polyoxyethylene stearates and distearates; (c) glycerol monostearate (e.g., Cithrol® GMS); (d) poorly water soluble, high melting point (mp=40 to 100° C.) waxes, and mixtures thereof, said compounds hereinafter referred to as “abuse deterrent, extended release” or “ADER”.

In some preferred embodiments, the present invention is directed to a novel method for reducing the peak concentration (Cₘₐₓ) of the opioid analgesic, said method comprising administering the opioid analgesic and a suitable amount of ADER.

In some preferred embodiments, the present invention is directed to a novel method for reducing the early post-dose partial area under the plasma opioid concentration time curve (e.g., AUC₀₋₂, AUC₀₋₄ and AUC₀₋₆) of the opioid analgesic, said method comprising administering the opioid analgesic and a suitable amount of ADER.

In some preferred embodiments, the present invention is directed to a novel method for reducing the early post-dose average plasma concentration time (Cave) of the opioid analgesic, said method comprising administering the opioid analgesic and a suitable amount of ADER.

In some preferred embodiments, the present invention is directed to a novel method for reducing the incidence of opioid toxicity upon tampering of the opioid analgesic, said method comprising administering the opioid analgesic and a suitable amount of ADER.

In some preferred embodiments, the present invention is directed to a novel method for reducing the intensity of opioid toxicity upon tampering of the opioid analgesic, said method comprising administering the opioid analgesic and a suitable amount of ADER.

In some preferred embodiments, the present invention is directed to a novel method for reducing the intensity or frequency of one or more signs and symptoms of opioid toxicity, including nausea, vomiting, somnolence, stupor, coma, respiratory depression, apnea, respiratory arrest, circulatory depression, bradycardia, hypotension, shock and skeletal muscle flaccidity, said method comprising administering the opioid analgesic and a suitable amount of ADER.

In some preferred embodiments, the present invention is directed to novel pharmaceutical compositions for use in reducing the peak concentration (Cₘₐₓ) of the opioid analgesic, said method comprising administering the opioid analgesic and a suitable amount of ADER.

In some preferred embodiments, the present invention is directed to novel pharmaceutical compositions for reducing the early post-dose partial area under the plasma opioid concentration time curve (e.g., AUC₀₋₂, AUC₀₋₄ and AUC₀₋₆) of the opioid analgesic, said method comprising administering the opioid analgesic and a suitable amount of ADER.

In some preferred embodiments, the present invention is directed to novel pharmaceutical compositions for reducing the early post-dose average plasma concentration time (Cave) of the opioid analgesic, said method comprising administering the opioid analgesic and a suitable amount of ADER.

In some preferred embodiments, the present invention is directed to novel pharmaceutical compositions for reducing the incidence of opioid toxicity, said method comprising administering the opioid analgesic and a suitable amount of ADER.

In some preferred embodiments, the present invention is directed to novel pharmaceutical compositions for reducing the intensity of opioid toxicity, said method comprising administering the opioid analgesic and a suitable amount of ADER.

In some preferred embodiments, the present invention is directed to novel pharmaceutical compositions for reducing the intensity or frequency of one or more symptoms, including nausea, vomiting, somnolence, stupor, coma, respiratory depression, apnea, respiratory arrest, circulatory depression, bradycardia, hypotension, shock and skeletal muscle flaccidity, said method comprising administering the opioid analgesic and a suitable amount of ADER.

In some preferred embodiments, the present invention is directed to a novel method and pharmaceutical compositions for preventing or minimizing excessive peak concentrations (dose dumping) of therapeutic doses of extended release opioids used for medical purposes, when they are co-ingested with alcohol.

In some preferred embodiments, the present invention is directed to a novel method and pharmaceutical compositions for reducing the solvent extraction efficiency of the dosage form upon tampering.

In some preferred embodiments, the present invention is directed to a novel method and pharmaceutical compositions for reducing the filtration efficiency of the dosage form upon tampering.

In some preferred embodiments, the present invention is directed to a novel method and pharmaceutical compositions for preventing the surreptitious adulteration of beverages.

In some preferred embodiments, the present invention is directed pharmaceutical compositions which include one or more opioids alone or in combination with other therapeutic agents, one or more ADER agents specified herein, and optionally one or more excipients (e.g., glidants, lubricants, disintegrants, etc) and inert carriers, said composition resisting, deterring, discouraging or preventing crushing, shearing, grinding, chewing, dissolving, melting, needle aspiration, inhalation, insufflation, solvent extraction and filtration of the opioid.

In some preferred embodiments, pharmaceutical compositions of the present invention provide a more extended release pharmacokinetic profile compared with formulations devoid of ADER.

In some preferred embodiments, pharmaceutical compositions and methods of the present invention can form a viscous substance upon contact with a solvent such that the opioid agent cannot be easily drawn into a syringe; crushed and powdered to facilitate or enhance nasal delivery (snorting or nasal insufflation), inhalation or rapid oral delivery of a larger than medically intended delivery of the opioid; extracted with solvents and filtered.

In some preferred embodiments, the pharmaceutical composition resists the rapid release of all or substantially all of the opioid content of the unit dose upon tampering. In another preferred embodiment of the invention, the pharmaceutical composition resists the rapid release of a portion of the opioid content of the unit dose upon tampering. In yet another preferred embodiment of the invention, upon tampering, the opioid formulated with ADER resists the release the opioid to a greater extent than when formulated without ADER.

In some preferred embodiment of the abuse deterrent pharmaceutical composition, the therapeutic pharmaceutical composition can be filled in a hard gelatin capsule without banding. In some preferred embodiment of the abuse deterrent pharmaceutical composition, the therapeutic pharmaceutical composition can be filled in a hard gelatin capsule with security banding. In another preferred embodiment of the abuse deterrent pharmaceutical composition, the therapeutic pharmaceutical composition can be filled in a soft shell capsules. In another preferred embodiment of the abuse deterrent pharmaceutical composition, the therapeutic pharmaceutical composition can be compressed into tablets.

The present invention is directed at oral pharmaceutical compositions of opioids or their pharmaceutically acceptable salts or mixtures thereof.

The present invention relates to oral opioid pharmaceutical compositions and methods for the prevention and treatment of pain and other maladies amenable to treatment with opioids.

It is an object of certain preferred embodiments of the present invention to substantially improve the efficiency and quality of pain management in human patients experiencing pain.

It is an object of certain preferred embodiments of the present invention to provide bioavailable oral opioid formulations suitable for up to once-daily administration which substantially improve the efficiency and quality of pain management.

It is an object of certain preferred embodiments of the present invention to provide bioavailable oral opioid formulations which provide a substantially increased duration of effect as compared to immediate release opioid formulations.

It is an object of certain preferred embodiments of the present invention to provide bioavailable oral opioid formulations which provide a substantially reduced abuse potential compared with immediate release opioid formulations.

It is an object of certain preferred embodiments of the present invention to provide bioavailable oral opioid formulations which provide a substantially reduced abuse potential compared with currently available extended release formulations.

It is an object of certain preferred embodiments of the present invention to provide bioavailable oral opioid formulations which provide a substantially reduced abuse potential compared with commercially available opioid formulations.

It is an object of certain preferred embodiments of the present invention to provide bioavailable oral immediate release opioid formulations which provide a substantially reduced abuse potential.

It is an object of certain preferred embodiments of the present invention to provide bioavailable oral immediate release opioid formulations which provide a substantially reduced variability in rate and extent of absorption when taken with food, compared with the fasted state.

It is an object of certain preferred embodiments of the present invention to provide bioavailable oral extended release opioid formulations which provide a substantially reduced variability in rate and extent of absorption when taken with food, compared with the fasted state.

It is an object of certain preferred embodiments of the present invention to provide bioavailable oral abuse resistant and abuse deterrent extended release opioid formulations which provide a substantially reduced variability in rate and extent of absorption when taken with food, compared with the fasted state.

It is an object of certain preferred embodiments of the present invention to provide bioavailable oral extended release formulations of opioids which provide a substantially reduced abuse potential compared with currently available extended release formulations.

It is an object of certain preferred embodiments of the present invention to provide bioavailable formulations for oral administration suitable for up to once-a-day administration (e.g., Q4H, Q6H, Q8H, Q12H, and Q24H).

It is an object of certain preferred embodiments of the present invention to provide bioavailable formulations for oral administration suitable for up to once-a-day administration which provide an early onset and sustained duration of therapeutic effect.

It is an object of certain preferred embodiments of the present invention to provide oral opioid formulations which provide pain relief for up to about 30 minutes. In other preferred embodiments, the opioid formulations which provide pain relief for up to about 1 hour, or up to about 2 hours, or up to about 4 hours, or up to about 6 hours, or up to about 8 hours, or up to about 10 hours, or up to about 12 hours, or up to about 16 hours, or up to about 18 hours, or up to about 24 hours or up to about 36 hours, or up to about 48 hours.

It is an object of certain preferred embodiments of the invention to provide a method and formulations of oral opioids for the prevention and treatment of pain.

It is an object of certain preferred embodiments of the invention to provide a method and formulations of oral opioids for the prevention and treatment of pain, said formulations and methods not having a propensity of substantial drug accumulation.

It is an object of certain preferred embodiments of the invention to provide a method and formulations of oral opioids for the prevention and treatment of pain, said formulations having a reduced potential for drug abuse and drug diversion.

It is an object of certain preferred embodiments of the invention to provide a method and formulations of oral opioids for the prevention and treatment of pain, said formulations having a reduced intrasubject and intrasubject pharmacokinetic variability.

It is an object of certain preferred embodiments of the invention to provide a method and formulations of oral opioids for the prevention and treatment of pain, said formulations having a reduced intersubject and intrasubject pharmacodynamic variability.

It is an object of certain preferred embodiments of the invention to provide a method and formulations of oral opioids for the prevention and treatment of pain, said formulations having a reduced peak to trough fluctuation.

It is an object of certain preferred embodiments of the invention to provide a method and formulations of oral opioids for the prevention and treatment of pain, said formulations having a shorter time to therapeutic concentrations and a shorter time to steady-state.

It is an object of certain preferred embodiments of the invention to provide a method and formulations of oral opioids for the prevention and treatment of pain, said formulations being in extended release dosage form, and said formulations providing an extended duration of action.

It is an object of certain preferred embodiments of the invention to provide a method and formulations of oral opioids for the prevention and treatment of pain, said formulations suitable for use in acute pain, including acute postsurgical pain. In other preferred embodiments, the invention provides a method and formulations of oral opioids for the prevention and treatment of chronic pain, cancer pain, neuropathic pain, somatic pain, visceral pain, idiopathic pain and breakthrough pain of various etiologies, including cancer, chronic pain and neuropathic pain.

Some or all of the above objects and others are achieved by embodiments of the present invention, which is directed in part to a dosage form of oral opioids.

It is an object of certain embodiments of the present invention to provide oral opioid formulations with both immediate release and controlled release forms.

It is an object of certain embodiments of the present invention to provide oral opioid formulations in pulsatile release form.

It is an object of certain embodiments of the present invention to provide opioids for oral administration wherein the opioids are dispersed within a matrix.

In certain preferred embodiments the oral dosage form of the present invention comprises a matrix which includes ADER and an opioid or a pharmaceutically acceptable salt thereof. In certain preferred embodiments, the matrix is compressed into a tablet and may be optionally overcoated with a coating that in addition to the sustained release material of the matrix may control the release of the opioid or pharmaceutically acceptable salt thereof from the formulation, such that blood levels of active ingredient are maintained within the therapeutic range over an extended period of time. In certain alternate embodiments, the matrix is encapsulated.

In certain preferred embodiments, the sustained release oral dosage form of the present invention comprises ADER and a plurality of pharmaceutically acceptable sustained release matrices comprising an opioid or a pharmaceutically acceptable salt thereof, the dosage form maintaining the plasma levels of opioid within the therapeutic range over an extended period of time when administered to patients.

In some preferred embodiments of the invention, the opioids are in a matrix that is in the form of pellets or beads.

In some preferred embodiments, the dosage form of the invention comprises a compressed tablet, compressed capsule or uncompressed capsule. In other embodiments, the dosage form comprises a liquid fill capsule.

In some preferred embodiments, the dosage form of the invention comprises an oral formulation (e.g., tablet or capsule) which is coated to prevent substantial direct contact of opioid with oral cavity (e.g. tongue, oral mucosa), oropharyngeal mucosal surface, esophagus or stomach. In some preferred embodiments, the dosage form of the invention comprises an oral formulation which is coated with a film or polymer. In some preferred embodiments, the dosage form of the invention comprises opioids in an enteric coating. In some preferred embodiments, the dosage form of the invention comprises opioids formulated with pharmaceutical excipients and auxiliary agents known in the art, such that the opioid is released after a approximately specific amount of time, or at an approximately specific anatomic location in the gastrointestinal tract, or when the dosage form is in contact with specific gastrointestinal conditions (e.g., pH range, osmolality, electrolyte content, food content).

In some preferred embodiments, the pharmaceutical compositions and methods of the invention are useful for the prevention and treatment of pain despite minimal or no systemic absorption of the opioid form the oral cavity (e.g., buccal, lingual, sublingual absorption).

In some preferred embodiments, the in vivo pharmacokinetic parameters of the specifications and claims are derived or determined under fed conditions. In other preferred embodiments, the in vivo pharmacokinetic parameters are derived or determined under fasted conditions.

Some or all of the above objects and others are achieved by embodiments of the present invention, which is directed in part to a dosage form of oral opioids and ADER.

Some or all of the above objects and others are achieved by embodiments of the present invention, which is directed in part to a dosage form of oral extended release opioids and ADER.

Some or all of the above objects and others are achieved by embodiments of the present invention, which is directed in part to a dosage form of abuse deterrent opioids and ADER.

Some or all of the above objects and others are achieved by embodiments of the present invention, which is directed in part to a dosage form which provides simultaneous abuse deterrence and extended release through the inclusion of ADER.

In another aspect, the invention relates to a method for prevention or treatment of pain comprising oral administration of a dosage form containing an opioid or a pharmaceutically acceptable salt of opioid or a mixture thereof.

In some preferred embodiments, the dosage form provides an oral pharmaceutical composition for the prevention and treatment of pain comprising a therapeutically effective amount of opioid or a pharmaceutically acceptable salt of opioid or a mixture thereof and ADER material to render said dosage form abuse deterrent, said dosage form suitable for up to every 24 hour (once-a-day) administration to a human patient, said dosage form providing at least 70% of the steady state concentration of opioid after administration of one dose at its intended dosing frequency. In other preferred embodiments, the dosage form provides at least about 75%, or at least about 80%, or at least about 85%, or at least about 87.5%, or at least about 90%, or at least about 92.5%, or at least about 95% or at least 98% of the steady state therapeutic concentration of opioid after administration of one dose at its intended dosing frequency.

In some preferred embodiments, the dosage form provides an oral pharmaceutical composition for the prevention and treatment of pain comprising a therapeutically effective amount of opioid or a pharmaceutically acceptable salt of opioid or a mixture thereof and ADER material to render said dosage form suitable for extended release, said dosage form suitable for up to every 24 hour (once-a-day) administration to a human patient, said dosage form providing at least 70% of the steady state concentration of opioid after administration of one dose at its intended dosing frequency. In other preferred embodiments, the dosage form provides at least about 75%, or at least about 80%, or at least about 85%, or at least about 87.5%, or at least about 90%, or at least about 92.5%, or at least about 95% or at least 98% of the steady state therapeutic concentration of opioid after administration of one dose at its intended dosing frequency.

In some preferred embodiments, the invention comprises an oral pharmaceutical composition for the treatment of pain comprising a therapeutically effective amount of opioid or a pharmaceutically acceptable salt of opioid, or a mixture thereof and ADER to render said dosage form suitable for three times a day administration (TID) or about every eight hours administration (Q8H).

In some preferred embodiments, the TID or Q8H oral pharmaceutical composition of an opioid provides a therapeutic effect for about 8 hours.

In some preferred embodiments, the TID or Q8H oral pharmaceutical composition of opioids provides a Cₘₐₓ of opioids at about 1 to about 6 hours.

In some preferred embodiments, the TID or Q8H oral pharmaceutical composition of opioids provide a Cₘᵢₙ of opioids at about 6 to 10 hours.

In some preferred embodiments, the TID or Q8H oral pharmaceutical composition of opioids provide a mean of opioids C₈/Cₘₐₓ ratio of 0.25 to about 0.95.

In some preferred embodiments, the TID or Q8H oral pharmaceutical composition of opioids provide an opioid percent fluctuation of less than 400%.

In some preferred embodiments, the TID or Q8H oral pharmaceutical composition of opioids provides an opioid of W₅₀ of 1.5 to about 6.5 hours.

In some preferred embodiments, the TID or Q8H oral pharmaceutical composition of an opioid provides an HVD of opioids of 2 to about 7 hours.

In some preferred embodiments, the TID or Q8H oral pharmaceutical composition of an opioid provide an HVD of about 2 to about 7 hours.

In some preferred embodiments, the TID or Q8H oral pharmaceutical composition of an opioid provides an AI of opioids of not more that 4.0.

In some preferred embodiments, the invention comprises an oral pharmaceutical composition for the treatment of pain comprising therapeutically effective amounts of opioid or pharmaceutically acceptable salts thereof, or mixtures thereof and ADER; said dosage from providing a Cₘₐₓ of opioid occurring from a mean of about 0.25 to about 30 hours. In other preferred embodiments, the dosage form provides a Cₘₐₓ of opioids occurring from a mean of about 0.5 to about 30 hours, or from a mean of about 1 to about 30 hours, or about 1 to about 26 hours, or about 1 to about 24 hours, or about 1 to about 20 hours, or about 1 to about 18 hours, or about 1 to about 16 hours, or about 1 to about 14 hours, or about 1 to about 12 hours, or about 1 to about 10 hours, or about 1 to about 8 hours, or about 1 to about 6 hours, or about 1 to about 4 hours, or about 1 to about 3 hours, or about 2 to about 30 hours, or about 4 to about 30 hours, or about 4 to about 24 hours, or about 6 to about 24 hours, or about 8 to about 24 hours, or about 10 to about 20 hours, or about 12 to about 24 hours, or about 18 to about 24 hours, or about 2 to about 12 hours, or about 3 to about 12 hours, or about 3 to about 8 hours, or about 4 to about 10 hours, or about 4 to about 12 hours, or about 4 to about 9 hours, or about 5 to about 8 hours.

In some preferred embodiments, the invention comprises an oral pharmaceutical composition for the treatment of pain comprising therapeutically effective amounts of opioid or pharmaceutically acceptable salts thereof, or mixtures thereof and ADER; said dosage from providing a Cₘᵢₙ of opioid occurring from a mean of about 0.5 to about 28 hours, or about 1 to about 28 hours, or about 1 to 24 hours, or about 1 to about 20 hours, or about 1 to about 18 hours, or about 1 to about 16 hours, or about 1 to about 12 hours, or about 1 to 10 hours, or about 1 to about 8 hours, or about 1 to about 6 hours, or about 1 to about 4 hours, about 2 to about 24 hours, or about 3 to 24 hours, or about 4 to about 24 hours, or about 6 to about 24 hours, or about 8 to about 24 hours, about 2 to about 12 hours, or about 3 to 10 hours, or about 3 to about 8 hours, or about 4 to about 8 hours, or about 6 to about 10 hours.

In some preferred embodiments, the invention comprises an oral pharmaceutical composition for the treatment of pain comprising therapeutically effective amounts of opioid or pharmaceutically acceptable salts thereof, or mixtures thereof and ADER; said dosage form providing a systemic exposure as assessed by the mean opioids area under the plasma concentration time curve (AUC₀₋₁) after first administration which is at least about 40% of the area under the plasma drug concentration-time curve from time zero to infinity (AUC_{0-∞}). In other preferred embodiments, the dosage from provides an AUC₀₋ₜ which is at least about 50%, or at least about 60%, or at least about 70%, or at least about 80%, or at least about 85%, or at least about 88%, or at least about 90%, or at least about 92%, or at least about 94%, or at least about 96% or at least about 98% of the AUC_{0-∞}.

In some preferred embodiments, the dosage form provides an oral pharmaceutical composition for the treatment of pain comprising a therapeutically effective amount of opioid or pharmaceutically acceptable salts thereof or mixtures thereof and ADER; said dosage form providing at least 80% of the steady state therapeutic concentration of opioid after administration of ≦three doses at their intended dosing frequency. In other preferred embodiments, said dosage form provides at least about 60%, or at least about 65%, or at least about 70%, or at least about 75%, or at least about 85%, or at least about 90%, or at least about 92%, or at least about 95%, or at least about 97%, or at least about 99% of the steady state therapeutic concentration of opioid after administration of ≦three doses at their intended dosing frequency.

In some preferred embodiments, the dosage form provides an oral pharmaceutical composition for the treatment of pain comprising a therapeutically effective amount of opioid or pharmaceutically acceptable salts thereof or mixtures thereof and ADER; said dosage form providing at least 80% of the steady state therapeutic concentration of opioid after administration of ≦two doses at their intended dosing frequency. In other preferred embodiments, said dosage form provides at least about 60%, or at least about 65%, or at least about 70%, or at least about 75%, or at least about 85%, or at least about 90%, or at least about 92%, or at least about 95%, or at least about 97%, or at least about 99% of the steady state therapeutic concentration of opioid after administration of ≦two doses at their intended dosing frequency.

In some preferred embodiments, the dosage form provides an oral pharmaceutical composition for the treatment of pain comprising a therapeutically effective amount of opioid or pharmaceutically acceptable salts thereof or mixtures thereof and ADER; said dosage form providing at least 80% of the steady state therapeutic concentration of opioid after administration of one dose at their intended dosing frequency. In other preferred embodiments, said dosage form provides at least about 60%, or at least about 65%, or at least about 70%, or at least about 75%, or at least about 85%, or at least about 0.90%, or at least about 92%, or at least about 95%, or at least about 97%, or at least about 99% of the steady state therapeutic concentration of opioid after administration of one dose at their intended dosing frequency.

In some preferred embodiments, the dosage form provides an oral pharmaceutical composition for the treatment of pain comprising a therapeutically effective amount of opioid or pharmaceutically acceptable salts thereof or mixtures thereof and ADER; said dosage form after administration to a human patient providing a Cₘᵢₙ/Cₘₐₓ ratio of opioid of 0.1 to about 1.0. In other preferred embodiments, the dosage form provides a Cₘᵢₙ/Cₘₐₓ ratio of opioid of about 0.1 to about 0.9, or about 0.1 to about 0.8, or about 0.1 to about 0.7, or about 0.1 to about 0.6, or about 0.1 to about 0.5, or about 0.1 to about 0.4, or about 0.1 to about 0.3, or about 0.2 to about 1.0, or about 0.25 to about 1.0, or about 0.4 to about 1.0, or about 0.5 to about 1.0, or about 0.65 to about 1.0, or about 0.75 to about 1.0, or about 0.2 to about 0.9, or about 0.3 to about 0.8, or about 0.4 to about 0.8, or about 0.4 to about 0.7, or about 0.4 to about 0.6.

In some preferred embodiments, the dosage form provides an oral pharmaceutical composition for the treatment of pain comprising a therapeutically effective amount of opioid or pharmaceutically acceptable salts thereof or mixtures thereof and ADER; said dosage form after administration to a human patient providing a percent fluctuation of opioid of less than 400%. In other preferred embodiments, the dosage form provides a percent fluctuation of opioid of less than 350%, or less than 300%, or less than 250%, or less than 200%, or less than 150%, or less than 100%, or less than 75%, or less than 50%, or less than 25%.

In some preferred embodiments, the dosage form provides an oral pharmaceutical composition for the treatment of pain comprising a therapeutically effective amount of opioid or pharmaceutically acceptable salts thereof or mixtures thereof and ADER; said dosage form after administration to a human patient providing a W₅₀ of opioid of about 1 to about 6 hours for each 6 hour time period of intended dosing frequency and intended duration of action. In other preferred embodiments, the dosage form provides a W₅₀ of opioid for each 6 hour time period of intended dosing frequency and intended duration of action of about 1 to about 5 hours, or about 1 to about 4 hours, or about 1 to about 3 hours, or about 1 to about 2 hours, or 2 to about 6 hours, or about 3 to about 6 hours, or about 4 to about 6 hours, or about 2 to about 4 hours.

In some preferred embodiments, the dosage form provides an oral pharmaceutical composition for the treatment of pain comprising a therapeutically effective amount of opioid or pharmaceutically acceptable salts thereof or mixtures thereof and ADER; said dosage form after administration to a human patient providing an HVD of opioid of about 1.5 to about 6 hours for each 6 hour time period of intended dosing frequency and intended duration of action. In other preferred embodiments, the dosage form provides a HVD of opioid for each 6 hour time period of intended dosing frequency and intended duration of action of about 1.5 to about 5 hours, or about 1.5 to about 4 hours, or about 1.5 to about 3 hours, or about 1.5 to about 2 hours, or 2 to about 6 hours, or about 3 to about 6 hours, or about 4 to about 6 hours, or about 2 to about 4 hours.

In some preferred embodiments, the dosage form provides an oral pharmaceutical composition for the treatment of pain comprising a therapeutically effective amount of opioid or pharmaceutically acceptable salts thereof or mixtures thereof and ADER; said dosage form after administration to a human patient providing an AI of opioid of not more than 3.0. In other preferred embodiments, the dosage form provides an AI of opioid of not more than about 2.5, or not more than about 2, or not more than about 1.75, or not more than about 1.5, or not more than about 1.25, or not more than about 1, or not more than about 0.75, or not more than about 0.5, or not more than about 0.25.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for twice-a-day administration to a human patient, said dosage form providing a Cₘₐₓ of opioid at 2 to about 10 hours; and said dosage form providing a therapeutic effect for at least about 12 hours. In other preferred embodiments, the dosage form provides a Cₘₐₓ of opioid at about 2 to about 8 hour or about 2 to about 6 hours, or about 2 to about 5 hours, or about 2 to about 7 hours, or about 2 to about 4.5 hours, or about 2 to about 4 hours, or 2 to about 3.5 hours, or about 2 to about 3 hours, or about 3 to about 10 hours, or about 3.5 to about 10 hours, or about 4 to about 10 hours, or about 4.5 to about 10 hours, or about 5 to about 10 hours, or 5 to about 10 hours, or about 6 to about 10 hours, or about 3 to about 8 hours, or about 3 to about 7 hours, or about 3 to about 6 hours, or about 4 to about 8 hours, or about 4 to about 6.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for twice-a-day administration to a human patient, said dosage form providing a C₁₂/Cₘₐₓ ratio of opioid 0.25 to about 0.95; and said dosage form providing a therapeutic effect for at least about 12 hours. In other preferred embodiments, the dosage form provides a C₁₂/Cₘₐₓ ratio of opioid of about 0.25 to about 0.9, or about 0.25 to about 0.8, or about 0.25 to about 0.75, or about 0.25 to about 0.6, or 0.25 to about 0.5, or about 0.25 to about 0.4, or about 0.25 to about 0.35, or about 0.3 to about 0.95, or about 0.4 to about 0.95, or about 0.5 to about 0.95, or about 0.65 to about 0.95, or about 0.75 to about 0.95, or about 0.3 to about 0.8, or about 0.4 to about 0.75, or about 0.5 to about 0.75.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for twice-a-day administration to a human patient, said dosage form providing a percent fluctuation of opioid of less than 400%; and said dosage form providing a therapeutic effect for at least about 12 hours. In other preferred embodiments, the dosage form provides a percent fluctuation of opioid of less than about 375%, or less than about 350%, or less than about 325%, or less than about 300%, or less than about 275%, or less than about 250%, or less than about 225%, or less than about 200%, or less than about 175%, or less than about 150%, or less than about 125%, or less than about 100%, or less than about 75%, or less than about 50%, or less than about 25%.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for twice-a-day administration to a human patient, said dosage form after administration to a human patient, providing a W₅₀ of opioid of 2 to about 11 hours; and said dosage form providing a therapeutic effect for at least about 12 hours. In other preferred embodiments, the dosage form provides a W₅₀ of opioid of about 2 to about 10 hours, or about 2 to about 9 hours, or about 2 to about 9 hours, or about 2 to about 8 hours, or 2 to about 7 hours, or about 2 to about 6 hours, or about 2 to about 5 hours, or about 2 to about 4 hours, or about 3 to about 10 hours, or about 4 to about 10 hours, or about 5 to about 10 hours, or about 6 to about 10 hours, or 7 to about 10 hours, or about 3 to about 8 hours, or about 4 to about 8 hours, or about 4 to about 7 hours, or about 3 to about 6 hours.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for twice-a-day administration to a human patient, said dosage form after administration to a human patient, providing a HVD of opioid of 1.5 to about 10 hours; and said dosage form providing a therapeutic effect for at least about 12 hours. In other preferred embodiments, the dosage form provides an HVD of opioid of about 1.5 to about 9 hours, or about 1.5 to 8 hours, or about 1.5 to about 7 hours, or about 1.5 to 6 hours, or about 1.5 to about 5 hours, or about 1.5 to about 4 hours, or about 2 to about 10 hours, or about 3 to 10 hours, or about 4 to about 10 hours, or about 5 to 10 hours, or about 6 to about 10 hours, or about 8 to 10 hours, about 3 to about 8 hours, or about 4 to 8 hours, or about 5 to about 7 hours, or about 3 to 6 hours, or about 3 to about 8 hours, or about 5 to about 8 hours.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for twice-a-day administration to a human patient, said dosage form after administration to a human patient, providing an AI of opioid of not more that 4.0; and said dosage form providing a therapeutic effect for at least about 12 hours. In other preferred embodiments, the dosage form provides an AI of opioid of not more than about 3.75, or not more than about 3.5, or not more than about 3.25, or not more than about 3, or not more than about 2.75, or not more than about 2.5, or not more than about 2, or not more than about 1.5, not more than about 1.25, or not more than about 1, or not more than about 0.75.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for twice-a-day administration to a human patient; said dosage form providing an in-vitro release rate by weight of opioid, when measured by the USP Basket and Paddle Methods at 100 rpm in 900 mL aqueous buffer at a pH of between 1.6 and 7.2 at 37° C. of from 0% to about 47.5% at 1 hour, from about 10% to about 65% at 2 hours, from about 15% to about 70% at 4 hours, from about 25% to about 77.5% at 6 hours, from about 35% to about 87.5% at 9 hours, and greater than about 65% at 12 hours. In other preferred embodiments, the dosage form provides said an in-vitro release rate of from 0% to about 40% at 1 hour, from about 5% to about 55% at 2 hours, from about 10% to about 60% at 4 hours, from about 15% to about 70% at 6 hours, from about 25% to about 80% at 9 hours, and greater than about 50% at 12 hours.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for twice-a-day administration to a human patient; said dosage form providing an in-vitro release rate by weight of opioid, when measured by the USP Basket and Paddle Methods at 100 rpm in 900 mL aqueous buffer at a pH of between 1.6 and 7.2 at 37° C. of from 0% to about 47.5% at 1 hour, from about 10% to about 65% at 2 hours, from about 15% to about 70% at 4 hours, from about 25% to about 77.5% at 6 hours, from about 35% to about 87.5% at 9 hours, and greater than about 65% at 12 hours; said dosage form providing a Cₘₐₓ from a mean of about 2 to about 10 hours after first administration or at steady state.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for twice-a-day administration to a human patient; said dosage form providing an in-vitro release rate by weight of opioid, when measured by the USP Basket and Paddle Methods at 100 rpm in 900 mL aqueous buffer at a pH of between 1.6 and 7.2 at 37° C. of from 0% to about 47.5% at 1 hour, from about 10% to about 65% at 2 hours, from about 15% to about 70% at 4 hours, from about 25% to about 77.5% at 6 hours, from about 35% to about 87.5% at 9 hours, and greater than about 65% at 12 hours; said dosage form providing a Cₘᵢₙ occurring from a mean of about 10 to about 14 hours after first administration or at steady state.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for twice-a-day administration to a human patient; said dosage form providing an in-vitro release rate by weight of opioid, when measured by the USP Basket and Paddle Methods at 100 rpm in 900 mL aqueous buffer at a pH of between 1.6 and 7.2 at 37° C. of from 0% to about 47.5% at 1 hour, from about 10% to about 65% at 2 hours, from about 15% to about 70% at 4 hours, from about 25% to about 77.5% at 6 hours, from about 35% to about 87.5% at 9 hours, and greater than about 65% at 12 hours; said dosage form providing a mean opioid AUC₀₋ₜ/AUC_{0-∞} ratio after first administration of about 0.4, or about 0.5, or about 0.6, or about 0.7, or about 0.75, or about 0.8, or about 0.85, or about 0.88, or about 0.90, or about 0.92, or about 0.95, or about 0.97 or about 0.99.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for twice-a-day administration to a human patient; said dosage form providing an in-vitro release rate by weight of opioid, when measured by the USP Basket and Paddle Methods at 100 rpm in 900 mL aqueous buffer at a pH of between 1.6 and 7.2 at 37° C. of from 0% to about 47.5% at 1 hour, from about 10% to about 65% at 2 hours, from about 15% to about 70% at 4 hours, from about 25% to about 77.5% at 6 hours, from about 35% to about 87.5% at 9 hours, and greater than about 65% at 12 hours; said in-vitro release rate being substantially independent of pH in that a difference, at any given time, between an amount of opioid released at one pH and an amount released at any other pH, when measured in-vitro using the USP Basket and Paddle Methods of USP Drug Release test of U.S. Pharmacopeia (2003) at 100 rpm in 900 ml aqueous buffer, is no greater than 30%.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for once-a-day administration to a human patient, said dosage form providing a Cₘₐₓ of opioid at about 3 to about 20 hours; and said dosage form providing a therapeutic effect for at least about 24 hours. In some preferred embodiments, the opioids dosage forms provide a Cₘₐₓ of opioid at about 3 to about 18 hours, or about 3 to about 15 hours, or about 3 to about 12 hours, or at about 3 to about 10 hours, or at about 3 to about 8 hours, or at about 3 to about 7 hours, or at about 3 to about 7 hours, or about 4 to about 20 hours, or about 5 to about 20 hours, or about 6 to about 20 hours, or at about 8 to about 20 hours, or at about 10 to about 20 hours, or at about 12 to about 20 hours, or at about 14 to about 20 hours, or about 18 to about 20 hours, or about 4 to about 18 hours, or about 4 to about 16 hours, or at about 4 to about 12 hours, or at about 4 to about 8 hours, or at about 4 to about 10 hours, or at about 3 to about 6 hours.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for once-a-day administration to a human patient, said dosage form providing a Cₘᵢₙ of opioid at about 20 to about 28 hours; and said opioids dosage forms providing a therapeutic effect for at least about 24 hours. In some preferred embodiments, the opioids dosage forms provide a Cₘᵢₙ of opioid at about 20 to about 26 hours, or about 20 to about 27 hours, or about 20 to about 25 hours, or about 20 to about 24 hours, or about 20 to about 23 hours, or about 21 to about 28 hours, or about 22 to about 28 hours, or about 23 to about 28 hours, or about 23.5 to about 28 hours, or about 22 to 26 hours.

In some preferred embodiments, the dosage form provides an oral pharmaceutical composition for the treatment of pain comprising a therapeutically effective amount of opioid or pharmaceutically acceptable salts thereof or mixtures thereof and ADER; said dosage from providing a Cₘₐₓ of opioid from about 0.25 hours to about 30 hours.

In some preferred embodiments, the dosage form provides an oral pharmaceutical composition for the treatment of pain comprising a therapeutically effective amount of opioid or pharmaceutically acceptable salts thereof or mixtures thereof and ADER; said dosage from providing a Cₘᵢₙ of opioid from about 0.5 hour to about 30 hours.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for once-a-day administration to a human patient, said dosage form providing a C₂₄/Cₘₐₓ ratio of opioid of 0.25 to about 0.95; and said dosage form providing a therapeutic effect for at least about 24 hours. In other preferred embodiments, the dosage form provides a C₂₄/Cₘₐₓ ratio of opioid of about 0.25 to about 0.9, or about 0.25 to about 0.8, or about 0.25 to about 0.75, or about 0.25 to about 0.6, or 0.25 to about 0.5, or about 0.25 to about 0.4, or about 0.25 to about 0.35, or about 0.3 to about 0.95, or about 0.4 to about 0.95, or about 0.5 to about 0.95, or about 0.65 to about 0.95, or about 0.75 to about 0.95, or about 0.3 to about 0.8, or about 0.4 to about 0.75, or about 0.5 to about 0.75.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for once-a-day administration to a human patient, said dosage form providing a percent fluctuation of opioid of less than 400%; and said dosage form providing a therapeutic effect for at least about 24 hours. In other preferred embodiments, the dosage form provides a percent fluctuation of opioid of less than about 375%, or less than about 350%, or less than about 325%, or less than about 300%, or less than about 275%, or less than about 250%, or less than about 225%, or less than about 200%, or less than about 175%, or less than about 150%, or less than about 125%, or less than about 100%, or less than about 75%, or less than about 50%, or less than about 25%.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for once-a-day administration to a human patient, said opioids dosage form after administration to a human patient, providing a W₅₀ of opioid of 4 to about 22 hours; and said dosage form providing a therapeutic effect for at least about 24 hours. In other preferred embodiments, the opioids dosage from provides a W₅₀ of opioid of about 4 to about 20 hours, or about 4 to about 19 hours, or about 4 to about 18 hours, or 4 to about 16 hours, or 4 to about 14 hours, or about 4 to about 12 hours, or about 4 to about 10 hours, or about 4 to about 8 hours, or about 6 to about 20 hours, or about 8 to about 20 hours, or about 10 to about 20 hours, or about 12 to about 20 hours, or 14 to about 20 hours, or about 6 to about 16 hours, or about 8 to about 16 hours, or about 8 to about 14 hours, or about 6 to about 12 hours.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for once-a-day administration to a human patient, said opioids dosage form after administration to a human patient, providing a HVD of opioid of 3 to about 20 hours; and said dosage form providing a therapeutic effect for at least about 24 hours. In other preferred embodiments, the opioids dosage from provides an HVD of opioid of about 3 to about 18 hours, or about 3 to 16 hours, or about 3 to about 14 hours, or about 3 to 12 hours, or about 3 to about 10 hours, or about 3 to about 8 hours, or about 4 to about 20 hours, or about 6 to 20 hours, or about 8 to about 20 hours, or about 10 to 20 hours, or about 12 to about 20 hours, or about 16 to 20 hours, about 6 to about 16 hours, or about 8 to 16 hours, or about 10 to about 14 hours, or about 6 to 12 hours, or about 6 to about 16 hours, or about 10 to about 16 hours.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for once-a-day administration to a human patient, said dosage form after administration to a human patient, providing an AI of opioid of not more that 4.0; and said opioids dosage form providing a therapeutic effect for at least about 24 hours. In other preferred embodiments, the opioids dosage from provides an AI of opioid of not more than about 3.75, or not more than about 3.5, or not more than about 3.25, or not more than about 3, or not more than about 2.75, or not more than about 2.5, or not more than about 2, or not more than about 1.5, not more than about 1.25, or not more than about 1, or not more than about 0.75.

In some preferred embodiments, the dosage form provides an oral pharmaceutical composition for the treatment of pain comprising a therapeutically effective amount of opioid or pharmaceutically acceptable salts thereof or mixtures thereof and ADER to render said opioid dosage form abuse deterrent and/or suitable for once-a-day administration to a human patient; said opioids dosage form providing an in-vitro release rate by weight of opioid, when measured by the USP Basket and Paddle Methods at 100 rpm in 900 mL aqueous buffer at a pH of between 1.6 and 7.2 at 37° C. of from 0% to about 30% at 1 hour, from about 10% to about 65% at 4 hours, from about 20% to about 70% at 8 hours, from about 25% to about 80% at 12 hours, from about 35% to about 95% at 18 hours, and greater than about 65% at 24 hours.

In some preferred embodiments, the dosage form provides an oral pharmaceutical composition for the treatment of pain comprising a therapeutically effective amount of opioid or pharmaceutically acceptable salts thereof or mixtures thereof and ADER to render said opioid dosage form abuse deterrent and/or suitable for once-a-day administration to a human patient; said opioids dosage form providing an in-vitro release rate by weight of opioid, when measured by the USP Basket and Paddle Methods at 100 rpm in 900 mL aqueous buffer at a pH of between 1.6 and 7.2 at 37° C. of from 0% to about 30% at 1 hour, from about 10% to about 65% at 4 hours, from about 20% to about 70% at 8 hours, from about 25% to about 80% at 12 hours, from about 35% to about 95% at 18 hours, and greater than about 65% at 24 hours; said dosage form providing a Cₘₐₓ from a mean of about 3 to about 20 hours after first administration or at steady state.

In some preferred embodiments; the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for once-a-day administration to a human patient; said dosage form providing an in-vitro release rate by weight of opioid, when measured by the USP Basket and Paddle Methods at 100 rpm in 900 mL aqueous buffer at a pH of between 1.6 and 7.2 at 37° C. of from 0% to about 30% at 1 hour, from about 10% to about 65% at 4 hours, from about 20% to about 70% at 8 hours, from about 25% to about 80% at 12 hours, from about 35% to about 95% at 18 hours, and greater than about 65% at 24 hours; said dosage form providing a Cₘᵢₙ of opioid occurring from a mean of about 20 to about 28 hours after first administration or at steady state.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for once-a-day administration to a human patient; said dosage form providing an in-vitro release rate by weight of opioid, when measured by the USP Basket and Paddle Methods at 100 rpm in 900 mL aqueous buffer at a pH of between 1.6 and 7.2 at 37° C. of from 0% to about 30% at 1 hour, from about 10% to about 65% at 4 hours, from about 20% to about 70% at 8 hours, from about 25% to about 80% at 12 hours, from about 35% to about 95% at 18 hours, and greater than about 65% at 24 hours; said dosage form providing a mean opioid AUC₀₋ₜ/AUC_{0-∞} ratio after first administration of about 0.4, or about 0.5, or about 0.6, or about 0.7, or about 0.75, or about 0.8, or about 0.85, or about 0.88, or about 0.90, or about 0.92, or about 0.95, or about 0.97 or about 0.99.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for once-a-day administration to a human patient; said dosage form providing an in-vitro release rate by weight of opioid, when measured by the USP Basket and Paddle Methods at 100 rpm in 900 mL aqueous buffer at a pH of between 1.6 and 7.2 at 37° C. of from 0% to about 30% at 1 hour, from about 10% to about 65% at 4 hours, from about 20% to about 70% at 8 hours, from about 25% to about 80% at 12 hours, from about 35% to about 95% at 18 hours, and greater than about 65% at 24 hours; said in-vitro release rate being substantially independent of pH in that a difference, at any given time, between an amount of opioid released at one pH and an amount released at any other pH, when measured in-vitro using the USP Basket and Paddle Methods of USP Drug Release test of U.S. Pharmacopeia (2003) at 100 rpm in 900 ml aqueous buffer, is no greater than 30%.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for extended release administration to a human patient; said dosage form after administration to a human patient providing a mean opioid Cₘₐₓ occurring from a mean of about 0.25 to about 22 hours; said dosage form providing a mean opioid Cₘᵢₙ occurring from a mean of about 0.5 to about 28 hours; said dosage form providing a mean opioid HVD of about 1 to about 5 hours for each 6 hour time period of intended dosing frequency and intended duration of action; said dosage form providing a mean opioid W₅₀ of about 1 to about 5.5 hours for each 6 hour time period of intended dosing frequency and intended duration of action; said dosage form providing a mean opioid AI of not more than 3.0; said dosage form providing a mean opioid percent fluctuation of less than 400%; said dosage form providing a mean opioid Cₘᵢₙ/Cₘₐₓ ratio of 0.1 to about 1.0; said dosage form providing at least 80% of the steady state opioid therapeutic concentration after administration of ≦three doses at their intended dosing frequency; said dosage form providing a mean opioid AUC₀₋ₜ to AUC_{0-∞} ratio of greater than 0.4; and said dosage form providing a mean time to 75% opioid Cₘₐₓ of about 100% to about 2000% of the time to 75% mean Cₘₐₓ of an oral immediate release opioid solution.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for extended release administration to a human patient; said dosage form after administration to a human patient providing a mean opioid Cₘₐₓ which is less than 65% of the Cₘₐₓ of an equivalent dose of an oral immediate release opioid solution; and said dosage form maintaining a mean opioid plasma concentration within 50% of Cₘₐₓ for about 1 to about 5.5 hours for each 6 hour time period of intended dosing frequency and intended duration of action.

In some preferred embodiments, the dosage form provides an oral pharmaceutical composition for the treatment of pain comprising a therapeutically effective amount of opioid or pharmaceutically acceptable salts thereof or mixtures thereof and ADER to render said dosage form abuse deterrent and/or suitable for extended release administration to a human patient; said dosage form after administration to a human patient providing a mean opioid Cₘₐₓ occurring from a mean of about 0.25 to about 22 hours; said dosage form providing a mean opioid Cₘᵢₙ occurring from a mean of about 0.5 to about 28 hours; said dosage form providing a mean opioid HVD of about 1 to about 5 hours for each 6 hour time period of intended dosing frequency and intended duration of action; said dosage form providing a mean opioid W₅₀ of about 1 to about 5.5 hours for each 6 hour time period of intended dosing frequency and intended duration of action; said dosage form providing a mean opioid AI of not more than 3.0; said dosage form providing a mean opioid percent fluctuation of less than 400%; said dosage form providing a mean opioid Cₘᵢₙ/Cₘₐₓ ratio of 0.1 to about 1.0; said dosage form providing at least 80% of the steady state opioid therapeutic concentration after administration of ≦three doses at their intended dosing frequency; said dosage form providing a mean opioid AUC₀₋ₜ to AUC_{0-∞} ratio of greater than 0.4; said dosage form providing a mean time to 75% opioid Cₘₐₓ of about 100% to about 2000% of the time to 75% mean Cₘₐₓ of an oral immediate release opioid solution; said dosage form after administration to a human patient providing a mean opioid Cₘₐₓ which is less than 65% of the Cₘₐₓ of an equivalent dose of an oral immediate release opioid solution; and said dosage form maintaining a mean opioid plasma concentration within 50% of Cₘₐₓ for about 1 to about 5.5 hours for each 6 hour time period of intended dosing frequency and intended duration of action.

In some preferred embodiments, the opioids dosage forms provide an in-vitro release of from 0% to about 50% by weight of the opioid or a pharmaceutically acceptable salt thereof from the dosage form at one hour when measured by the USP Basket and Paddle Methods at 100 rpm in 700 ml of Simulated Gastric Fluid (SGF) at 37° C. In other preferred embodiments, said in-vitro release rate by weight of the opioid or a pharmaceutically acceptable salt thereof from said dosage form is from about 5% to about 45%, or about 10% to about 50%, or about 5% to about 60%, or about 5% to about 706%, or about 5% to about 80%, or about 5% to about 90%, or about 5% to about 100%, or about 10% to about 20%, or about 10% to about 35%, or about 10% to about 50%, or about 10% to about 60%, or about 10% to about 70%, or about 10% to about 80%, or about 10% to about 90%, or about 10% to about 100%, or about 20% to about 40%, or about 20% to about 50%, or about 20% to about 60%, or about 20% to about 70%, or about 20% to about 80%, or about 20% to about 90%, or about 20% to about 100%, or about 30% to about 50%, or about 30% to about 60%, or about 30% to about 70%, or about 30% to about 80%, or about 30% to about 90%, or about 40% to about 80%, or about 40% to about 90%, or about 60% to about 100%, or greater than about 5%, or greater than about 10%, or greater than about 15%, or greater than about 20%, or greater than about 30%, or greater than about 40%, or greater than about 50%, or greater than about 60%, or greater than about 80%, or greater than about 90%, or greater than about 95%, at one hour, when measured by the USP Basket and Paddle Methods at 100 rpm in 700 ml of Simulated Gastric Fluid (SGF) at 37° C.

In some preferred embodiments, the opioids dosage form provides a Cₘₐₓ of opioid which is less than 65% of the Cₘₐₓ of an equivalent dose of an oral immediate release opioid solution. In other preferred embodiments, said dosage form provides a Cₘₐₓ which is less than about 85%, or less than about 75%, or less than about 60%, or less than about 55%, or less than about 50%, or less than about 45%, or less than about 40%, or less than about 30%, or less than about 20% of the Cₘₐₓ of an equivalent dose of an oral immediate release opioid solution.

In some preferred embodiments, the dosage form provides a time to 75% mean Cₘₐₓ of opioid which is about 100% to about 2000% of the time to 75% mean Cₘₐₓ of an oral immediate release opioid solution.

In some preferred embodiments, the dosage form provides a time to 30% mean Cₘₐₓ of opioid which is about 100% to about 2000% of the time to 30% mean Cₘₐₓ of an oral immediate release opioid solution.

In some preferred embodiments, the dosage from maintains a plasma opioid concentration within 50% of Cₘₐₓ for about 1 to about 9 hours during a 12 hour dosing interval. In other preferred embodiments, said dosage form maintains plasma opioid concentration within 50% of Cₘₐₓ, for about 2 to about 9 hours, or about 3 to about 9 hours, or about 4 to about 9 hours, or about 5 to about 9 hours, or about 6 to about 9 hours, or about 1 to about 11 hours, or about 2 to about 11 hours, or about 3 to about 11 hours or about 4 to about 11 hours, or about 5 to about 11 hours, or about 6 to about 11 hours, or about 7 to about 11 hours, or about 8 to about 11 hours, or about 1 to about 10 hours, or about 2 to about 10 hours, or about 3 to about 10 hours or about 4 to about 10 hours, or about 5 to about 10 hours, or about 6 to about 10 hours, or about 7 to about 10 hours, or about 8 to about 10 hours, or about 1 to about 7 hours, or about 2 to about 7 hours, or about 3 to about 7 hours or about 4 to about 7 hours, or about 5 to about 7 hours, or about 6 to about 7 hours, or about 1 to about 4 hours, or about 1 to about 5 hours, during a 12 hour dosing interval.

In some preferred embodiments, the dosage from maintains a plasma opioid concentration within 30% of Cₘₐₓ for about 1.5 to about 9 hours during a 12 hour dosing interval. In other preferred embodiments, said dosage form maintains plasma opioid concentration within 30% of Cₘₐₓ for about 2 to about 9 hours, or about 3 to about 9 hours, or about 4 to about 9 hours, or about 5 to about 9 hours, or about 6 to about 9 hours, or about 1 to about 11 hours, or about 2 to about 11 hours, or about 3 to about 11 hours or about 4 to about 11 hours, or about 5 to about 11 hours, or about 6 to about 11 hours, or about 7 to about 11 hours, or about 8 to about 11 hours, or about 1 to about 10 hours, or about 2 to about 10 hours, or about 3 to about 10 hours or about 4 to about 10 hours, or about 5 to about 10 hours, or about 6 to about 10 hours, or about 7 to about 10 hours, or about 8 to about 10 hours, or about 1 to about 7 hours, or about 2 to about 7 hours, or about 3 to about 7 hours or about 4 to about 7 hours, or about 5 to about 7 hours, or about 6 to about 7 hours, or about 1 to about 4 hours, or about 1 to about 5 hours, during a 12 hour dosing interval.

In some preferred embodiments, the dosage from maintains a plasma opioid concentration within 65% of Cₘₐₓ for about 1 to about 9 hours during a 12 hour dosing interval. In other preferred embodiments, said dosage form maintains plasma opioid concentration within 65% of Cₘₐₓ for about 2 to about 9 hours, or about 3 to about 9 hours, or about 4 to about 9 hours, or about 5 to about 9 hours, or about 6 to about 9 hours, or about 1 to about 11 hours, or about 2 to about 11 hours, or about 3 to about 11 hours or about 4 to about 11 hours, or about 5 to about 11 hours, or about 6 to about 11 hours, or about 7 to about 11 hours, or about 8 to about 11 hours, or about 1 to about 10 hours, or about 2 to about 10 hours, or about 3 to about 10 hours or about 4 to about 10 hours, or about 5 to about 10 hours, or about 6 to about 10 hours, or about 7 to about 10 hours, or about 8 to about 10 hours, or about 1 to about 7 hours, or about 2 to about 7 hours, or about 3 to about 7 hours or about 4 to about 7 hours, or about 5 to about 7 hours, or about 6 to about 7 hours, or about 1 to about 4 hours, or about 1 to about 5 hours, during a 12 hour dosing interval.

In some preferred embodiments, the dosage from maintains a plasma opioid concentration within 55% of Cₘₐₓ for about 3 to about 22 hours during a 24 hour dosing interval. In other preferred embodiments, said dosage form maintains plasma opioid concentration within 50% of Cₘₐₓ, for about 1 to about 9 hours, or about 4 to about 9 hours, or about 6 to about 9 hours, or about 1 to about 20 hours, or about 2 to about 20 hours, or about 3 to about 20 hours, or about 1 to about 18 hours, or about 1 to about 16 hours or about 2 to about 18 hours, or about 2 to about 16 hours, or about 1 to about 14 hours, or about 1 to about 12 hours, or about 4 to about 16 hours, or about 4 to about 18 hours, or about 4 to about 20 hours, or about 3 to about 15 hours or about 6 to about 15 hours, or about 6 to about 12 hours, or about 6 to about 18 hours, or about 6 to about 20 hours, or about 5 to about 12 hours, or about 5 to about 14 hours, or about 3 to about 22 hours, or about 3 to about 9 hours or about 3 to about 12 hours, or about 1 to about 6 hours, or about 2 to about 8 hours, or about 2 to about 10 hours, or about 3 to about 16 hours, during a 24 hour dosing interval.

In some preferred embodiments, the dosage from maintains a plasma opioid concentration within 30% of Cₘₐₓ for about 2 to about 22 hours during a 24 hour dosing interval. In other preferred embodiments, said dosage form maintains plasma opioid concentration within 30% of Cₘₐₓ for about 1 to about 9 hours, or about 4 to about 9 hours, or about 6 to about 9 hours, or about 1 to about 20 hours, or about 2 to about 20 hours, or about 3 to about 20 hours, or about 1 to about 18 hours, or about 1 to about 16 hours or about 2 to about 18 hours, or about 2 to about 16 hours, or about 1 to about 14 hours, or about 1 to about 12 hours, or about 4 to about 16 hours, or about 4 to about 18 hours, or about 4 to about 20 hours, or about 3 to about 15 hours or about 6 to about 15 hours, or about 6 to about 12 hours, or about 6 to about 18 hours, or about 6 to about 20 hours, or about 5 to about 12 hours, or about 5 to about 14 hours, or about 3 to about 22 hours, or about 3 to about 9 hours or about 3 to about 12 hours, or about 1 to about 6 hours, or about 2 to about 8 hours, or about 2 to about 10 hours, or about 3 to about 16 hours, during a 24 hour dosing interval.

In some preferred embodiments, the dosage from maintains a plasma opioid concentration within 65% of Cₘₐₓ for about 2 to about 22 hours during a 24 hour dosing interval. In other preferred embodiments, said dosage form maintains plasma opioid concentration within 65% of Cₘₐₓ for about 1 to about 9 hours, or about 4 to about 9 hours, or about 6 to about 9 hours, or about 1 to about 20 hours, or about 2 to about 20 hours, or about 3 to about 20 hours, or about 1 to about 18 hours, or about 1 to about 16 hours or about 2 to about 18 hours, or about 2 to about 16 hours, or about 1 to about 14 hours, or about 1 to about 12 hours, or about 4 to about 16 hours, or about 4 to about 18 hours, or about 4 to about 20 hours, or about 3 to about 15 hours or about 6 to about 15 hours, or about 6 to about 12 hours, or about 6 to about 18 hours, or about 6 to about 20 hours, or about 5 to about 12 hours, or about 5 to about 14 hours, or about 3 to about 22 hours, or about 3 to about 9 hours or about 3 to about 12 hours, or about 1 to about 6 hours, or about 2 to about 8 hours, or about 2 to about 10 hours, or about 3 to about 16 hours, during a 24 hour dosing interval.

In some preferred embodiments, the dosage form provides a Tₘₐₓ of opioid at a time point 1 to 18 times later than the Tₘₐₓ provided by an equivalent dose of an oral immediate release opioid solution. In the dosage form provides a Tₘₐₓ at a time point about 1 to 15 times late, or about of 1 to 10 times later, or about of 1 to 7 times later, or about of 1 to 4 times later, or about of 3 to 20 times later, or about of 3 to 10 times later, or about of 3 to 5 times later, or about 1.5 to 15 times later, or about of 1.5 to 10 times later, or about of 1.5 to 7 times later, or about of 1.5 to 3 times later, or about of 2 to 20 times later, or about of 2 to 10 times later, or about of 2 to 5 times later, or about of 2 to 3 times later, or about of 2.5 to 20 times later, or about of 2.5 to 8 times later, or about of 2.5 to 5 times later, or about of 2.5 to 4 times later, or about of 3 to 20 times later, or about of 3 to 10 times later, or about of 3 to 5 times later.

In some preferred embodiments, the dosage form provides a mean in vivo extent of absorption of opioid from 0 to 4 hours which is at least 20% of the mean in vivo extent of absorption from to 0 to 12 hours, wherein the mean in vivo extent of absorption is the area under the plasma or serum opioid concentration time curve from the time of drug administration to the specified time point. In other preferred embodiments, said in vivo extent of absorption from 0 to 4 hours is at least about 5%, or at least about 10%, or at least about 15%, or at least about 25%, or at least about 30%, or at least about 40%, or at least about 50%, or at least about 60%, or at least about 70%, or at least about 80%, at least about 90%, or about 100% of the mean in vivo extent of absorption from to 0 to 12 hours.

In some preferred embodiments, the dosage form provides a mean in vivo extent of absorption of opioid from 0 to 8 hours which is at least 20% of the mean in vivo extent of absorption from to 0 to 24 hours, wherein the mean in vivo extent of absorption is the area under the plasma or serum opioid concentration time curve from the time of drug administration to the specified time point. In other preferred embodiments, said in vivo extent of absorption from 0 to 8 hours is at least about 5%, or at least about 10%, or at least about 15%, or at least about 25%, or at least about 30%, or at least about 40%, or at least about 50%, or at least about 60%, or at least about 70%, or at least about 80%, at least about 90%, or about 100% of the mean in vivo extent of absorption from to 0 to 24 hours.

In some preferred embodiments, the dosage form provides a mean in vivo extent of absorption of opioid from 0 to 12 hours which is at least 20% of the mean in vivo extent of absorption from to 0 to 24 hours, wherein the mean in vivo extent of absorption is the area under the plasma or serum opioid concentration time curve from the time of drug administration to the specified time point. In other preferred embodiments, said in vivo extent of absorption from 0 to 12 hours is at least about 5%, or at least about 10%, or at least about 15%, or at least about 25%, or at least about 30%, or at least about 40%, or at least about 50%, or at least about 60%, or at least about 70%, or at least about 80%, at least about 90%, or about 100% of the mean in vivo extent of absorption from to 0 to 24 hours.

In some preferred embodiments, the dosage form provides a mean in vivo extent of absorption of opioid over the dosing interval, AUC₀₋ₜ (e.g., from 0 to 8 hours, or from 0 to 12 hours or from 0 to 24 hours) which is at least 40% of the mean in vivo extent of absorption from to 0 to infinity (AUC_{0-∞}). In other preferred embodiments, said AUC₀₋ₜ is at least about 50%, or at least about 60%, or at least about 70%, or at least about 80%, or at least about 90% of the mean in vivo extent of absorption from to 0 to infinity (AUC_{0-∞}).

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release rate by weight of an opioid agonist, when measured by the USP Basket and Paddle Methods at 100 rpm in 900 mL aqueous buffer at a pH of between 1.6 and 7.2 at 37° C. of between 0% to about 100% at 0.5 hours, and greater than about 60% at 1 hour.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release rate by weight of an opioid agonist, when measured by the USP Basket and Paddle Methods at 100 rpm in 900 mL aqueous buffer at a pH of between 1.6 and 7.2 at 37° C. of between 0% to about 40% at 1 hour, from about 5% to about 60% at 2 hours, from about 10% to about 75% at 4 hours, from about 20% to about 75% at 6 hours, from about 30% to about 80% at 9 hours, and greater than about 70% at 12 hours.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release rate by weight of an opioid agonist, when measured by the USP Basket and Paddle Methods at 100 rpm in 900 mL aqueous buffer at a pH of between 1.6 and 7.2 at 37° C. of between 1% and about 45% at 1 hour, between about 5% and about 70% at 2 hours, between about 10% and about 90% at 4 hours, between about 20% and about 90% at 8 hours, greater than about 60% at 12 hours, greater than about 80% at 18 hours, and greater than about 85% at 24 hours.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release rate by weight of an opioid agonist, when measured by the USP Basket and Paddle Methods at 100 rpm in 900 mL aqueous buffer at a pH of between 1.6 and 7.2 at 37° C. of between 5% and about 60% at 1 hour, between about 12.5% and about 80% at 2 hours, between about 25% and about 95% at 4 hours, between about 45% and about 100% at 8 hours, greater than about 55% at 12 hours, greater than about 65% at 18 hours, and greater than about 70% at 24 hours.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release rate by weight of an opioid agonist, when measured by the USP Basket and Paddle Methods at 100 rpm in 900 mL aqueous buffer at a pH of between 1.6 and 7.2 at 37° C. of between 0% and about 40% at 1 hour, between about 0% and about 70% at 2 hours, between about 5% and about 95% at 4 hours, between about 12.5% and about 100% at 8 hours, between about 20% and about 100% at 12 hours, between about 35% and about 100% at 16 hours, between about 55% and about 100% at 24 hours, and greater than about 75% at 36 hours.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release rate by weight of an opioid agonist, when measured by the USP Basket and Paddle Methods at 100 rpm in 900 mL aqueous buffer at a pH of between 1.6 and 7.2 at 37° C. of between 0% and about 60% at 1 hour, between about 0% and about 75% at 2 hours, between about 5% and about 95% at 4 hours, between about 12.5% and about 100% at 8 hours, between about 15% and about 100% at 12 hours, between about 25% to about 100% at 16 hours, between about 30% and about 100% hours at 24 hours and greater than 60% at 36 hours.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release from the dosage form at one hour when measured by the USP Basket and Paddle Methods at 100 rpm in 700 ml of Simulated Gastric Fluid (SGF) at 37° C. of between 0% to about 50% by weight of the opioid. In other preferred embodiments, said release rate is between 0% to about 1%, or 0% to about 3%, or 0% to about 5%, or 0% to about 10%, or 0% to about 15%, or 0% to about 20%, 0% to about 30%, or 0% to about 40%, or 0% to about 60%, or 0% to about 70%, or 0% to about 80%, or 0% to about 90%, 0% to about 100%.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release from the dosage form at one hour when measured by the USP Basket and Paddle Methods at 100 rpm in 900 mL aqueous buffer at a pH of between 1.6 and 7.2 at 37° C. of between 0% and about 60% at 1 hour, between about 0% and about 80% at 2 hours, between about 3% and about 95% at 4 hours and between about 10% and about 100% at 8 hours. In other preferred embodiments, said release rate is between 0% and about 10% at 1 hour, between about 0% and about 20% at 2 hours, between about 2% and about 80% at 4 hours and between about 5% and about 100% at 8 hours; or between 0% and about 20% at 1 hour, between about 0% and about 40% at 2 hours, between about 0% and about 80% at 4 hours and between about 2% and about 100% at 8 hours; or between 0% and about 4.0% at 1 hour, between about 0% and about 60% at 2 hours, between about 5% and about 85% at 4 hours and between about 5% and about 90% at 8 hours and greater than 20% at 12 hours; or between 0% and about 50% at 1 hour, between about 0% and about 50% at 2 hours, between about 10% and about 90% at 4 hours and between about 15% and about 90% at 8 hours and greater than 30% at 12 hours; or between 0% and about 70% at 1 hour, between about 0% and about 70% at 2 hours, between about 10% and about 75% at 4 hours and between about 15% and about 90% at 8 hours and greater than 30% at 12 hours.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release from the dosage form at one hour when measured by the USP Basket and Paddle Methods at 100 rpm in 900 mL aqueous buffer at a pH of between 1.6 and 7.2 at 37° C. of between 10% and about 65% at 1 hour, between about 20% and about 75% at 2 hours, between about 30% and about 95% at 4 hours and between about 40% and about 100% at 8 hours. In other preferred embodiments, said release rate is between 2% and about 70% at 1 hour, between about 5% and about 80% at 2 hours, between about 10% and about 90% at 4 hours and between about 20% and about 100% at 8 hours; or between 5% and about 60% at 1 hour, between about 10% and about 75% at 2 hours, between about 15% and about 85% at 4 hours and between about 30% and about 100% at 8 hours; or between 20% and about 70% at 1 hour, between about 20% and about 75% at 2 hours, between about 20% and about 90% at 4 hours and between about 40% and about 100% at 8 hours; or between 30% and about 80% at 1 hour, between about 40% and about 85% at 2 hours, between about 40% and about 90% at 4 hours and between about 60% and about 100% at 8 hours; or between 1% and about 20% at 1 hour, between about 5% and about 20% at 2 hours, between about 10% and about 40% at 4 hours and between about 20% and about 40% at 8 hours and greater than 40% at 12 hours.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release rate by weight of the opioid, when measured by the USP Basket and Paddle Methods at 100 rpm in 900 mL aqueous buffer at a pH of between 1.6 and 7.2 at 37° C. of between 0% to about 47.5% at 1 hour, from about 10% to about 65% at 2 hours, from about 15% to about 70% at 4 hours, from about 25% to about 77.5% at 6 hours, from about 35% to about 87.5% at 9 hours, and greater than about 65% at 12 hours. In other preferred embodiments, said release rate is between 0% to about 30% at 1 hour, from about 5% to about 45% at 2 hours, from about 10% to about 60% at 4 hours, from about 15% to about 70% at 6 hours, from about 25% to about 80% at 9 hours, and greater than about 50% at 12 hours; or between 0% to about 20% at 1 hour, from about 2% to about 35% at 2 hours, from about 5% to about 50% at 4 hours; from about 10% to about 60% at 6 hours, from about 15% to about 70% at 9 hours, and greater than about 40% at 12 hours; or between 0% to about 10% at 1 hour, from about 1% to about 30% at 2 hours, from about 5% to about 40% at 4 hours, from about 10% to about 60% at 6 hours, from about 15% to about 70% at 9 hours, and greater than about 40% at 12 hours; or between 0% to about 5% at 1 hour, from about 0% to about 10% at 2 hours, from about 2% to about 20% at 4 hours, from about 5% to about 30% at 6 hours, from about 10% to about 40% at 9 hours, and greater than about 30% at 12 hours; or between 0% to about 50% at 1 hour, from about 15% to about 70% at 2 hours, from about 20% to about 75% at 4 hours, from about 30% to about 80% at 6 hours, from about 30% to about 90% at 9 hours, and greater than about 70% at 12 hours; or between 0% to about 60% at 1 hour, from about 15% to about 80% at 2 hours, from about 25% to about 85% at 4 hours, from about 35% to about 90% at 6 hours, from about 40% to about 90% at 9 hours, and greater than about 80% at 12 hours; or between 0% to about 70% at 1 hour, from about 20% to about 80% at 2 hours, from about 25% to about 80% at 4 hours, from about 35% to about 80% at 6 hours, from about 40% to about 80% at 9 hours, and greater than about 60% at 12 hours; or between 0% to about 75% at 1 hour, from about 30% to about 80% at 2 hours, from about 35% to about 90% at 4 hours, from about 50% to about 90% at 6 hours, from about 55% to about 95% at 9 hours, and greater than about 70% at 12 hours.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release rate by weight of the opioid, when measured by the USP Basket and Paddle Methods at 100 rpm in 900 mL aqueous buffer at a pH of between 1.6 and 7.2 at 37° C. of between 5% and about 50% at 1 hour, between about 10% and about 75% at 2 hours, between about 20% and about 95% at 4 hours, between about 40% and about 100% at 8 hours, greater than about 50% at 12 hours, greater than about 70% at 18 hours, and greater than about 80% at 24 hours. In other preferred embodiments, said release rate is between 2% and about 50% at 1 hour, between about 5% and about 75% at 2 hours, between about 15% and about 75% at 4 hours, between about 30% and about 90% at 8 hours, greater than about 40% at 12 hours, greater than about 60% at 18 hours, and greater than about 70% at 24 hours; or between 1% and about 40% at 1 hour, between about 2% and about 60% at 2 hours, between about 10% and about 65% at 4 hours, between about 20% and about 80% at 8 hours, greater than about 30% at 12 hours, greater than about 40% at 18 hours, and greater than about 60% at 24 hours; or between 5% and about 60% at 1 hour, between about 15% and about 80% at 2 hours, between about 25% and about 95% at 4 hours, between about 45% and about 100% at 8 hours, greater than about 60% at 12 hours, greater than about 80% at 18 hours, and greater than about 90% at 24 hours; or between 10% and about 65% at 1 hour, between about 20% and about 85% at 2 hours, between about 30% and about 100% at 4 hours, between about 60% and about 100% at 8 hours, greater than about 70% at 12 hours, greater than about 90% at 18 hours, and greater than about 95% at 24 hours.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release rate by weight of the opioid, when measured by the USP Basket and Paddle Methods at 100 rpm in 900 mL aqueous buffer at a pH of between 1.6 and 7.2 at 37° C. of between 0% to about 30% at 1 hour, from about 10% to about 65% at 4 hours, from about 20% to about 70% at 8 hours, from about 25% to about 80% at 12 hours, from about 35% to about 95% at 18 hours, and greater than about 65% at 24 hours. In other preferred embodiments, said release rate is between 0% to about 20% at 1 hour, from about 5% to about 50% at 4 hours, from about 10% to about 60% at 8 hours, from about 15% to about 70% at 12 hours, from about 25% to about 90% at 18 hours, and greater than about 55% at 24 hours; or between 0% to about 10% at 1 hour, from about 5% to about 40% at 4 hours, from about 8% to about 50% at 8 hours, from about 10% to about 60% at 12 hours, from about 0.22% to about 80% at 18 hours, and greater than about 45% at 24 hours; or between 0% to about 35% at 1 hour, from about 15% to about 70% at 4 hours, from about 25% to about 75% at 8 hours, from about 30% to about 85% at 12 hours, from about 40% to about 100% at 18 hours, and greater than about 75% at 24 hours; or between 0% to about 40% at 1 hour, from about 20% to about 70% at 4 hours, from about 30% to about 80% at 8 hours, from about 35% to about 90% at 12 hours, from about 45% to about 100% at 18 hours, and greater than about 80% at 24 hours; or between 0% to about 45% at 1 hour, from about 25% to about 75% at 4 hours, from about 35% to about 85% at 8 hours, from about 40% to about 90% at 12 hours, from about 50% to about 100% at 18 hours, and greater than about 90% at 24 hours; or between 0% to about 50% at 1 hour, from about 30% to about 80% at 4 hours, from about 40% to about 90% at 8 hours, from about 45% to about 95% at 12 hours, from about 60% to about 100% at 18 hours, and greater than about 95% at 24 hours; or between 0% to about 60% at 1 hour, from about 40% to about 80% at 4 hours, from about 45% to about 90% at 8 hours, from about 50% to about 100% at 12 hours, from about 70% to about 100% at 18 hours, and greater than about 80% at 24 hours.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release rate by weight of the opioid, when measured by the USP Basket and Paddle Methods at 100 rpm in 900 mL aqueous buffer at a pH of between 1.6 and 7.2 at 37° C. of between 0% and about 50% at 1 hour, between about 0% and about 75% at 2 hours, between about 3% and about 95% at 4 hours, between about 10% and about 100% at 8 hours, between about 25% and about 100% at 12 hours, between about 30% and about 100% at 16 hours, between about 50% and about 100% at 24 hours, and greater than about 80% at 36 hours. In other preferred embodiments, said release rate is between 0% and about 40% at 1 hour, between about 0% and about 65% at 2 hours, between about 2% and about 85% at 4 hours, between about 8% and about 90% at 8 hours, between about 20% and about 95% at 12 hours, between about 25% and about 95% at 16 hours, between about 40% and about 90% at 24 hours, and greater than about 70% at 36 hours; or between 0% and about 30% at 1 hour, between about 0% and about 50% at 2 hours, between about 1% and about 75% at 4 hours, between about 5% and about 80% at 8 hours, between about 10% and about 85% at 12 hours, between about 15% and about 90% at 16 hours, between about 30% and about 80% at 24 hours, and greater than about 70% at 36 hours; or between 0% and about 60% at 1 hour, between about 0% and about 80% at 2 hours, between about 5% and about 100% at 4 hours, between about 15% and about 100% at 8 hours, between about 35% and about 100% at 12 hours, between about 40% and about 100% at 16 hours, between about 60% and about 100% at 24 hours, and greater than about 85% at 36 hours; or between 0% and about 65% at 1 hour, between about 0% and about 85% at 2 hours, between about 10% and about 100% at 4 hours, between about 20% and about 100% at 8 hours, between about 40% and about 100% at 12 hours, between about 50% and about 100% at 16 hours, between about 70% and about 100% at 24 hours, and greater than about 90% at 36 hours; or between 0% and about 70% at 1 hour, between about 0% and about 90% at 2 hours, between about 20% and about 100% at 4 hours, between about 30% and about 100% at 8 hours, between about 50% and about 100% at 12 hours, between about 60% and about 100% at 16 hours, between about 80% and about 100% at 24 hours, and greater than about 95% at 36 hours.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release rate by weight of the opioid, when measured by the USP Basket and Paddle Methods at 100 rpm in 900 mL aqueous buffer at a pH of between 1.6 and 7.2 at 37° C. of between 20% and about 50% at 1 hour, between about 40% and about 75% at 2 hours, between about 60% and about 95% at 4 hours, between about 80% and about 100% at 8 hours and between about 90% and about 100% at 12 hours. In other preferred embodiments, said release rate is between 15% and about 45% at 1 hour, between about 35% and about 70% at 2 hours, between about 55% and about 90% at 4 hours, between about 75% and about 90% at 8 hours and between about 80% and about 95% at 12 hours; or between 10% and about 40% at 1 hour, between about 30% and about 65% at 2 hours, between about 50% and about 85% at 4 hours, between about 70% and about 85% at 8 hours and between about 75% and about 90% at 12 hours; or between 5% and about 35% at 1 hour, between about 25% and about 60% at 2 hours, between about 45% and about 80% at 4 hours, between about 65% and about 80% at 8 hours and between about 70% and about 85% at 12 hours; or between 25% and about 55% at 1 hour, between about 45% and about 80% at 2 hours, between about 65% and about 95% at 4 hours, between about 85% and about 100% at 8 hours and between about 95% and about 100% at 12 hours; or between 30% and about 60% at 1 hour, between about 50% and about 80% at 2 hours, between about 70% and about 95% at 4 hours, between about 90% and about 100% at 8 hours and between about 95% and about 100% at 12 hours; or between 35% and about 60% at 1 hour, between about 50% and about 80% at 2 hours, between about 80% and about 95% at 4 hours, between about 90% and about 100% at 8 hours and between about 95% and about 100% at 12 hours; or between 20% and about 40% at 1 hour, between about 40% and about 65% at 2 hours, between about 60% and about 85% at 4 hours, between about 70% and about 90% at 8 hours and between about 80% and about 100% at 12 hours.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release rate by weight of the opioid, when measured by the USP Basket and Paddle Methods at 100 rpm in 900 mL aqueous buffer at a pH of between 1.6 and 7.2 at 37° C. of between 0% and about 50% at 1 hour, between about 0% and about 75% at 2 hours, between about 10% and about 95% at 4 hours, between about 35% and about 100% at 8 hours, between about 55% and about 100% at 12 hours, between about 70% to about 100% at 16 hours, and greater than about 90% at 24 hours. In other preferred embodiments, said release rate is between 0% and about 40% at 1 hour, between about 0% and about 65% at 2 hours, between about 8% and about 85% at 4 hours, between about 30% and about 90% at 8 hours, between about 45% and about 100% at 12 hours, between about 60% to about 100% at 16 hours, and greater than about 80% at 24 hours; or between 0% and about 30% at 1 hour, between about 0% and about 55% at 2 hours, between about 5% and about 75% at 4 hours, between about 20% and about 80% at 8 hours, between about 35% and about 100% at 12 hours, between about 50% to about 100% at 16 hours, and greater than about 70% at 24 hours; or between 0% and about 20% at 1 hour, between about 0% and about 45% at 2 hours, between about 5% and about 65% at 4 hours, between about 10% and about 70% at 8 hours, between about 25% and about 80% at 12 hours, between about 40% to about 100% at 16 hours, and greater than about 60% at 24 hours; or between 0% and about 60% at 1 hour, between about 0% and about 80% at 2 hours, between about 15% and about 95% at 4 hours, between about 40% and about 100% at a hours, between about 60% and about 100% at 12 hours, between about 75% to about 100% at 16 hours, and greater than about 90% at 24 hours; or between 0% and about 65% at 1 hour, between about 0% and about 85% at 2 hours, between about 20% and about 90% at 4 hours, between about 45% and about 100% at 8 hours, between about 65% and about 100% at 12 hours, between about 80% to about 100% at 16 hours, and greater than about 90% at 24 hours; or between 0% and about 40% at 1 hour, between about 0% and about 50% at 2 hours, between about 10% and about 80% at 4 hours, between about 25% and about 70% at 8 hours, between about 40% and about 80% at 12 hours, between about 60% to about 100% at 16 hours, and greater than about 90% at 24 hours.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release rate by weight of the opioid, when measured by the USP Basket and Paddle Methods at 100 rpm in 900 mL aqueous buffer at a pH of between 1.6 and 7.2 at 37° C. of between 0% and about 30% at 1 hour, between about 0% and about 45% at 2 hours, between about 3% and about 55% at 4 hours, between about 10% and about 65% at 8 hours, between about 20% and about 75% at 12 hours, between about 30% to about 88% at 16 hours, between about 50% and about 100% hours at 24 hours and greater than 80% at 36 hours. In other preferred embodiments, said release rate is between 0% and about 25% at 1 hour, between about 0% and about 40% at 2 hours, between about 2% and about 50% at 4 hours, between about 8% and about 60% at 8 hours, between about 10% and about 70% at 12 hours, between about 25% to about 80% at 16 hours, between about 45% and about 100% hours at 24 hours and greater than 75% at 36 hours; or between 0% and about 20% at 1 hour, between about 0% and about 35% at 2 hours, between about 1% and about 45% at 4 hours, between about 5% and about 55% at 8 hours, between about 8% and about 65% at 12 hours, between about 20% to about 75% at 16 hours, between about 40% and about 100% hours at 24 hours and greater than 70% at 36 hours; or between 0% and about 15% at 1 hour, between about 0% and about 30% at 2 hours, between about 0% and about 40% at 4 hours, between about 5% and about 50% at 8 hours, between about 8% and about 60% at 12 hours, between about 15% to about 70% at 16 hours, between about 35% and about 100% hours at 24 hours and greater than 60% at 36 hours; or between 0% and about 10% at 1 hour, between about 0% and about 25% at 2 hours, between about 0% and about 35% at 4 hours, between about 5% and about 45% at 8 hours, between about 10% and about 50% at 12 hours, between about 10% to about 60% at 16 hours, between about 30% and about 90% hours at 24 hours and greater than 70% at 36 hours; or between 0% and about 35% at 1 hour, between about 0% and about 50% at 2 hours, between about 5% and about 60% at 4 hours, between about 15% and about 70% at 8 hours, between about 25% and about 80% at 12 hours, between about 35% to about 90% at 16 hours, between about 55% and about 100% hours at 24 hours and greater than 85% at 36 hours; or between 0% and about 40% at 1 hour, between about 0% and about 55% at 2 hours, between about 10% and about 65% at 4 hours, between about 20% and about 75% at 8 hours, between about 30% and about 85% at 12 hours, between about 40% to about 100% at 16 hours, between about 55% and about 100% hours at 24 hours and greater than 90% at 36 hours; or between 0% and about 45% at 1 hour, between about 0% and about 60% at 2 hours, between about 15% and about 70% at 4 hours, between about 25% and about 80% at 8 hours, between about 35% and about 90% at 12 hours, between about 45% to about 100% at 16 hours, between about 60% and about 100% hours at 24 hours and greater than 60% at 36 hours; or between 0% and about 50% at 1 hour, between about 5% and about 65% at 2 hours, between about 20% and about 75% at 4 hours, between about 30% and about 85% at 8 hours, between about 40% and about 95% at 12 hours, between about 50% to about 100% at 16 hours, between about 70% and about 100% hours at 24 hours and greater than 70% at 36 hours; or between 0% and about 30% at 1 hour, between about 5% and about 40% at 2 hours, between about IQ % and about 60% at 4 hours, between about 20% and about 70% at 8 hours, between about 30% and about 100% at 12 hours, between about 40% to about 100% at 16 hours, between about 60% and about 100% hours at 24 hours and greater than 90% at 36 hours; or between 0% and about 30% at 1 hour, between about 0% and about 30% at 2 hours, between about 0% and about 30% at 4 hours, between about 5% and about 70% at 8 hours, between about 10% and about 80% at 12 hours, between about 20% to about 100% at 16 hours, between about 40% and about 100% hours at 24 hours and greater than 50% at 36 hours; or between 0% and about 20% at 1 hour, between about 0% and about 20% at 2 hours, between about 0% and about 20% at 4 hours, between about 0% and about 20% at 8 hours, between about 5% and about 40% at 12 hours, between about 10% to about 80% at 16 hours, between about 40% and about 100% hours at 24 hours and greater than 60% at 36 hours; or between 0% and about 10% at 1 hour, between about 0% and about 20% at 2 hours, between about 0% and about 40% at 4 hours, between about 5% and about 60% at 8 hours, between about 10% and about 80% at 12 hours, between about 20% to about 100% at 16 hours, between about 40% and about 100% hours at 24 hours and greater than 50% at 36 hours.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release rate by weight of the opioid, when measured by the USP Basket and Paddle Methods at 100 rpm in 900 mL aqueous buffer at a pH of between 1.6 and 7.2 at 37° C. of between 0% and about 50% at 1 hour, between about 0% and about 75% at 2 hours, between about 3% and about 95% at 4 hours, between about 10% and about 100% at 8 hours, between about 20% and about 100% at 12 hours, between about 30% to about 100% at 16 hours, between about 50% and about 100% hours at 24 hours and greater than 80% at 36 hours. In other preferred embodiments, said release rate is between 0% and about 45% at 1 hour, between about 0% and about 70% at 2 hours, between about 3% and about 90% at 4 hours, between about 8% and about 100% at 8 hours, between about 15% and about 100% at 12 hours, between about 25% to about 100% at 16 hours, between about 45% and about 100% hours at 24 hours and greater than 80% at 36 hours; or between 0% and about 40% at 1 hour, between about 0% and about 65% at 2 hours, between about 0% and about 80% at 4 hours, between about 5% and about 80% at 8 hours, between about 100% and about 90% at 12 hours, between about 20% to about 100% at 16 hours, between about 40% and about 100% hours at 24 hours and greater than 70% at 36 hours; or between 0% and about 35% at 1 hour, between about 0% and about 60% at 2 hours, between about 0% and about 70% at 4 hours, between about 3% and about 70% at 8 hours, between about 5% and about 80% at 12 hours, between about 15% to about 100% at 16 hours, between about 30% and about 100% hours at 24 hours and greater than 40% at 36 hours; or between 0% and about 60% at 1 hour, between about 0% and about 80% at 2 hours, between about 5% and about 100% at 4 hours, between about 15% and about 100% at 8 hours, between about 30% and about 100% at 12 hours, between about 40% to about 100% at 16 hours, between about 60% and about 100% hours at 24 hours and greater than 70% at 36 hours; or between 0% and about 50% at 1 hour, between about 0% and about 75% at 2 hours, between about 5% and about 95% at 4 hours, between about 25% and about 80% at 8 hours, between about 30% and about 100% at 12 hours, between about 40% to about 100% at 16 hours, between about 60% and about 100% hours at 24 hours and greater than 60% at 36 hours; or between 0% and about 60% at 1 hour, between about 0% and about 85% at 2 hours, between about 5% and about 100% at 4 hours, between about 10% and about 100% at 8 hours, between about 20% and about 100% at 12 hours, between about 30% to about 100% at 16 hours, between about 50% and about 100% hours at 24 hours and greater than 80% at 36 hours.

In some preferred embodiments, the dosage form provides an oral pharmaceutical composition for the treatment of pain comprising a therapeutically effective amount of opioid or pharmaceutically acceptable salts thereof or mixtures thereof and ADER, said opioids dosage form providing an in-vitro release rate by weight of the opioid, when measured by the USP Basket and Paddle Methods at 100 rpm in 900 mL aqueous buffer at a pH of between 1.6 and 7.2 at 37° C. of between 15% and about 25% at 1 hour, between about 25% and about 35% at 2 hours, between about 30% and about 45% at 4 hours, between about 40% and about 60% at 8 hours, between about 55% and about 70% at 12 hours and between about 60% to about 75% at 16 hours. In other preferred embodiments, said release rate is between 10% and about 20% at 1 hour, between about 20% and about 30% at 2 hours, between about 25% and about 40% at 4 hours, between about 30% and about 50% at 8 hours, between about 50% and about 65% at 12 hours and between about 55% to about 65% at 16 hours; or between 5% and about 15% at 1 hour, between about 15% and about 25% at 2 hours, between about 20% and about 35% at 4 hours, between about 25% and about 45% at 8 hours, between about 45% and about 60% at 12 hours and between about 50% to about 60% at 16 hours; or between 15% and about 30% at 1 hour, between about 20% and about 40% at 2 hours, between about 20% and about 50% at 4 hours, between about 30% and about 70% at 8 hours, between about 60% and about 80% at 12 hours and between about 70% to about 90% at 16 hours; or between 0% and about 50% at 1 hour, between about 5% and about 50% at 2 hours, between about 5% and about 70% at 4 hours, between about 10% and about 80% at 8 hours, between about 20% and about 100% at 12 hours and between about 40% to about 100% at 16 hours; or between 15% and about 40% at 1 hour, between about 15% and about 45% at 2 hours, between about 20% and about 60% at 4 hours, between about 20% and about 80% at 8 hours, between about 30% and about 90% at 12 hours and between about 40% to about 100% at 16 hours.

In some preferred embodiments, the dosage form provides an oral pharmaceutical composition for the treatment of pain comprising a therapeutically effective amount of opioid or pharmaceutically acceptable salts thereof or mixtures thereof and ADER, said in-vitro release rate being substantially independent of pH in that a difference, at any given time, between an amount of opioid released at one pH and an amount released at any other pH, when measured in-vitro using the USP Basket and Paddle Methods of USP Drug Release test of U.S. Pharmacopeia (2003) at 100 rpm in 900 ml aqueous buffer, is no greater than 30%. In other preferred embodiments, the difference, at any given time, between an amount of opioid released at one pH and an amount released at any other pH using the aforementioned methods is no greater than 50%, or no greater than 40%, or no greater than 35%, or no greater than 25%, or no greater than 20%, or no greater than 15%, or no greater than 10%, or no greater than 5%.

In some preferred embodiments, the dosage form provides an oral pharmaceutical composition for the treatment of pain comprising a therapeutically effective amount of opioid or pharmaceutically acceptable salts thereof or mixtures thereof and ADER, said dosage forms of opioid providing in-vitro release rates by weight of between 0% to about 50% by weight of the opioid from the dosage form at one hour when measured by the USP Basket and Paddle Methods at 100 rpm in 700 ml of Simulated Gastric Fluid (SGF) at 37° C. In other preferred embodiments, said release rate at one hour is between 0% to about 10% by weight, or 0% to about 20% by weight, or is between 0% to about 30% by weight, or 0% to about 40% by weight, or between 0% to about 60% by weight, or 0% to about 70% by weight, or 0% to about 80% by weight, or 0% to about 90% by weight, or 10% to about 50% by weight, or 10% to about 60% by weight, or 10% to about 70% by weight, or 10% to about 90% by weight, or 10% to about 100% by weight, or 30% to about 100% by weight, or 50% to about 100% by weight.

In some preferred embodiments, the dosage form provides an oral pharmaceutical composition for the treatment of pain comprising a therapeutically effective amount of opioid or pharmaceutically acceptable salts thereof or mixtures thereof and ADER, said dosage forms of opioid providing in-vitro release rates by weight of opioid, when measured by the USP Basket and Paddle Methods at 100 rpm in 900 mL aqueous buffer at a pH of between 1.6 and 7.2 at 37° C. of between 0% to about 80% at 0.5 hours, and greater than about 40% at 1 hour. In other preferred embodiments, said release rate is between 0% to about 40% at 0.5 hours, and greater than about 60% at 1 hour; or between 0% to about 20% at 0.5 hours, and greater than about 40% at 1 hour; or between 0% to about 20% at 0.5 hours, and greater than about 20% at 1 hour; or between 0% to about 90% at 0.5 hours, and greater than about 60% at 1 hour; or between 0% to about 100% at 0.5 hours, and greater than about 60% at 1 hour; or between 0% to about 90% at 1 hour, and greater than about 40% at 2 hours; or between 0% to about 100% at 1 hour, and greater than about 60% at 2 hours; or between 0% to about 60% at 1 hour, and greater than about 40% at 2 hours; or between 0% to about 40% at 1 hour, and greater than about 30% at 2 hours; or between 0% to about 50% at 1 hour, and greater than about 40% at 2 hours; or between 0% to about 30% at 1 hour, and greater than about 20% at 2 hours; or between 0% and about 50% at 1 hour, between about 0% and about 80% at 2 hours, between about 5% and about 100% at 4 hours and between about 10% and about 100% at 8 hours; or between 10% and about 60% at 1 hour, between about 15% and about 75% at 2 hours, between about 20% and about 95% at 4 hours and between about 30% and about 100% at 8 hours.

In some preferred embodiments, the oral dosage form of the present invention is directed to an oral dosage form comprising: (i) an opioid and (ii) ADER, such that the ratio of the mean Cₘₐₓ of the opioid after single dose oral administration of the dosage form after tampering to the mean Cₘₐₓ of opioid agonist after single dose oral administration of an intact dosage form is not more than about 20:1. In other embodiments of the invention, the mean Cₘₐₓ ratio using the aforementioned test method is not more than about 15:1, or about 10:1, or about 7.5:1, or about 6:1, or about 5:1, or about 4:1, or about 3:1, or about 2:1, or about 1.5:1, or about 1.25:1.

In some preferred embodiments, the oral dosage form of the present invention is directed to an oral dosage form comprising: (i) an opioid agonist and (ii) ADER, such that the ratio of the mean Cₘₐₓ of the opioid agonist after single dose oral administration of an immediate release reference product containing an equivalent amount of opioid agonist to the mean Cₘₐₓ of opioid agonist after single dose oral administration of an intact dosage form of the invention is at least about 1.25:1. In other embodiments of the invention, the mean Cₘₐₓ ratio using the aforementioned test method is at least about 1.5:1, or about 2:1, or about 3:1, or about 4:1, or about 5:1, or about 6:1, or about 10:1, or about 15:1 or about 20:1.

In some preferred embodiments, the oral dosage form of the present invention is directed to an oral dosage form comprising: (i) an opioid agonist and (ii) ADER, such that the ratio of the mean AUC₀₋₂ of the opioid agonist after single dose oral administration of the dosage form after tampering to the mean AUC₀₋₂ of opioid agonist after single dose oral administration of an intact dosage form is not more than about 20:1. In other embodiments, the mean AUC ratio using the aforementioned test method is measured from time 0 to up to 1, 2.5, 3, 4, 5 or 6 hours post dose (i.e., AUC₀₋₁, AUC_{0-2.5}, AUC₀₋₃, AUC₀₋₄, AUC₀₋₅ and AUC₀₋₆, respectively). In other embodiments of the invention, the mean AUC₀₋₁, AUC₀₋₂, AUC_{0-2.5}, AUC₀₋₃, AUC₀₋₄, AUC₀₋₅ and AUC₀₋₆ ratios using the aforementioned test method are not more than about 15:1, or about 10:1, or about 7.5:1, or about 6:1, or about 5:1, or about 4:1, or about 3:1, or about 2:1 or about 1.5:1.

In some preferred embodiments, the oral dosage form of the present invention is directed to an oral dosage form comprising: (i) an opioid agonist and (ii) ADER, such that the ratio of the mean AUC₀₋₂ of the opioid agonist after single dose oral administration of an immediate release reference product containing an equivalent amount of opioid agonist to the mean AUC₀₋₂ of opioid agonist after single dose oral administration of an intact dosage form of the invention is at least about 1.25:1. In other embodiments, the mean AUC ratio using the aforementioned test method is measured from time 0 to up to 1, 2.5, 3, 4, 5 or 6 hours post dose (i.e., AUC₀₋₁, AUC_{0-2.5}, AUC₀₋₃, AUC₀₋₄, AUC₀₋₅ and AUC₀₋₆, respectively). In other embodiments of the invention, the mean AUC₀₋₁, AUC₀₋₂, AUC_{0-2.5}, AUC₀₋₃, AUC₀₋₄, AUC₀₋₅ and AUC₀₋₆ ratios using the aforementioned test method are not more than about 15:1, or about 10:1, or about 7.5:1, or about 6:1, or about or about 5:1, or about 4:1, or about 3:1, or about 2:1 or about 1.5:1.

In some preferred embodiments, the oral dosage form of the present invention is directed to an oral dosage form comprising: (i) an opioid agonist and (ii) ADER, such that the ratio of the mean Tₘₐₓ of the opioid agonist after single dose oral administration of the intact dosage form to the mean Tₘₐₓ of opioid agonist after single dose oral administration of an dosage form after tampering is not more than about 20:1. In other embodiments of the invention, the mean Tₘₐₓ ratio using the aforementioned test method is not more than about 15:1, or not more than about 10:1, or not more than about 7.5:1, or not more than about 6:1, or not more than about 5:1, or not more than about 4:1, or not more than about 3:1, or not more than about 2:1, or not more than about 1.5:1, or not more than about 1.25:1.

In some preferred embodiments, the oral dosage form of the present invention is directed to an oral dosage form comprising: (i) an opioid agonist and (ii) ADER, such that the ratio of the mean Tₘₐₓ of the opioid agonist after single dose oral administration of an immediate release reference product containing an equivalent amount of opioid agonist to the mean Tₘₐₓ of opioid agonist after single dose oral administration of an intact dosage form of the invention is at least about 1.25:1. In other embodiments of the invention, the mean Tₘₐₓ ratio using the aforementioned test method is at least about 1.5:1, or at least about 2:1, or at least about 3:1, or at least about 4:1, or at least about 5:1, or at least about 6:1, or at least about 10:1, or at least about 115:1 or at least about 20:1.

In some preferred embodiments, the invention is directed to an oral dosage form comprising (i) an opioid agonist and (ii) ADER, such that less than 70% of the opioid agonist is released from the intact dosage form after 1 hour based on the in-vitro dissolution of the dosage form in 900 mL of 40% ethanol in water using the USP Basket and Paddle Methods at 50 rpm and 37° C. In other embodiments of the invention, the release rate of the opioid agonist from the intact dosage form by the aforementioned USP basket method at 1 hours is 60% or less, 50% or less, 45% or less, 40% or less, 35% or less, 33% or less, 30% or less, 25% or less, 20% or less or 15% or less.

In certain preferred embodiments of the invention, the mean ratio of the amount of opioid agonist released from the dosage form after mechanical tampering (e.g., after crushing with a single crush of a spatula or in the case of a capsule containing a solid, cutting into two pieces) to the amount of opioid agonist released from the intact dosage form based on the dissolution at 0.5 hours of the dosage form in 900 mL of Simulated Gastric Fluid using the USP Basket and Paddle Methods at 50 rpm at 37 degrees ° C. is less than 20:1. In other embodiments of the invention, the mean ratio by the aforementioned USP basket method at 0.5 hours is 15:1 or less, 10:1 or less, 7.5:1 or less, 5:1 or less. 3:1 or less, 2:1 or less, 1.5:1 or less.

In certain preferred embodiments of the invention, the mean ratio of the amount of opioid agonist released from the dosage form after mechanical tampering (e.g., after crushing with a single crush of a spatula or in the case of a capsule containing a solid, cutting into two pieces) to the amount of opioid agonist released from the intact dosage form based on the dissolution at 1 hour of the dosage form in 900 mL of Simulated Gastric Fluid using the USP Basket and Paddle Methods at 50 rpm at 37 degrees ° C. is less than 20:1. In other embodiments of the invention, the mean ratio by the aforementioned USP basket method at 1 hour is 15:1 or less, 10:1 or less, 7.5:1 or less, 5:1 or less. 3:1 or less, 2:1 or less, 1.5:1 or less.

In certain preferred embodiments of the invention, the mean ratio of the amount of opioid agonist released from the dosage form after mechanical tampering (e.g., after crushing with a single crush of a spatula or in the case of a capsule containing a solid, cutting into two pieces) to the amount of opioid agonist released from the intact dosage form based on the dissolution at 2 hours of the dosage form in 900 mL of Simulated Gastric Fluid using the USP Basket and Paddle Methods at 50 rpm at 37 degrees ° C. is less than 20:1. In other embodiments of the invention, the mean ratio by the aforementioned USP basket method at 2 hours is 15:1 or less, 10:1 or less, 7.5:1 or less, 5:1 or less. 3:1 or less, 2:1 or less, 1.5:1 or less.

In some preferred embodiments, the present invention is directed to an oral dosage form comprising (i) an opioid agonist and (ii) ADER, such that the ratio of the mean Cₘₐₓ of the opioid agonist after single dose oral administration of the dosage form after tampering to the mean Cₘₐₓ of opioid agonist after single dose oral administration of an intact dosage form is less than about 20:1. In other embodiments of the invention, said mean ratio using the aforementioned test method is less than about 15:1 or less than about 10:1, or less than about 7:1, or less than about 5:1, or less than about 4:1, or less than about 3:1, or less than 2.5:1, or less than about 2:1, or less than about 1.75:1, or less than about 1.5:1, or less than about 1.25:1 or less than about 1.25:1

In some preferred embodiments, the present invention is directed to an oral dosage form comprising (i) an opioid agonist and (ii) ADER, such that the ratio of the mean AUC₀₋₂ of the opioid agonist after single dose oral administration of an immediate release dosage form containing an equivalent amount of opioid agonist to the mean AUC₀₋₂ of opioid agonist after single dose oral administration of an intact dosage form of the invention is at least 1.25:1. In other embodiments of the invention, the mean AUC₀₋₂ ratio using the aforementioned test method is at least about 1.5:1, or at least about 1.75:1, or at least about 2:1, or at least about 2.5:1, or at least about 3:1, or at least about 3.5:1, or at least about 4:1, or at least about 5:1, or at least about 6:1, or at least about 10:1 or at least about 15; 1 or at least about 20:1.

The invention is also directed to methods of preventing abuse and misuse of an opioid agonist utilizing the dosage forms disclosed herein. The method can comprise providing the opioid agonist in an oral dosage form together with ADER, wherein the opioid agonist is present in a form which is partially or substantially resistant to tampering (e.g., crushing, shear forces which break up the dosage form, solvent extraction, etc.).

In certain preferred embodiments of the invention, the release for the opioid agonist component of the formulation is expressed in terms of a ratio of the release achieved after tampering, relative to the amount released from the intact formulation. The ratio is therefore expressed as [Crushed]/[Whole], and it is desired that this ratio have a numerical range of not more than 20:1 (crushed release in 1 hour/intact release in 1 hour), based on in-vitro dissolution of the dosage form in 900 ml of Simulated Gastric Fluid using the USP Basket and Paddle Methods at 50 rpm and 37° C. In other embodiments of the invention, the mean ratio using the aforementioned test method is less than about 15:1, or less than about 10:1, or less than about 7:1, or less than about 5:1, or less than about 3:1, or less than about 2:1, or less than about 1.5:1, or less than about 1.25:1.

In certain preferred embodiments of the invention, the mean ratio of the time to confirmed perceptible pain relief after administration of the intact dosage form to the time to confirmed perceptible pain relief after administration of the tampered dosage form is less than 20:1. In other embodiments of the invention, the mean ratio using the aforementioned test method is less than about 15:1, or less than about 10:1, or less than about 7:1, or less than about 5:1, or less than about 3:1, or less than about 2:1, or less than about 1.5:1, or less than about 1.25:1.

In certain preferred embodiments of the invention, the mean ratio of the time to meaningful pain relief after administration of the intact dosage form to the time to meaningful pain relief after administration of the tampered dosage form is less than 20:1. In other embodiments of the invention, the mean ratio using the aforementioned test method is less than about 15:1, or less than about 10:1, or less than about 7:1, or less than about 5:1, or less than about 3:1, or less than about 2:1, or less than about 1.5:1, or less than about 1.25:1.

In certain preferred embodiments of the invention, the mean ratio of the peak pain intensity difference score after administration of the tampered dosage form to the peak pain intensity difference score after administration of the intact dosage form is less than 10:1. In other embodiments of the invention, the mean ratio using the aforementioned test method is less than about 8:1, or less than about 7:1, or less than about 5:1, or less than about 3:1, or less than about 2:1, or less than about 1.5:1, or less than about 1.25:1.

In certain preferred embodiments of the invention, the mean ratio of the peak pain relief score after administration of the tampered dosage form to the peak pain relief score after administration of the intact dosage form is less than 10:1. In other embodiments of the invention, the mean ratio using the aforementioned test method is less than about 8:1, or less than about 7:1, or less than about 5:1, or less than about 3:1, or less than about 2:1, or less than about 1.5:1, or less than about 1.25:1.

In certain preferred embodiments of the invention, the mean ratio of change from baseline to two hours post-dose in pain intensity score after administration of the tampered dosage form to the change from baseline to two hours post-dose in pain intensity score after administration of the intact dosage form is less than 20:1. In other embodiments of the invention, the mean ratio using the aforementioned test method is less than about 15:1, or less than about 10:1, or less than about 7:1, or less than about 5:1, or less than about 3:1, or less than about 2:1, or less than about 1.5:1, or less than about 1.25:1.

In certain preferred embodiments of the invention, the mean ratio of the number of patients with pain who need to be treated to obtain ≧50% pain relief in one patient (i.e., number needed to treat or NNT) one hour after administration of the tampered dosage form to the NNT one hour after administration of the intact dosage form is less than 20:1. In other embodiments of the invention, the mean ratio using the aforementioned test method is less than about 15:1, or less than about 10:1, or less than about 7:1, or less than about 5:1, or less than about 3:1, or less than about 2:1, or less than about 1.5:1, or less than about 1.25:1.

In certain preferred embodiments of the invention, the mean ratio of the number needed to harm (NNH) due to moderate or severe nausea in opioid naïve healthy subjects after administration of the tampered dosage form to the number needed to harm (NNH) due to moderate or severe nausea after administration of the intact dosage form is less than 20:1. In other embodiments of the invention, the mean ratio using the aforementioned test method is less than about 15:1, or less than about 10:1, or less than about 7:1, or less than about 5:1, or less than about 3:1, or less than about 2:1, or less than about 1.5:1, or less than about 1.25:1.

In certain preferred embodiments of the invention, the mean ratio of the number needed to harm (NNH) due to moderate or severe sedation or drowsiness in opioid naïve healthy subjects after administration of the tampered dosage form to the number needed to harm (NNH) due to moderate or severe sedation or drowsiness after administration of the intact dosage form is less than 20:1. In other embodiments of the invention, the mean ratio using the aforementioned test method is less than about 15:1, or less than about 10:1, or less than about 7:1, or less than about 5:1, or less than about 3:1, or less than about 2:1, or less than about 1.5:1, or less than about 1.25:1.

In certain preferred embodiments of the invention, the mean ratio of the drug liking score in drug abusers and recreational drug users without pain after administration of the tampered dosage form to the drug liking score after administration of the of the intact dosage form is less than 20:1. In other embodiments of the invention, the mean ratio using the aforementioned test method is less than about 15:1, or less than about 10:1, or less than about 7:1, or less than about 5:1, or less than about 3:1, or less than about 2:1, or less than about 1.5:1, or less than about 1.25:1.

In certain preferred embodiments of the invention, the mean ratio of the drug effect score in drug abusers and recreational drug users without pain after administration of the tampered dosage form to the drug effect score after administration of the intact dosage form is less than 20:1. In other embodiments of the invention, the mean ratio using the aforementioned test method is less than about 15:1, or less than about 10:1, or less than about 7:1, or less than about 5:1, or less than about 3:1, or less than about 2:1, or less than about 1.5:1, or less than about 1.25:1.

The invention is also directed to a method of treating or preventing diseases and disorders amenable to treatment with opioid agonists, including pain with the dosage forms disclosed herein. The method can comprise providing an oral dosage form containing an opioid agonist and ADER, said dosage form an immediate release formulation, an extended release formulation or a formulation comprising both immediate release and extended release.

The oral dosage form containing an opioid agonist in combination with ADER includes, but is not limited to tablets or capsules. The dosage forms of the present invention may include any desired pharmaceutical excipients known to those skilled in the art. The oral dosage forms may further provide an immediate release of the opioid agonist. In certain preferred embodiments, the oral dosage forms of the present invention provide a sustained release of the opioid agonist contained therein. Oral dosage forms providing sustained release of the opioid agonist may be prepared in accordance with formulations/methods of manufacture known to those skilled in the art of pharmaceutical formulation.

The benefits of the abuse-resistant dosage form are especially great in connection with oral dosage forms of potent opioid agonists, which can provide valuable therapeutic benefits but are prone to being abused. This is particularly true for sustained release opioid agonist products which have a large dose of a desirable opioid agonist intended to be released over a period of time in each dosage unit. Drug abusers take such sustained-release product and crush, grind, extract or otherwise damage the product so that the full contents of the dosage form become available for immediate absorption. Since such tampering of the dosage form of the invention results in the opioid agonist also becoming available for absorption, the present invention provides a means for deterring such abuse. In addition, the present invention addresses the risk of overdose to non-abusing patients from “dumping” effect of the full dose of the opioid agonist if the product is accidentally chewed or crushed or co-ingested with a significant amount of alcohol.

In certain preferred embodiments, a combination of two opioid agonists is included in the formulation with the ADER. In further embodiments, one or more opioid agonist and ADER are included and a further non-opioid drug is also included for the treatment of the same medical condition as the opioid agonist or for the treatment of a different medical condition.

Another embodiment of the invention is directed to a method of preventing or treating pain with the disclosed dosage forms. In certain preferred embodiments, the method of treating pain in patients with a dosage form having less abuse potential comprises providing an oral dosage form containing an opioid agonist and ADER; and orally administering the dosage form to provide a plasma level of opioid agonist greater than the minimum analgesic concentration of the opioid agonist.

Another embodiment of the invention is directed to a method of preventing or treating diseases and disorders amenable to treatment with opioid agonists with the disclosed dosage forms. In certain preferred embodiments, the method of preventing or treating such diseases and disorders in patients with a dosage form having less abuse potential comprises providing an oral dosage form containing an opioid agonist and ADER; and orally administering the dosage form to provide a plasma level of the opioid agonist greater than the minimum therapeutic concentration of the opioid agonist.

The invention is also directed to methods of preparing the dosage forms disclosed herein.

The benefits of the abuse-resistant dosage form are especially great in connection potent opioid agonists, which would provide valuable therapeutic benefits but would be prone to being abused. This is particularly true for oral dosage forms, including, in some preferred embodiments, sustained release dosage forms of opioid agonists which would have a large dose of a desirable opioid agonist intended to be released over a period of time in each dosage unit. Drug abusers may tamper the dosage form of the invention so that the full contents of the dosage form become available for immediate and maximal mood altering effects. The dosage form of the present invention would reduce the mood altering effects of the opioid agonists upon tampering and as such the invention provides pharmaceutical compositions, dosage forms and methods of deterring misuse, abuse, tampering and diversion of the dosage form.

In certain preferred embodiments, a combination of two opioid agonists is included in the dosage form. In further embodiments, one or more opioid agonist and a non-opioid drug are included for the treatment of the same medical condition as the opioid agonist or for the treatment of a different medical condition.

When the dosage form of the invention is tampered, the invention reduces the amount of opioid agonist released in immediate release form, which in turn reduces the euphoric, pleasurable, reinforcing, rewarding, mood altering and toxic effects of the opioid agonist of the dosage form.

When the dosage form of the present invention is orally administered as intended to humans, the opioid agonist is released into systemic circulation as intended and is therefore available for absorption into the body. However, if the dosage forms of the present invention is tampered (e.g., chemical, solvent, thermal or mechanical extraction, followed by administration into the body) the ADER of the invention would reduce the amount of opioid agonist available in immediate release form. Additionally, the dosage form of the invention substantially reduces the efficiency of drug aspiration into syringes, drug filtration after solvent extraction and drug extraction after attempts at chemical, mechanical or thermal extraction from both immediate and sustained release dosage form of the invention. These characteristic decrease the potential for abuse or diversion of the opioid agonist in the dosage form by blocking the mood altering, euphoric, pleasurable, reinforcing, rewarding or toxic effects of any co-abused opioid agonist.

The term “tampering” means any manipulation by mechanical, thermal and/or chemical means which changes the physical properties of the dosage form, e.g., to liberate the opioid agonist for immediate release if it is in sustained release form, or to make the opioid agonist available for inappropriate use such as administration by an alternate route, e.g., parenterally. The tampering can be, e.g., by means of crushing, shearing, grinding, mechanical extraction, solvent extraction, solvent immersion, combustion, heating or any combination thereof.

The term “abuse”, “opioid agonist abuse” or “opioid abuse” in the context of the present invention, when it refers to the effects of opioid agonists in causing such, includes intermittent use, recreational use and chronic use of opioid agonists alone or in conjunction with other drugs: (i) in quantities or by methods and routes of administration that do not conform to standard medical practice; (ii) outside the scope of specific instructions for use provided by a qualified medical professional; (iii) outside the supervision of a qualified medical professional; (iv) outside the approved instructions on proper use provided by the drug's legal manufacturer; (v) which is not in specifically approved dosage forms for medical use as pharmaceutical agents; (vi) where there is an intense desire for and efforts to procure same; (vii) with evidence of compulsive use; (viii) through acquisition by manipulation of the medical system, including falsification of medical history, symptom intensity, disease severity, patient identity, doctor shopping, prescription forgeries; (ix) where there is impaired control over use; (x) despite harm; (xi) by procurement from non-medical sources; (xii) by others through sale or diversion by the individual into the non-medical supply chain; (xiii) for medically unapproved or unintended mood altering purposes.

The term “mood altering” is defined for purposes of the present invention to mean that the “high”, “liking”, pleasurable, euphoric, calming, anxiolytic, auditory and visual perceptual alterations, relaxing, analgesic, psychotomimetic, rewarding, reinforcing and toxic effects of the co-abused opioid agonist.

The term “abuse resistant”, “abuse deterrent” and “deter abuse” are used interchangeably in the context of the present invention and include pharmaceutical compositions and methods that (i) resist, deter, discourage, diminish, delay and/or frustrate the intentional, unintentional or accidental physical manipulation or tampering of the dosage form (e.g., crushing, shearing, grinding, chewing, dissolving, melting, needle aspiration, inhalation, insufflation, extraction by mechanical, thermal and chemical means, and/or filtration); (ii) resist, deter, discourage, diminish, delay and/or frustrate the intentional, unintentional or accidental use or misuse of the dosage form outside the scope of specific instructions for use provided by a qualified medical professional, outside the supervision of a qualified medical professional and outside the approved instructions on proper use provided by the drug's legal manufacturer (e.g., intravenous use, intranasal use, inhalational use and oral ingestion to provide high peak concentrations); (iii) resist, deter, discourage, diminish, delay and/or frustrate the intentional, unintentional or accidental conversion of an extended release dosage form of the invention into a more immediate release form; (iv) resist, deter, discourage, diminish, delay and/or frustrate the intentional and iatrogenic increase in physical and psychic effects sought by recreational drug users, addicts, and patients with pain who have an addiction disorder; (v) resist, deter, discourage, diminish, delay and/or frustrate the attempts at surreptitious administration of the dosage form to a third party (e.g., in a beverage); (vi) resist, deter, discourage, diminish, delay and/or frustrate attempts to procure the dosage form by manipulation of the medical system and from non-medical sources; (vii) resist, deter, discourage, diminish, delay and/or frustrate the sale or diversion of the dosage form into the non-medical supply chain and for medically unapproved or unintended mood altering purposes; (viii) resist, deter, discourage, diminish, delay and/or frustrate intentional, unintentional or accidental attempts at otherwise changing the physical, pharmaceutical, pharmacological and/or medical properties of the dosage form from what was intended by the manufacturer.

As used herein, the term “ADER” or “abuse deterrent, extended release” refers to compounds selected from the group consisting of: (a) hydrogenated Type I or Type II vegetable oils; (b) polyoxyethylene stearates and distearates; (c) glycerol monostearate; (d) poorly water soluble, high melting point (mp=40 to 100° C.) waxes, and mixtures thereof.

As used herein, references to the term “the invention”, “the present invention”, “the pharmaceutical composition of the invention”, “the dosage form of the invention”, “the current invention” and embodiments of the invention in the embodiments, claims and specifications refer to pharmaceutical compositions, dosage forms, methods, processes and other innovations that comprise (i) one or more opioids agonists, in unsalified form or their pharmaceutically acceptable salts, prodrugs, esters, analogs, derivatives, solvates, complexes, polymorphs, hydrates and metabolites, as racemates or an individual diastereoisomers or enantiomeric isomers thereof or mixtures thereof; (ii) one or more compounds selected from the group consisting of: (a) hydrogenated Type I or Type II vegetable oils; (b) polyoxyethylene stearates and distearates; (c) glycerol monostearate; (d) poorly water soluble, high melting point (mp=40 to 100° C.) waxes, and mixtures thereof, said compounds also referred to as ADER; and optionally (iii to v), (iii) other non-opioid analgesics; and/or (iv) non-analgesic therapeutic agents; and/or (v) pharmaceutical excipients, adjuvants and auxiliary agents including binders, disintegrants, fillers, diluents, anti-adherents or glidants, lubricants, stabilizers, wetting agents, pharmaceutically compatible carriers and dissolution rate modifiers, and channel and pore formers.

In some preferred embodiments, the dosage form may optionally also contain hydrophobic polymers, hydrophilic polymers, gums, protein derived materials, other waxes, shellac, other oils and mixtures thereof.

In some preferred embodiments, the invention is directed at an opioid dosage form, said opioid form having flotation capabilities to deter surreptitious attempts at intoxication of another subject (e.g., in an alcoholic or non-alcoholic beverage).

In some preferred embodiments, the in vivo pharmacokinetic parameters of the specifications and claims are derived or determined from first administration. In other preferred embodiments, the in vivo pharmacokinetic parameters are derived or determined from steady state administration.

In some preferred embodiments, the in vivo pharmacokinetic parameters of the specifications and claims are derived or determined under fed conditions. In other preferred embodiments, the in vivo pharmacokinetic parameters are derived or determined under fasted conditions.

In some preferred embodiments, the in vivo pharmacokinetic parameters of the specifications and claims are derived or determined from an individual subject. In other preferred embodiments, the in vivo pharmacokinetic parameters are derived or determined from a population of subjects.

In some preferred embodiments, the in vivo pharmacokinetic parameters of the specifications and claims are derived or determined in subjects having a Body Mass Index (BMI) between 18 and 26 kg/m², inclusive (BMI=[weight in kg/height in m²]×10,000). In some preferred embodiments, the in vivo pharmacokinetic parameters of the specifications and claims are derived or determined in subjects having a Body Mass Index (BMI)≧38 kg/m².

Also disclosed are methods for preventing and treating pain in a human patient suffering comprising a therapeutically effective amount of oral opioids or pharmaceutically acceptable salts thereof or mixtures thereof.

All pain states are contemplated by this invention, regardless of etiology, mechanisms, duration, prior treatment response and anatomic location, including acute pain, inflammatory pain, chronic pain, cancer pain, visceral pain and neuropathic pain.

Also disclosed are methods of providing relief in a human patient suffering from neuropathic and chronic pain comprising a therapeutically effective amount of oral opioids or pharmaceutically acceptable salts thereof or mixtures thereof. In some preferred embodiments, the dosage form of the invention is intended for the treatment of neuropathic pain, peripheral neuropathic pain, central neuropathic pain, chronic pain, osteoarthritis, back pain, cancer pain, fibromyalgia, and chronic inflammatory pain.

Also disclosed are methods of providing relief in a human patient suffering from acute pain comprising a therapeutically effective amount of oral opioids or pharmaceutically acceptable salts thereof or mixtures thereof.

All kinds of kits of the present invention are contemplated. In some preferred embodiments, also provided are kits for use in treating or preventing the pain with the oral administration of opioid or pharmaceutically acceptable salts thereof or mixtures thereof for a subject in need of such treatment, comprising: (i) a dosage form of the invention; (ii) a container for the dosage form; and optionally, any of (iii) to (vi): (iii) a container for individual units of the dosage form (e.g., individual tablets or capsules in blisters); (iv) educational instructions in any media about various medical conditions, their etiology, pathophysiology, consequences and treatment, including information on the potential for abuse and diversion and methods for prevention of same and information on the proper use and disposal of the medication; (v) containers or bags for the safe disposal of any used or remaining unused dosage form, preferably child proof and flushable; (vi) tamper evident and child proof packaging for the kit and its contents.

The amount of opioid in the oral dosage form will vary depending on variety of physiologic, pharmacologic, pharmacokinetic, pharmaceutical and physicochemical factors, including: (i) the choice of opioid as the base, pharmaceutically acceptable salt or mixtures thereof; (ii) the nature of the oral dosage form (e.g., immediate release or extended release); (iii) the anatomical location of the pain relieving target; (iv) the intensity and intractability of the pain; (v) the contribution of different mechanism to the initiation, propagation, summation and maintenance of the pain; (vi) the absorption, metabolism, distribution and excretion of orally administered opioids in healthy subjects and in patients with various diseases and disorders, including renal and hepatic impairment; (vii) the presence of comorbid pathology; (viii) the patient's risk of iatrogenic side effects; (ix) the tolerability of the dose, including the patient's propensity for opioids associated side effects; (x) use of concurrent analgesics; (xi) the efficiency of the dosage form; (xii) the physicochemical properties of the opioid, including its solubility and hydrophilicity.

The invention is also directed to methods of preparing the dosage forms disclosed herein.

In certain preferred embodiments, the opioids in the dosage form is combined with one or more other drugs for the treatment of the same medical condition as the opioids or for the treatment of a different medical condition. All modes of co-administration are contemplated, including via an oral, subcutaneous, direct intravenous, slow intravenous infusion, continuous intravenous infusion, intravenous or epidural patient controlled analgesia (PCA and PCEA), intramuscular, intrathecal, epidural, intracisternal, intramuscular, intraperitoneal, transdermal, topical, transmucosal, buccal, sublingual, transmucosal, inhalation, intranasal, epidural, intra-articular, intranasal, rectal or ocular routes.

The term “first administration” means administration of a dose of the present invention at the initiation of therapy to an individual patient or a patient population.

The term “steady state” means that the amount of the drug reaching the system is approximately the same as the amount of the drug leaving the system. Thus, at “steady-state”, the patient's body eliminates the drug at approximately the same rate that the drug becomes available to the patient's system through absorption into the blood stream.

As used herein the terms: (i) “AUC₀₋ₜ” means area under the plasma drug concentration-time curve from time zero to the “t”, where t is the time point of the maximum intended dosing frequency of the dosage form (e.g., 4 hours, 6 hours, 8 hours, 12 hours or 24 hours for dosage forms intended to be administered every 4 hours, every 6 hours, every 8 hours, every 12 hours and every 24 hours, respectively, thereby providing an AUC₀₋ₜ time interval of 0 to 4 hours, 0 to 6 hours, 0 to 8 hours, 0 to 12 hours and 0 to 24 hours, respectively); (ii) “AUC_{0-∞}” means area under the plasma drug concentration-time curve from time zero to infinity; (iii) “AUC₀₋₈” means area under the plasma drug concentration-time curve from time zero to 8 hours after dosing; (iv) “AUC₀₋₁₂” means area under the plasma drug concentration-time curve from time zero to 12 hours after dosing; (v) “AUC₀₋₂₄” means area under the plasma drug concentration-time curve from time zero to 24 hours after dosing; (vi) “Cₘₐₓ” means the maximum observed plasma drug concentration; (vii) “C₈” means the plasma drug concentration at 8 hours after dosing; (viii) “C₁₂” means the plasma drug concentration at 12 hours after dosing; (ix) “C₂₄” means the plasma drug concentration at 24 hours after dosing; (x) “tₘₐₓ” or “Tₘₐₓ” means the time of the observed maximum drug concentration (also known as the time at which Cₘₐₓ occurs); (xi) “Cₘᵢₙ” means the minimum observed drug concentration following the maximum plasma concentration or the concentration at the end of the intended dosing interval; (xii) “time at which Cₘᵢₙ occurs” means the time at when the minimum observed drug concentration occurs; (xiii) “half value duration” or “HVD” means the duration over the dosing interval during which plasma concentration of drug are greater than or equal to one-half of Cₘₐₓ, obtained by calculating the time interval beginning when the interpolated concentration first equals or exceeds one-half of Cₘₐₓ and ending at the first time point for which the interpolated concentration falls below one-half of Cₘₐₓ; (xiv) “W₅₀” means the duration of the dosing interval over which the plasma concentrations are equal to or greater than 50% of the peak concentration; (xv) “steady state” is a state of equilibrium wherein the amount of the drug reaching the system is approximately the same as the amount of the drug leaving the system or put another way, the patient's body eliminates the drug at approximately the same rate that the drug becomes available to the patient's system through absorption into the blood stream, said “time to steady state” measured by calculating the Cₘᵢₙ after each sequential dosing of drug administered at the intended dosing frequency until two consecutive Cₘᵢₙ's are not statistically different at a 10% significance level (p=0.10); (xvi) “percent fluctuation” means the variation in plasma concentrations of the drug computed as: (a) (Cₘₐₓ−Cₘᵢₙ)/Cₘᵢₙ×100 (for an individual patient) and (mean Cₘₐₓ−mean Cₘᵢₙ)/mean Cₘᵢₙ×100 (for a population); or (b) (Cₘₐₓ−Cₘᵢₙ)/Cₐᵥ×100 (for an individual patient) and (mean Cₘₐₓ−mean Cₘᵢₙ)/mean Cₐᵥ×100 (for a population); (xvii) “accumulation index” or “AI” means the ratio of the plasma concentration of the drug at the end of the intended dosing interval (i.e., 8 hours for a Q8H dosage form, 12 hours for a Q12H dosage form, and 24 hours for a Q24H dosage form) after administration, determined at steady-state (Cₛₛₘᵢₙ) to the plasma concentration of the drug at the end of the intended dosing interval determined at first administration (i.e., after the first dose).

Pharmacokinetic parameters of the invention are be computed from first administration and steady state pharmacokinetic studies conducted in an individual subject or in a population of subjects in the fasted or fed states. The AI and percent of steady state computations requires both single dose (i.e., first administration) and steady state pharmacokinetic assessment.

In certain preferred embodiments of the present invention, an effective amount of opioid in immediate release form is included in the controlled release unit dose opioid formulation to be administered. The immediate release form of the opioid is preferably included in an amount which is effective to shorten the time to Cₘₐₓ or increase the magnitude of the Cₘₐₓ of the opioids in the blood (e.g., plasma). In such embodiments, an effective amount of the opioid in immediate release form may be coated onto the substrates of the present invention. For example, where the extended release opioid from the formulation is due to a controlled release coating, the immediate release layer would be overcoated on top of the controlled release coating. On the other hand, the immediate release layer may be coated onto the surface of substrates wherein the opioid is incorporated in a controlled release matrix. Where a plurality of the sustained release substrates comprising an effective unit dose of the opioid are incorporated into a hard gelatin capsule, the immediate release portion of the opioids dose may be incorporated into the gelatin capsule via inclusion of the sufficient amount of immediate release opioid as a powder or granulate within the capsule. Alternatively, the gelatin capsule itself may be coated with an immediate release layer of the opioids. In some other embodiments, the immediate release opioid is in liquid form, for example as a capsule within a capsule or as a liquid in contact with an extended release dosage form within a capsule. One skilled in the art would recognize still other alternative manners of incorporating the immediate release opioid into the unit dose. Such alternatives are deemed to be encompassed by the appended claims. By including such an effective amount of immediate release opioid in the unit dose, the experience of relatively higher levels of pain in patients may be significantly reduced.

For purposes of the invention, the term “a patient” in reference to pharmacokinetic parameters means that the discussion (or claim) is directed to the pharmacokinetic parameters of an individual patient or subject.

The term “population of patients” or “patient population” means that the discussion (or claim) is directed to the mean pharmacokinetic parameters of at least two patients or subjects.

In certain preferred embodiments, any one or all of the above in-vivo parameters are achieved after a first administration (often referred to as “single dose administration”) of the dosage form to a human patient or a population of human patients.

In certain alternative embodiments, any one or all of the above in-vivo parameters are achieved after steady state administration of the dosage form to a human patient or a population of human patients.

Perceptible Pain Relief, Confirmed Perceptible Pain Relief and Meaningful Pain Relief are assessed and defined as follows: At the time of dosing with the study medication, a trained member of study staff starts two stopwatches for each patient. The patient is instructed to stop the first stopwatch at the time of perceptible pain relief and the second stopwatch at the time when they first experience meaningful pain relief. The usual definitions of the perceptible and meaningful pain relief are as follows: Perceptible Pain Relief is when the patient begins to feel any pain relieving effect from the drug. The patient is typically instructed as follows: “I would like you to stop the first stopwatch when you first feel any pain relief whatsoever. This does not mean you feel completely better, although you might, but when you first feel any difference in the pain that you have had”. Meaningful Pain Relief is when the patient feels their pain relief is meaningful to them. The patient is typically instructed as follows: “I would like you to stop the second stopwatch when you have meaningful pain relief. That is, when the relief from the pain is meaningful to you”. Confirmed Perceptible Pain Relief is Perceptible Pain Relief in those patients who go on to also have Meaningful Pain Relief.

As used herein, “NNT” or “the number needed to treat” is the number of patients who need to be treated in order for one patient to obtain ≧50% pain relief or ≧50% reduction in pain intensity.

The “NNH” or “number needed to harm” is a measure that indicates how many patients would require a specific treatment to cause harm in one patient. As used herein, the “NNH or “number needed to harm” is a measure that indicates: (i) how many patients would require treatment to cause moderate or severe sedation (or drowsiness) in one patient, where moderate to severe sedation or drowsiness is defined as a VAS score of ≧50 mm on a 100 mm scale bounded on the left by “no sedation or drowsiness” and on the right by “extreme sedation or drowsiness” and (ii) how many patients would require treatment to cause moderate or severe nausea in one patient, where moderate to severe nausea is defined as a VAS score of ≧50 mm on a 100 mm scale bounded on the left by “no nausea” and on the right by “extreme nausea”

The “drug effects” questionnaire assesses the extent to which subjects currently felt a drug effect, on a scale of 1 to 5 (1=“I feel no effect from it at all”; 2=“I think I feel a mild effect, but I'm not sure”; 3=“I feel an effect, but it is not real strong”; 0.4=“I feel a strong effect”; 5=“I feel a very strong effect”). This questionnaire can be used to examine the overall drug effects of abusable drugs given intact and upon tampering, preferably in drug abusers and recreational drug users without pain.

The “drug liking” questionnaire assesses the extent to which subjects currently like the effects of the drug on a 100-mm VAS, bounded on the left by “0=dislike a lot”, bounded on the right by “100=like a lot”. This questionnaire can be used to examine the overall drug liking of abusable drugs given intact and upon tampering, preferably in drug abusers and recreational drug users without pain.

For the purposes of in vivo testing, unless specified otherwise, pain intensity is measured on a VAS or categorical scale. On the categorical scale, the patient is asked “My pain at this time is: None=0, Mild=1, Moderate=2, Severe=3. On the VAS, the patient is asked “My pain at this time is” (with VAS anchors: “No Pain” and “Extreme Pain”).

For the purposes of in vivo testing, unless specified otherwise, pain relief is measured on a categorical scale. The patient is asked “My relief from starting pain is: None=0, A little=1, Some=2, A lot=3, Complete=4.

In certain preferred embodiments, the amount of opioid in the dosage form is about 0.01 μg to 1500 mg. In other more preferred embodiments, the amount of opioid in the dosage form is about 0.1 μg to 1000 mg or about 0.1 μg to 1500 mg. In most preferred embodiments, the amount of opioid in the dosage form is about 0.01 μg to 750 mg or about 0.01 μg to about 500 mg or about 0.01 μg to about 250 mg or about 0.1 μg to about 500 mg or 0.1 μg to about 250 or about 0.1 μg to about 250 mg or about 1 μg to about 1500 mg or 1 μg to about 1000 mg or about 1 μg to about 100 mg or about 5 μg to about 1500 mg or about 5 μg to about 1000 mg or about 5 μg to about 500 mg or about 10 μg to about 1000 mg or about 10 μg to about 500 mg or about 100 μg to about 1000 mg.

In certain preferred embodiments, the amount of ADER in the claimed composition may be about 1 mg to 1500 mg. In most preferred embodiments, the amount of ADER in the claimed composition may be about 10 mg to 800 mg.

In certain preferred embodiments of the present invention, the ratio of the opioid agonist and the ADER is about 1:10,000 to about 10000:1 by weight, preferably about 1:1000 to about 1000:1 by weight, more preferably 1:250 to 250:1.

The term “USP Basket and Paddle Methods” is the Basket and Paddle Method described, e.g., in specified in the United States Pharmacopeia, USP-28 NF-23 (2005), published by the United States Pharmacopeial Convention, Inc, and herein incorporated by reference.

The term “pH-dependent” for purposes of the present invention is defined as having characteristics (e.g., dissolution) which vary according to environmental pH.

The term “pH-independent” for purposes of the present invention is defined as having characteristics (e.g., dissolution) which are substantially unaffected by pH.

The term “bioavailability” is defined for purposes of the present invention as the extent to which the drug (e.g., opioids) is absorbed from the unit dosage forms.

As used herein with respect to the opioids dosage forms of the invention, the term “oral”, “oral dosage form”, “oral pharmaceutical dosage form”, “oral administration”, and “oral route”, refer to any method of administration involving contact with the mouth and oral mucosa, including the ingestion of intact drugs (e.g., capsules, tablets, liquids swallowed whole), lingual, sublingual administration, buccal administration and transmucosal administration. Particularly preferred embodiments involve oral ingestion of intact drugs (e.g., capsules, tablets, liquids swallowed whole).

As used herein with respect to the opioids dosage forms of the invention, the term “oral”, “oral administration”, “oral dosage form”, “oral pharmaceutical composition” and “oral route” refer to any method of oral, lingual, sublingual, buccal administration, transmucosal administration and rectal routes of administration.

All oral pharmaceutical dosage forms of the invention are contemplated, including oral suspensions, tablets, capsules, lozenges, effervescent tablets, effervescent powders, powders, solutions, powders for reconstitution, transmucosal films, buccal products, oral mucoretentive products, oral gastroretentive tablets and capsules, orally disintegrating tablets, fast dissolving tablets, fast dispersing tablets, fast disintegrating dosage forms, administered as immediate release, modified release, enteric coated, sustained release, controlled release, pulsatile release and extended release dosage form.

As used herein, “controlled release” is interchangeable with “extended release”, “sustained release”, “modified release”, “delayed release” and the like. Such products provide a longer duration of action than conventional immediate release formulations of the same drugs and are usually administered every 8, 12 or 24 hours.

Controlled release dosage forms of the present invention release of opioid from the oral dosage form at slower rate than immediate release formulations. In some preferred embodiments, controlled release dosage forms of release opioids at such a rate that blood (e.g., plasma) concentrations (levels) or therapeutic effects are maintained within the therapeutic range (above the minimum effective therapeutic concentration) but below toxic levels for intended duration (e.g., over a period of 1 to 24 hours, preferably over a period of time indicative of Q4, Q6, Q8, Q12 or Q24H administration). Notwithstanding the foregoing, in some preferred embodiments, the controlled release formulations of the present invention provide therapeutic effects for a duration that is longer or substantially longer than the duration of meaningful or detectable plasma concentrations of opioid. Controlled release dosage forms can also involve PRN administration, e.g., Q3 PRN, Q4 PRN, Q6 PRN, Q8 PRN, Q12 PRN or Q24H PRN administration.

The term “immediate release opioid” for purposes of the present invention, is opioid for oral administration in a dosage form which formulated to release the active drug from the dosage form immediately (i.e., without an attempt to delay or prolong the release of the active drug from the dosage form as is the case for extended release dosage forms). In the absence of a commercially available oral immediate release opioid product, an available parenteral formulation of opioid or a salt thereof may be used orally or a solution of opioid or a salt thereof may be prepared for the purpose of in vivo testing requiring immediate release opioid.

For purposes of the invention, the controlled release formulations disclosed herein and the immediate release control formulations are dose proportional. In such formulations, the pharmacokinetic parameters (e.g., AUC and Cₘₐₓ) increase linearly from one dosage strength to another. Therefore the pharmacokinetic parameters of a particular dose can be inferred from the parameters of a different dose of the same formulation.

The term “agonist” means a ligand that binds to a receptor and alters the receptor state resulting in a biological response. Conventional agonists increase receptor activity, whereas inverse agonists reduce it (See Neubig et al, IUPHAR Committee on Receptor Nomenclature and Classification, Pharmacol Rev, 2003; Howlett et al., Mol Pharmacol, 1988).

The term “opioid agonist” means a molecule that causes a specific physiologic, pathophysiologic or pharmacologic effect after binding to an opioid receptor.

An “antagonist” is a drug or ligand that reduces the action of another drug or ligand, generally an agonist. Many antagonists act at the same receptor macromolecule as the agonist. (See Neubig et al, IUPHAR Committee on Receptor Nomenclature and Classification, Pharmacol Rev, 2003; Howlett et al., Mol Pharmacol, 1988).

The term “receptor” means a molecule within a cell, on a cell surface, on a membrane, in tissue, in fluid or otherwise found in humans that serve as a recognition or binding site to cause specific physiologic, pathophysiologic or pharmacologic effects. The term “receptor” also means a cellular macromolecule, or an assembly of macromolecules, that is concerned directly and specifically in chemical signaling between and within cells. Combination of a hormone, neurotransmitter, drug, ligand, or intracellular messenger with its receptor(s) initiates a change in cell function (Neubig et al, IUPHAR Committee on Receptor Nomenclature and Classification, Pharmacol Rev, 2003).

The term “opioid receptor” includes mu (μ), delta (δ), kappa (κ) and FQ (N/OFQ) peptide (NOP) receptors, their subtypes and splice variants such as μ₁, μ₂, δ₁, δ₂, κ₁, κ₂ and κ₃, etc, regardless of whether they also bind to or influence other receptor systems (e.g., norepinephrine reuptake inhibition, serotonin reuptake inhibition, NMDA receptor antagonism).

Opioid antagonists are known or readily determined by individuals who practice the art. Preferably, the opioid antagonists useful for the present invention may be selected from the group consisting of naltrexone, methylnaltrexone, nalbuphine, naloxone, nalmefene, cyclazocine, cyclorphan, oxilorphan nalorphine, nalorphine dinicotinate, nalmefene, nadide and levallorphan.

For the purposes of this invention, the term “opioid” is interchangeable with the term “opioid agonist”, except when there is a specific reference to an opioid antagonist.

Opioid agonists) include alfentanil, allylprodine, alphaprodine, anileridine, apomorphine, apocodeine, benzylmorphine, bezitramide, brifentanil, buprenorphine, butorphanol, carfentanil, clonitazene, codeine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, mirfentanil, morphine, myrophine, nalbuphine, narceine, nicomorphine, norlevorphanol, normethadone, nalorphine, nociceptin/orphanin FQ (N/OFQ), normorphine, norpipanone, ohmefentanyl, opium, oxycodone, oxymorphone, papavereturn, pentazocine, phenadoxone, phenomorphan, phenazocine, phenoperidine, pholcodine, piminodine, piritramide, propheptazine, promedol, profadol, properidine, propiram, propoxyphene, remifentanil, sufentanil, tapentadol, tramadol, trefentanil, tilidine, nalbuphine, or an opioid agonist having the same pentacyclic nucleus as nalmefene, naltrexone, buprenorphine, levorphanol, meptazinol, pentazocine, dezocine, any opioid having agonist activity at an opioid receptor belonging to the phenanthrene, morphinan, benzomorphan, methadone, phenylpiperidine, propionanilide 4-anilidopiperidine, 4-aryl piperidines, and 4-Heteroarylpiperidines class, any opioid having agonist activity at an opioid receptor having the same pentacyclic nucleus as nalmefene, naltrexone, buprenorphine, levorphanol, meptazinol, pentazocine and dezocine, any opioid having agonist activity at an opioid receptor which is a fentanyl analog, or their pharmaceutically acceptable salts, prodrugs, esters, analogs, derivatives, solvates, complexes, polymorphs, hydrates and metabolites, as racemates or an individual diastereoisomers or enantiomeric isomers thereof or mixtures thereof.

The term “opioid” with respect to the dosage form of the invention refers to drugs having opioid agonist properties.

In a preferred embodiment, the opioid of the invention is selected from a group consisting of alfentanil, anileridine, buprenorphine, brifentanil, butorphanol, carfentanil, codeine, dextromoramide, dezocine, dihydrocodeine, dihydromorphine, fentanyl, heroin, hydrocodone, hydromorphone, hydroxypethidine, isomethadone, ketobemidone, levorphanol, levomethadone, lofentanil, meperidine, meptazinol, metazocine, methadone, 4-methoxymethylfentanyl, 3-methylfentanil, metopon, mirfentanil, morphine, morphine-6-glucuronide, nalbuphine, norlevorphanol, normethadone, ohmefentanyl, opium, oxycodone, oxymorphone, pentazocine, phenazocine, propiram, propoxyphene, remifentanil, sufentanil, tapentadol, trefentanil, tramadol, tilidine, any opioid having agonist activity at an opioid receptor belonging to the phenanthrene, morphinan, benzomorphan, methadone, phenylpiperidine, propionanilide 4-anilidopiperidine, 4-aryl piperidines, and 4-Heteroarylpiperidines class, any opioid having agonist activity at an opioid receptor having the same pentacyclic nucleus as nalmefene, naltrexone, buprenorphine, levorphanol, meptazinol, pentazocine and dezocine, any opioid having agonist activity at an opioid receptor which is a fentanyl analog, or their pharmaceutically acceptable salts, prodrugs, esters, analogs, derivatives, solvates, complexes, polymorphs, hydrates and metabolites, as racemates or an individual diastereoisomers or enantiomeric isomers thereof or mixtures thereof.

The present invention anticipates the use of more than one opioid in some embodiments, given in the same formulation or in a different formulation, for use to treat, prevent or ameliorate the same disease or a different disease.

In certain preferred embodiments of the present invention, the invention allows for the use of lower doses of opioid by virtue of the inclusion or co-administration of an additional drug for the prevention or treatment of pain. By using lower amounts of either or both drugs, the side effects associated with treatment in humans are reduced.

The term “opioid” means an opioid base, a pharmaceutically acceptable salt, prodrugs, esters, analogs, derivatives, solvates, complexes, polymorphs, hydrates and metabolites, as racemates or an individual diastereoisomers or enantiomeric isomers thereof or mixture thereof.

The singular forms “a,” “an” and “the” include plural referents unless the context clearly dictates otherwise. Thus, for example, reference to “ADER” includes an ADER compound as well as a mixture of two or more different ADER compounds, reference to “opioid” includes an opioid as well as two or more different opioids in combination, and the like.

As used herein, the term “pain” includes: (i) peripheral neuropathic pain, e.g., acute and chronic inflammatory demyelinating polyradiculopathy, alcoholic polyneuropathy, chemotherapy-induced polyneuropathy, complex regional pain syndrome (CRPS) Type I and Type II, entrapment neuropathies (e.g., carpal tunnel syndrome), HIV sensory neuropathy, iatrogenic neuralgias (e.g., postthoracotomy pain, postmastectomy pain), idiopathic sensory neuropathy, painful diabetic neuropathy, phantom limb pain, postherpetic neuralgia, trigeminal neuralgia, radiculopathy (e.g., cervical thoracic, lumbosacral), sciatica, acute herpes zoster pain, temporomandibular joint disorder pain and postradiation plexopathy; and (ii) central neuropathic pain, e.g., compressive myelopathy from spinal stenosis, HIV myelopathy, multiple sclerosis pain, Parkinson's disease pain, postischemic myelopathy, post postradiation myelopathy, poststroke pain, posttraumatic spinal cord injury and syringomyelia; and (iii) cancer associated neuropathic pain, e.g., chemotherapy induced polyneuropathy, neuropathy secondary to tumor infiltration or nerve compression, phantom breast pain, postmastectomy pain, postradiation plexopathy and myelopathy; (iv) chronic pain, e.g., back pain, rheumatoid arthritis, osteoarthritis, inflammatory pain, non-inflammatory pain, myofascial pain, fibromyalgia, cancer pain, visceral pain, somatic pain, pelvic pain, musculoskeletal pain, post-traumatic pain, bone pain and idiopathic pain; (v) acute pain, e.g., acute postsurgical pain (including laparoscopic, laparotomy, gynecologic, urologic, cardiothoracic, arthroscopic, gastrointestinal, neurologic, orthopedic, oncologic, maxillofacial, ophthalmic, otolaryngologic, soft tissue, plastic, cosmetic, vascular and podiatric surgery, including abdominal surgery, abdominoplasty, adenoidectomy, amputation, angioplasty, appendectomy, arthrodesis, arthroplasty, arthroscopy, bilateral cingulotomy, biopsy, brain surgery, breast biopsy, cauterization, cesarean section, cholecystectomy, circumcision, commissurotomy, cordotomy, corneal transplantation, cricothoracotomy, discectomy, diverticulectomy, episiotomy, endarterectomy, endoscopic thoracic sympathectomy, foreskin restoration, fistulotomy, frenectomy, frontalis lift, fundectomy, gastrectomy, grafting, heart transplantation, hemicorporectomy, hemorrhoidectomy, hepatectomy, hemia repair, hypnosurgery, hysterectomy, kidney transplantation, laminectomy, laparoscopy, laparotomy, laryngectomy, lithotripsy, lobotomy, lumpectomy, lung transplantation, mammectomy, mammoplasty, mastectomy, mastoidectomy, mentoplasty, myotomy, mryingotomy, nephrectomy, nissen fundoplication, oophorectomy, orchidectomy, parathyroidectomy, penectomy, phalloplasty, pneumotomy, pneumonectomy, prostatectomy, psychosurgery, radiosurgery, ritidoplasty, rotationplasty, sigmoidostomy, sphincterotomy, splenectomy, stapedectomy, thoracotomy, thrombectomy, thymectomy, thyroidectomy, tonsillectomy, tracheotomy, tracheostomy, tubal ligation, ulnar collateral ligament reconstruction, ureterosigmoidostomy, vaginectomy, vasectomy, vulvectomy; renal colic; incisional pain; inflammatory incisional pain; nociceptive incisional pain; acute neuropathic incisional pain following surgery), renal colic, trauma, acute back pain, burn pain, burn dressing change pain, migraine pain, tension headache pain, acute musculoskeletal pain, acute exacerbation or flare of chronic back pain, acute exacerbation or flare of osteoarthritis, acute exacerbation or flare of chronic pain, breakthrough chronic non-cancer pain, breakthrough cancer pain, acute exacerbation or flare of fibromyalgia, acute exacerbation or flare of rheumatoid arthritis, acute exacerbation or flare of myofascial pain, acute exacerbation or flare of chronic idiopathic pain, acute exacerbation or flare of neuropathic pain, procedure related pain (e.g., arthroscopy, laparoscopy, endoscopy, intubation, bone marrow biopsy, soft tissue biopsy, catheterization), and other self-limiting pain states.

As used herein, the term “acute pain” refers to self-limiting pain that subsides over time and usually lasting less that about 30 days and more preferably lasting less than about 21 days. Acute pain does not include chronic conditions such as chronic neuropathy, chronic neuropathic pain and chronic cancer and non-cancer pain.

As used herein, “neuropathic pain” is pain initiated or caused by a primary lesion or dysfunction of the nervous system and includes (i) peripheral neuropathic pain and (ii) central neuropathic pain.

As used herein, the term “chronic pain” includes all non-neuropathic pain lasting more than 30 days, including inflammatory pain, non-inflammatory pain, muscle pain, joint pain, fascia pain, visceral pain, bone pain and idiopathic pain.

The term “analgesic effectiveness” is defined for purposes of the present invention as a satisfactory prevention, reduction in or elimination of pain, along with a tolerable level of side effects, as determined by the human patient.

The term “therapeutic effectiveness” is defined for purposes of the present invention as a satisfactory prevention, reduction in or elimination of neuropathy, pain and chronic pain, along with a tolerable level of side effects, as determined by the human patient.

The term “abuse resistant” and “abuse deterrent” are used interchangeably

“Drug”, “drug substance”, “substance”, “therapeutic agent”, “pharmacological agent”, “pharmaceutical agent”, “active agent” and “agent” are used interchangeably and are intended to have their broadest interpretation as to any therapeutically active substance which is delivered to a living organism to produce a desired, usually beneficial effect. In general, this includes therapeutic agents in all of the major therapeutic areas.

The term “subject” for purposes of treatment is used interchangeably with “patient”, “male”, “female”, and includes any human subject.

“Pharmaceutically or therapeutically acceptable excipient or carrier” or “excipient” refers to a substance which does not interfere with the effectiveness or the biological activity of the active ingredients and which is not toxic to the subject. In some preferred embodiments of the present invention, pharmaceutically or therapeutically acceptable excipients or carriers may play a role in imparting or optimizing the rate and extent of absorption of opioid or additional drugs in the pharmaceutical composition. In some preferred embodiments of the present invention, pharmaceutically or therapeutically acceptable excipients or carriers may play a role in stabilizing the opioids or additional drugs in the pharmaceutical composition.

In certain preferred embodiments of the present invention, the dosage form may include, in addition to opioids or a pharmaceutically acceptable salt thereof and ADER, other abuse deterrent or abuse resistant substances, process or technologies known in the art, including aversive agents. All kinds of aversive agents are contemplated, including, without limitation, opioid antagonists, laxatives, flushing agents, emetics, emetogenic compound, nausea producing compounds, drugs that cause burning on irritation when in contact with tissue or mucous membranes, drugs that precipitate withdrawal effects. Such aversive agents may be in a releasable, partially releasable or a non-releasable form, the latter being released on tampering the dosage form (e.g., mechanical, thermal, chemical, solvent tampering, ingestion in ways not recommended, and the like).

In one preferred embodiment of the invention, the dosage form includes both an immediate release and extended release component.

In one preferred embodiment of the invention, the dosage form includes a capsule within a capsule, each capsule containing a different drug or the same drug intended for treating the same or a different malady. In some preferred embodiments, the outer capsule may be an enteric coated capsule or a capsule containing an immediate release formulation to provide rapid plasma concentrations or a rapid onset of effect or a loading dose and the inner capsule contains an extended release formulation. In some preferred embodiments, up to 3 capsules within a capsule are contemplated as part of the invention. In one preferred embodiment of the invention, the dosage form involves one or more tablets within a capsule, wherein the opioid is either in the tablet and/or in one of the capsules.

In one preferred embodiment of the invention, the formulation is ingested orally as a tablet or capsule, preferably as a capsule. In another preferred embodiment of the invention, the formulation is administered bucally. In yet another preferred embodiment of the invention, the formulation is administered sublingually.

“Therapeutically effective amount” or “therapeutically-effective” refers to the amount of an active agent sufficient to induce a desired biological result. That result may be alleviation of the signs, symptoms, or causes of a disease, or any other desired alteration of a biological system.

The term “effective amount” means the quantity of a compound according to the invention necessary to prevent, to cure, or at least partially arrest a symptom for which the opioids has been prescribed to a subject.

The term “pharmaceutically acceptable salt” as used herein refers to a salt which is toxicologically safe for human and animal administration. Nonlimiting examples of salts include hydrochlorides, hydrobromides, hydroiodides, sulfates, bisulfates, nitrates, citrates, tartrates, bitartrates, phosphates, malates, maleates, napsylates, fumarates, succinates, acetates, terephthalates, pamoates and pectinates.

It is contemplated that the present invention may be used alone or in combination with other drugs to provide additive, complementary, or synergistic therapeutic effects or for the treatment of entirely different medical conditions.

Other pharmaceutically active ingredients from various therapeutic classes may also be used in combination with the present invention. They include, but are not limited to decongestants, analgesics, analgesic adjuvants, antihistamines, expectorants, antitussives, diuretics, anti-inflammatory agents, antipyretics, antirheumatics, antioxidants, laxatives, proton pump inhibitors, motility modifying agents, vasodilators, inotropes, beta blockers, beta adrenergic agonists, drugs to treat asthma and COPD, antiinfectives, antihypertensives, antianginal agents, anticoagulants, lipid and cholesterol lowering drugs, anti-diabetic drugs, hormones, smooth muscle relaxants, skeletal muscle relaxants, bronchodilators, vitamins, trace minerals, amino acids, and biological peptides. The drug being used in combination therapy with the present invention can be administered by any route, including parenterally, orally, topically, transdermally, sublingually, and the like.

The terms “medical condition”, “malady”, “disease”, “disorder” and “pathological states” are used interchangeably and are intended to have their broadest interpretation to refer to any physiologic, pathologic or pathophysiologic state in a human that can be prevented, treated, managed or altered to produce a desired, usually beneficial effect.

In some preferred embodiments, the oral opioid is intended to prevent or treat pain. A co-administered drug (in the same or different dosage form, by any route of administration) may be used to provide additive, complementary, superadditive or synergistic therapeutic analgesic effects, including other NSAIDs, NO-NSAIDs, COX-2 selective inhibitors, acetaminophen, nitroparacetamol, nitric oxide donors, beta adrenergic agonists, alpha-2 agonists, selective prostanoid receptor antagonists, cannabinoid agonists, opioid receptor agonists, NMDA receptor antagonists, gabapentin, pregabalin, gabapentinoids, neuronal nicotinic receptor agonists, calcium channel antagonists, sodium channel blockers, superoxide dismutase mimetics, p38 MAP kinase inhibitors, TRPV1 agonists, dextromethorphan, dextrorphan, ketamine, glycine receptor antagonists and antiepileptics and any other drugs that can be shown by a person proficient in the art to prevent or treat pain.

In other preferred embodiments, particularly preferred combinations include opioids with acetaminophen.

In other preferred embodiments, particularly preferred combinations include opioids with an NSAID. Nonsteroidal anti-inflammatory drugs typically have analgesic, anti-inflammatory, and antipyretic properties. Their mode of action appears to involve inhibition of cyclooxygenases (COX-1 and COX-2), leukotriene biosynthesis, and antibradykinin activity. NSAIDs may be non-selective (inhibit COX-1 and COX-2 isozymes) or COX-2 selective (preferentially inhibit the COX-2 isozymes). Non-limiting examples of NSAIDs or COX-2 selective inhibitor include ibuprofen, tiaprofenic acid, diclofenac, piroxicam, loxoprofen, fenoprofen, indoprofen, oxaprozin, tenoxicam, lomoxicam, acetylsalicylic acid, mefenamic acid, naproxen, flurbiprofen, flubufen, ketoprofen, indoprofen, carprofen, pramoprofen, muroprofen, trioxaprofen, aminoprofen, tiaprofenic acid, fluprofen, niflumic acid, tolfenamic acid, diflunisal, etodolac, fenbufen, indomethacin, isoxicam, sudoxicam, pirprofen, sulindac, tolmetin, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflunisal, flufenisal, meloxicam and nabumetone, celecoxib, valdecoxib, etoricoxib, rofecoxib, and lumiracoxib, and as well as their pharmaceutically acceptable salts, prodrugs, esters, analogs, derivatives, solvates, complexes, polymorphs, hydrates and metabolites, as racemates or an individual diastereoisomers or enantiomeric isomers thereof or mixture thereof.

In other preferred embodiments, particularly preferred combinations include opioids with NMDA antagonists.

In other preferred embodiments, particularly preferred combinations include opioids with antiepileptics.

Non-limiting examples of anti-epileptic compounds include gabapentin, pregabalin, carbamazepine, oxcarbazepine, lamotrigine, phenyloin, fosphenyloin, valproate, valproic acid, tiagabine, topiramate, divalproex, harkoseride, and levetiracetam, in unsalified form or as pharmaceutically acceptable salts, prodrugs, esters, analogs, derivatives, solvates, complexes, polymorphs, hydrates and metabolites, as racemates or an individual diastereoisomers or enantiomeric isomers thereof or mixture thereof.

In other preferred embodiments, particularly preferred combinations include opioids with antidepressants.

Antidepressants are well known in the art. Non-limiting examples of antidepressants include drugs from the following classes: tricyclic antidepressants, tetracyclic antidepressants, SRI's, SSRI's, SNRI's and NSRI's. Non-limiting examples of specific antidepressants include amitriptyline, bupropion, citalopram, protriptyline, nortriptyline, desipramine, doxepin, imipramine, clomipramine, fluoxetine, paroxetine, sertraline, venlafaxine, duloxetine, trazodone, nefazodone, maprotiline and mirtazapine in unsalified form or as pharmaceutically acceptable salts, prodrugs, esters, analogs, derivatives, solvates, complexes, polymorphs, hydrates and metabolites, as racemates or an individual diastereoisomers or enantiomeric isomers thereof or mixture thereof.

In other preferred embodiments, particularly preferred combinations include opioids with calcium channel blockers.

In other preferred embodiments, particularly preferred combinations include opioids with sodium channel modulators.

In other preferred embodiments, particularly preferred combinations include opioids with cannabinoid agonists. The term “cannabinoid agonist” means a substance that binds to one or more cannabinoid receptor to exert an agonist or partial agonist effect. A number of assays are available to determine whether a drug is a cannabinoid agonist, using in vivo and in vitro bioassay systems (Howlett et al., Mol Pharmacol, 1988; International Union of Pharmacology [IUPHAR], http://www.iuphar.org/index.html; Subcommittees on Cannabinoid Receptors The International Committee of Pharmacology Committee on Receptor Nomenclature and Classification [NC-IUPHAR], http://www.iuphar.org/nciuphar.html).

The term “cannabinoid receptor” means a molecule that causes a specific physiologic, pathophysiologic or pharmacologic effect after binding to CB₁, CB₂, non-CB₁/CB₂ cannabinoid sites, TRPV₁ receptors, as well as other G protein-coupled receptors (GPCRs) that form part of the endocannabinoid system (Wiley and Martin, Chemistry Physics of Lipids, 2002; Begg et al., Pharmacol Ther, 2005; Howlett et al., Neuropharmacol, 2004; Pertwee, AAPS Journal, 2005; International Union of Pharmacology (IUPHAR) Receptor Database; Howlett et al., Mol Pharmacol, 1988; International Union of Pharmacology [IUPHAR], http://www.iuphar.org/index.html; Subcommittees on Cannabinoid Receptors The International Committee of Pharmacology Committee on Receptor Nomenclature and Classification [NC-IUPHAR], http://www.iuphar.org/nciuphar.html).

Notwithstanding the above definitions, for the purposes of the present invention, drugs that enhance the effect of cannabinoid agonists by inhibiting their metabolism or reuptake (for example, anandamide amidase inhibitors) are also considered to be cannabinoid agonists.

In other preferred embodiments, particularly preferred combinations include opioids with muscle relaxants, including cyclobenzaprine.

In other preferred embodiments, particularly preferred combinations include opioids with drugs selected from the class of benzodiazepine agonists. Benzodiazepine agonist are known or readily determined by individuals who practice the art. All benzodiazepine agonists are contemplated by the invention, including benzodiazepine BZ1 (omega 1) receptor agonists. Preferably, the benzodiazepine agonist useful for the present invention may be selected from the group consisting of alprazolam, bromazepam, brotizolam, camazepam, chlordiazepoxide, cinolazepam, clobazam, clonazepam, clorazepate, desalkylflurazepam, diazepam, estazolam, flunitrazepam, flurazepam, halazepam, indiplon, ketazolam, loprazolam, lorazepam, lormetazepam, medazepam, metaclazepam, midazolam, nitrazepam, nordazepam, oxazepam, phenazepam, pinazepam, prazepam, quazepam, temazepam, tetrazepam, triazolam, zaleplone, zolpidem and zopiclone.

The term “benzodiazepine agonist” means a substance that binds to one or more benzodiazepine receptors or recognition sites, their subtypes and splice variants to exert an agonist or partial agonist effect.

For the purposes of the present invention, the term “benzodiazepine receptor” or “benzodiazepine recognition site” includes one or more benzodiazepine receptors or recognition sites, the benzodiazepine BZ1 (omega 1) receptor, the gamma-aminobutyric acid (GABA)-benzodiazepine receptor complex, the gamma-aminobutyric acid type A (GABAA) receptor and their respective subtypes and splice variants.

Compositions and methods of the present invention provide (i) abuse deterrence; (ii) extended release; and (iii) simultaneous abuse deterrence and extended release, prepared using compounds selected from the group consisting of: (a) hydrogenated Type I or Type II vegetable oils; (b) polyoxyethylene stearates and distearates; (c) glycerol monostearate; (d) poorly water soluble, high melting point (mp=40 to 100° C.) waxes, and mixtures thereof, said compounds hereinafter referred to as “abuse deterrent, extended release” or “ADER”.

In a most preferred embodiment, the dosage form includes more than one ADER selected from the group consisting of (a) hydrogenated Type I or Type II vegetable oils; (b) polyoxyethylene stearates and distearates; (c) glycerol monostearate; and (d) poorly water soluble, high melting point (mp=40 to 100° C.) waxes.

Hydrogenated vegetable oils of the present invention may include hydrogenated cottonseed oil (e.g., Akofine®; Lubritab®; Sterotex® NP), hydrogenated palm oil (Dynasan® P60; Softisan® 154), hydrogenated soybean oil (Hydrocote®; Lipovol HS-K®; Sterotex® HM) and hydrogenated palm kernel oil (e.g., Hydrokote® 112).

Polyoxyethylene stearates and distearates of the present invention include Polyoxyl 2, 4, 6, 8, 12, 20, 30, 40, 50, 100 and 150 stearates (e.g., Hodag® DGS; PEG-2 stearate; Acconon® 200-MS; Hodag® 20-S; PEG-4 stearate; Cerasynt® 616; Kessco® PEG 300 Monostearate; Acconon® 400-MS; Cerasynt® 660; Cithrol® 4MS; Hodag® 60-S; Kessco® PEG 600 Monostearate; Cerasynt® 840; Hodag® 100-S; Myrj® 51; PEG-30 stearate; polyoxyethylene (30) stearate; Crodet® S40; E431; Emerest® 2672; Atlas G-2153; Crodet® S50) and polyoxyl 4, 8, 12, 32 and 150 distearates (e.g., Lipo-PEG® 100-S; Myrj® 59; Hodag® 600-S; Ritox® 59; Hodag® 22-S; PEG4 distearate; Hodag® 42-S; Kessco® PEG 400 DS; Hodag® 62-S; Kessco® PEG 600 Distearate; Hodag® 154-S; Kessco® PEG 1540 Distearate; Lipo-PEG® 6000-DS; Protamate® 6000-DS).

In one preferred embodiment of the present invention, the opioid is combined with beeswax, hydroxypropyl methyl cellulose (e.g., HPMC K15M), silicon dioxide (alone or in combination with Al₂O₃; e.g., Aerosil®, Aerosil® 200, Aerosil® COK84).

In one embodiment of the present invention, the opioid is combined with hydrogenated cottonseed oil (e.g., Sterotex® NF), hydroxypropyl methyl cellulose (e.g., HPMC K15M), coconut oil and silicon dioxide (alone or in combination with Al₂O₃; e.g., Aerosil®, Aerosil® 200, Aerosil® COK84).

In another preferred embodiment of the present invention, the opioid is combined with glycerol monostearate (e.g., Cithrol® GMS), hydroxypropyl methyl cellulose (e.g., HPMC K100M) and silicon dioxide (alone or in combination with Al₂O₃; e.g., Aerosil®, Aerosil® 200, Aerosil® COK84).

In yet another preferred embodiment of the present invention, the opioid is combined with hydrogenated palm kernel oil (e.g., Hydrokote® 112), hydroxypropyl methyl cellulose (e.g., HPMC K15M) and silicon dioxide (alone or in combination with Al₂O₃; e.g., Aerosil®, Aerosil® 200, Aerosil® COK84).

In one preferred embodiment of the present invention, release rate modifiers, including hydroxypropyl methyl cellulose (e.g., HPMC K15M) may be incorporated. Release rate modifiers can also have additional useful properties that optimize the formulation.

A variety of agents may be incorporated into the ADER invention as thixotropes (e.g., fumed silicon dioxides, Aerosil®, Aerosil® COK84, Aerosil® 200, etc.). Thixotropes enhance the pharmaceutical formulations of the invention by increasing the viscosity of solutions during attempted extraction, complementing the action of HPMCs. They may also provide a tamper resistance by helping to retain the structure of dosage units that have been heated to temperatures greater than the melting point of the base excipient (Aerosils are unaffected by heat).

As described above, the present invention can include one or more ADER agents. Any amount of ADER may be used. In some embodiments, the total amount of ADER agent is about 5 to about 98 percent, preferably 7 to 90 percent and more preferably 10 to 85 percent on a dry weight basis of the composition.

Upon contact with a solvent (e.g., water), the ADER agents absorb the solvent and swell, thereby forming a viscous or semiviscous substance that significantly reduces and/or minimizes the amount of free solvent which can contain an amount of solubilized drug. This can also reduce the overall amount of drug extractable with solvent by entrapping the drug in a matrix.

In one preferred embodiment, the ADER can prevent less than or equal to about 98%, 90%, 80% 75%, 60%, 50%, 45%, 40%, 33%, 30%, 25%, 15%, 10%, 8%, 5%, or 2% of the total amount of drug in a dosage form from being recovered from a solvent in contact with a dosage form of the present invention.

In some preferred embodiments, the dosage form is substantially devoid of hydrogenated Type I vegetable oils. In other embodiments, the dosage form is substantially devoid of hydrogenated Type II vegetable oils. In other embodiments, the dosage form is substantially devoid of polyoxyethylene stearates. In other embodiments, the dosage form is substantially devoid of polyoxyethylene distearates; in other embodiments, the dosage form is substantially devoid of glycerol monostearate. In other embodiments, the dosage form is substantially devoid of poorly water soluble, high melting point (mp=40 to 100° C.) waxes.

The present invention can also optionally include other ingredients to enhance dosage form manufacture from a pharmaceutical composition of the present invention and/or alter the release profile of a dosage form including a pharmaceutical composition of the present invention.

Some embodiments of the present invention include one or more pharmaceutically acceptable fillers, diluents, glidants and lubricants of various particle sizes and molecular weights.

The dosage form according to the invention may also comprise a coating which is resistant to gastric juices and dissolves as a function of the pH value of the release environment.

By means of this coating, it is possible to ensure that, when correctly administered, the dosage form according to the invention passes through the stomach undissolved and the active ingredient is only released in the intestines.

In one preferred embodiment, the opioid of the invention is in immediate release form, said dosage form having abuse deterrent properties, said dosage form selected from the group comprising alfentanil, anileridine, buprenorphine, brifentanil, butorphanol, carfentanil, codeine, dextromoramide, dezocine, dihydrocodeine, dihydromorphine, fentanyl, heroin, hydrocodone, hydromorphone, hydroxypethidine, isomethadone, ketobemidone, levorphanol, levomethadone, lofentanil, meperidine, meptazinol, metazocine, methadone, 4-methoxymethylfentanyl, 3-methylfentanil, metopon, mirfentanil, morphine, morphine-6-glucuronide, nalbuphine, norlevorphanol, normethadone, nalorphine, ohmefentanyl, opium, oxycodone, oxymorphone, pentazocine, phenazocine, propiram, propoxyphene, remifentanil, sufentanil, tapentadol, trefentanil, tramadol, tilidine, any opioid having agonist activity at an opioid receptor belonging to the phenanthrene, morphinan, benzomorphan, methadone, phenylpiperidine, propionanilide 4-anilidopiperidine, 4-aryl piperidines, and 4-Heteroarylpiperidines class, any opioid having agonist activity at an opioid receptor having the same pentacyclic nucleus as nalmefene, naltrexone, buprenorphine, levorphanol, meptazinol, pentazocine and dezocine, any opioid having agonist activity at an opioid receptor which is a fentanyl analog, or their pharmaceutically acceptable salts, prodrugs, esters, analogs, derivatives, solvates, complexes, polymorphs, hydrates and metabolites, as racemates or an individual diastereoisomers or enantiomeric isomers thereof or mixtures thereof.

In one preferred embodiment, the opioid of the invention is in extended release form, said dosage form having abuse deterrent properties, said dosage form selected from the group comprising alfentanil, anileridine, buprenorphine, brifentanil, butorphanol, carfentanil, codeine, dextromoramide, dezocine, dihydrocodeine, dihydromorphine, fentanyl, heroin, hydrocodone, hydromorphone, hydroxypethidine, isomethadone, ketobemidone, levorphanol, levomethadone, lofentanil, meperidine, meptazinol, metazocine, methadone, 4-methoxymethylfentanyl, 3-methylfentanil, metopon, mirfentanil, morphine, morphine-6-glucuronide, nalbuphine, norlevorphanol, normethadone, nalorphine, ohmefentanyl, opium, oxycodone, oxymorphone, pentazocine, phenazocine, propiram, propoxyphene, remifentanil, sufentanil, tapentadol, trefentanil, tramadol, tilidine, any opioid having agonist activity at an opioid receptor belonging to the phenanthrene, morphinan, benzomorphan, methadone, phenylpiperidine, propionanilide 4-anilidopiperidine, 4-aryl piperidines, and 4-Heteroarylpiperidines class, any opioid having agonist activity at an opioid receptor having the same pentacyclic nucleus as nalmefene, naltrexone, buprenorphine, levorphanol, meptazinol, pentazocine and dezocine, any opioid having agonist activity at an opioid receptor which is a fentanyl analog, or their pharmaceutically acceptable salts, prodrugs, esters, analogs, derivatives, solvates, complexes, polymorphs, hydrates and metabolites, as racemates or an individual diastereoisomers or enantiomeric isomers thereof or mixtures thereof.

In one preferred embodiment, the opioid of the invention is in extended release form, said dosage form devoid of substantial abuse deterrent properties, said dosage form selected from the group comprising alfentanil, anileridine, buprenorphine, brifentanil, butorphanol, carfentanil, codeine, dextromoramide, dezocine, dihydrocodeine, dihydromorphine, fentanyl, heroin, hydrocodone, hydromorphone, hydroxypethidine, isomethadone, ketobemidone, levorphanol, levomethadone, lofentanil, meperidine, meptazinol, metazocine, methadone, 4-methoxymethylfentanyl, 3-methylfentanil, metopon, mirfentanil, morphine, morphine-6-glucuronide, nalbuphine, norlevorphanol, normethadone, nalorphine, ohmefentanyl, opium, oxycodone, oxymorphone, pentazocine, phenazocine, propiram, propoxyphene, racemorphan, remifentanil, sufentanil, tapentadol, trefentanil, tramadol, tilidine, any opioid having agonist activity at an opioid receptor belonging to the phenanthrene, morphinan, benzomorphan, methadone, phenylpiperidine, propionanilide 4-anilidopiperidine, 4-aryl piperidines, and 4-Heteroarylpiperidines class, any opioid having agonist activity at an opioid receptor having the same pentacyclic nucleus as nalmefene, naltrexone, buprenorphine, levorphanol, meptazinol, pentazocine and dezocine, any opioid having agonist activity at an opioid receptor which is a fentanyl analog, or their pharmaceutically acceptable salts, prodrugs, esters, analogs, derivatives, solvates, complexes, polymorphs, hydrates and metabolites, as racemates or an individual diastereoisomers or enantiomeric isomers thereof or mixtures thereof.

In one preferred embodiment, the opioid of the invention is in extended release form, said dosage form selected from the group comprising alfentanil, anileridine, buprenorphine, brifentanil, butorphanol, carfentanil, codeine, dextromoramide, dezocine, dihydrocodeine, dihydromorphine, fentanyl, heroin, hydrocodone, hydromorphone, hydroxypethidine, isomethadone, ketobemidone, levorphanol, levomethadone, lofentanil, meperidine, meptazinol, metazocine, methadone, 4-methoxymethylfentanyl, 3-methylfentanil, metopon, mirfentanil, morphine, morphine-6-glucuronide, nalbuphine, norlevorphanol, normethadone, nalorphine, ohmefentanyl, opium, oxycodone, oxymorphone, pentazocine, phenazocine, propiram, propoxyphene, racemorphan, remifentanil, sufentanil, tapentadol, trefentanil, tramadol, tilidine, any opioid having agonist activity at an opioid receptor belonging to the phenanthrene, morphinan, benzomorphan, methadone, phenylpiperidine, propionanilide 4-anilidopiperidine, 4-aryl piperidines, and 4-Heteroarylpiperidines class, any opioid having agonist activity at an opioid receptor having the sane pentacyclic nucleus as nalmefene, naltrexone, buprenorphine, levorphanol, meptazinol, pentazocine and dezocine, any opioid having agonist activity at an opioid receptor which is a fentanyl analog, or their pharmaceutically acceptable salts, prodrugs, esters, analogs, derivatives, solvates, complexes, polymorphs, hydrates and metabolites, as racemates or an individual diastereoisomers or enantiomeric isomers thereof or mixtures thereof.

In one preferred embodiment of the invention, the formulation is ingested orally as a tablet or capsule, preferably as a capsule. In another preferred embodiment of the invention, the formulation is administered bucally. In yet another preferred embodiment of the invention, the formulation is administered sublingually.

Opioids are used primarily to treat pain of various etiologies, intensities and duration. However opioids can also be used for non-painful conditions such as restless leg syndrome and urinary incontinence. The present invention contemplates all medical uses of opioids by the oral route of administration, preferably the oral route.

In one preferred embodiment, the formulation is used to treat restless leg syndrome. In another preferred embodiment of the invention, the formulation is used to treat urinary incontinence. In another preferred embodiment of the invention, the formulation is used to addiction disorders.

In one preferred embodiment, the invention provides for methods and pharmaceutical compositions to prevent or minimizing excessive peak concentrations (dose dumping) of therapeutic doses of extended release opioids used for medical purposes, when they are co-ingested with alcohol.

In one preferred embodiment, the invention provides for methods and pharmaceutical compositions to achieve an extended release opioid formulation.

In one preferred embodiment, the invention provides for methods and pharmaceutical compositions to achieve an abuse deterrent formulation.

In one preferred embodiment, the invention provides for methods and pharmaceutical compositions to simultaneously achieve an extended release opioid formulation and an abuse deterrence formulation.

In one preferred embodiment, the invention provides for methods and pharmaceutical compositions to simultaneously achieve an extended release opioid formulation and an abuse deterrence formulation, without the use of aversive agents.

In one preferred embodiment, the invention provides for methods and pharmaceutical compositions to simultaneously achieve an extended release opioid formulation and an abuse deterrence formulation, using substantially the same ADER agents.

In one preferred embodiment, the invention provides for methods and pharmaceutical compositions to simultaneously achieve an extended release opioid formulation and an abuse deterrence formulation, using substantially the same ADER agents without the use of aversive agents.

To further evaluate this invention, in initial testing, tramadol was selected. Tramadol: i) has been implicated in drug abuse; ii) is an unscheduled opioid and consequently not well monitored unlike other opioids; iii) is water soluble and therefore prone to easy extraction and gastrointestinal absorption; iii) is available in extended release formulations, which if tampered with may dump an entire days contents into the systemic circulation, thereby resulting in toxicity from both opioid and non-opioid mechanisms.

Tramadol is a synthetic, centrally acting analgesic which exerts its analgesic effects by inhibiting reuptake of norepinephrine and serotonin and by activation of μ-opioid receptors. Tramadol binds to the μ-opioid receptor, although its principal active (M1) metabolite, mono-O-demethyl-tramadol is up to 6 times more potent in producing analgesia and 200 times more potent in t-opioid binding (Ultram® Package Insert). During its intentional or inadvertent non-medical use, tramadol, especially the extended release tramadol is likely to be crushed. Since tramadol produces dose dependent seizures and dose dependent serotonin syndrome, there is the potential for a compounded risk.

Data from FDA, the Drug Enforcement Administration (DEA), the Drug Abuse Warning Network (DAWN), the American Association of Poison Control Centers Toxic Exposure Surveillance System (TESS), National Survey on Drug Use and Health (NSDUH) and other sources indicate that tramadol is widely abused.

Tramadol avidly binds to the μ-receptor. Its principal active metabolite, mono-O-demethyl-tramadol (M1) is up to 6 times more potent than the parent drug in producing analgesia and 200 times more potent in μ-opioid binding (Desmeules et al., Br J Clin Pharmacol. 1996; 41:7-12). Replacement of tramadol with the opioid antagonist's levallorphan and naloxone precipitate weight loss in rats and characteristic signs of opioid withdrawal in mice and rats (Friederichs et al., Arzneim Forsch 1978; 28:122-134; Murano et al., 1978; 28:152-158; Wakasa et al., 1994; Japan J Alcohol & Drug Depend 1994; 29:40-51).

Tramadol fully substitutes for morphine at high doses in a rat model of dependence and this effect is antagonized by the opioid antagonist, naltrexone (Ren and Zheng, Acta Pharmacol Sin 2000; 21:924-26). Other studies have suggested that tramadol only partially substitutes for morphine in animal models. An important consideration is the selection of tramadol dose and the role its principal active metabolite (M1), which accumulates with repeated dosing and would undoubtedly be a C-II scheduled drug if it were commercialized today.

According to the Drug Enforcement Agency, “Tramadol is abused for its opiate effects. The current pattern of tramadol abuse in the US involves street drug addicts, chronic pain patients, and health professionals. As an uncontrolled substance, there are no Controlled Substance Act regulations regarding manufacturing, distribution, or prescription of this medication.” (http://www.deadiversion.usdoj.gov/drugs_concern/tramadol.htm). DEA cites the Drug Abuse Warning Network (DAWN) data for drug related hospital emergency room episodes. In 2002, there were 1,714 episodes for tramadol and a total of 7,890 episodes from 1998 through 2002. DAWN medical examiners reported that tramadol was involved in 95 drug-related deaths in 2002 and a total of 382 deaths from 1998 through 2002.

According to the 2002 National Survey on Drug Use and Health (NSDUH), approximately one million individuals have taken tramadol (Ultram®) for non-medical use. This is approximately the same incidence of non-medical use reported for Dilaudid® and approximately 50% of the incidence reported for OxyContin®. Among non-medical OxyContin® users, 18.3% also reported consuming Ultram® for non-medical reasons.

In the two most recent annual reports of the American Association of Poison Control Centers Surveillance System (TESS), tramadol ranked only second to oxycodone in the number of opioid exposure cases (Watson et al, 2002; Am J Emerg Med 2003; 21; 353-421).

A study published in the September 2004 issue of the Journal of Forensic Sciences of 66 deaths in which short-acting tramadol was detected in the decedent's blood notes that “ . . . tramadol may be a significant contributor to lethal intoxication when taken in excess with other drugs . . . ”. The study discusses the role of opioid and serotonin effects in such deaths (Clarkson et al, J Forensic Sci 2004; 49; 1101-5).

According to a recent report in JAMA regarding drugs abused by physicians, tramadol was the third most frequently mentioned abused opioid. It was more frequently mentioned than was fentanyl, oxycodone or hydromorphone (Skipper et al, JAMA 2004; 292:1818-19).

The FDA's Medwatch system has received approximately a 1000 domestic adverse-event reports for tramadol coded as “drug dependence”, “drug withdrawal” or “drug abuse” (Brinker et al, Am J Psychiatry 2002; 159; 881-82).

There are also numerous reports in the literature of drug abuse, addiction, physical dependence, seizures and withdrawal on abrupt cessation with tramadol. Tramadol has also been found to be effective in substituting for treating moderate heroin withdrawal, with efficacy comparable to buprenorphine and superior to clonidine (Tamaskar et al, J Addict Dis 2003; 22:5-12; Sobey et al, J Addict Dis 2003; 22:13-25).

Commercially available I.R. tramadol (Ultram®) releases 50 mg of tramadol per tablet into the systemic circulation over several hours. New, extended release formulations are designed to gradually release their much larger tramadol content over a 24-hour period. Experience with OxyContin® would suggest that if formulations of Tramadol ER are tampered, the entire 24-hour drug supply may be released into the bloodstream, with resulting potential for toxic effects. The 24-hour supply of tramadol contained in one tablet, instead of 4 to 6 tablets means that there is a risk that such formulations may be highly sought by drug addicts and recreational drug users alike for non-medical use. Intentional tampering from Tramadol ER formulations has the potential to rapidly deliver a massive dose and produce neurological toxicity, including agitation, seizures, coma and respiratory failure.

### EXAMPLES

Non-limiting examples for preparing the dosage form are set forth below.

### Determination of Analgesic and Other Pharmacologic Effects

The pharmacologic effects (e.g., analgesia, opioid toxicity) of the pharmaceutical compositions of the present invention can be evaluated using analgesic methods well established in the art. A wide variety of pain states and study designs may be used to evaluate the therapeutic effects of intact and tampered dosage forms of the invention. This invention therefore contemplates the use of test methods other than those specifically disclosed herein, including those which may hereafter become known to the art to be capable of performing the necessary functions. Sample sizes in the studies are sufficient to demonstrate the objectives of the testing. A non-limiting list of methods to evaluate the analgesic and other effects of the invention is provided below:

### Third Molar Extraction Model

Male and female patients with acute postsurgical pain following the removal of one or more bony impacted third molars are participants. Within 4 to 6 hours after completion of surgery, patients who are experiencing moderate or severe pain, as measured by a visual analog pain intensity scale (VAS≧50 mm) and by a categorical pain intensity scale (moderate or severe pain descriptor), and who meet all other inclusion/exclusion criteria are admitted to the study. Patients are randomly assigned to receive the dosage form of the invention given intact or placebo, in some preferred embodiments, and the dosage form of the invention given intact or dosage form of the invention given in tampered form in other embodiments. Both single and multiple (repeated) dose studies may be conducted. Pain intensity (VAS and categorical), pain relief (categorical) and whether pain is half-gone is recorded by the patient under the supervision of the investigator study coordinator at the various time points: Baseline (0 hour—pain intensity only), 15, 30 and 45 minutes, and at 1, 1.5, 2, 3, 4, 5, 6, 7, 8 and 12 hours after administration of study medication, and immediately prior to the first rescue dose. Sedation and nausea may be evaluated using VAS or categorical scales. Time to onset of perceptible and meaningful pain relief is evaluated using the two stopwatch method. Patients record their global evaluation of study medication at the completion of the 8-hour assessment or at the time of first rescue medication use. Efficacy endpoints include Total Pain Relief (TOTPAR), Sum of Pain Intensity Difference (SPID) and Sum of Pain Relief Intensity Difference (SPRID) at various time points, Time to First Rescue, Time Specific Pain Intensity Difference (PID), Time Specific Pain Relief (PR), Peak Pain Intensity Difference (PPID), Peak Pain Relief (PPR), Time to Confirmed Perceptible Pain Relief (stopwatch), Time to Meaningful Pain Relief (stopwatch), Patient Global Evaluation, Time to Change in Categorical PID≧1, Percent Change in Pain Intensity Score from Baseline, Mean Change in Pain Intensity Score From Baseline, Percent Change in Pain Relief Score from Baseline, Mean Change in Pain Relief Score From Baseline, Percent of Responders, Number of Patients Needed to Treat to Obtain One Patient with ≧50% Response (NNT).

### Bunionectomy Surgery

Male or female patients requiring primary unilateral first metatarsal bunionectomy surgery alone or with ipsilateral hammertoe repair (without additional collateral procedures) under regional anesthesia (Mayo block) are participants.

Patients who experience moderate or severe pain on a categorical scale (moderate or severe descriptor) and on a visual analog pain intensity scale (VAS; ≧50 mm) within 6 hours following completion of bunionectomy surgery are randomly assigned to receive the dosage form of the invention given intact or placebo In some preferred embodiments, and the dosage form of the invention given intact or dosage form of the invention given in tampered form in other embodiments. Both single and multiple (repeated) dose studies may be conducted. Patients are encouraged to wait at least 60 minutes before requesting remedication for pain. At the completion of the single-dose phase (8 hours) or at first request for remedication (whichever is earlier), patients enter into a multiple-dose phase lasting approximately 72 hours. During the multiple dose phase patients receive study medication or placebo at a fixed dose interval (e.g., every 8, 12 or 24 hours). Once the multiple dose phase of the study has begun, patients experiencing pain between scheduled doses of study medication are provided access to supplemental open-label (rescue) analgesia. Patients whose pain cannot be adequately managed on a combination of study medication and rescue medication or who develop unacceptable side effects during the study are discontinued from further study participation and their pain managed conventionally.

Pain intensity (VAS and categorical), pain relief (categorical) and whether pain is half-gone is recorded by the patient under the supervision of the investigator study coordinator at representative time points, e.g., Baseline (pain intensity only), 15, 30 and 45 minutes and 1, 1.5, 2, 3, 4, 5, 6, 7 and 8 hours after administration of study medication and immediately prior to the first remedication. Sedation and nausea may be evaluated using VAS or categorical scales. Time to onset of perceptible and meaningful pain relief is evaluated using the double-stopwatch method. Patients complete a global evaluation of study medication at the completion of the 8-hour assessment or just prior to the first remedication. Following completion of the single-dose phase (8 hours or just prior to first remedication, if ≦8 hours), patients begin the multiple dose phase of the study. During the multiple dose phase, patients record their overall pain intensity since the previous scheduled dose, their current pain intensity and a patient global, immediately prior to each scheduled dose of study medication and at early termination.

Measures of efficacy in the single-dose phase include Sum of Pain Intensity Difference (SPID), Total Pain Relief (TOTPAR), Sum of Pain Relief Intensity Difference (SPRID), Time to First Remedication, Time Specific Pain Intensity Difference (PID), Time Specific Pain Relief (PR), Peak Pain Intensity Difference (PPID), Peak Pain Relief (PPR), Time to Confirmed Perceptible Pain Relief (stopwatch), Time to Meaningful Pain Relief (stopwatch), Patient Global Evaluation, Time to Change in Categorical PID≧1, Percent Change in Pain Intensity Score from Baseline, Mean Change in Pain Intensity Score From Baseline, Percent Change in Pain Relief Score from Baseline, Mean Change in Pain Relief Score From Baseline, Percent of Responders, Number of Patients Needed to Treat to Obtain One Patient with ≧50% Response (NNT).

Measures of efficacy in the multiple-dose phase include the time specific overall pain intensity, current pain intensity and patient global at the time of scheduled remedication, the average of overall pain intensity, current pain intensity and patient global over 0-24, 2448 and 48-72 and number of doses of rescue analgesic over 0-24, 24-48 and 48-72 and 0-72 hours.

### Chronic Pain of Osteoarthritis

The analgesic efficacy of the invention may be demonstrated in single or repeated dose randomized double-blind, controlled studies. Patients are randomized to receive the dosage form of the invention given intact or placebo, in some preferred embodiments, and the dosage form of the invention given intact or dosage form of the invention given in tampered form in other embodiments. In repeated dose studies, typically, patients who meet the American College of Rheumatology criteria for knee and/or hip OA are washed off their analgesics for 2 to 7 days to allow for pain of moderate to severe intensity to return. Once a stable baseline pain score is established, patients are randomized to treatment, usually for a period of 1 to 12 weeks. Pain, joint stiffness and physical function can be measured with a multidimensional instrument, such as the WOMAC, quality of life with the SF-12 or SF-36 and adverse events with a non-directed questionnaire at baseline and at post-baseline return visits. Response to pain, stiffness, physical function, quality of life and adverse events are calculated as change from baseline and compared between treatments. Sedation and nausea may be evaluated using VAS or categorical scales.

### Migraine

The analgesic efficacy of the invention may be demonstrated in single or repeated dose randomized double-blind, controlled studies. Patients are randomized to receive the dosage form of the invention given intact or placebo, in some preferred embodiments, and the dosage form of the invention given intact or dosage form of the invention given in tampered form in other embodiments. Patients with migraine headaches are typically evaluated in prospective, randomized, double-blind, parallel group, single-dose studies. Crossover studies are also possible. The study population consists of male and non-pregnant female subjects, 18 to 65 years of age with a primary headache diagnosis of either migraine attack without aura or migraine attack with aura, as diagnosed according to the International Classification of Headache Disorders-2 criteria. To qualify, the subject must typically have a history, on average, of at least one migraine attack per month, but an average of no more than 6 migraine attacks each month during the past year. Using a headache diary subjects are instructed to treat and evaluate the headache pain and symptoms associated with one eligible migraine attack, with or without aura, with at least moderate headache pain intensity. Eligible subjects are randomly assigned to receive the drug to treat one migraine attack, with or without aura, with headache pain of at least moderate pain intensity as determined by them migraine questionnaire they are asked to take a single dose of study drug, according to their randomized treatment assignment. Headache pain intensity, nausea, photophobia, phonophobia, vomiting, and ability to function are assessed at baseline, 0.25, 0.5, 0.75, 1, 1.5, 2, 2.5, 3, 4, 8, 16 and 24 hours post-dose. In addition, the recurrence of pain and use of any rescue mediation is documented. Primary efficacy variables typically consist of the percent of subjects who are without: (i) pain; (ii) nausea; (iii) photophobia and, (iv) phonophobia, each at 2 hours post-dosing. Secondary efficacy variables typically consist of headache pain intensity and associated symptoms at each evaluation time point, incidence of vomiting, patient function, sum of pain intensity difference at each evaluation time (SPID), percent of subjects who experience headache recurrence up to 24 hours, and the median time to recurrence. Sedation may be evaluated using VAS or categorical scales. Recurrence is defined as the reduction in pain from moderate or severe pain to none at 2 hours after taking study drug, followed by: (i) an increase to mild, moderate or severe pain within 24 hours after taking the study drug, or (ii) consuming a rescue medication within 24 hours after taking the study drug.

The preparation of oral immediate release dosage forms is well known in the art—see Remington: the science of Pharmacy Practice, 21^{st} Edition, 2006, Lippincott, Williams & Wilkins, Baltimore, Md.; Pharmaceutical Preformulation and Formulation: A Practical Guide from Candidate Drug Selection to Commercial Dosage Form. Gibson, M (ed). CRC Press, 2001; Niazi, S. Handbook of Pharmaceutical Manufacturing Formulations: Uncompressed Solid Products (Volume 2 of 6), CRC Press, 2004; Niazi, S. Handbook of Pharmaceutical Manufacturing Formulations: Compressed Solid Products (Volume 1 of 6), CRC Press, 2004; Mollet, H, Grubenmann A, Payne H. Formulation Technology: Emulsions, Suspensions, Solid Forms, Wiley-VCH, 2001; Niazi S and Niazi S K, Pharmaceutical Capsules, 2^{nd} Ed., Podczeck, F and Jones B E (eds)., Pharmaceutical Press, 2004, London (all of which are hereby incorporated by reference). A majority of oral dosage forms commercially available world wide are formulated as immediate release products.

### Tamper Resistance and Tamper Deterrence Testing

The popularity of immediate release formulations of opioids among drug addicts and recreational drug users is in part due to the mood altering and reinforcing effects of the drug. The popularity of extended release formulations of opioids among drug addicts and recreational drug users is in part due to the pharmacologic properties of the drug therein (e.g., mood altering and reinforcing effects) and in part due to the large amount of drug per tablet or capsule (e.g., a 12 or 24 hour supply). For example commercially available immediate release opioid tablets and capsules are usually administered every 4 to 6 hours and they release their dose into the systemic circulation over one to two hours. New, extended release formulations are designed to gradually release their much larger opioid content over a 12 or 24-hour period. Most recreational drug users and addicts have a unit of use which is one tablet or capsule. The 12 or 24-hour supply of an opioid agonist typically contained in one extended release tablet or capsule, instead of in 4 to 6 tablets or capsules means that there is a greater risk that such formulations may be highly sought by drug addicts and recreational drug users alike, for non-medical use. Intentional or inadvertent tampering from extended release formulations will rapidly deliver a massive dose and produce profound pharmacologic effects.

Addicts and recreational drug users commonly use abusable drugs by a variety of routes of administration. Commonly used methods include 1) parenteral (e.g., intravenous injection, where the drug is crushed and extracted or melted and the contents of a dosage unit then injected), 2) intranasal (e.g., snorting, where the drug is inhaled as powdered dosage unit), and 3) episodic or repeated oral ingestion of crushed product, where the drug is chewed to increase the surface area and permit rapid release of drug substance. All of these strategies are intended to more efficiently get the opioid into the CNS, both in terms of total amount of drug, peak concentration of drug and time to peak concentration of drug.

One mode of abuse involves the extraction of the drug component from the dosage form by first mixing the table or capsule with a suitable solvent (e.g., water or alcohol), and then filtering and/or extracting the drug component from the mixture for intravenous injection. Another mode of abuse of extended release drugs involves dissolving the drug in water, alcohol or another “recreational solvent” to hasten its release and to ingest the contents orally, in order to provide high peak concentrations and maximum euphoriant effects.

It is necessary to be able to measure resistance ore deterrence to the likely routes of abuse in a meaningful and relevant way. The in vitro tests below are provided for illustration of some testing methods and are intended to be non-limiting examples. This invention therefore contemplates the use of test methods other than those specifically disclosed herein, including those which may hereafter become known to the art to be capable of performing the necessary functions.

Extraction with Alcohol on Whole Dosage Unit

Method: Place a whole dosage unit in 18 mL of 0.1N HCl in a 60 mL amber bottle and shake at 240 rpm on an orbital shaker for 30 min. After 30 min add 12 mL of ethanol (95-96%) to each bottle. Swirl by hand and remove a 1 mL sample from each bottle (T₀). Place the solutions back in the orbital shaker for further shaking at 240 rpm. Take 1 mL samples after 10, 20, 30, 40, 60 and 180 min of further shaking for each bottle. Analyze and graph the results on a linear scale of cumulative release (%) vs. time (min).

Extraction with Alcohol on a Crushed or Cut Dosage Unit

Extension of the above test. Method: Place a tablet (after crushing with a single crush with a spatula) or a capsule (cut in half) in 18 mL of 0.1N HCl in a 60 mL amber bottle and shake at 240 rpm on an orbital shaker for 30 min. Continue the test as in 1) above.

Extraction into Water

Method: Crush with a mortar and pestle and grind in 5 mL of water for 5 minutes. The resulting suspension is filtered through a 0.45 micron filter into a flask and diluted to 50 mL with water. Quantify drug concentration by HPLC.

### Freeze and Crush

Method: Freeze the dosage unit in a domestic freezer for 24 hr, then grind with a mortar and pestle for five minutes. Sieve through a suitable sieve (ca 600 micron) and, by weighing, measure the percentage passing the sieve.

### Taste of Base Excipient Mix (Organoleptic Test)

Method: Chew a placebo mix for five minutes and rate the taste on a 0-10 scale with 0 as bland to repulsive at 10. This method is relevant only to dosage units containing taste modifiers.

Extraction into Acid

Method: Crush with a mortar and pestle and heat to boiling in 5 mL of vinegar. The resulting suspension is filtered through a 0.45 micron filter into a flask and diluted to 50 mL with water. Quantify drug concentration by HPLC.

### Application of Heat (Melting Temperature>50° C. or 55° C.)

Method: Heat the squashed contents of a dosage unit on a hot plate until melted. Determine the temperature of melting and test whether the mix becomes sufficiently fluid to be drawn up into a syringe via a 1.2 mm needle then expelled.

Materials in the series of experiments below included the following: Aerosil® 200, Lot 1412033, ex Degussa Huls, Aerosil® COK84, Lot 2258, ex Degussa Huls, Beeswax, Lot A018035701, ex Acros Organics, Cetyl alcohol (1-hexadecanol), Lot A019258301, ex Acros Organics, Cithrol® GMS 0400, Lot 6483-0103, ex Croda, Fractionated coconut oil, Lot 165544, ex A E Connock Gelucire® 44/14, Lot 22009, ex Gattefosse, Gelucire® 50/02, Lot 19255, ex Gattefosse, Gelucire® 50/13, Lot 20529, ex Gattefosse, Hydrokote 112 Lot 048M3, ex Abitech Corp, Hydrokote AP5, Lot 340J1, ex Abitech Corp, Hydrokote M, Lot 126J2, ex Abitech Corp, Methocel® AM4, Lot Q101012N01, ex Colorcon, Methocel® K100M, Lot QA15012N01, ex Colorcon, Methocel® K15M, Lot QK02012N11, ex Colorcon, Paraffin wax, Lot P/0680/90, ex Fisher Scientific, PEG 400, Lot 310354, ex NOF Corp, Pluriol E6005 (PEG 6000), Lot 97193, ex BASF, Pharmacoat 606 (hypromellose USP), Lot 308522, ex Shin-Etsu Chemical Co Ltd., Poloxamer 124 (Pluronic L44), Lot WPWV-645B, ex BASF, Poloxamer 188 (Lutrol F68), Lot 0306043523, ex BASF, Propoylene glycol, Lot 09521H0, ex Aldrich, Propranolol HCl, Lot 044K1219, ex Sigma, Shellac, Lot 4010 2465 2056, ex Syntapharm, Size 1 clear/clear gelatin capsules, Lot C14893, ex Capsugel, Starch 1500, Lot IN 500578, ex Colorcon, Sterotex® N F, Lot 324M2, ex Abitech Corp., Tramadol HCl, Lot 3TRMDN0D105 & 3TRMDN0E056, ex Chemagis Ltd, Zein (Paroxite), Lot 5041C, ex Variati & Co.

Equipment in the series of experiments below included the following: Caleva 9ST dissolution apparatus with ERWEKA P thermostatically controlled water heater, Copley ZT54 disintegration apparatus, Haake DC5 water bath, Heidolph bench mixer, HiBar bench filling machine, Qualiseal bench banding machine, Silverson SL2 bench high shear mixer, Thermo Electron Vision uv/visible spectrometry data acquisition program with Vision Security, Unicam UV2-400 spectrophotometer, Watson Marlow 205U peristaltic pump 650μ nominal s/s Laboratory test sieve, 600μ s/s certified Laboratory test sieve from Endecotts Ltd, London, Whatman 25 mm 45μ filters used in combination with a 5 ml Luer lock syringe.

### Example 1

### Binary Mix Compatibility Trials

Binary mixes were prepared of tramadol HCL in potential excipients (in some instances a third material, fractionated coconut oil was used to bring two non melting materials into intimate contact). The mixes were stored in sealed amber glass bottles under conditions of 40° C./75% RH for four weeks then examined by HPLC for signs of interaction or degradation. Excipients were chosen from materials considered to potentially cover the range of material properties that were likely to be required by this project. Materials were chosen for properties such as dissolution rate i.e. from materials that are relatively soluble in aqueous media to totally insoluble materials; their potential as viscosity/release rate modifiers, including such materials as different HPMC (viscosity) grades and Aerosils for contributing thixotropic properties. Mixes containing 25% w/w tramadol HCL were prepared for each excipient. Samples were prepared by mixing tramadol HCl with the melted excipient or for non melting excipients materials were placed in contact by blending with a 50/50 mix of excipient and fractionated coconut oil. Samples of each excipient were also stored in sealed amber glass bottles at 40° C./75% RH as control samples. The project objective describes a target of 15 binary mixes; however, 25 different mixes were made during this trial to maximize the range of excipients available for formulation.

### Dissolution Testing

Initially two test formulations were prepared as noted below. The capsules for this and all other small scale capsule preparations were manufactured by the melting and mixing of the ingredients in a water bath or on a hot plate then hand filling capsules to the target weight. All capsules used were size 1 gelatin capsules.

Formulation 052/014

| | | Quantity per |
|---|---|---|
| Material | % w/w | cap mg |
| Poloxamer 188 | 62.8 | 282.7 |
| HPMC K100M | 17.9 | 80.3 |
| Aerosol COK 84 | 2.7 | 12.0 |
| Tramadol HCl | 16.6 | 74.9 |
| Capsule fill weight | | 450 |

Formulation 052/015

| Material | % w/w | Quantity per cap mg |
|---|---|---|
| Gelucire 50/02 | 58.3 | 233.3 |
| HPMC Pharmacoat 606 | 19.9 | 79.8 |
| Aerosil ® COK 84 | 3.0 | 12.0 |
| Tramadol HCl | 18.7 | 74.9 |
| Capsule fill weight | | 400 |

The target fill weight was set as 400 mg for a size 1 capsule. Formulation 052/014 was initially targeted on a 400 mg fill, however, the mix proved too viscous to fill. Additional poloxamer 188 had to be added to reduce the mix viscosity to a level that could be encapsulated. The addition of extra poloxamer 188 required that the fill weight be increased to 450 mg. This quantity could be hand filled into a capsule and would meet the requirements of this preliminary trial; however, such a quantity would be excessive for machine filling into a size 1 capsule.

The tramadol HCl dissolution release profile was determined, for each formulation. Full dissolution testing is carried out using six individual capsule sets. Preliminary screening trials used between two and six capsules per test. This permitted several candidate formulations to be screened at once and clearly unsuitable formulations eliminated quickly. Potentially useful formulations could be modified further first before going on to six capsule sample dissolution testing.

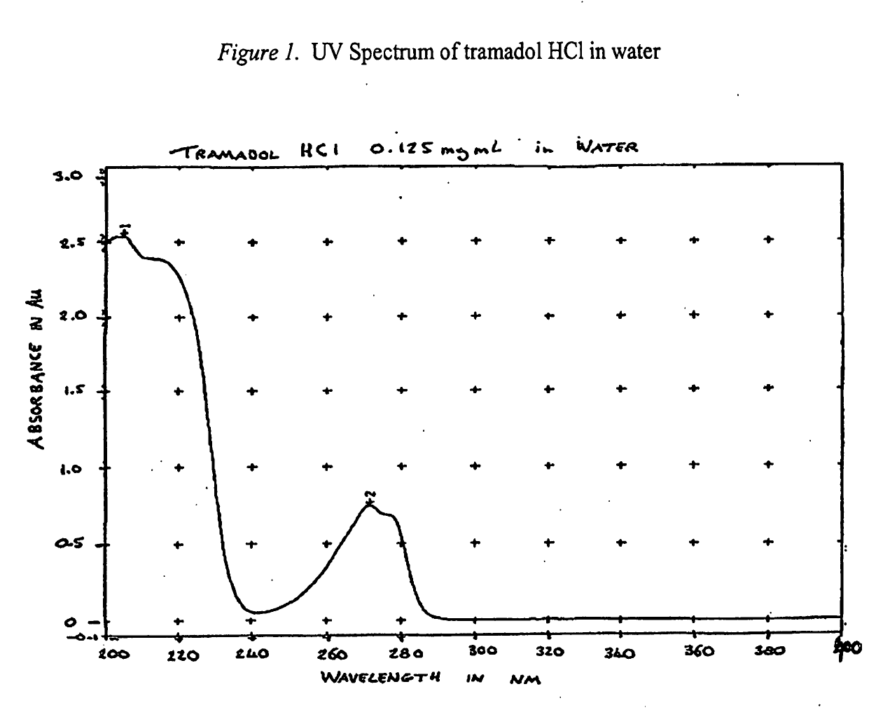

Tramadol HCl in aqueous solution shows an absorbance maximum between 240 nm and 290 nm with the maximum at 271 nm. It starts to show increasingly strong absorbance below the minimum at 240 nm to 200 nm (the limit of the instrument) however absorbance in this area is shown by many compounds so observation in the more definitive region of 240 nm to 290 was selected with 270-272 nm chosen as the preferential wavelength of observation. A plot of the UV spectrum of tramadol HCl in water is shown in FIG. 1.

Dissolution testing was carried out using the USP paddle method on a Caleva 9ST dissolution apparatus with an ERWEKA P, thermostatically controlled, water heater. Each solution was continuously cycled through a Unicam UV2-400 spectrophotometer using a Watson Marlow 205U peristaltic pump and the solution absorbance in a 1 cm silica cell, at 271 nm, recorded against the absorbance of a placebo or SIF blank with the data captured by Thermo Electron Vision UV/visible spectrometry data acquisition software protected by Vision Security. The spectrophotometer was fitted with a six cell autochanger permitting continuous automatic recording of cell solution absorbances. The capsules were weighed down with 316 stainless steel sinking wire, wrapped round each capsule. Each solution passed through a filter as it was pumped from the dissolution bath. Except where otherwise specified, the dissolution medium was 600 ml of Simulated Intestinal Fluid (SIF) USP without the inclusion of enzyme. This dissolution set up was selected to give a final absorbance value, with full release of tramadol HCl, of not more than 1.5 absorbance units (au). Typically, the final absorbance of a test solution did not exceed 1.0 au. A placebo blank was used in the reference cell. This comprised of a capsule containing the same proportion and quantity of each material used in the active test capsules but without the tramadol HCl. This ensured that the reference solution contained the same quantity (and thus gave the same background absorbance) as the excipients in the active capsules.

### Binary Mix Compatibility Study

25 Different materials were tested for compatibility with Tramadol HCl. The results of storage in sealed amber glass bottles under conditions of 40° C./75% RH for four weeks then subsequent analysis by HPLC for degradants or impurities are as below.

| | | | | Impurities/ | |
|---|---|---|---|---|---|
| | | | Peaks from | Degradants | |
| | | | stressed | % area | |
| | Material | Assay % | excipient | normalized | Comments |
| 1 | Gelucire 44/14 | 127.9 | none | none | |
| 1 | Gelucire 44/14 REPEAT | 71.2 | none | none | Mean 2 samples 99.5% |
| | SAMPLE | | | | |
| 2 | Gelucire 50/13 | 106.3 | none | none | |
| 3 | Gelucire 43/01 | | | | Not available |
| 4 | Poloxamer 188 | 101.9 | none | none | |
| 5 | Poloxamer 124 (Pluronic L44) | 98.6 | none | none | Separated suspension |
| | | | | | re-mixed before sampling |
| 6 | PEG 6000 | 96.6 | none | none | |
| 7 | PEG 400 | 100.7 | none | none | |
| 8 | Propylene glycol | 96.5 | none | none | |
| 9 | Beeswax (refined yellow) | 2.1 | none | none | Material insoluble in sample |
| | | | | | diluent |
| 10 | Starch 1500 (+ Miglyol) | 97.3 | none | none | Separated suspension |
| | | | | | re-mixed before sampling |
| 11 | Cetyl alcohol 1-hexadecanol | 4.5 | none | none | Solution produced was a thick |
| | | | | | slime. Very hard to take |
| | | | | | HPLC sample |
| 12 | Paraffin wax | 15.0 | none | none | Material insoluble in sample |
| | | | | | diluent |
| 13 | Miglyol (fractionated coconut | 102.3 | none | none | Separated suspension |
| | oil) | | | | re-mixed before sampling |
| 14 | HPMC Methocel ® K15MP | 104.0 | none | none | |
| | (+ Miglyol) | | | | |
| 15 | HPMC Methocel ® K100MP | 98.9 | none | none | Separated of components |
| | (+ Miglyol) | | | | re-mixed before sampling |
| 16 | Methocel ® A (+ Miglyol) | 101.1 | none | none | |
| 17 | Hydrokote 112 | 104.2 | None | None | |
| 18 | Hydrokote AP5 | 101.2 | None | None | |
| 19 | Hydrokote M | 102.8 | None | none | |
| 20 | Shellac (+ Miglyol) | 99.8 | Peaks at | RT 5.057 = | Yellow semisolid |
| | | | 5.065, 10.702 | 0.1% - | Excipient insoluble in diluent |
| | | | and 12.491 | excipient | |
| | | | minutes | RT 10.436 = | |
| | | | | 0.1% | |
| | | | | RT 10.704 = | |
| | | | | 0.5% - | |
| | | | | excipient | |
| | | | | RT 12.488 = | |
| | | | | 0.3% - | |
| | | | | excipient | |
| | | | | RT 15.043 = | |
| | | | | 0.1% | |
| | | | | RT 15.402 = | |
| | | | | 0.1% | |
| 20 | Shellac UNSTRESSED | N/A | Main peaks: | N/A | Conclude: peaks present in |
| | | | 5.035, | | stressed Shellac were present |
| | | | 10.393, | | before stress test |
| | | | 10.656, | | |
| | | | 12.455 | | |
| | | | Several small | | |
| | | | peaks in time | | |
| | | | zone 14 to 18 | | |
| | | | minutes | | |
| 21 | Zein (+ Miglyol) | 100.5 | Peak at 7.083 | RT 7.080 = | Yellow semisolid |
| | | | minutes | 0.1% - | |
| | | | | excipient | |
| 22 | Aerosil ® COK 84 (+Miglyol) | 100.2 | none | None | |
| 23 | Aerosil ® 200 (+Miglyol) | 101.9 | none | none | |
| 24 | Cithrol ® GMS | 99.3 | Not available | none | Solution produced a viscous |
| | | | Control | | mix |
| | | | sample 96.4% | | |
| | | | assay | | |
| 25 | Sterotex ® | 62.9 | none | none | Solution produced a viscous |
| | | | | | mix |
| 25 | Sterotex ® REPEAT SAMPLE | 32.7 | none | none | Mean 2 samples 47.8% |
| 26 | Gelucire 50/02 | 104.1 | none | none | Solution produced a viscous |
| | | | | | mix |

The results above show that none of the excipients tested show any detectable signs of degradation or interaction after one month storage under conditions of 40° C./75% RH. It was therefore possible to use any of these materials as formulation ingredients.

### Initial Test Formulation Dissolution Testing

Preliminary test formulations were prepared based on poloxamer 188 and Gelucire 50/02. The formulation compositions are as below.

Formulation 052/014

| Material | % w/w | Quantity per cap mg |
|---|---|---|
| Poloxamer 188 | 62.8 | 282.7 |
| HPMC K100M | 17.9 | 80.3 |
| Aerosol COK 84 | 2.7 | 12.0 |
| Tramadol HCl | 16.6 | 74.9 |
| Capsule fill weight | | 450 |

Formulation 052/015

| Material | % w/w | Quantity per cap mg |
|---|---|---|
| Gelucire 50/02 | 58.3 | 233.3 |
| HPMC Pharmacoat 606 | 19.9 | 79.8 |
| Aerosil ® COK 84 | 3.0 | 12.0 |
| Tramadol HCl | 18.7 | 74.9 |
| Capsule fill weight | | 400 |

Placebo for 052/014

| Material | % w/w | Quantity per cap mg |
|---|---|---|
| Poloxamer 188 | 75.4 | 282.4 |
| HPMC K100M | 21.4 | 80.0 |
| Aerosol COK 84 | 3.2 | 12.0 |
| Capsule fill weight | | 374.4 |

Placebo for 052/015

| Material | % w/w | Quantity per cap mg |
|---|---|---|
| Gelucire 50/02 | 71.5 | 232.2 |
| HPMC Pharmacoat 606 | 24.8 | 80.6 |
| Aerosil ® COK 84 | 3.7 | 12.1 |
| Capsule fill weight | | 325 |

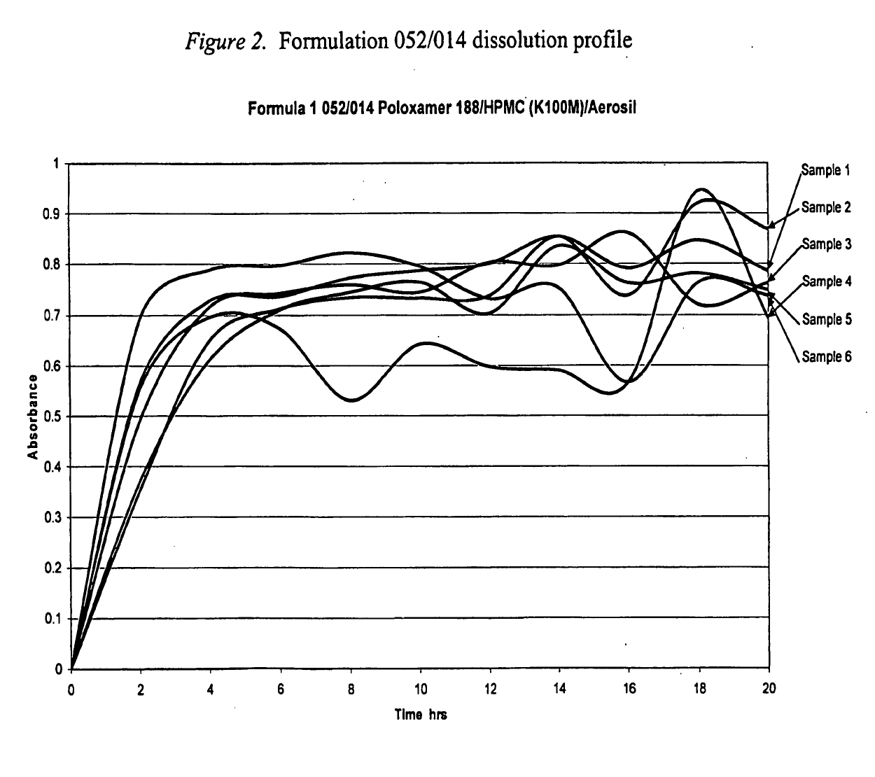

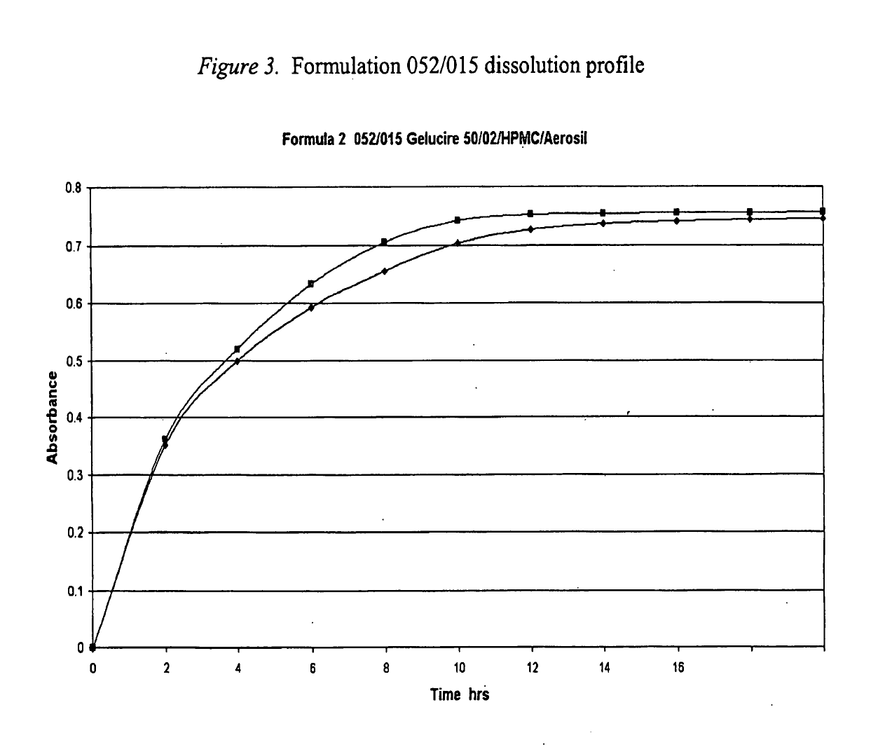

The release profiles, determined from dissolution testing in SIF are shown in FIGS. 2 and 3. Some HPMC gel remained at the end of the trial in sample 052/014 (poloxamer 188 based) but all poloxamer 188 and tramadol HCl had dissolved very quickly. Plot 2 shows that release took place over a 2-5 hr time span. This release rate is too fast to be useable in this project so the use of poloxamer 188 as a base excipient was discarded. The material of formulation 052/015 remained as a plug at the end of dissolution testing. It appears that the tramadol HCl and HPMC dissolved and migrates out through the Gelucire 50/02 over a period of 10-12 hr. This is shorter than the project targeted release time of 18-24 hr but Gelucire 50/02 was retained as a material worth testing further.

### Example 2

### Dissolution Testing of a Modified Gelucire 50/02 Formulation

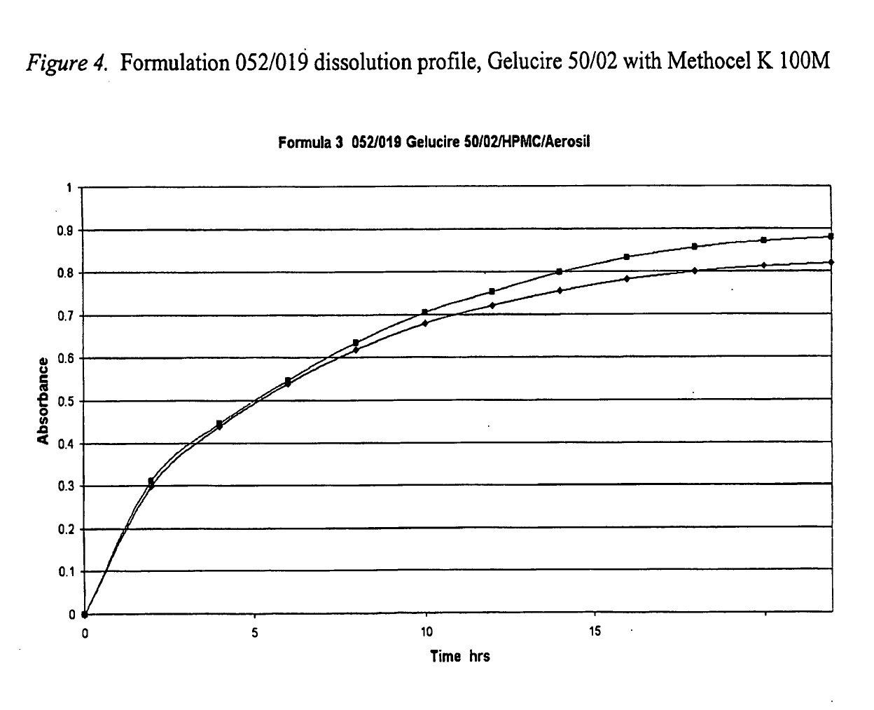

Methocel® K100M, a very high viscosity HPMC, was substituted for Pharmacoat 606, a very low viscosity HPMC, to investigate whether this substitution using a much higher viscosity HPMC would significantly slow the release rate of tramadol HCl from the formulation. The active and reference placebo capsules' formulations are shown in FIG. 4. It should be noted that the relative viscosity of HPMC is based on the viscosity of a 2% aqueous solution at 20° C. measured in mPas (millipascal Seconds). The numbers and letters in the HPMC's designation indicate (different manufacturers use slightly different conventions) the HPMC's 2% viscosity in mPas (1 mPas=1 centipoise (cps)), e.g. Pharmacoat 606 (Pharmacoat 6 is the HPMC type with the final 6 referring to the 2% viscosity) has a viscosity of 6 mPas (6 centipoise) as a 2% solution while Methocel® K100M (Methocel® K is the HPMC type and 100M is the 2% viscosity using the letter M as the convention for a multiplication factor of 1000) has a viscosity of 100,000 mPas (100 Pascal Seconds) as a 2% solution.

Formulation 052/019

| Material | % w/w | Quantity per cap mg |
|---|---|---|
| Gelucire 50/02 | 58.2 | 232.9 |
| Methocel ® K 100M | 19.9 | 79.4 |
| Aerosil ® COK 84 | 3.0 | 12.0 |
| Tramadol HCl | 18.7 | 75.0 |
| Capsule fill weight | | 400 |

Placebo for 052/019

| Material | % w/w | Quantity per cap mg |
|---|---|---|
| Gelucire 50/02 | 71.6 | 232.8 |
| HPMC Pharmacoat 606 | 24.6 | 79.8 |
| Aerosil ® COK 84 | 3.8 | 12.4 |
| Capsule fill weight | | 325 |

The dissolution rate had been slowed down slightly compared with 052/015 from 10-12 hr to approximately 15-18 hr. however, this mix was a thick cream and was probably too viscous to machine fill as this exact formulation.

### Example 3

### Dissolution Testing of Tramadol HCl in Gelucire 50/02 without Additional Excipients

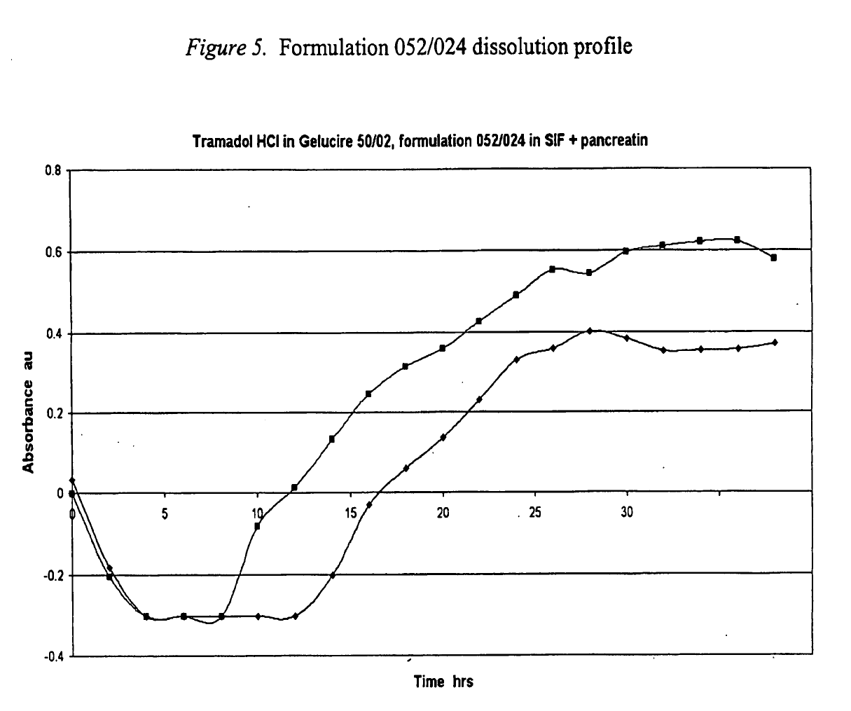

Initial dissolution trials on formulations were performed as ‘sighting’ trials to give some idea of the range of profiles possible for 75 mg of tramadol HCl in a matrix made up to 400 mg. The two major excipients used poloxamer 188 and Gelucire 50/02 are at opposite ends of the water solubility/dispersibility scale so would give a good indication of the range of release rates potentially available. Poloxamer 188 is readily water soluble while Gelucire 50/02 is highly lipophilic and only very slowly dispersible in water. The Gelucire 50/02 formulation 052/019 dissolution release rate, shown in FIG. 5, is close to that desired for this project. This formulation does incorporate materials which would modify (increase) the release rate so samples were prepared containing only tramadol HCl and Gelucire 50/02 to determine the slowest release rate that could be achieved with Gelucire 50/02. Samples were prepared according to the formulation below and their release rate determined.

Formulation 052/024

| Material | % w/w | Quantity per cap mg |
|---|---|---|
| Gelucire 50/02 | 81.2 | 325.0 |
| Tramadol HCl | 18.8 | 75.0 |
| Capsule fill weight | | 400 |

Placebo for 052/024

| Material | % w/w | Quantity per cap mg |
|---|---|---|
| Gelucire 50/02 | 100 | 325 |

A single capsule was initially tested then a further five capsules were also tested. All the data has been incorporated into the single plot shown below. The profile with the extended time scale is that of the first capsule tested.

These experiments indicate that full release takes place in the order of 30 hr. The outlying profiles was considered to be potentially due to uneven distribution of tramadol HCl in these hand mixed preparations but it was not deemed worthwhile to investigate this further at this stage. Gelucire 50/02 melts over a range centered on 50° C. and is hard enough to be crumbled into a powder. This makes formulations susceptible to abuse (by powdering, extraction, dose dumping, snorting etc) and it would be essential to include abuse deterrent materials such as HPMC and Aerosils in the final formulation. The release rate indicated by these profiles fall within the acceptable range of release rates worthy of further consideration at this stage of the project, however, as only two materials had been examined (with one rejected) by this stage it was decided to investigate other materials before narrowing the selection of potential formulations.

### Example 4

### Dissolution Testing of Tramadol HCl in Gelucire 50/02 in SIF Containing Pancreatin

The Gelucire range of materials is described as polyglycolized glycerides consisting of mono-, di- and triglycerides and of mono- and di-fatty acid esters of polyethylene glycol (PEG) with a range of HLB (hydrophilic lipophilic balance) values from 1 to 14. A material with a value of 14 is at the hydrophilic end of the scale where the material is easily water dispersible; 1 or 2 is at the other end of the scale and the material is extremely slowly water dispersible, at best.

Gelucire 50/02 (the 02 suffix shows the HLB value to be 2) is highly lipophilic and only disperses very slowly in aqueous media. These materials are potentially digestible so it is possible that a formulation that shows very slow release in vitro, in purely aqueous media such as SIF, could show dramatically faster release due to digestion, as opposed to dispersion, in vivo in the presence of enzymes.

An experiment was performed to look for any indications that the presence of an enzyme, pancreatin, modified the release rate of tramadol HCl in Gelucire 50/02. This experiment encountered difficulties as pancreatin in solution absorbs strongly over a range exceeding that of tramadol HCl's 240 nm to 290 nm band and pancreatin in suspension tended to block the solution filters.

The dissolution profile of capsules containing formulation 052/024 was recorded using UV absorbance determination. The pancreatin level was reduced to one fifth of that specified in the USP method so that solution absorbance values did not significantly exceed 1 au. The results shown below were very erratic, however, as this was intended as no more than a check on whether this family of materials (atypical of future excipients) was susceptible to acceleration of release rate by digestion it was decided not to divert the project into the development of an HPLC assay for tramadol HCL in the presence of pancreatin at this stage.

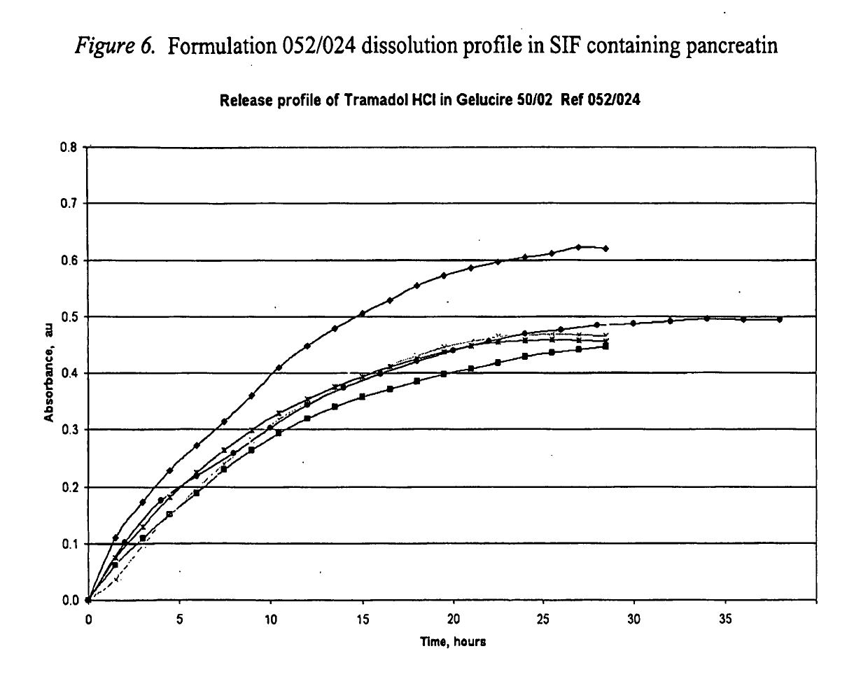

The profile (FIG. 6) shows an initial dip due to suspended/dissolved pancreatin affecting the reference cell. The absorbance of the mix appears to stop increasing after approximately 30 hr which does indicate that the tramadol HCl is fully released after this time. This corresponds well with the release time of tramadol HCl in this excipient tested in SIF in the absence of pancreatin (FIG. 5). This suggests that, at the level of pancreatin used, no major variation in dissolution release rate is observed in the presence of pancreatin. The Gelucire 50/02 units were allowed to be stirred in this medium for a further two days. The units maintained their shape and size for the entire period adding some confirmatory evidence that the Gelucire 50/02 content remained substantially unchanged (undigested).

### Example 5

### Dissolution Testing of Propranolol HCl in Gelucire 50/02 in SIF Containing Pancreatin

The above trial using Gelucire 50/02, as the base excipient, in SIF containing pancreatin suffered from the pancreatin UV absorbance overlapping and being of greater intensity than the tramadol HCl absorbance in the monitored 290 nm region. An alternative model compound was found in propranolol HCl, as a substitute for the tramadol HCl. Propranolol HCl has similar solubility and similar UV specific absorbance to tramadol HCl but has its UV absorbance maximum at 319 nm, just outside the absorbance window of pancreatin. This allowed the testing of the propranolol HCl analogue of the above formulation, 052/024, to be tested in the presence of pancreatin with reduced interference.

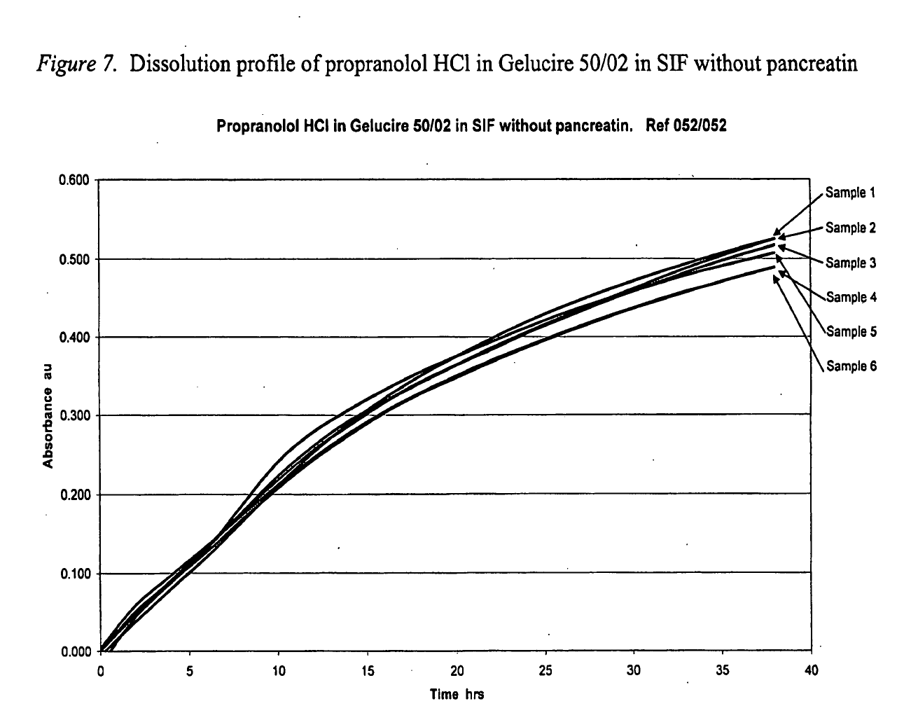

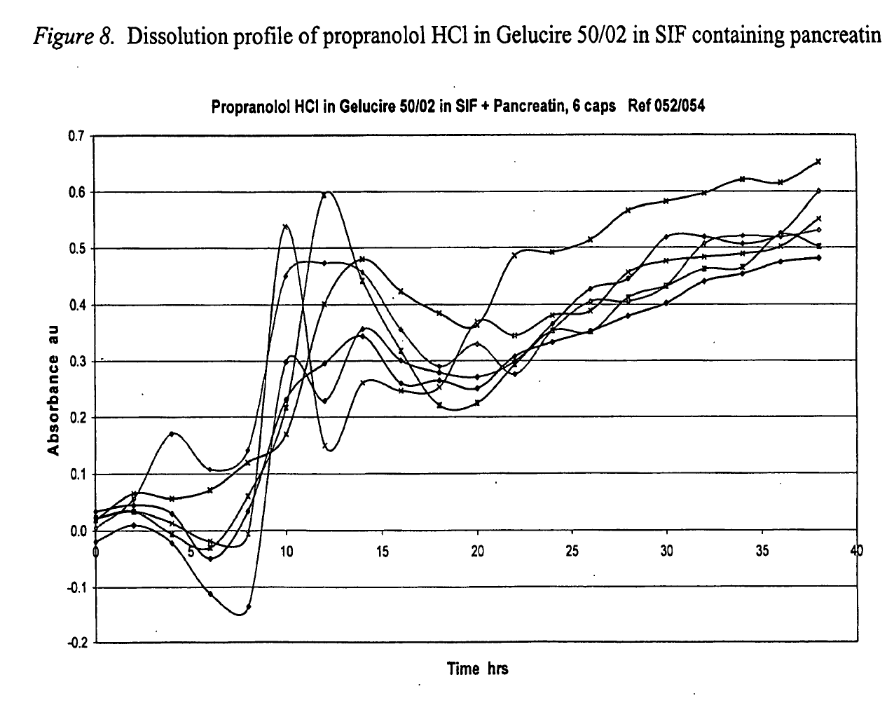

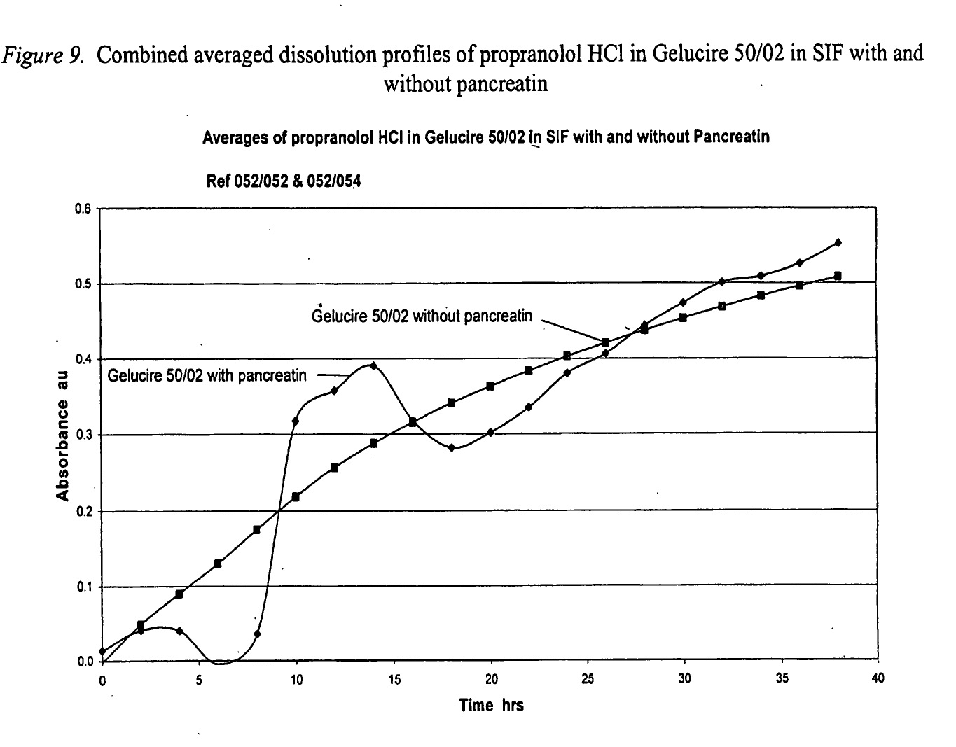

The propranolol HCl analogue was subjected to dissolution testing in 600 ml of SIF, with and without (full strength) pancreatin. Six capsule samples were tested in each case. FIGS. 7 and 8 shows data for dissolution with and without pancreatin while FIG. 9 shows the combined averaged data of dissolution in the absence and presence of pancreatin.

The pancreatin in suspension caused difficulties with filter blockage in both test and reference vessels leading to irregularities appearing in the data for propranolol HCl in SIF in the presence of pancreatin. Overall, despite the irregularities in the data, it is concluded that there is no difference detected in the overall rate of release for Gelucire 50/02 between dissolution in SIF in the absence or presence of pancreatin. This supports the conclusion reached for the similar experiment carried out using tramadol HCl in Gelucire 50/02.

### Example 6

### Dissolution Testing of Current Tramadol HCl Extended Release Products

Tramadol HCl is available in commercial extended release products. These products contain different doses of tramadol HCl, typically 150 mg, from the dosage unit under development in this project but it was considered useful to broaden our knowledge of such products and to obtain a dissolution release profile using our current conditions. It was also intended that proprietary products such as these were used later in this project as comparators during product tampering and extraction tests.

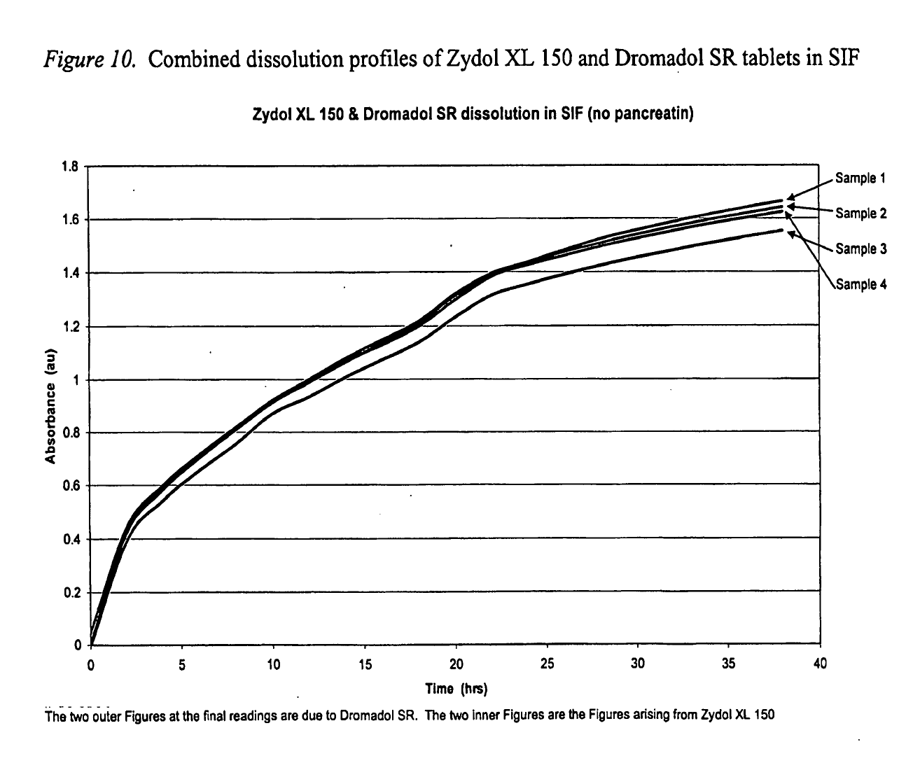

Zydol® XL 150 from Pfizer for once a day administration and Dromadol® SR by IVAX for twice a day administration are two proprietary products which both contain 150 mg of tramadol HCl in an extended release formulation. Two tablets of each product had their dissolution profile determined in 600 ml of SIF without added enzyme with UV monitoring at 271 nm according to the standard method used in this development project. The combined release profiles are shown in FIG. 10. All tablets were substantially whole at the end of the test period. The release profiles match so closely that it is not possible to distinguish visually one tablet type from the other. Under the above conditions full release takes of the order of 40 hr and, as the tablets contain double the dose of the experimental formulations, the final absorbance is approximately double that shown in earlier plots. The slight dip in the plot about 17 hr is considered to be an artifact of the method.

### Example 7

### Indicative Dissolution Testing of Potential Dosage Unit Base Excipients

Previous trials demonstrated that the hard fats and slowly dissolving materials were the best choice of base material (a base excipient is the predominant excipient in a dosage unit) for a 75 mg tramadol HCl extended release dosage unit. This identified seven other materials, from those tested in the compatibility trial, as potential base excipients. Six of these were formulated as binary mixtures with tramadol HCl and filled into capsules to a fill weight of 400 mg containing 75 mg tramadol HCl as had been carried out previously. The final material, beeswax, was formulated with the additional presence of HPMC as an unmodified formulation was unlikely to show any significant release due to the known insolubility of beeswax in aqueous media. All formulations had their dissolution profiles determined using single capsule samples for initial screening. The materials and formulations used are as below. The reference cell contained 600 mL of SIF.

Formulation 052/034-1

| Material | % w/w | Quantity per cap mg |
|---|---|---|
| Cetyl alcohol | 81.2 | 325.0 |
| Tramadol HCl | 18.8 | 75.0 |
| Capsule fill weight | | 400 |

Formulation 052/035-2

| Material | % w/w | Quantity per cap mg |
|---|---|---|
| Hydrokote 112 | 81.2 | 324.8 |
| Tramadol HCl | 18.8 | 75.2 |
| Capsule fill weight | | 400 |

Formulation 052/035-3

| Material | % w/w | Quantity per cap mg |
|---|---|---|
| Hydrokote AP5 | 81.3 | 325.2 |
| Tramadol HCl | 18.7 | 74.8 |
| Capsule fill weight | | 400 |

Formulation 052/035-4

| Material | % w/w | Quantity per cap mg |
|---|---|---|
| Hydrokote M | 81.3 | 325.4 |
| Tramadol HCl | 18.7 | 74.6 |
| Capsule fill weight | | 400 |

Formulation 052/035-5

| Material | % w/w | Quantity per cap mg |
|---|---|---|
| Cithrol ® GMS | 81.6 | 326.2 |
| Tramadol HCl | 18.4 | 73.8 |
| Capsule fill weight | | 400 |

Formulation 052/035-6

| Material | % w/w | Quantity per cap mg |
|---|---|---|
| Sterotex ® NF | 81.2 | 324.9 |
| Tramadol HCl | 18.8 | 75.1 |
| Capsule fill weight | | 400 |

Formulation 052/035-7

| Material | % w/w | Quantity per cap mg |
|---|---|---|
| Beeswax | 61.2 | 244.8 |
| Methocel ® K 100M | 20.1 | 80.5 |
| Tramadol HCl | 18.7 | 74.7 |
| Capsule fill weight | | 400 |

The above tests were carried out using only filtered SIF in the reference cell. Absorbance values obtained may be composed of two components, namely, absorbance due to tramadol HCl and absorbance due to dissolved excipient. 75 mg of Tramadol HCl in SIF gives an absorbance of 0.74 au therefore the absorbance must reach 0.7 au (allowing for inter capsule variation) before it is possible for all the tramadol HCl to have been dissolved. Absorbances significantly in excess of 0.7 au will have some contribution from excipient dissolution.

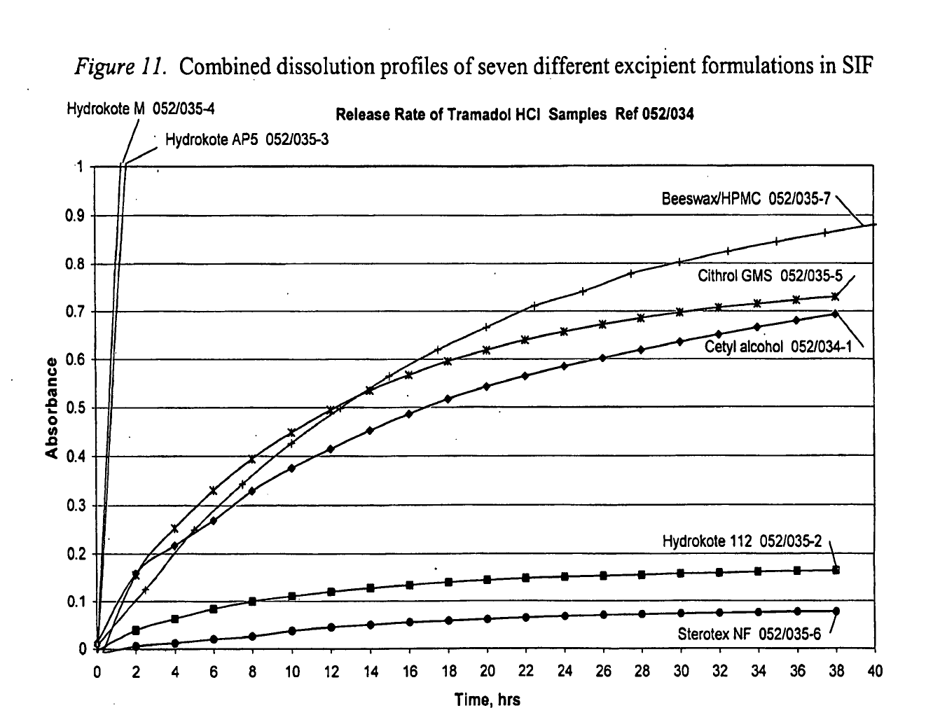

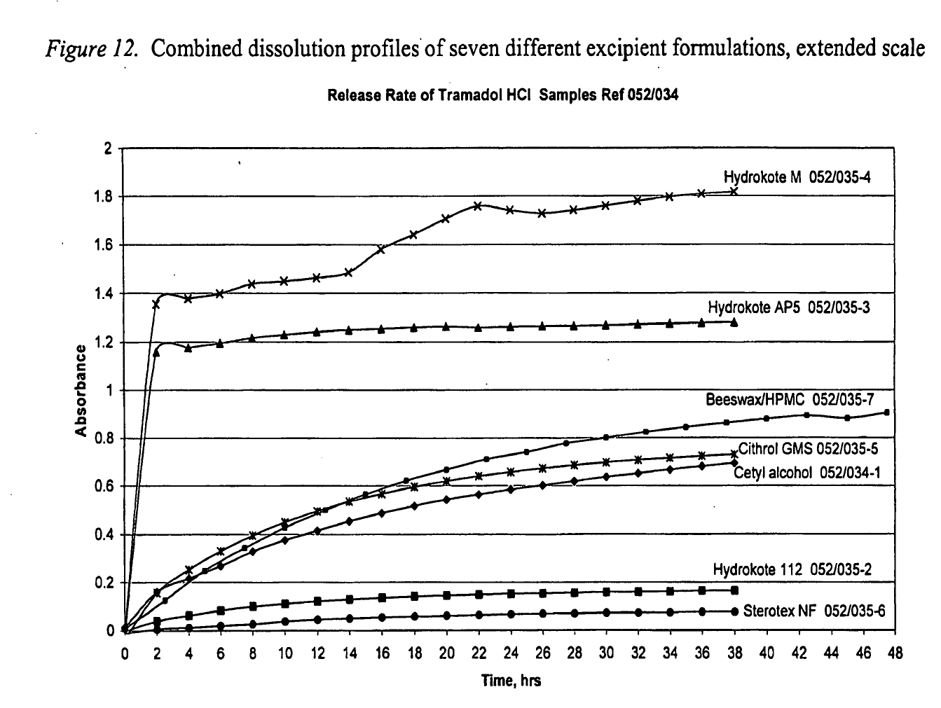

FIGS. 11 and 12 show that Hydrokote and Hydrokote AP5 dissolve rapidly and release their tramadol HCl in approximately 2 hours. This is too fast a release rate for the requirements of this project so these excipients were not able to be used as base excipients.

The other excipients were in two groups. Cithrol® GMS, Cetyl alcohol and the beeswax/HPMC combination showed release rates that were slightly slower than the target of total release in 18-24 hr while the Hydrokote 112 and Sterotex® NF were significantly slower. One of the requirements of this project is to develop dosage units with demonstrable deterrence to physical or solvent based tampering. Materials were to be incorporated into formulations to enhance abuse resistance. As it was likely that these materials would accelerate release then all of the materials mentioned in this paragraph were suitable for further consideration.

### Example 8

### Dissolution Testing of Modified Tramadol HCl Formulations

The base excipients Cithrol® GMS, Hydrokote 112, Cetyl alcohol, Sterotex® NF and beeswax showed potential as formulation base excipients in the trial above. These materials, in binary combination (beeswax as a ternary combination), gave dissolution release rates slower than the 18-24 hr target.

In this trial HPMCs were incorporated into the formulations to accelerate release and provide a level of tamper deterrence. Up to this point formulations contained tramadol HCl, a water soluble material, with a water insoluble base excipient which could make separation by extraction relatively easy. HPMC has been chosen as a material which might enhance tamper resistance as it has the property of being water soluble and thus would ‘follow’ tramadol HCl during attempted aqueous extraction, making separation of the tramadol HCl more difficult. HPMC comes in high viscosity grades which can impart a viscous nature to aqueous extracts of dosage units i.e. if anyone tries to extract the tramadol HCl with a small amount of water in a small spoon then, at best, they will produce an unpleasant mixture with a ‘gummy’ appearance which will tend to block attempts at filtration. Additionally, HPMC behaves in an unusual manner in aqueous solution. Most water soluble materials increase in solubility as the water temperature rises. HPMC is most soluble in cold water, becoming less soluble with temperature increase until, at about 40° C., it becomes totally insoluble. Solutions of HPMC that are heated to 40° C. or above turn into solid gels. This means that although an HPMC may be added to increase release rates from a dosage unit, it can actively deter abuse by extraction. If an individual tries to extract tramadol HCl with warm or hot water then the HPMC will become completely insoluble and actively resist the diffusion of tramadol HCl through the relatively impermeable base excipient.

Several formulations were produced incorporating a high viscosity HPMC, Methocel® K 100M, into the matrix. The formulations tested and the release profiles obtained are shown below.

Formulation 052/039-1

| Material | % w/w | Quantity per cap mg |
|---|---|---|
| Cetyl alcohol | 71.2 | 284.9 |
| Methocel ® K 100M | 10.0 | 40.0 |
| Tramadol HCl | 18.8 | 75.1 |
| Capsule fill weight | | 400 |

Formulation 052/039-2

| Material | % w/w | Quantity per cap mg |
|---|---|---|
| Hydrokote 112 | 57.0 | 227.9 |
| Methocel ® K 100M | 24.5 | 97.9 |
| Tramadol HCl | 18.6 | 74.2 |
| Capsule fill weight | | 400 |

Formulation 052/040-5

| Material | % w/w | Quantity per cap mg |
|---|---|---|
| Hydrokote 112 | 66.1 | 264.4 |
| Methocel ® K 100M | 15.1 | 60.3 |
| Tramadol HCl | 18.8 | 75.3 |
| Capsule fill weight | | 400 |

Formulation 052/039-3

| Material | % w/w | Quantity per cap mg |
|---|---|---|
| Cithrol ® GMS | 71.0 | 284.0 |
| Methocel ® K 100M | 10.2 | 40.8 |
| Tramadol HCl | 18.8 | 75.2 |
| Capsule fill weight | | 400 |

Formulation 052/040-4

| Material | % w/w | Quantity per cap mg |
|---|---|---|
| Sterotex ® NF | 56.5 | 225.8 |
| Methocel ® K 100M | 25.1 | 100.4 |
| Tramadol HCl | 18.4 | 73.8 |
| Capsule fill weight | | 400 |

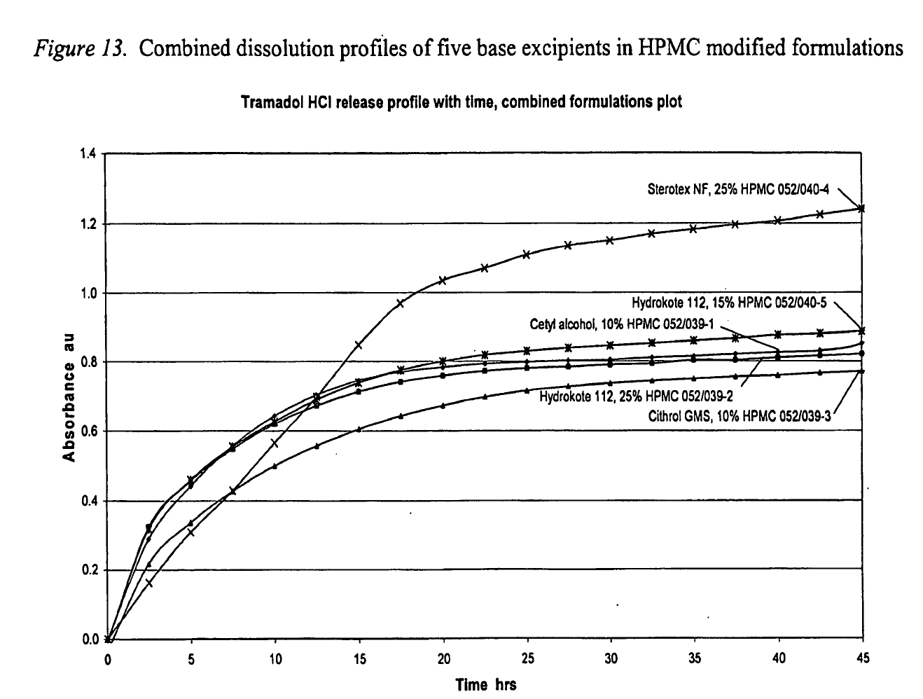

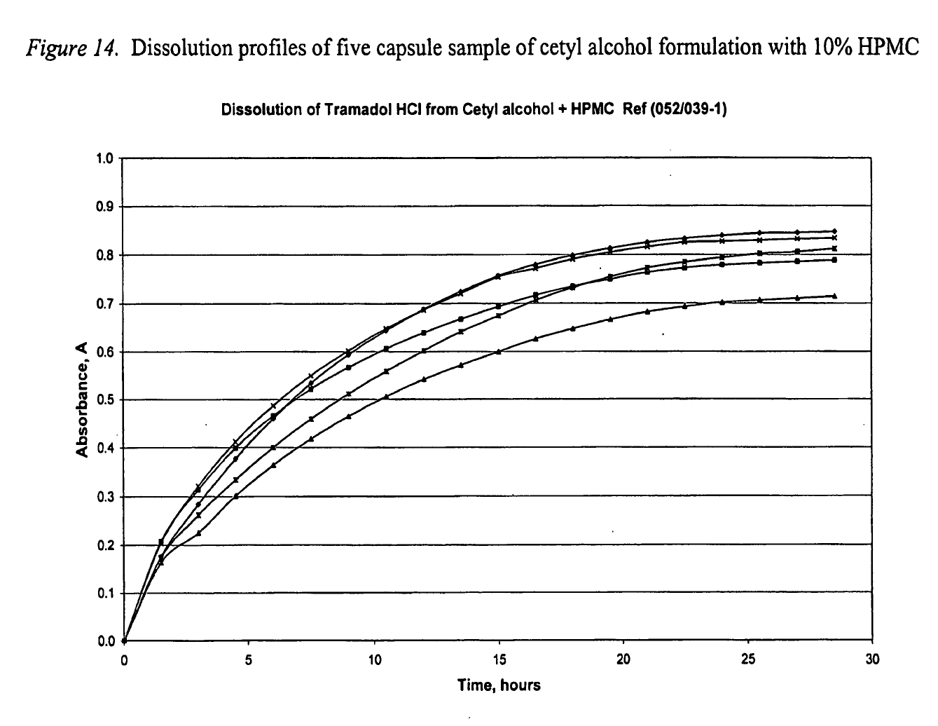

FIG. 13 is based on using only SIF in the reference cell. As described previously, the flattening of the curve, having reached an absorbance of at least 0.7 au, indicates full release of tramadol HCl from the dosage unit. Materials dissolving or suspending in the dissolution media may increase the recorded absorbance significantly above 0.7 as is clearly seen above for the Sterotex® NF plot. FIG. 13 shows that all formulations release all/almost all tramadol HCl within approximately 17-27 hr. This is satisfactory at this stage in the project. An example of the data and scatter for a five capsule dissolution set of results produced using one of the formulations used in the combined plot above (cetyl alcohol 052/039-1) is shown in FIG. 14.

### Example 9

### Dissolution Testing of Modified Tramadol HCl in Sterotex® NF Formulations

The future processing of formulations at manufacturing scale required to be considered at this stage. Some formulations had too low a viscosity, as a melt, to maintain insoluble excipients in suspension and others were so viscous that, although they could be hand filled for the purposes of these trials, they were so viscous that they would cause great difficulty during manufacture on full scale machinery. Formulations, unstable due to low viscosity, could have their viscosity increased using low levels of thixotrope but formulations of excessive viscosity required that excipients were reduced or substituted.

An Aerosil® was chosen as both a thixotrope and contributor to abuse deterrence. Aerosil® is the commercial name for fumed silicon dioxide manufactured by Degussa Hüls. They produce a range of Aerosils with differing properties. These include different particle size, hydrophobic or hydrophilic characteristics or blended with additional materials such as aluminum oxide for specific purposes. Aerosil® COK84 was chosen as the Aerosil® of choice for this project. Aerosil® COK 84 is a mixture of fumed silicon dioxide and highly dispersed aluminum oxide in a 5:1 ratio. This material effectively thickens aqueous systems and other polar liquids. In this project Aerosil® COK 84 will increase viscosity in a formulation, however, if attempts are made to add a small quantity of water to produce a solution (e.g. for injection) the Aerosil® COK 84 will contribute to increase the viscosity of any solution produced as it is specifically designed to thicken aqueous systems. Silicon dioxide and aluminum oxide, additionally, do not melt below 100° C. (or even 1000° C.) and are insoluble. The thickening effect of this Aerosil® is unaffected by heat thus an abuser attempting to melt a dosage unit will find that the structure and shape of the dosage unit tends to remain unchanged when sufficient Aerosil® is incorporated even though the melting point of all other excipients has been exceeded.

Formulations were modified by having Aerosil® COK 84 added in some instances to improve process characteristics and enhance abuse resistance while others had the HPMC grade substituted to bring the dissolution release rate towards the target range or to adjust the formulation properties to that required for commercial production.

The Sterotex® NF formulation above, 052/0404, contained 25% of a very high viscosity HPMC which produced a mix that could be hand filled but was excessively viscous for machine encapsulation. This formulation was modified with a lower quantity of a lower viscosity grade HPMC with the aim of producing a machine fillable formulation of similar release rate

Formulation 052/058

| Material | % w/w | Quantity per cap mg |
|---|---|---|
| Sterotex ® NF | 66.2 | 264.9 |
| Methocel ® K 15M | 15.0 | 60.0 |
| Tramadol HCl | 18.8 | 75.0 |
| Capsule fill weight | | 400 |

Placebo for 052/058

| Material | % w/w | Quantity per cap mg |
|---|---|---|
| Sterotex ® NF | 81.5 | 265 |
| Methocel ® K 15M | 18.5 | 60.0 |
| Capsule fill weight | | 325 |

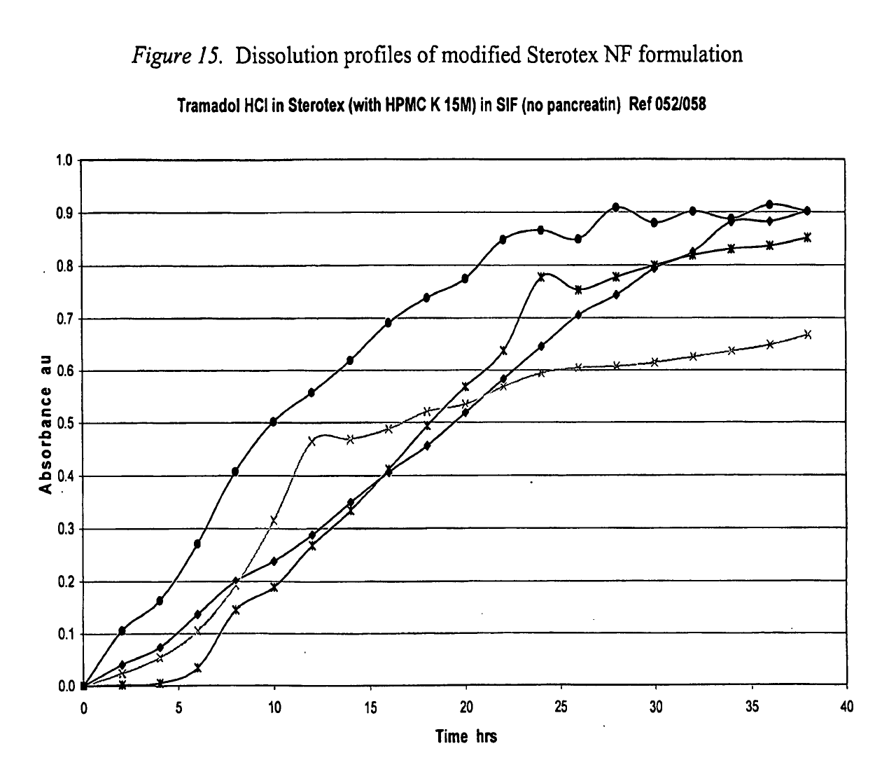

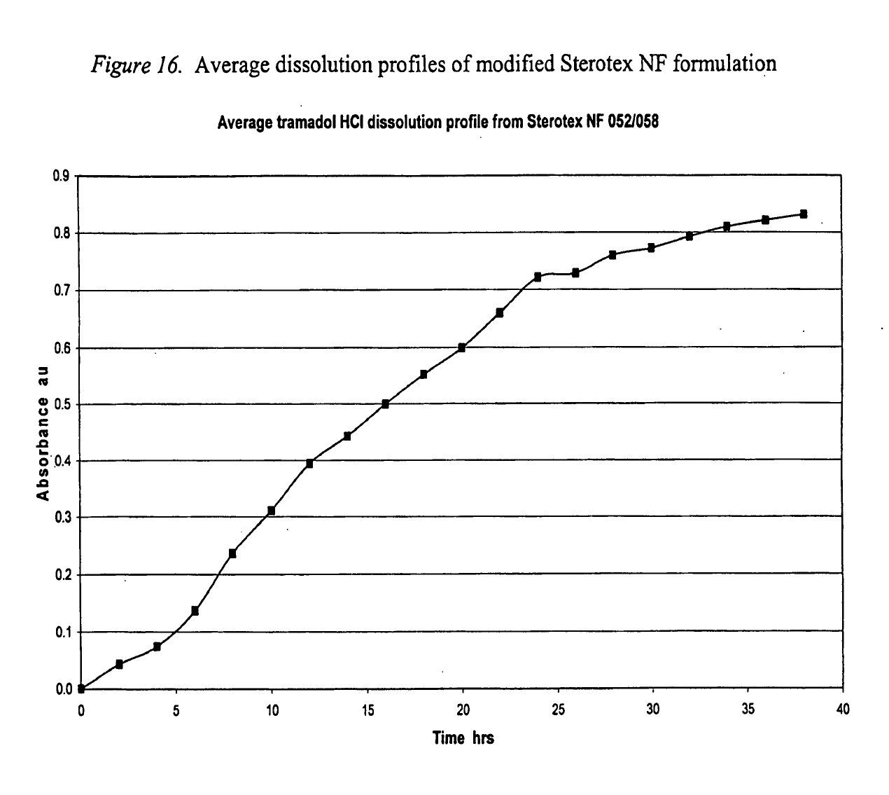

The dissolution profile of a four capsule sample is shown in FIGS. 15 and 16. The above profiles indicate release in 25-30 hr. (Later data will demonstrate that full release of 75 mg tramadol HCl from Sterotex® NF results in an absorbance of approximately 0.8 au under the above conditions). This formulation was quite thin with fast separation of the insoluble ingredients and required an increase in viscosity. This undoubtedly contributed to the variation between individual profiles. The dosage unit was swollen after dissolution testing but retained its original shape and was tough to break up. This demonstrated that the tramadol HCl has diffused out from the dosage unit rather than released after dosage unit dissolution or disintegration.

### Example 10

### Dissolution Testing of Further Modified Tramadol HCl in Sterotex® NF Formulations

Aerosil® COK 84 was added to the tramadol HCl in Sterotex® NF formulations. Formulations containing quantities of Aerosil® COK 84 in excess of 2% w/w were too viscous for machine filling so formulation 052/058 was modified to contain 2% Aerosil® COK 84 and subjected to dissolution testing against a placebo without tramadol HCl but which contained the same quantities of all other ingredients.

Formulation 052/060

| Material | % w/w | Quantity per cap mg |
|---|---|---|
| Sterotex ® NF | 63.9 | 255.4 |
| Methocel ® K 15M | 15.2 | 61.0 |
| Aerosil ® COK 84 | 2.1 | 8.6 |
| Tramadol HCl | 18.9 | 75.5 |
| Capsule fill weight | | 400 |

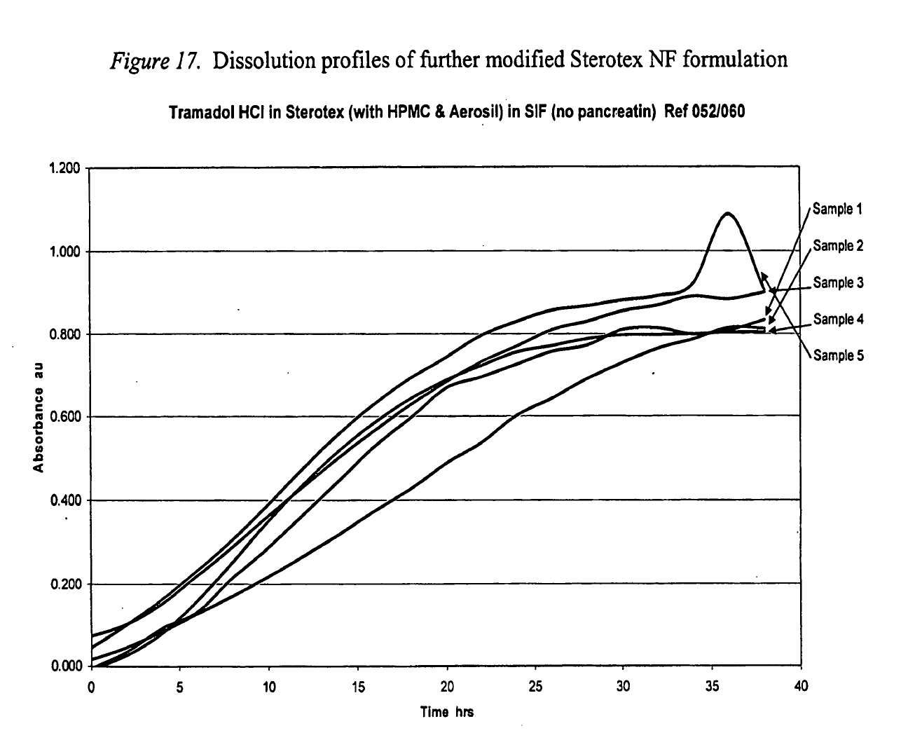

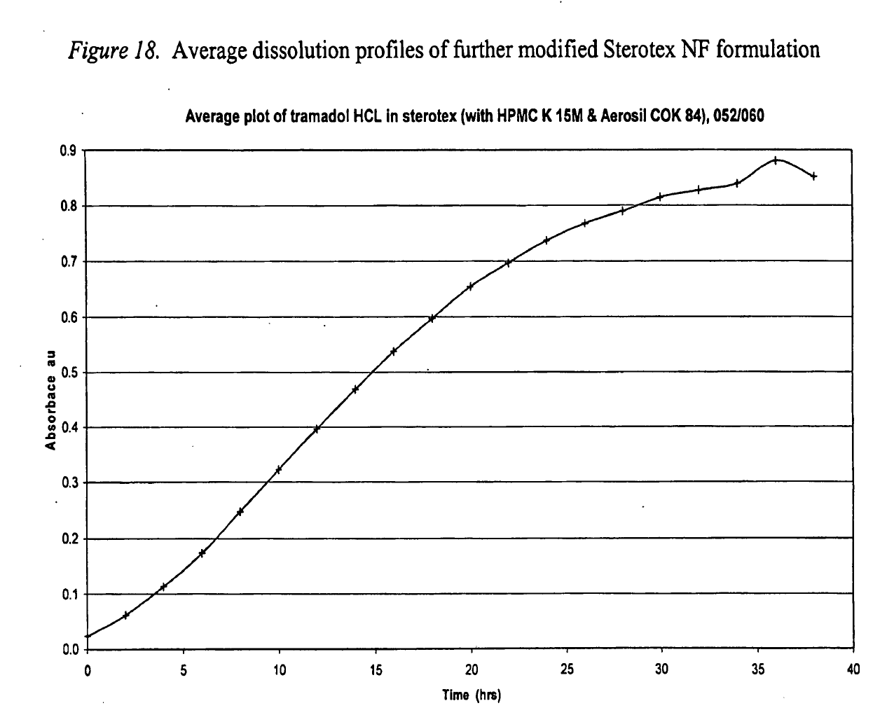

The dosage units had expanded and were soft and easily broken up after dissolution testing. The average release profile was not significantly different from that of formulation 052/058, with release in approximately 25-30 hr, however, there was less variation between individual samples indicating that low viscosity of 052/058 was a major contributor to individual sample variation (FIGS. 17 and 18).

### Example 11

### Dissolution Testing of Tramadol HCl in Hydrokote 112 with HPMC and Aerosil® COK 84

FIG. 13 shows the plot for a formulation based on Hydrokote 112 containing 15% Methocel® K 100M, formulation 052/040-5. Trials indicated that Aerosil® COK 84 could be incorporated at 1.5% w/w to produce a flowing light cream. The above formulation was modified to contain 1.5% Aerosil® COK 84 and to compare release profiles for formulations containing equal quantities of Methocel® K 15M or the much higher viscosity grade Methocel® K 100M. Formulations were prepared as below.

Formulation 052/062-1

| Material | % w/w | Quantity per cap mg |
|---|---|---|
| Hydrokote 112 | 64.7 | 258.7 |
| Methocel ® K 100M | 15.0 | 60.1 |
| Aerosil ® COK 84 | 2.1 | 8.6 |
| Tramadol HCl | 1.6 | 6.3 |
| Capsule fill weight | | 400 |

Formulation 052/062-2

| Material | % w/w | Quantity per cap mg |
|---|---|---|
| Hydrokote 112 | 64.7 | 258.6 |
| Methocel ® K 15M | 15.0 | 60.2 |
| Aerosil ® COK 84 | 2.1 | 8.6 |
| Tramadol HCl | 1.5 | 6.2 |
| Capsule fill weight | | 400 |

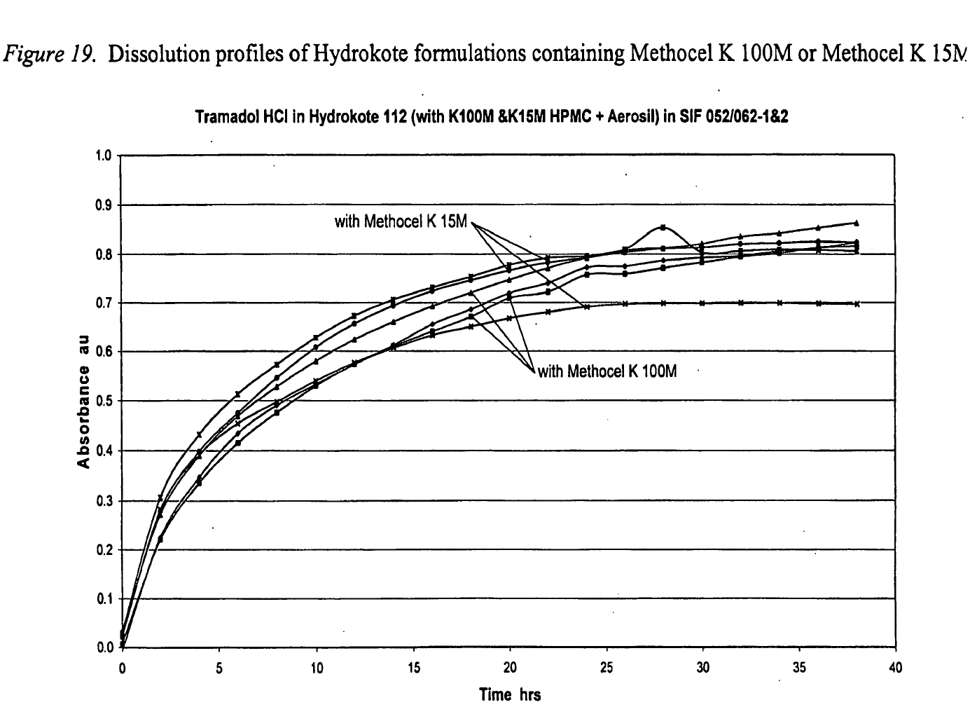

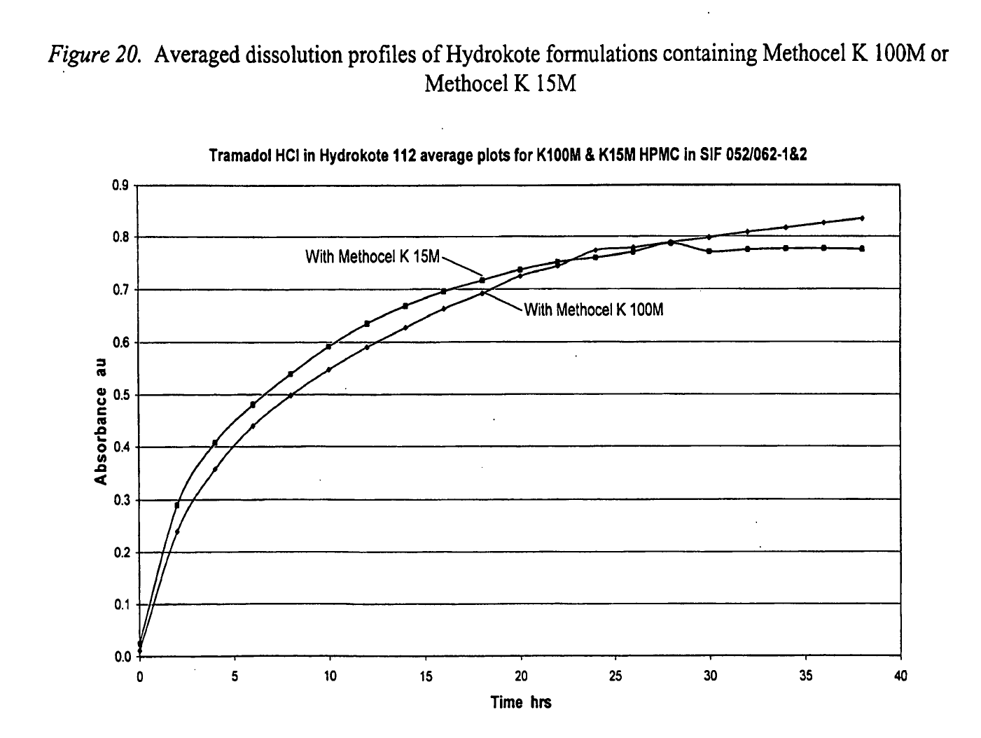

Three capsule samples of each formulation had their dissolution absorbance profiles measured in 600 mL of SIF, without enzyme at 271 nm, using the USP paddle apparatus, at 75 rpm, as carried out previously. The combined individual and averaged profiles are shown in FIGS. 19 and 20. Both dosage units were soft and crumbling at the end of dissolution testing. Both gave acceptable release times for the tramadol HCl of 25-30 hr. As would be expected, the lower viscosity grade dissolution was slightly faster than that of the formulation containing the higher viscosity grade.

### Example 12

### Dissolution Testing of a Formulation Containing 250 mg Tramadol HCl in Sterotex® NF

A dosage unit containing 250 mg of tramadol HCl was considered as a future possibility for this type of slow release dosage form so a preliminary investigation was carried out to estimate the likelihood of this being achievable.

Tramadol HCl is highly water soluble. This can lead to difficulty in producing a slow release formulation as, with the preferred largest capsule size as a size 0, the largest quantity of formulated material that can be filled as a liquid fill is approximately 550 mg. This means that the formulation will contain approximately 45% as the very soluble tramadol HCl.

The objective of this exercise was to determine whether 250 mg tramadol HCl could be formulated to 500-550 mg in a mix, with the properties to enable machine filling, and having a release rate that delivered the tramadol HCl into solution over at least 18-24 hr. If the formulation released tramadol at a much slower rate then this was completely acceptable as the release rate could be accelerated by the incorporation of materials such as HPMC. Difficulties would arise if the release rate could not achieve 18-24 hr release with only the base excipient.

Sterotex® NF was chosen as the base excipient for this trial as, at the 18.8% w/w tramadol HCl level (FIG. 12), it was the ‘slowest’ of the excipients under examination and able to deliver extremely slow release. A formulation targeted on 500 mg dosage was too viscous to be filled. Diluting to a total mass of 550 mg and the addition of a small quantity of Aerosil® COK 84 gave a flowing cream that could be machine filled.

Formulation 052/066

| Material | % w/w | Quantity per cap mg |
|---|---|---|
| Sterotex ® NF | 52.8 | 290.5 |
| Aerosil ® COK 84 | 1.8 | 10.0 |
| Tramadol HCl | 45.4 | 249.6 |
| Capsule fill weight | | 550 |

The dissolution profile of a six capsule set was obtained in the previous manner. The only difference from previous conditions was that the dissolution medium volume had been increased to 1 liter. At this level, total release of the 250 mg of tramadol HCl would give an absorbance of at least 1.5 au. A placebo containing all materials in identical quantities without tramadol HCl was used as the reference.

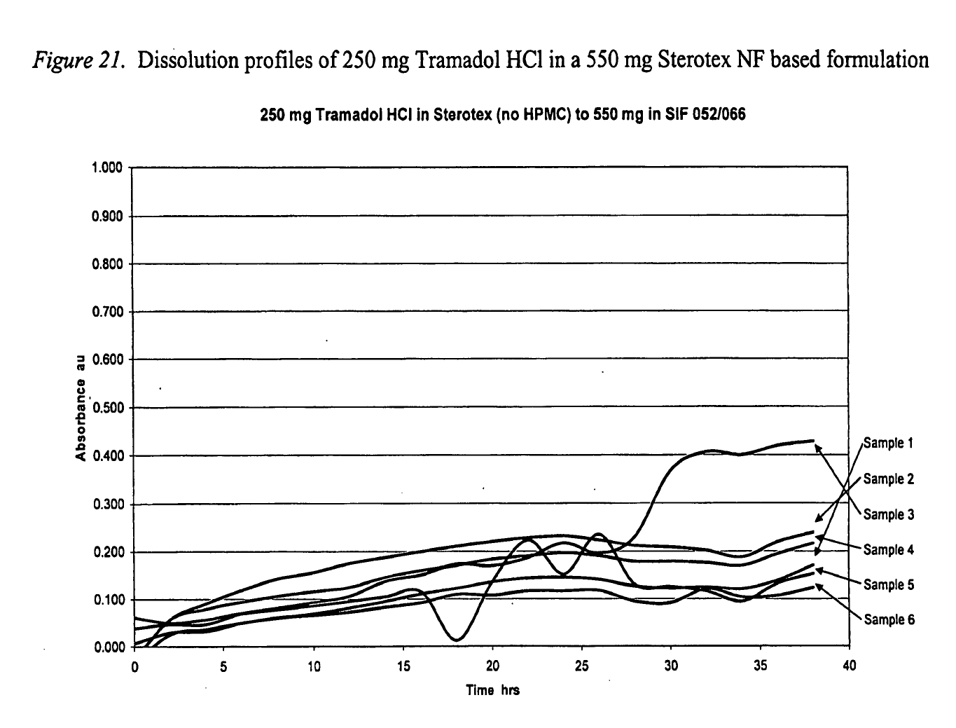

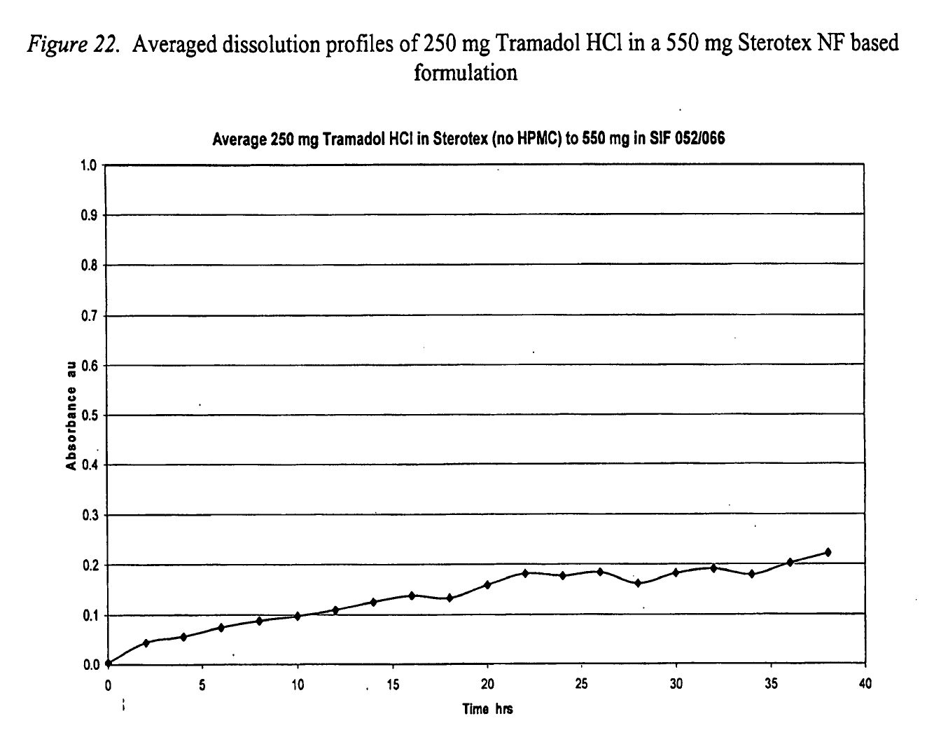

The individual plots (FIGS. 21 and 22) showed some atypical behavior due to bubble generation in the flow through cells. Despite this, the clear observation is that this formulation released less than a quarter of its tramadol HCl content over the 38 hr period of the dissolution trial. This release time and the percentage released comfortably exceeds the minimum requirement of release of all tramadol HCl in not less than 18-24 hr. This trial demonstrates that it should be feasible to produce a similar slow release, liquid filled dosage unit to the objective of this project, containing up to 250 mg tramadol HCl in a total formulated mass of up to 550 mg.

### Example 13

### Dissolution Testing of Tramadol HCl in Beeswax Based Formulations

Previous beeswax based formulations (052/035-7), containing 20% Methocel® K 100M released in a period of approximately 40 hr. This exceeded the 18-24 hr target range of the study, however, it was considered useful to include a slightly slower, in vitro, formulation to broaden the range of formulations that would eventually be subject to an in vivo trial.

Two other beeswax formulations were prepared to compare the quantity and type of HPMC that should be incorporated and the effect of Aerosil® COK 84 inclusion. It was found that up to 2% Aerosil® COK 84 could be included and the material remained as a potentially machine tillable mix. 25% HPMC was found to produce an excessively viscous mix. Two formulas were tested containing 20 and 23% w/w of the lower viscosity Methocel® K 15M HPMC. The formulations subjected to dissolution testing were as below.

Formulation 052/068

| Material | % w/w | Quantity per cap mg |
|---|---|---|
| Beeswax | 59.4 | 237.6 |
| Methocel ® K 15M | 19.9 | 79.5 |
| Aerosil ® COK 84 | 2.0 | 8.2 |
| Tramadol HCl | 18.7 | 74.7 |
| Capsule fill weight | | 400 |

Formulation 052/070

| Material | % w/w | Quantity per cap mg |
|---|---|---|
| Beeswax | 56.3 | 225.0 |
| Methocel ® K 15M | 23.0 | 92.0 |
| Aerosil ® COK 84 | 2.0 | 8.0 |
| Tramadol HCl | 18.7 | 75.0 |
| Capsule fill weight | | 400 |

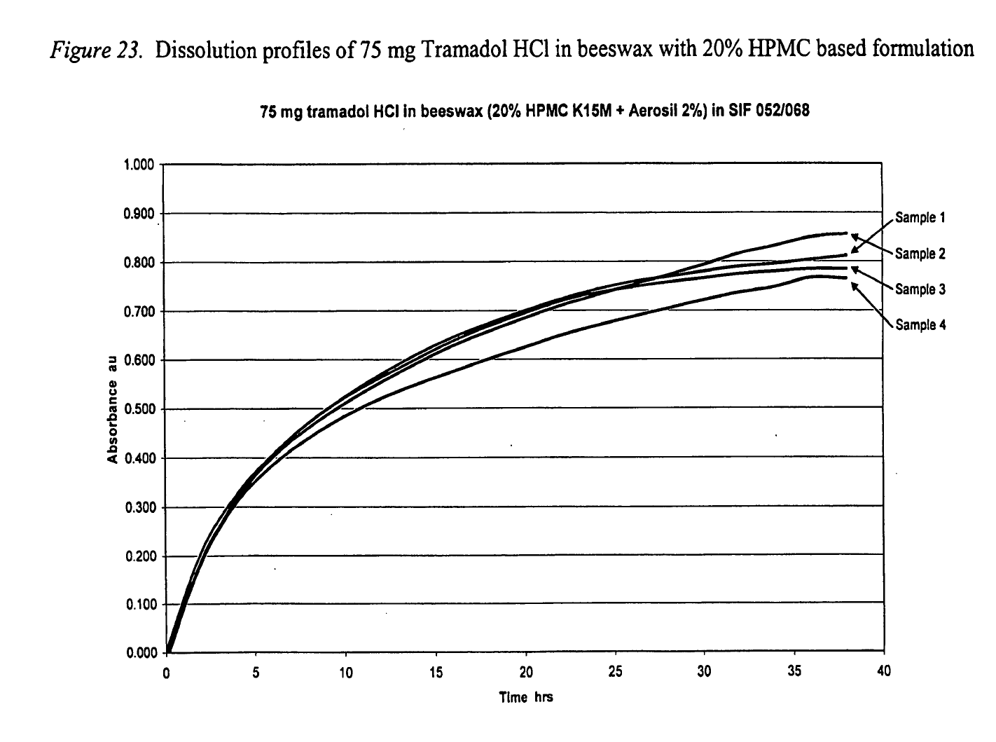

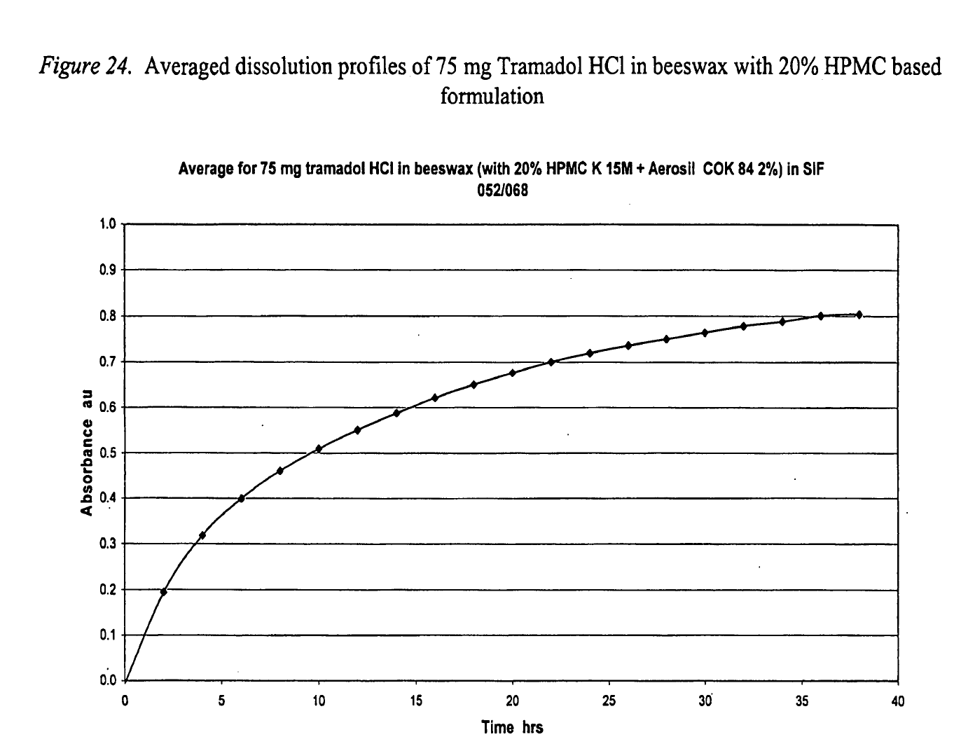

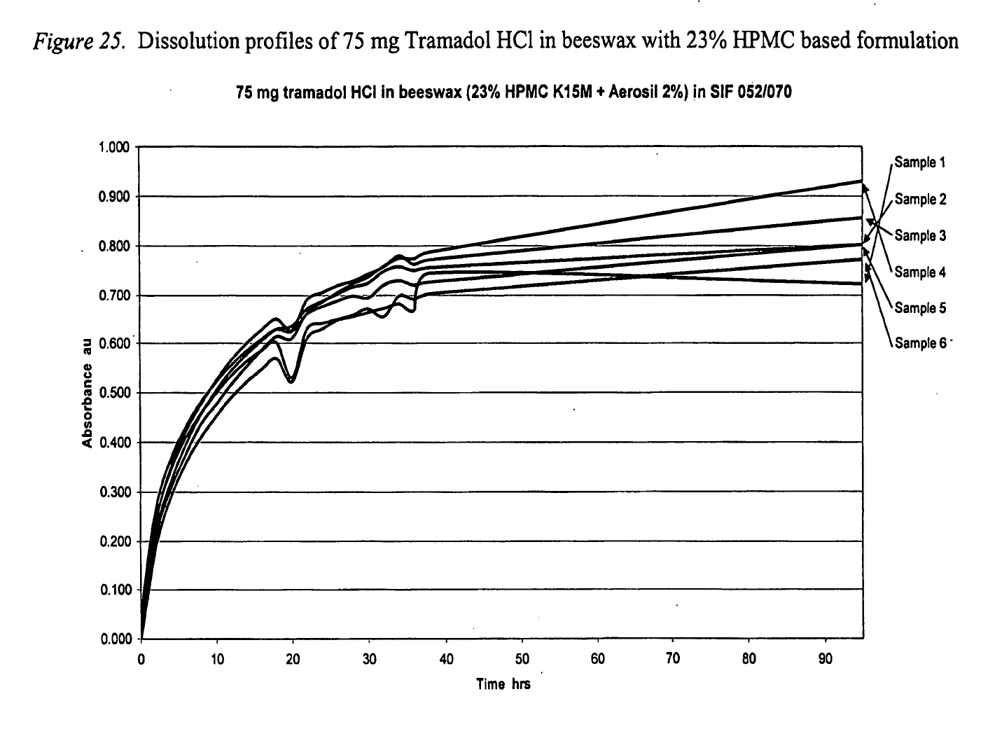

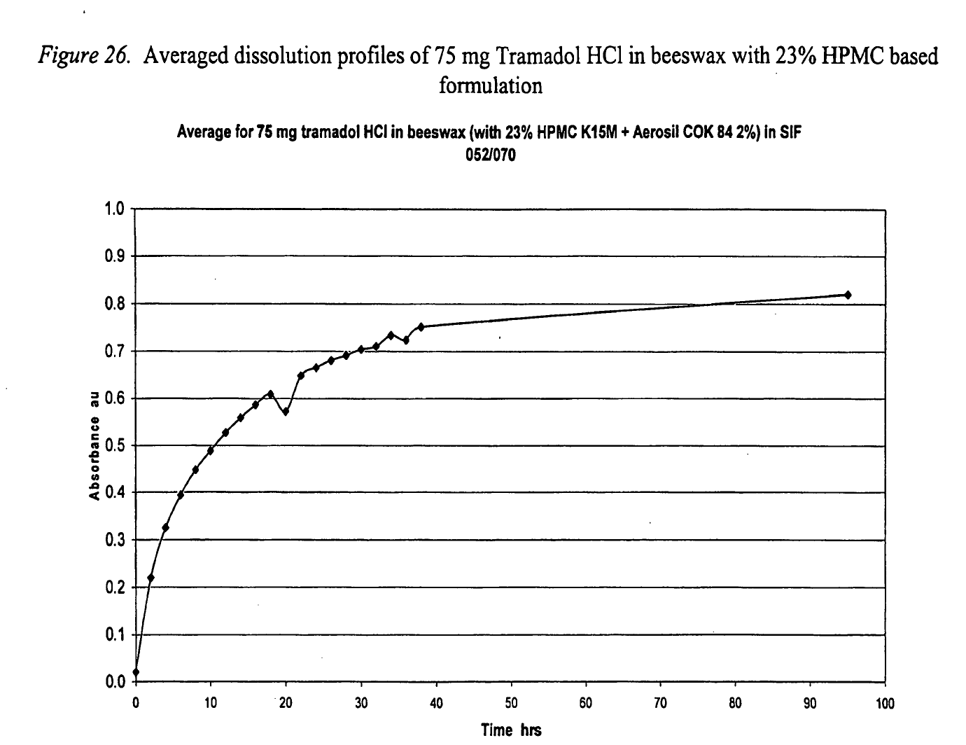

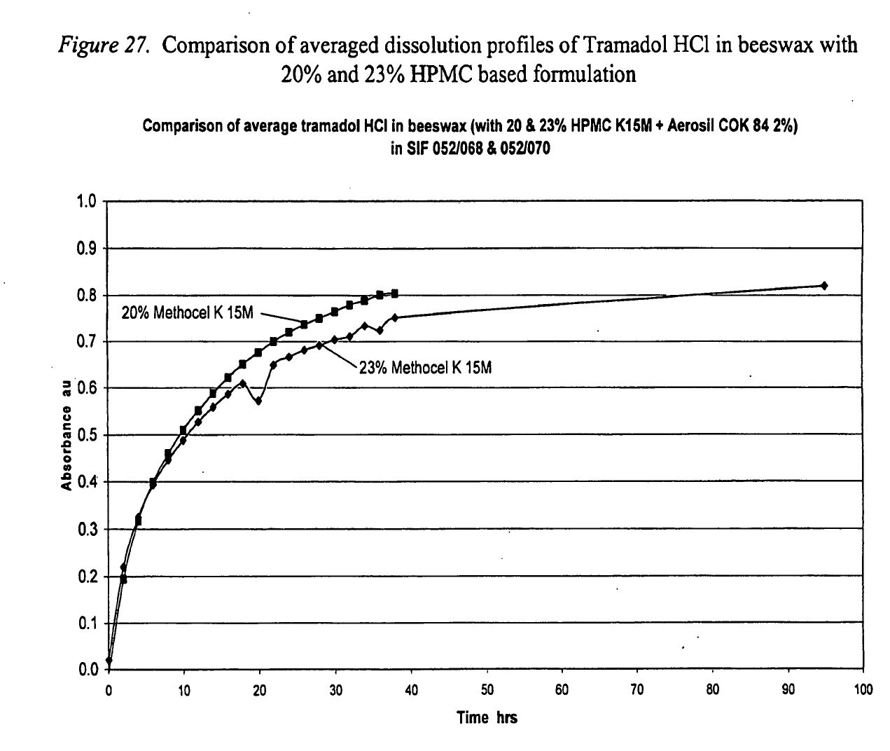

The dissolution profiles of both formulations were obtained using 600 mL of SIF and the USP paddle method with monitoring at 271 nm, unchanged from previous dissolution trials. Placebos containing all materials in identical quantities without tramadol HCl were used as the reference in each case. The dissolution profiles obtained shown in FIGS. 23, 24, 25, 26 and 27.

Tramadol HCl was released over approximately 40 hr in both cases. The dissolution of 052/070, containing 23% Methocel® K 15M, was allowed to continue running for 95 hr to confirm the final absorbance achieved. It would have been expected that formulation 052/070, containing slightly more soluble matter, would have shown the faster release. It appears that there is little real difference in release rates at this level of HPMC content so the formulation containing 20% Methocel® K 15M was selected for use.

### Example 14

### HPLC Analysis of Tramadol HCl During Dissolution Testing

Tramadol HCl release during dissolution testing had been monitored to this point using the absorbance of the dissolution media at 271 nm (absorbance maximum for tramadol HCl at longest wavelength) as a function of the quantity of tramadol HCl released into solution. This approach was reasonable as the excipients used in formulations were either almost insoluble or had negligible absorbance at this wavelength. It was considered that tramadol HCl was fully released when the absorbance of the solution became constant. For 75 mg tramadol formulations and the system used, this meant that the absorbance would be in excess of 0.7 au. The absorbance profile would be composed of absorbance from tramadol HCl plus a small contribution from absorbance/scattering from the other excipients.

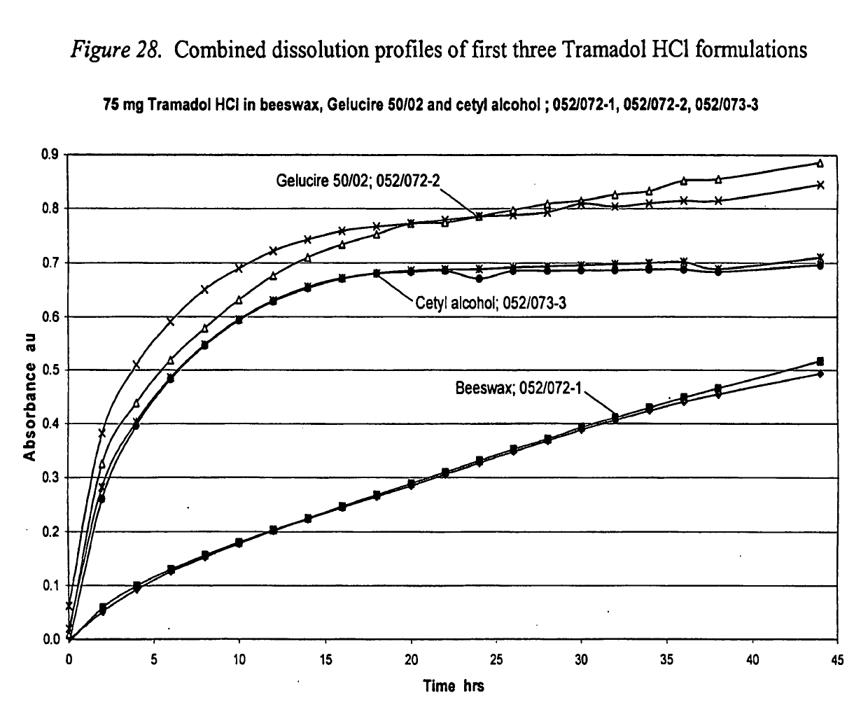

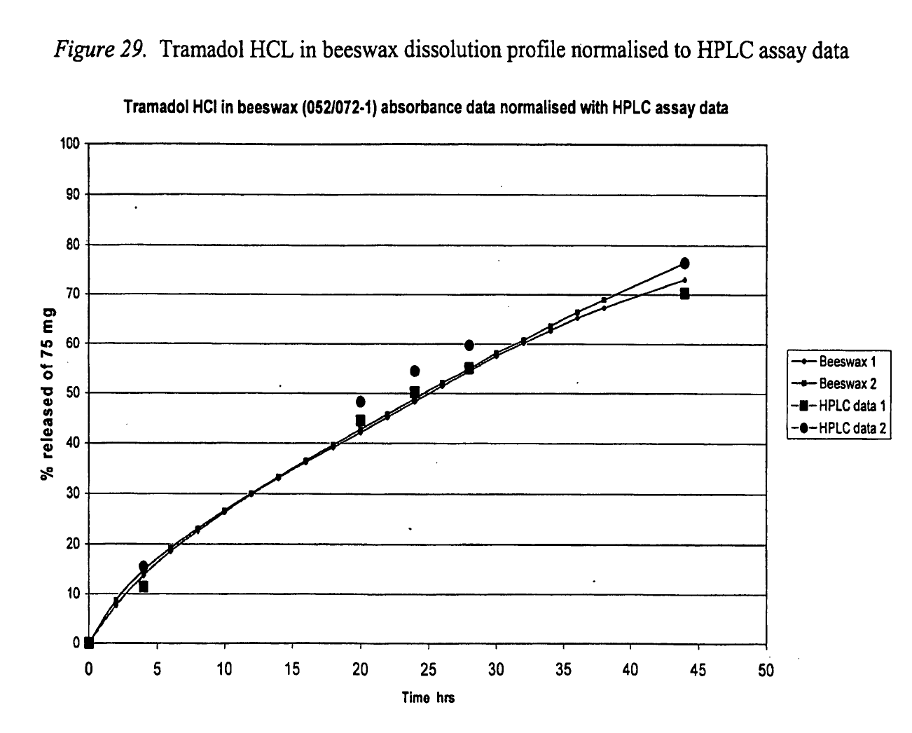

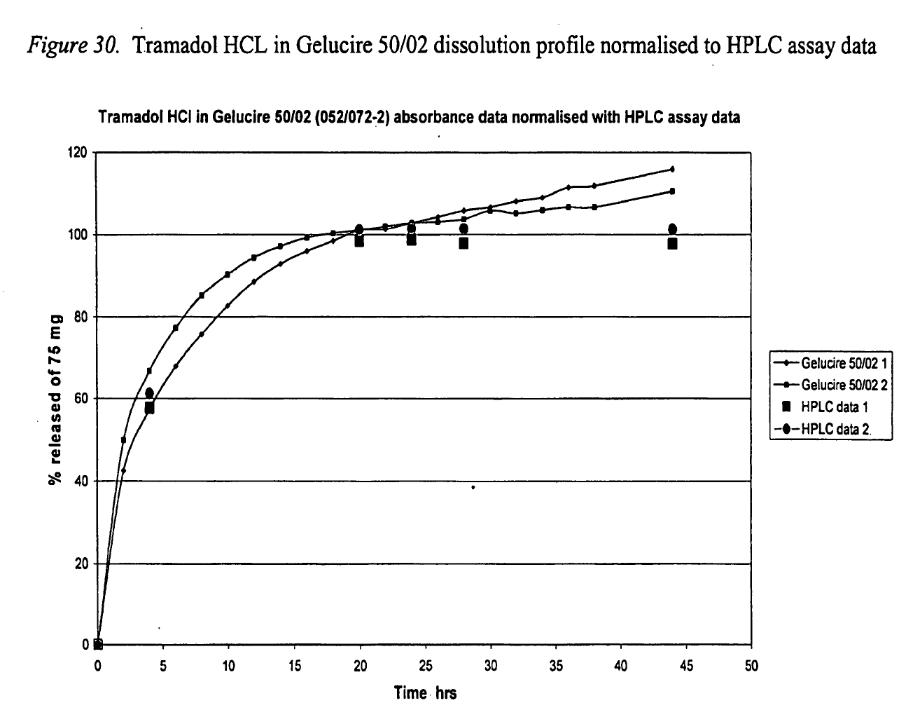

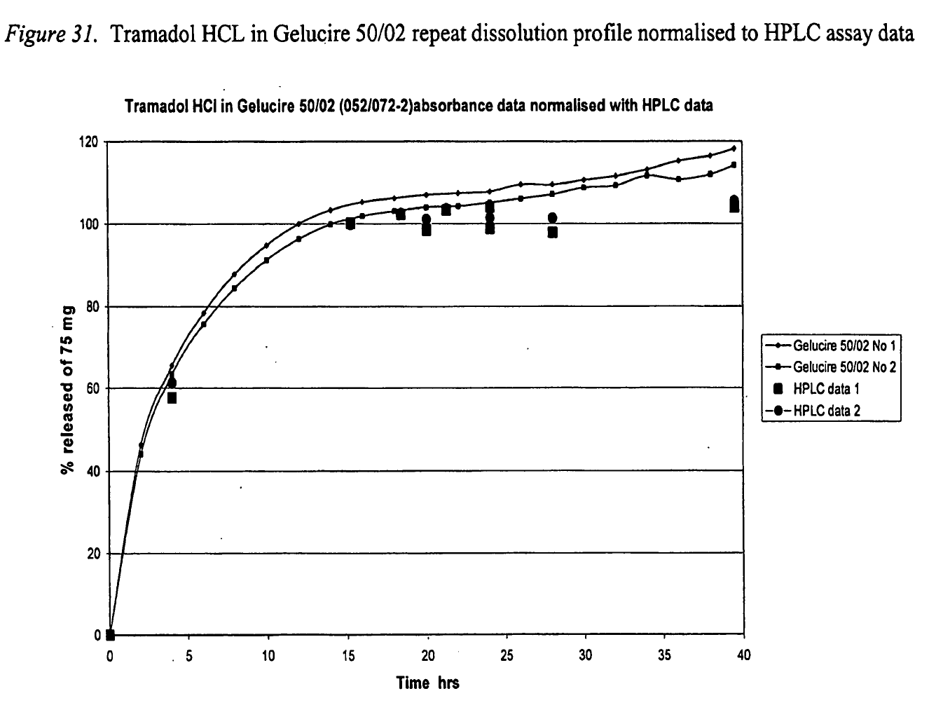

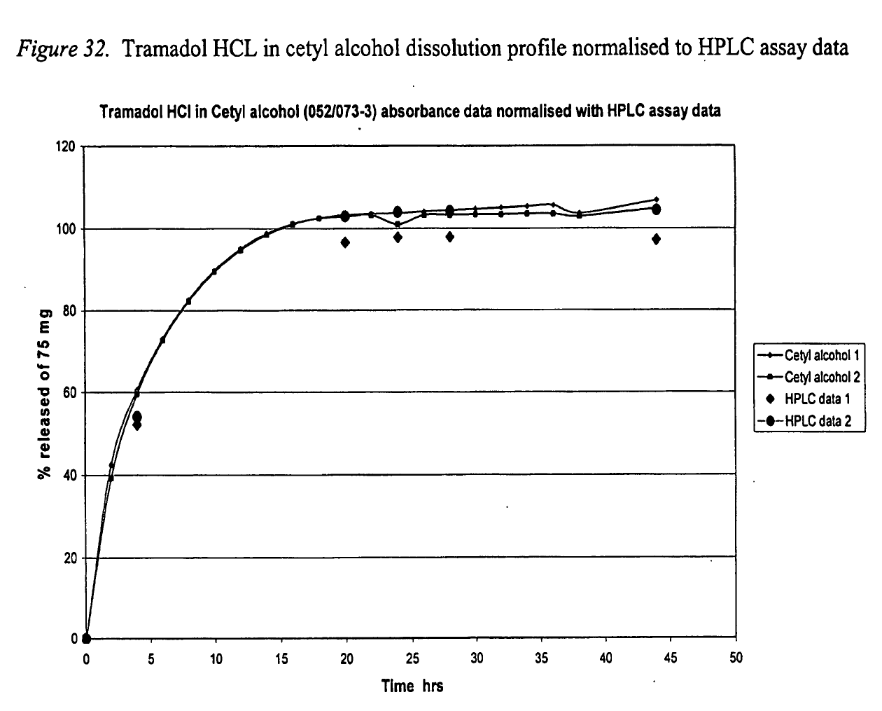

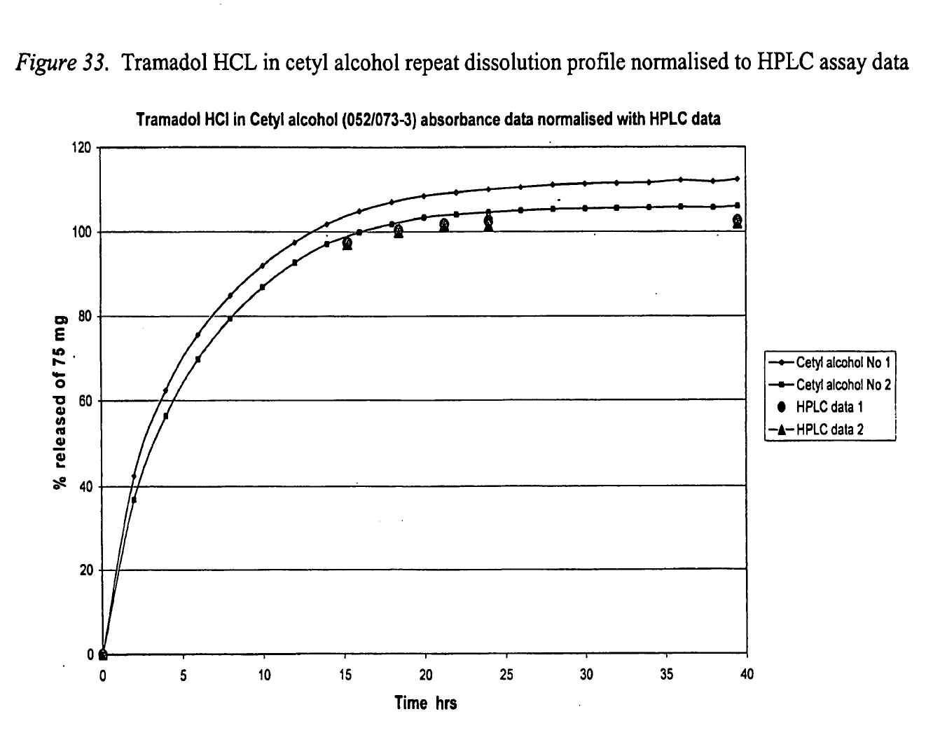

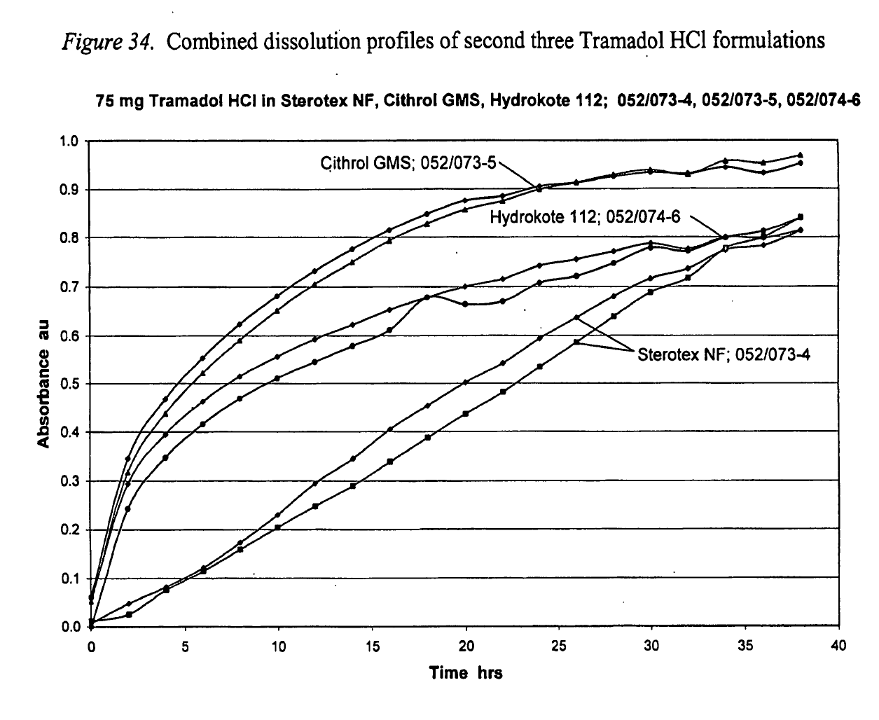

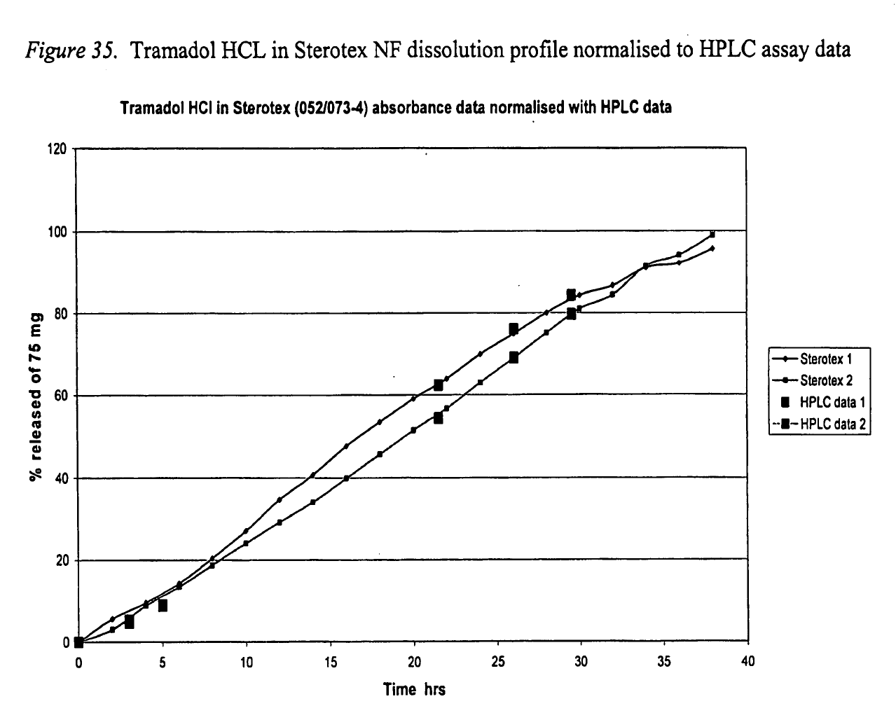

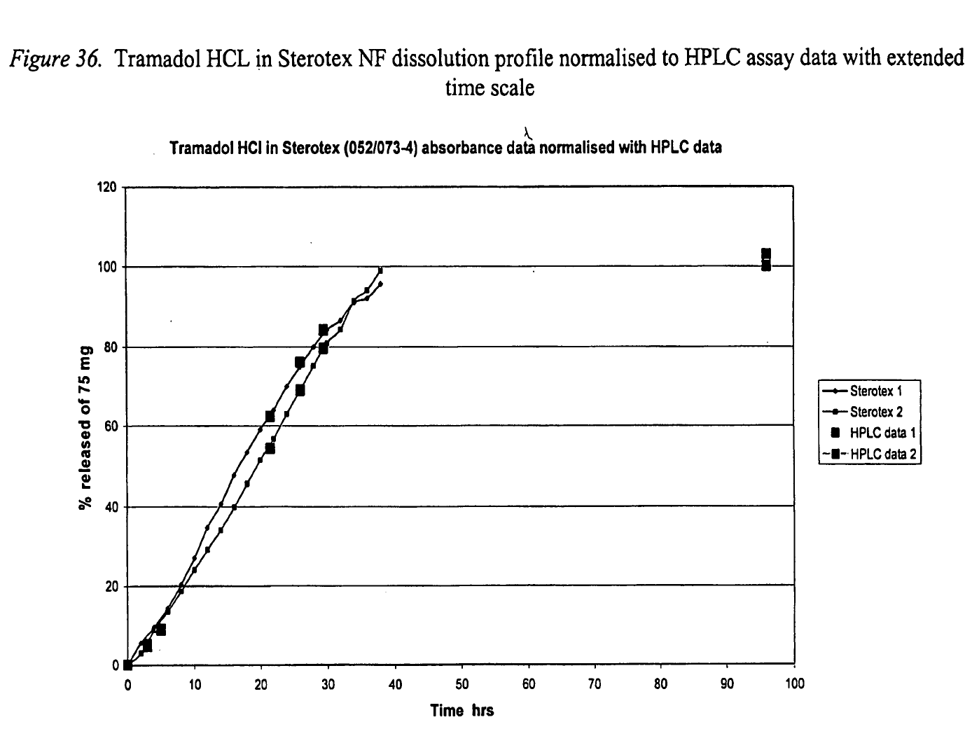

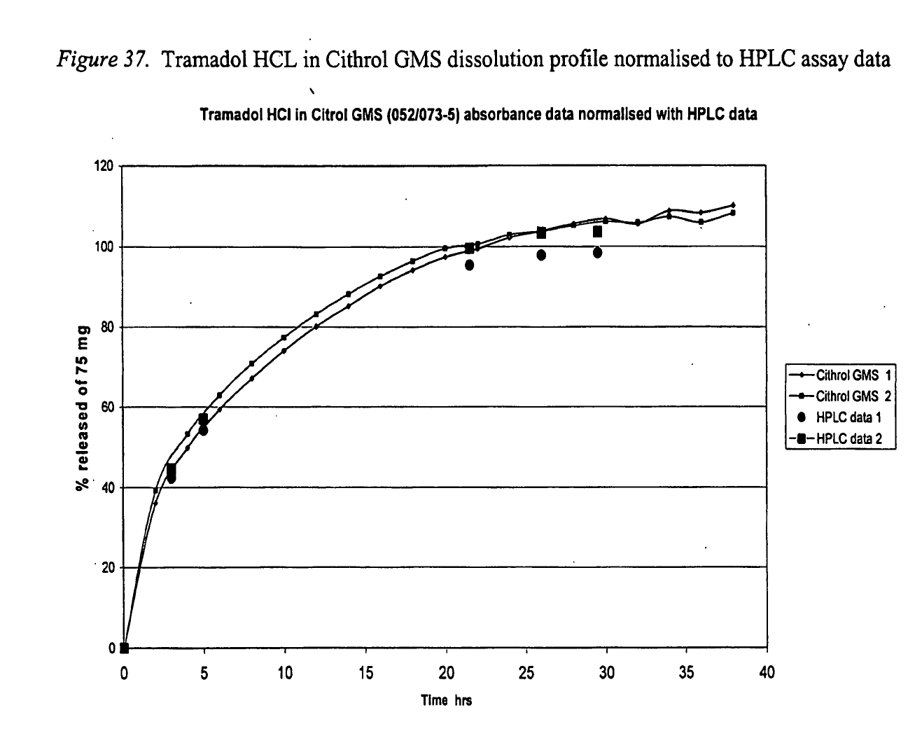

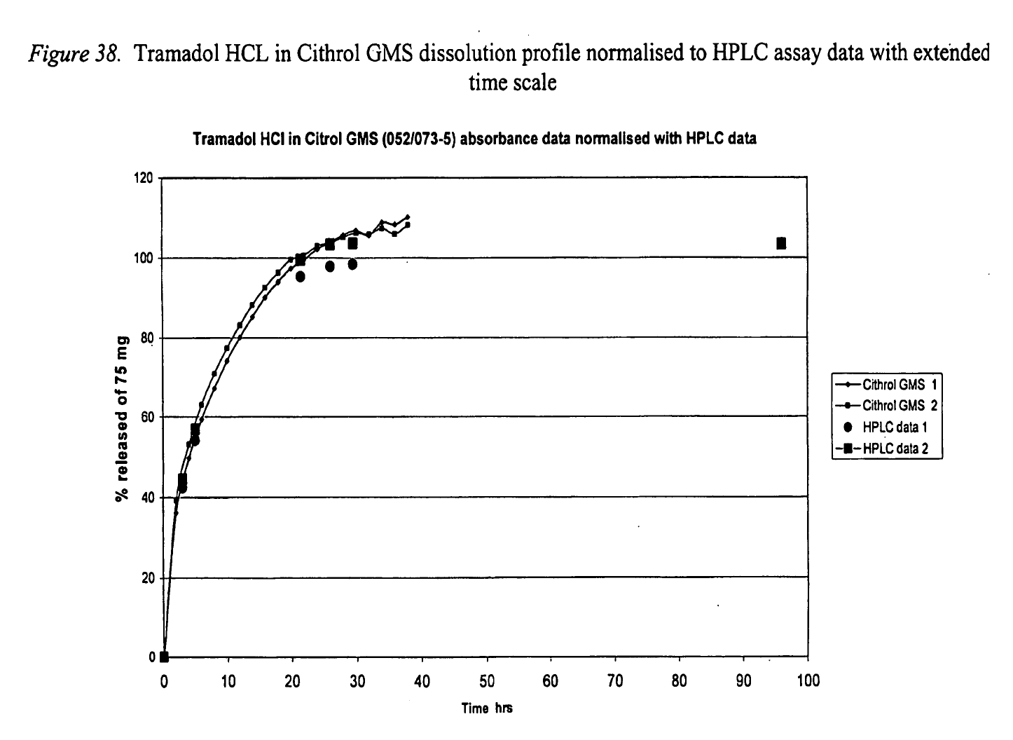

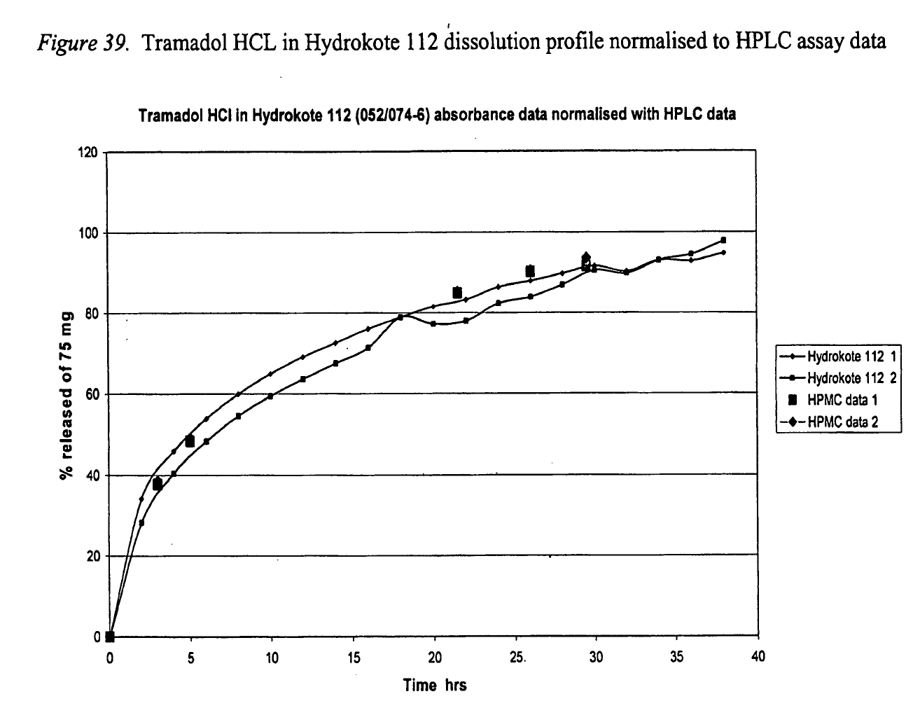

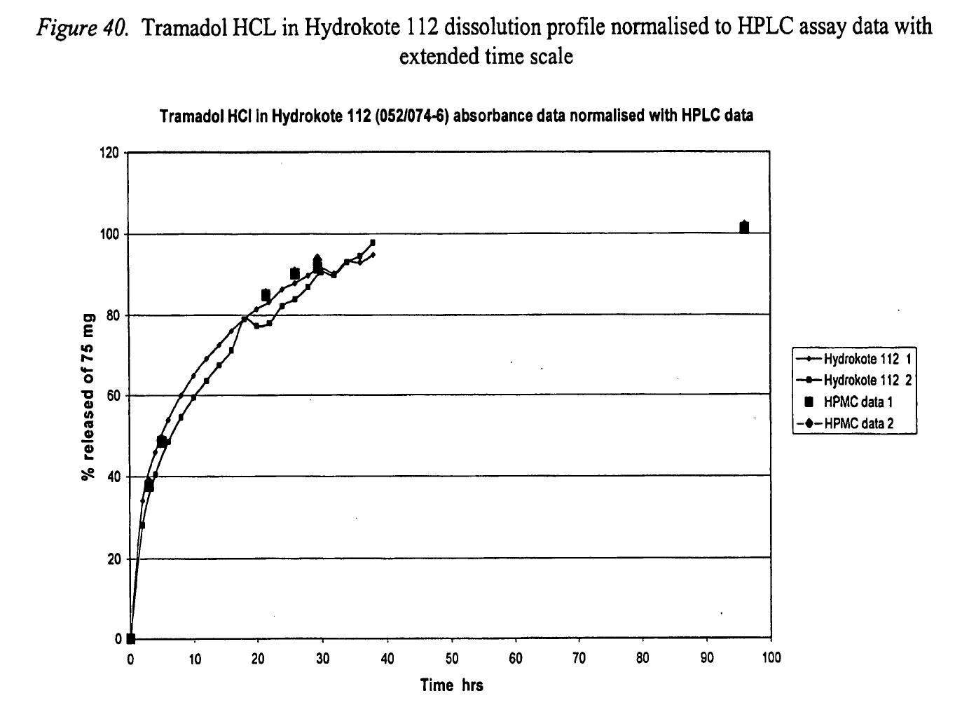

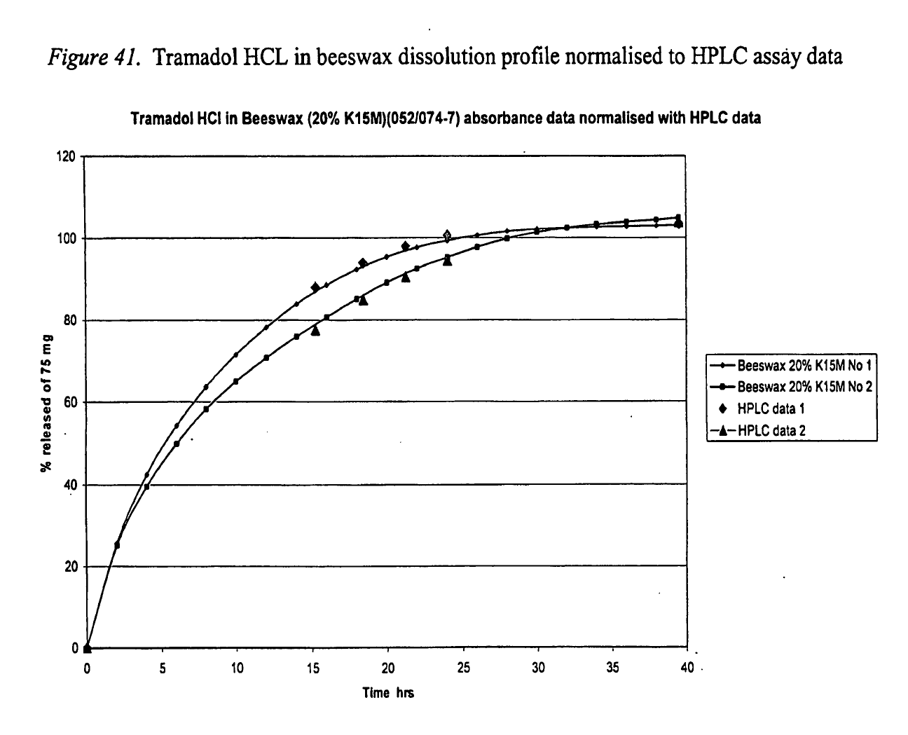

This trial subjected all of the formulations under consideration, at this point, to dissolution testing of two capsule samples (or two×two) with concurrent sampling and HPLC analysis for tramadol HCL. Sufficient samples for HPLC analysis were taken over the course of a dissolution run to allow a plot of absorbance profile versus quantity of tramadol HCl released to be constructed. This permitted the assumptions on absorbance profile versus release profile to be tested. The formulations tested are detailed below. FIG. 28 shows the combined absorbance profiles for three formulation followed by individual plots combining the percentage (of 75 mg) released into solution as determined by HPLC with the initial absorbance plot overlaid and normalized on the first or nearest position to 100% tramadol HCl release by HPLC (FIGS. 29, 30, 31, 32, 33, 34, 35, 36, 37, 38, 39, 40 and 41). This allows comparison of the quantity of tramadol HCl released and the quantity that would have been estimated from the absorbance plot as having been released. Note: The formulation reference details the exact quantities used in a particular set of samples. The same basic formula e.g. 55% of X plus 20% of Y plus 18% of Z, may appear as different formulation references as the quantities in a particular set vary slightly due to weighing variations.

Formulation 052/072-1

| Material | % w/w | Quantity per cap mg |
|---|---|---|
| Beeswax | 59.3 | 237.0 |
| HPMC Pharmacoat 606 | 20.0 | 79.8 |
| Aerosil ® COK 84 | 2.0 | 8.0 |
| Tramadol HCl | 18.8 | 75.1 |
| Capsule fill weight | | 400 |

Formulation 052/072-2 (Same as 052/019)

| Material | % w/w | Quantity per cap mg |
|---|---|---|
| Gelucire 50/02 | 68.2 | 272.6 |
| Methocel ® K 100M | 10.0 | 40.1 |
| Aerosil ® COK 84 | 3.0 | 12.1 |
| Tramadol HCl | 18.7 | 74.8 |
| Capsule fill weight | | 400 |

Formulation 052/073-3

| Material | % w/w | Quantity per cap mg |
|---|---|---|
| Cetyl alcohol | 67.9 | 271.5 |
| Methocel ® K 100M | 9.8 | 39.2 |
| Aerosil ® COK 84 | 3.9 | 15.8 |
| Tramadol HCl | 18.4 | 73.6 |
| Capsule fill weight | | 400 |

Formulation 052/073-4 (Similar to 052/060)

| Material | % w/w | Quantity per cap mg |
|---|---|---|
| Sterotex ® NF | 64.2 | 256.8 |
| Methocel ® K 15M | 15.0 | 60.1 |
| Aerosil ® COK 84 | 2.0 | 7.9 |
| Tramadol HCl | 18.8 | 75.2 |
| Capsule fill weight | | 400 |

Formulation 052/073-5

| Material | % w/w | Quantity per cap mg |
|---|---|---|
| Cithrol ® GMS | 68.3 | 273.0 |
| Methocel ® K 100M | 10.0 | 40.1 |
| Aerosil ® COK 84 | 3.0 | 12.0 |
| Tramadol HCl | 18.7 | 74.9 |
| Capsule fill weight | | 400 |

Formulation 052/074-6

| Material | % w/w | Quantity per cap mg |
|---|---|---|
| Hydrokote 112 | 63.2 | 252.7 |
| Methocel ® K 15M | 15.1 | 60.2 |
| Aerosil ® COK 84 | 3.0 | 12.2 |
| Tramadol HCl | 18.7 | 74.9 |
| Capsule fill weight | | 400 |

Formulation 052/074-7

| Material | % w/w | Quantity per cap mg |
|---|---|---|
| Beeswax | 59.2 | 236.9 |
| Methocel ® K 15M | 20.0 | 80.1 |
| Aerosil ® COK 84 | 2.0 | 8.1 |
| Tramadol HCl | 18.7 | 74.9 |
| Capsule fill weight | | 400 |

TABLE 2
Formulation Release Data Summary from HPLC

| | | | 100% release after | |
|---|---|---|---|---|
| | | | approx | |
| Formula | Base excipient | HPMC and % w/w | (ex HPLC data) | |
| 052/072-1 | Beeswax | 20% Pharmacoat | 70-75% in 45 | hr |
| | | 606 | | |
| 052/072-2 | Gelucire 50/02 | 10% Methocel ® K | 15 | hr |
| | | 100M | | |
| 052/073-3 | Cetyl alcohol | 10% Methocel ® K | 15 | hr |
| | | 100M | | |
| 052/073-4 | Sterotex ® NF | 15% Methocel ® K | 38 | hr |
| | | 15M | | |
| 052/073-5 | Cithrol ® GMS | 10% Methocel ® K | 20 | hr |
| | | 100M | | |
| 052/074-6 | Hydrokote 112 | 15% Methocel ® K | 40 | hr |
| | | 15M | | |
| 052/074-7 | Beeswax | 20% Methocel ® K | 25 | hr |
| | | 15M | | |

Overall the HPLC data correlated well with absorbance data confirming that the modification of formulations based on their absorbance profiles, minimizing delays that HPLC analysis would cause if applied to every sample, was a viable and acceptable approach. The above formulations cover a broad range of release profiles exceeding the 18-24 hr guide value for this project. At the present stage only the first beeswax formulation (52/072-1) is to be discontinued. Further modifications may arise during tamper resistance testing.

### Example 15

Formulations 052/074-7, 052/093-3, 052/073-5 and 052/074-6 were remanufactured with Aerosil® COK 84 replaced in each with Aerosil® 200. The change in Aerosil® did not modify the dissolution profile or the tamper deterrence of the drug.

### Tamper Resistance Testing

The popularity of extended release oxycodone among addicts and recreational drug users is due to a large amount of drug per tablet (12 hour supply). Commercially available immediate release opioid tablets and capsules are usually administered every 4 to 6 hours and they release their dose into the systemic circulation over one to two hours. New, extended release formulations are designed to gradually release their much larger opioid content over a 12 or 24-hour period.

Most recreational drug users and addicts have a unit of use which is one tablet or capsule. The 12 or 24-hour supply of opioid contained in one tablet or capsule, instead of 4 to 6 tablets or capsules means that there is a greater risk that such formulations may be highly sought by drug addicts and recreational drug users alike, for non-medical use. Intentional or inadvertent tampering from extended release formulations will rapidly deliver a massive dose and produce profound a variety of serious and life threatening side effects, including respiratory depression and failure, sedation, cardiovascular collapse, coma and death.

Addicts and recreational drug users commonly use extended release opioids by a variety of routes of administration. Commonly used methods include 1) parenteral (e.g., intravenous injection, where the drug is crushed and extracted or melted and the contents of a dosage unit then injected), 2) intranasal (e.g., snorting, where the drug is inhaled as powdered dosage unit), and 3) episodic or repeated oral ingestion of crushed product, where the drug is chewed to increase the surface area and permit rapid release of drug substance. All of these strategies are intended to more efficiently get the opioid into the CNS, both in terms of total amount of drug, peak concentration of drug and time to peak concentration of drug.

One mode of abuse involves the extraction of the opioid component from the dosage form by first mixing the table or capsule with a suitable solvent (e.g., water or alcohol), and then filtering and/or extracting the opioid component from the mixture for intravenous injection. Another mode of abuse of extended release opioids involves dissolving the drug in water, alcohol or another “recreational solvent” to hasten its release and to ingest the contents orally, in order to provide high peak concentrations and maximum euphoriant effects.

It is necessary to be able to measure resistance to the likely routes of abuse in a meaningful and relevant way. No standard set of tests exist with companies, interested in abuse resistance, generating their own particular set of tests. The series of tests chosen to evaluate abuse resistance and the source of the test were:

### Extraction with Alcohol on Whole Dosage Unit

This method is based on US patent application 2004/0161382 A1 (P 11, [0122]). Method: Place a whole dosage unit in 18 mL of 0.1N HCl in a 60 mL amber bottle and shake at 240 rpm on an orbital shaker for 30 min. After 30 min add 12 mL of ethanol (95-96%) to each bottle. Swirl by hand and remove a 1 mL sample from each bottle (T₀). Place the solutions back in the orbital shaker for further shaking at 240 rpm. Take 1 mL samples after 10, 20, 30, 40, 60 and 180 min of further shaking for each bottle. Analyze and graph the results on a linear scale of cumulative release (%) vs. time (min).

### Extraction with Alcohol on a Crushed or Cut Dosage Unit

Extension of test in above patent. Method: Place a tablet (after crushing with a single crush with a spatula) or a capsule (cut in half) in 18 mL of 0.1N HCl in a 60 mL amber bottle and shake at 240 rpm on an orbital shaker for 30 min. Continue the test as in 1) above.

### Extraction into Water

This method is based on US patent application 2004/0161382 A1 (P12, [0130]). Method: Crush with a mortar and pestle and grind in 5 mL of water for 5 minutes. The resulting suspension is filtered through a 0.45 micron filter into a flask and diluted to 50 mL with water. Quantify Tramadol HCl concentration by HPLC.

### Freeze and Crush

Method: Freeze the dosage unit in a domestic freezer for 24 hr, then grind with a mortar and pestle for five minutes. Sieve through a suitable sieve (ca 600 micron) and, by weighing, measure the percentage passing the sieve.

### Taste of Base Excipient Mix

### Organoleptic Test

Method: Chew a placebo mix for five minutes and rate the taste on a 0-10 scale with 0 as bland to repulsive at 10. This method is relevant only to dosage units containing taste modifiers.

### Extraction into Acid

Method: Crush with a mortar and pestle and heat to boiling in 5 mL of vinegar. The resulting suspension is filtered through a 0.45 micron filter into a flask and diluted to 50 mL with water. Quantify tramadol HCl concentration by HPLC.

### Application of Heat

### Melting Temperature>50° C. or 55° C.

Method: Heat the squashed contents of a dosage unit on a hot plate until melted. Determine the temperature of melting and test whether the mix becomes sufficiently fluid to be drawn up into a syringe via a 1.2 mm needle then expelled. The formulations tested were the last six of those listed in Table 2 (omits the first sample 052/072-1). Dromadol® SR tablets were included into the testing for to allow comparison of the liquid filled dosage units with a commercial tramadol HCl prolonged release preparation. The results of testing are presented below.

### Example 16

### Extraction with Alcohol on Whole Dosage Unit

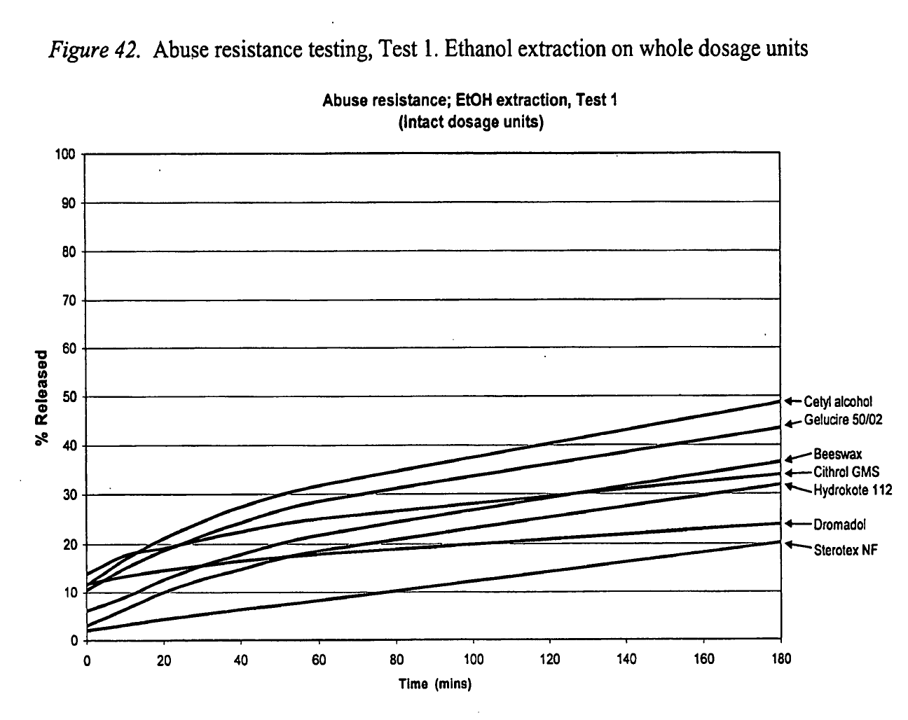

The results of this test are shown in FIG. 42.

### Example 17

### Extraction with Alcohol on Cut or Crushed Dosage Unit

The samples under test were reduced to four formulations plus the Dromadol® SR comparator at this point. The Cetyl alcohol based formulation (052/073-3) and Gelucire 50/02 (052/072-2) were deselected due to their dissolution release time of approx 15 hr to 100% release and their high extractable fraction, as seen in FIG. 42. Formulations showing a slower than target in vitro release profile may possibly show more rapid release in vivo due to the presence of digestion materials but is seems unlikely that formulations showing a faster than desirable in vitro dissolution rate will show a retarded rate in vivo.

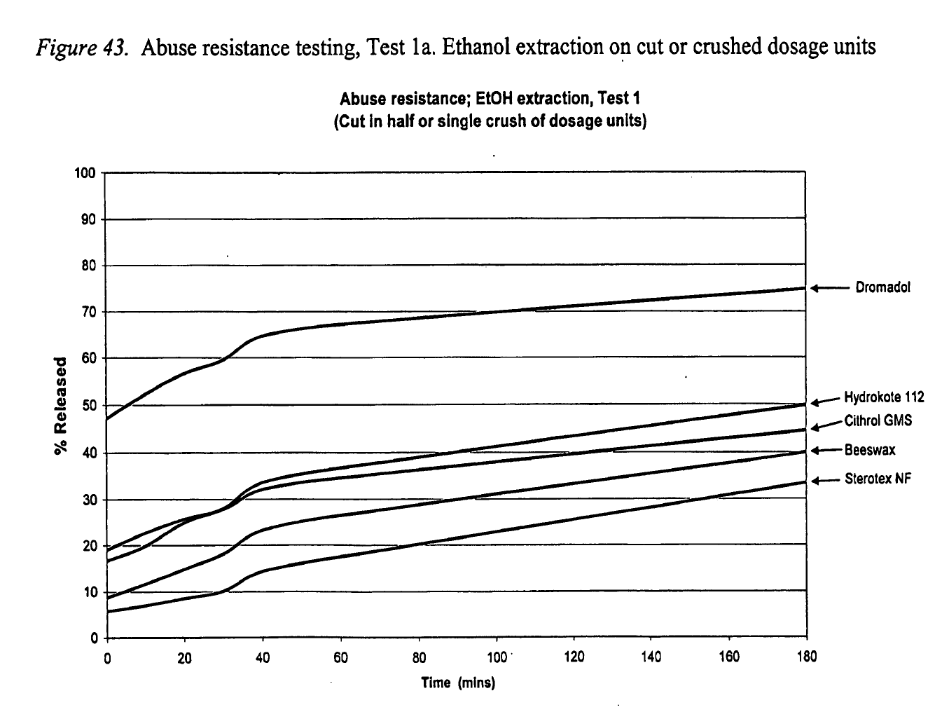

The above two tests demonstrate that whole dosage units release their contents into alcohol relatively slowly but once crushed or cut the waxy liquid fill dosage unit is much harder to extract than the tablet. One single crush turns the Dromadol® tablet into an easily extractable powder. This feature would apply to any tablet. It should be noted that the apparent high quantity released at T₀ is due to the conditions specified in the method. The method requires an initial 30 min of shaking in 18 mL of 0.1N HCl before the addition of ethanol. The time is defined in the method as starting from the addition of ethanol. The tramadol HCl, shown as released at T₀, has dissolved during the 30 min pre ethanol addition sample preparation. This test demonstrates that the liquid fill formulations are clearly superior in abuse resistance by ethanol extraction to an extended release tablet (FIG. 43).

### Example 18

### Extraction into Water Via Crushing and Grinding in Water

The four formulations continuing under test plus Dromadol® SR tablets were crushed and ground for 5 minutes in 5 mL of water to simulate extraction in preparation for swallowing or injection. The material was then filtered (by pressurizing a 45μ filter using an attached syringe) and diluted before quantifying by HPLC. The results are presented in Table 3 and 4 below with comments on the mix produced after grinding given below.

TABLE 3

| Product | Observations |
|---|---|
| Dromadol ® SR tablet | Ground easily and formed a mobile easily |
| | filtered solution. |
| Sterotex ® NF formulation | Difficult to grind, forms a light paste that |
| 052/073-4 | filtered slowly. |
| Cithrol ® GMS formulation | Difficult to grind, forms a light paste that |
| 052/073-5 | filtered very slowly. |
| Hydrokote 112 formulation | Difficult to grind, forms a light paste that |
| 052/074-6 | filtered very slowly. |
| Beeswax formulation | Difficult to grind, forms a light paste that |
| 052/074-7 | filtered relatively easily |

The Dromadol® SR tablet crushed easily and produced a solution that filtered in a matter of seconds while the beeswax formed a light paste, with difficulty, which took approximately five minutes to filter. This difficulty of preparation was common to the other capsule samples with filtration time graduating from the five minutes of the beeswax sample to over 60 minutes for the Cithrol® GMS sample. All liquid fill samples gave much greater difficulty in grinding and filtering than the tablet sample.

TABLE 4
Percentage release on extraction into water.

| Base excipient | Formulation | % released on extraction |
|---|---|---|
| Dromadol ® SR | n/a | 84.0 |
| tablets | | |
| Sterotex ® NF | 052/073-4 | 38.7 |
| Cithrol ® GMS | 052/073-5 | 17.1 |
| Hydrokote 112 | 052/074-6 | 24.5 |
| Beeswax | 052/074-7 | 30.1 |

The HPLC data shows that tramadol HCl was easily extracted from the tablet, as would be expected as a tablet crushes easily to give a large surface area from which extraction can take place. Extraction from the liquid fill formulation was reduced considerably due to the waxy nature of the base excipients and the inclusion of HPMC which caused the liquid extracts to turn into a filtration resistant light paste.

### Example 19

### Extraction into Acid Water Via Crushing and Grinding in Dilute Acetic Acid

Dilute acetic acid (6% w/w glacial acetic in water) was used to simulate the vinegar that drug abusers may use when extracting dosage units for injection. Dosage units were crushed forcibly 2-3 times in a mortar and pestle then transferred to a small beaker where 5 mL of the above dilute acetic acid was added. The mix was heated to boiling on a hotplate and held boiling for 5-10 s. The mix was allowed to cool to room temperature, the resulting solution filtered through a 45μ filter, as above, the solution diluted to volume and the content of tramadol HCl determined by HPLC. The assay results are shown below expressed as a percentage of the contents released into solution.

TABLE 5
Percentage release on extraction into dilute acid.

| Base excipient | Formulation | % released on extraction |
|---|---|---|
| Dromadol ® SR | n/a | 83.9 |
| tablets | | |
| Sterotex ® NF | 052/073-4 | 29.3 |
| Cithrol ® GMS | 052/073-5 | 41.7 |
| Hydrokote 112 | 052/074-6 | 30.2 |
| Beeswax | 052/074-7 | 17.6 |

Tramadol HCl was easily extracted from the tablet. All liquid fill formulations showed appreciably better resistance to extraction. The waxy mass of the four test formulations coalesced on melting and floated as a mass on the surface. The HPMC content of the mass is insoluble above 40° C. so, instead of its normal property of assisting release at room temperature, it actively prevents release at this temperature by helping to hold the molten mass together. The tramadol HCl migrates relatively slowly to the surface when boiling agitates the mass while the powdered tablet releases most of its content instantly. It is easily understood why the formulated capsule dosages give superior extraction resistance to that of tablets.

### Example 20

### Effect of Heat on Dosage Units

Tablets can be crushed and extracted easily while soft gel contents have been known to be liquefied by slight warming (to about 40° C.) and the contents injected directly. This test records the temperature at which the meltable excipients in a formulation have liquefied and tests whether this material can be sucked into a syringe and ejected as would take place during an injection. Formulated material was placed in a beaker then slowly warmed in a water bath. The mix temperature was recorded with a calibrated thermocouple. The results are listed in Table 5 below.

TABLE 6
Melting point range and potential for direct injection

| Base | Excipient | For- | Formulation | |
|---|---|---|---|---|
| excipient | mp | mulation | melted | Comment |
| Sterotex ® | 61-66° C. | 052/073-4 | 65° C. | Light cream, can't |
| NF | | | | suck into syringe, |
| | | | | sets instantly in |
| | | | | needle tip |
| Cithrol ® | 55-60° C. | 052/073-5 | 58° C. | Light cream, can't |
| GMS | | | | suck into syringe, |
| | | | | sets instantly in |
| | | | | needle tip |
| Hydrokote | 43-46° C. | 052/074-6 | °45 C. | Viscous paste, can |
| 112 | | | | suck and eject |
| | | | | about 5 mm of |
| | | | | material from |
| | | | | needle |
| Beeswax | 61-66° C. | 052/074-7 | 66° C. | Viscous paste, |
| | | | | can't suck into |
| | | | | syringe, sets |
| | | | | instantly in needle |
| | | | | tip |

All of the mixes melted around the melting points of the base excipients and, due to this elevated melting point, none could be effectively introduced into a syringe nor could be ejected (or injected).

### Example 21

### Modification to Increase Resistance to Powdering

It was observed during this trial that the Sterotex NF formulation can be powdered with careful crushing. This occurs to a lesser extent with the Cithrol® GMS and Hydrokote 112 formulations. It was desirable to decrease the ease with which this formulation could be powdered. Both the Sterotex® NF and Hydrokote 112 formulations gave full release of tramadol HCl in 38-40 hr during dissolution testing. It would therefore be acceptable to add modifiers that decrease the ease of crumbling formulated material into a powder even if these accelerated release. Several materials were tested including small levels of beeswax, adding hydrophilic liquids such as maltitol or glucose syrup or adding surfactants such as Crillet 4. The addition of hydrophilic liquids or surfactants immediately turned the mix into a lumpy unfillable mass by binding the powder content together. The use of these liquids was discontinued.

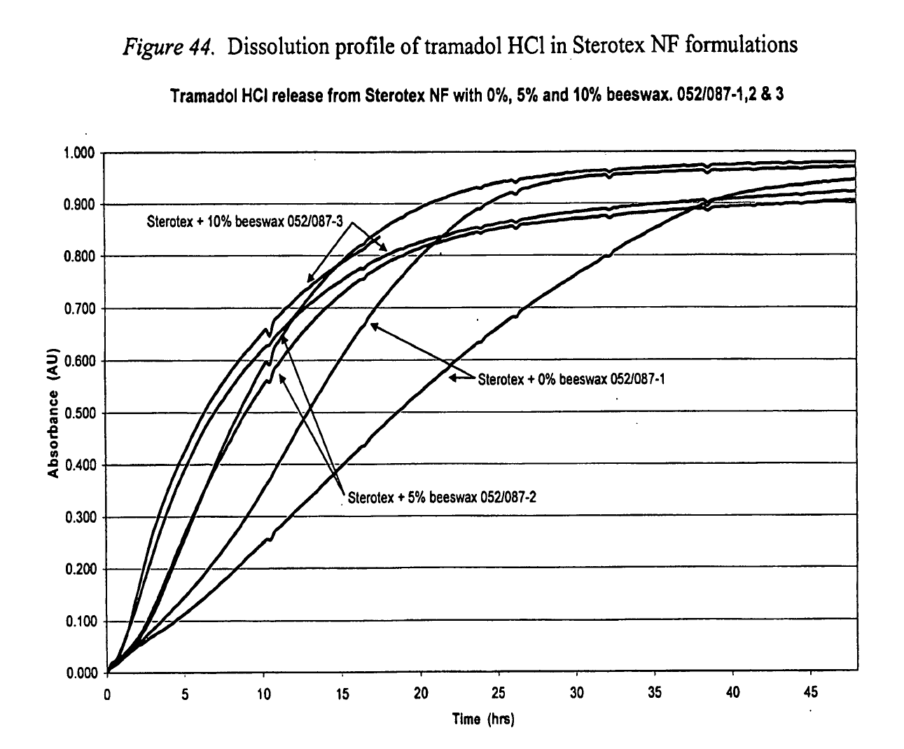

Formulations containing Sterotex® NF with increased level of HPMC to accelerate dissolution plus 0, 5% and 10% beeswax were produced for examination of any change in resistance to powdering. The dissolution profiles of each formulation were recorded as the absorbance curve via UV monitoring at 271 nm as previously. The formulas used are show below. The dissolution results are show in FIG. 44.

Formulation 052/087-1

| Material | % w/w | Quantity per cap mg |
|---|---|---|
| Sterotex ® NF | 60.3 | 241.0 |
| Methocel ® K 15M | 20.0 | 80.0 |
| Aerosil ® COK 84 | 1.0 | 4.0 |
| Beeswax | 0.0 | 0.0 |
| Tramadol HCl | 18.8 | 75.0 |
| Capsule fill weight | | 400 |

Formulation 052/087-2

| Material | % w/w | Quantity per cap mg |
|---|---|---|
| Sterotex ® NF | 55.3 | 221.0 |
| Methocel ® K 15M | 20.0 | 80.0 |
| Aerosil ® COK 84 | 1.0 | 4.0 |
| Beeswax | 5.0 | 20.0 |
| Tramadol HCl | 18.8 | 75.0 |
| Capsule fill weight | | 400 |

Formulation 052/087-3

| Material | % w/w | Quantity per cap mg |
|---|---|---|
| Sterotex ® NF | 50.3 | 201.2 |
| Methocel ® K 15M | 20.0 | 79.9 |
| Aerosil ® COK 84 | 1.0 | 4.0 |
| Beeswax | 10.0 | 40.0 |
| Tramadol HCl | 18.8 | 74.9 |
| Capsule fill weight | | 400 |

The Sterotex® formulation without beeswax showed considerable variability. The addition of 5% or 10% beeswax significantly increased the rate of release to an approximate time for full release of 25 hr. There was no meaningful difference in release rate between either formulation containing added beeswax so the formulation containing 10% beeswax (052/087-3) was selected for inclusion in subsequent trials.

### Example 22

### Ease of Powdering and Percentage of Resultant Particles of 650 Micron or Less

Capsules were initially powdered at room temperature as an indicative guide and for comparison with subsequent frozen samples. The contents were removed from the capsules and ground until the finest powder achievable had been formed. The stated period of five minutes was not normally required and it was observed that excessive grinding could cause the particles to start to coalesce. The data obtained is shown in Table 7.

TABLE 7
Powder generation by grinding of formulated material at RT

| | | | % as 650μ |
|---|---|---|---|
| Base Excipient | Formulation | Comment | or less |
| Dromadol ® SR | | | 64.2% |
| tablet | | | |
| Dromadol ® SR | | Repeat | 79.9% |
| tablet | | sample | |
| Sterotex ® NF | 052/087-1 | 0% beeswax | 84.7% |
| Sterotex ® NF | 052/087-3 | Plus 10% | 84.8% |
| | | beeswax | |
| Cithrol ® GMS | 052/073-5 | | 86.9% |
| Hydrokote 112 | 052/074-6 | | 2.1% |
| Beeswax | 052/074-7 | | 1.9% |

The test was repeated using capsules that had been cooled in a domestic freezer. The results of this trial are shown in Table 8.

TABLE 8
Powder generation by grinding of formulated material cooled
to domestic freezer temperatures

| | | | % as 650μ |
|---|---|---|---|
| Base Excipient | Formulation | Comment | or less |
| Dromadol ® SR | | | 70.6% |
| tablet | | | |
| Sterotex ® NF | 052/073-4 | | 78.8% |
| Sterotex ® NF | 052/087-3 | Plus 10% | 82.1% |
| | | beeswax | |
| Cithrol ® GMS | 052/073-5 | | 85.7% |
| Hydrokote 112 | 052/074-6 | | 5.5% |
| Beeswax | 052/074-7 | | 1.5% |

There was little significant difference, within experimental variation, between the results obtained at room temperature and that obtained from dosage units frozen to domestic freezer temperature (−20° C.). The Dromadol® SR tablet ground to a fine powder relatively easily. The Sterotex® NF and Cithrol® GMS formulations also produced similar amounts of fine powder. The incorporation of 10% beeswax in one of the Sterotex® NF formulations made to detectable difference. The beeswax and Hydrokote 112 formulations provided excellent resistance against powdering.

### Example 23

### Sterotex® NF Formulation Modification to Enhance Resistance to Powdering

Further modifications were made to the Sterotex® NF based formulation, using fractionated coconut oil, to improve resistance to powdering. Samples were prepared substituting 15, 20 and 25% of Sterotex® NF for fractionated coconut oil. The formulations used were as listed below.

Formulation 052/093-1

| Material | % w/w | Quantity per cap mg |
|---|---|---|
| Sterotex ® NF | 45.2 | 180.8 |
| Fractionated coconut oil | 15.0 | 59.9 |
| Methocel ® K 15M | 20.0 | 80.1 |
| Aerosil ® COK 84 | 1.0 | 4.1 |
| Tramadol HCl | 18.8 | 75.1 |
| Capsule fill weight | | 400 |

Formulation 052/093-2

| Material | % w/w | Quantity per cap mg |
|---|---|---|
| Sterotex ® NF | 40.2 | 160.8 |
| Fractionated coconut oil | 20.0 | 79.9 |
| Methocel ® K 15M | 20.0 | 79.9 |
| Aerosil ® COK 84 | 1.0 | 4.2 |
| Tramadol HCl | 18.8 | 75.1 |
| Capsule fill weight | | 400 |

Formulation 052/094-3

| Material | % w/w | Quantity per cap mg |
|---|---|---|
| Sterotex ® NF | 35.3 | 141.0 |
| Fractionated coconut oil | 25.0 | 100.0 |
| Methocel ® K 15M | 19.9 | 79.8 |
| Aerosil ® COK 84 | 1.0 | 4.1 |
| Tramadol HCl | 18.8 | 75.0 |
| Capsule fill weight | | 400 |

### Example 24

The test to quantify the ease of powdering, Test 3, was repeated using capsules that had been cooled in a domestic freezer. The results of this trial are shown in table 8 below.

TABLE 9
Powder generation from Sterotex ® NF formulations
containing fractionated coconut oil by grinding of formulated
material cooled to domestic freezer temperatures

| | | | % as 650μ or |
|---|---|---|---|
| Base Excipient | Formulation | Comment | less |
| Sterotex ® NF | 052/073-4 | Data from Table 6 | 78.8% |
| Sterotex ® NF | 052/093-1 | Plus 15% fractionated | 49.7% |
| | | coconut oil | |
| Sterotex ® NF | 052/093-2 | Plus 20% fractionated | 33.7% |
| | | coconut oil | |
| Sterotex ® NF | 052/094-4 | Plus 25% fractionated | 8.3% |
| | | coconut oil | |

The addition of fractionated coconut oil produced the desired effect in decreasing the ability to grind cooled formulated mix into a powder. The hot mix remained a machine fillable light cream. The melting point of the 25% mix had decreased from the 65° C. melting point of a Sterotex® NF mix with zero added fractionated coconut oil to an acceptable 62° C. for the mix containing 25%.

### Example 25

### Abuse Resistance Testing, Reevaluation of Modified Sterotex® NF Combinations

Further testing was required, after revising the Sterotex® NF formulation by substituting part of the Sterotex® NF for fractionated coconut oil, to determine how this change had affected the other parameters.

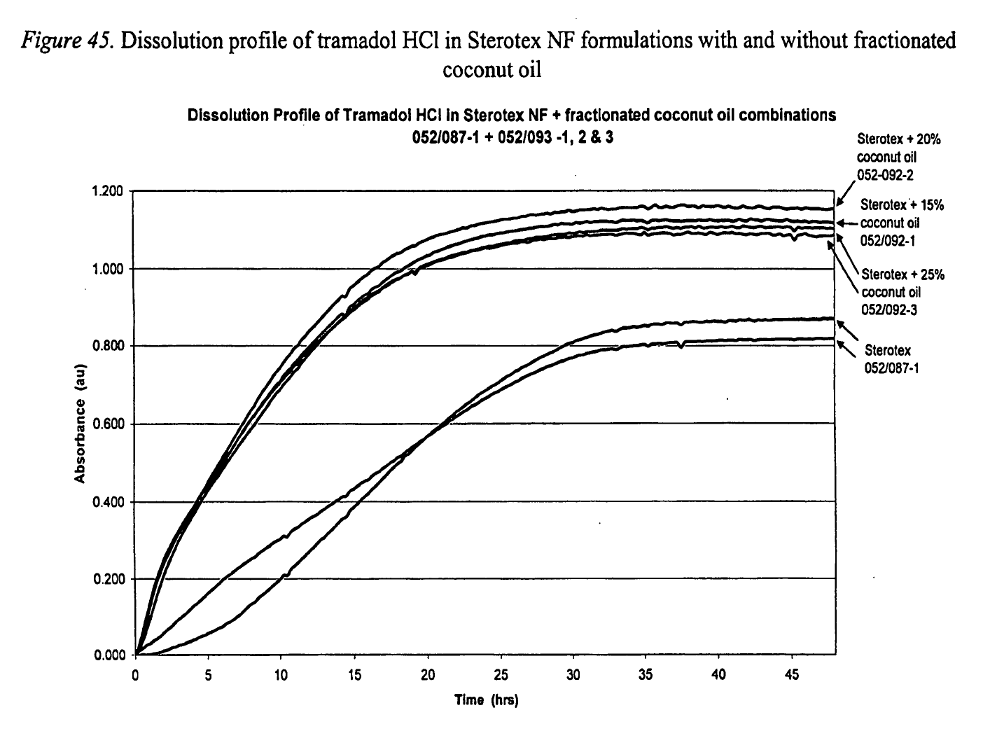

Dissolution testing was carried out, in the same manner as previously; using the USP paddle method to obtain the dissolution profiles of the Sterotex® NF formulations with and without additional fractionated coconut oil. This plot is shown below in FIG. 45

### Example 26

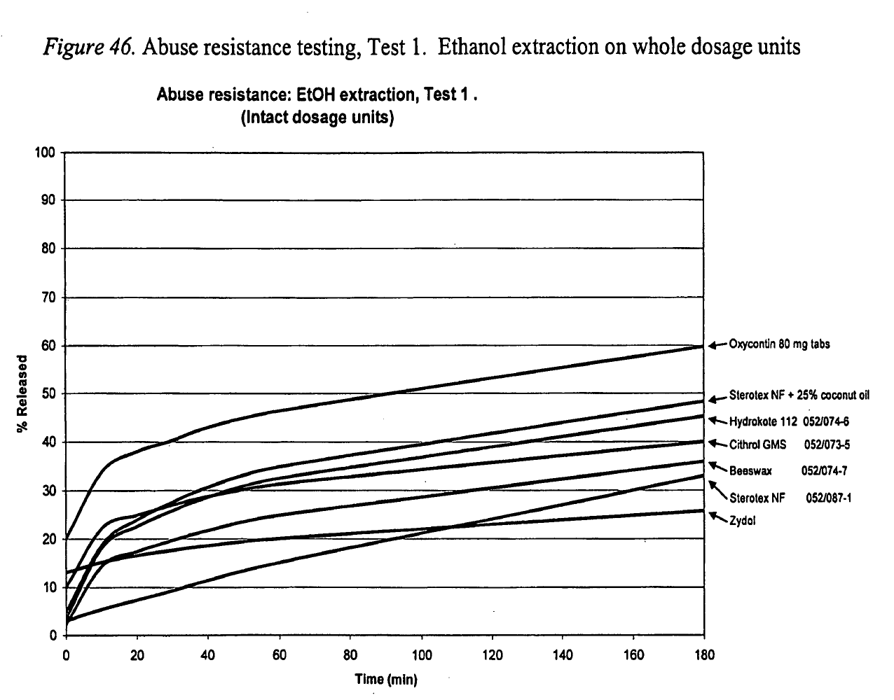

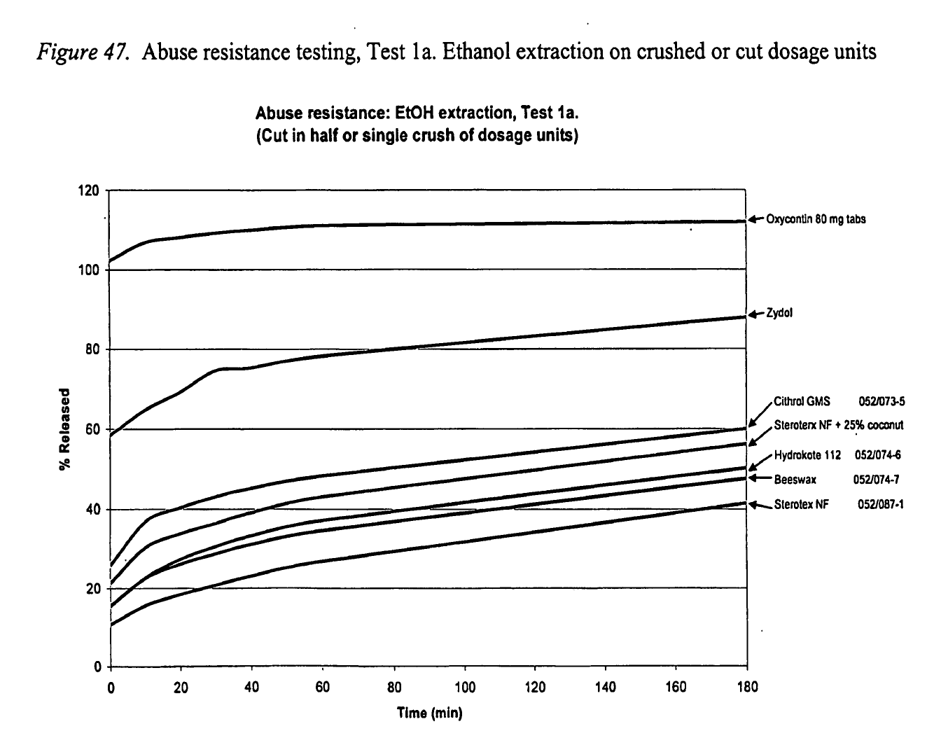

Tests for ethanol extraction of whole and crushed or cut dosage units were also repeated. Sterotex® NF with 25% fractionated coconut oil (052/094-3) was tested alongside the fractionated coconut oil free analogue (052/087-1). The opportunity was taken to test some additional relevant samples. The three previously tested formulations based on Cithrol® GMS (052/073-5), Hydrokote 112 (052/074-6) and the beeswax formulation (052/074-7) were retested. Zydol® XL 150 tablets were substituted for the previously used Dromadol® SR tablets. Both of these are slow release formulations containing 150 mg of tramadol HCl. OxyContin® extended release 80 mg tablets were included for comparison purposes as oxycodone extended release tablets are the subject of current concerns over tablet abuse and they provide another tablet comparator containing a similar quantity of water soluble active in a slow release formula. The results of ethanol extraction of whole dosage units and cut/crushed dosage units are shown below in FIGS. 46 and 47, respectively.

The Sterotex® NF formulation containing 25% fractionated coconut oil did show increased susceptibility to ethanol extraction compared with the formulation without fractionated coconut oil however this was demonstrably much better than the tablets or the Cithrol® GMS formulation so was considered as acceptable. The quantities extracted were broadly in line with that determined in the earlier ethanol extraction tests, shown in FIGS. 42 and 43. The Zydol® XL 150 tablets showed comparable release to the Dromadol® SR tablets in the earlier test. The OxyContin® tablets showed much greater and faster release than any of the dosage units in either of these sets of tests.

### Example 27

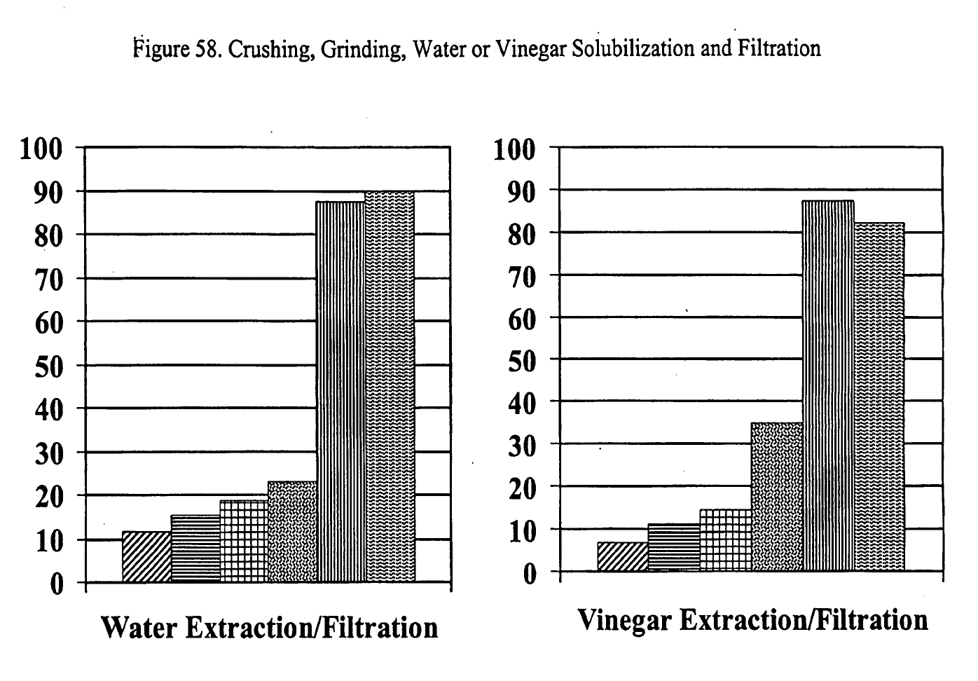

The abuse resistance test involving extraction into water by grinding a dosage unit in a mortar and pestle with subsequent filtration was repeated. All of the samples included in the above ethanol extraction tests were included. Table 10 shows the results of HPLC analysis of the filtrate expressed as the percentage of drug substance released. The results are also depicted in Left Panel of FIG. 58 (the bars from left to right are Formulation 052/094-3, Formulation 052/073-5, Formulation 052/074-7, Formulation 052/074-6, Zydol XL® 150 mg and OxyContin® 80 mg, respectively).

TABLE 10
Percentage release on extraction into water.

| Base excipient | Formulation | % released on extraction |
|---|---|---|
| Zydol ® XL 150 | n/a | 87.4 |
| OxyContin ® 80 mg | n/a | 90.0 |
| Sterotex ® NF | 052/087-1 | 28.1 |
| Sterotex ® NF with | 052/094-3 | 11.6 |
| 25% fr. coconut oil | | |
| Cithrol ® GMS | 052/073-5 | 15.3 |
| Hydrokote 112 | 052/074-6 | 23.1 |
| Beeswax | 052/074-7 | 18.6 |

### Example 28

The abuse resistance test involving extraction into dilute acetic acid by heating to boiling was repeated. The same samples as immediately above were tested and the results of HPLC analysis of the resulting filtrates are shown in Table 11. The results are also depicted in Right Panel of FIG. 58 (the bars from left to right are Formulation 052/094-3, Formulation 052/074-6, Formulation 052/074-7, Formulation 052/073-5, Zydol XL® 150 mg and OxyContin® 80 mg, respectively)

TABLE 11
Percentage release on extraction into dilute acid.

| Base excipient | Formulation | % released on extraction |
|---|---|---|
| Zydol ® XL 150 | n/a | 87.4 |
| OxyContin ® 80 mg | n/a | 82.2 |
| Sterotex ® NF | 052/087-1 | 10.8 |
| Sterotex ® NF with | 052/094-3 | 7.0 |
| 25% fr. coconut oil | | |
| Cithrol ® GMS | 052/073-5 | 34.9 |
| Hydrokote 112 | 052/074-6 | 11.1 |
| Beeswax | 052/074-7 | 14.5 |

Both sets of results gave similar results for comparable formulations in this and the earlier set of tests. All liquid fill formulations were significantly superior to any of the three commercial tablets formulations.

### Example 29

### Ease of Powdering and Percentage of Resultant Particles of 600 Micron or Less

Initial powdering tests were carried out using a laboratory stainless steel sieve of nominal 650 micron size. The sieve size used had been qualitatively determined as a size that could differentiate between the powders generated. Initially much finer sieves had been tested but were found to be too fine e.g. a 45 micron sieve was tested but this was too fine resulting in almost zero powder passing through the sieve from any samples. As result of the initial tests, a certified sieve was obtained of 600 micron size for further trials. All of the above samples were subjected to the powdering test. The results are shown in Table 12.

TABLE 12
Powder generation of formulations and comparator tablets by
grinding of dosage units cooled to domestic freezer temperatures

| | | | % as 600μ | % as 600μ |
|---|---|---|---|---|
| | | | or less. | or less. |
| Base Excipient | Formulation | Comment | Sample 1 | Sample 2 |
| Dromadol ® SR | n/a | | 48.1% | 51.9% |
| Zydol ® XL | n/a | | 52.6% | 41.2% |
| 150 | | | | |
| OxyContin ® | n/a | | 66.6% | Not tested |
| 80 mg | | | | |
| Sterotex ® NF | 052/094-3 | With 25% | 2.2% | 0.6% |
| with 25% fr. | | fractionated | | |
| coconut oil | | coconut oil | | |
| Cithrol ® GMS | 052/073-5 | | 40.3% | 72.4% |
| Hydrokote 112 | 052/074-6 | | 7.3% | 2.6% |
| Beeswax | 052/074-7 | | 0.7% | 0.6% |

It should be noted that the lower results found in this trial than those reported previously are due to a slightly finer sieve size being used. The tablets all powdered relatively easily while the Sterotex® NF, Hydrokote 112 and beeswax were very resistant to powdering. The Cithrol® GMS gave a high quantity of powder. The same approach of adding a room temperature oil could be used on the Cithrol® GMS as used on Sterotex® NF however, with the Cithrol® GMS formulation showing a release rate of approximately 20 hr, on the fast size of the target 24 hr, it was decided not to amend it at this stage.

### Example 30

### Dissolution Testing of Stored Samples

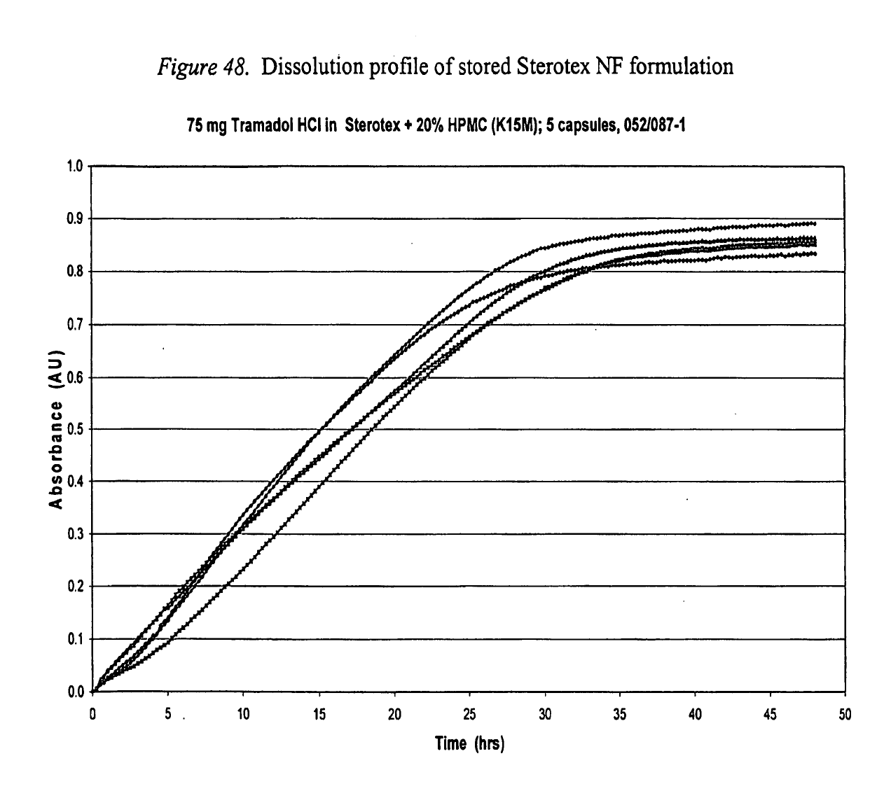

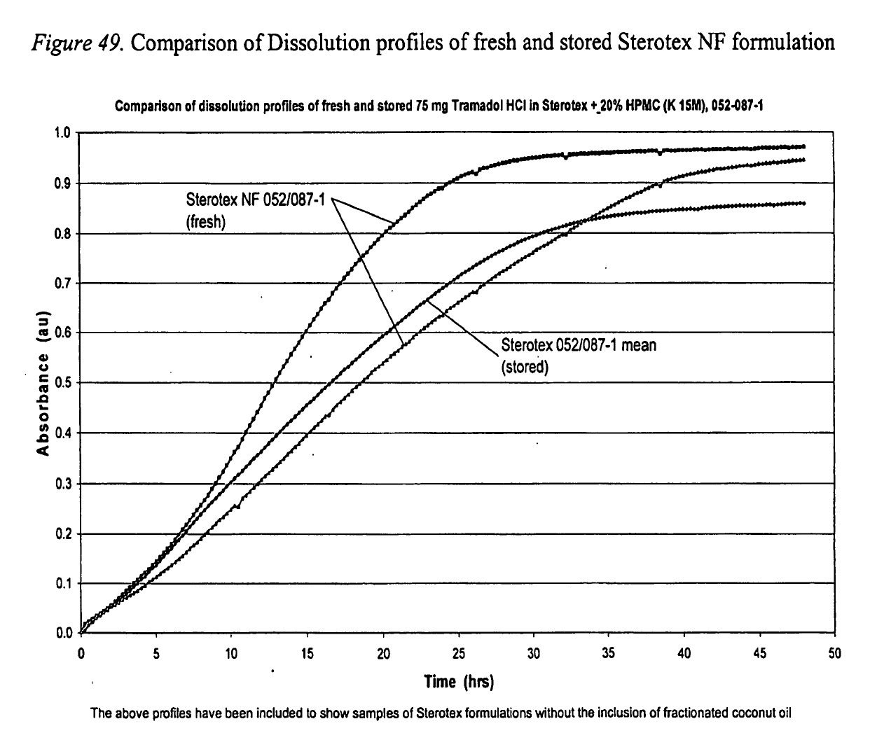

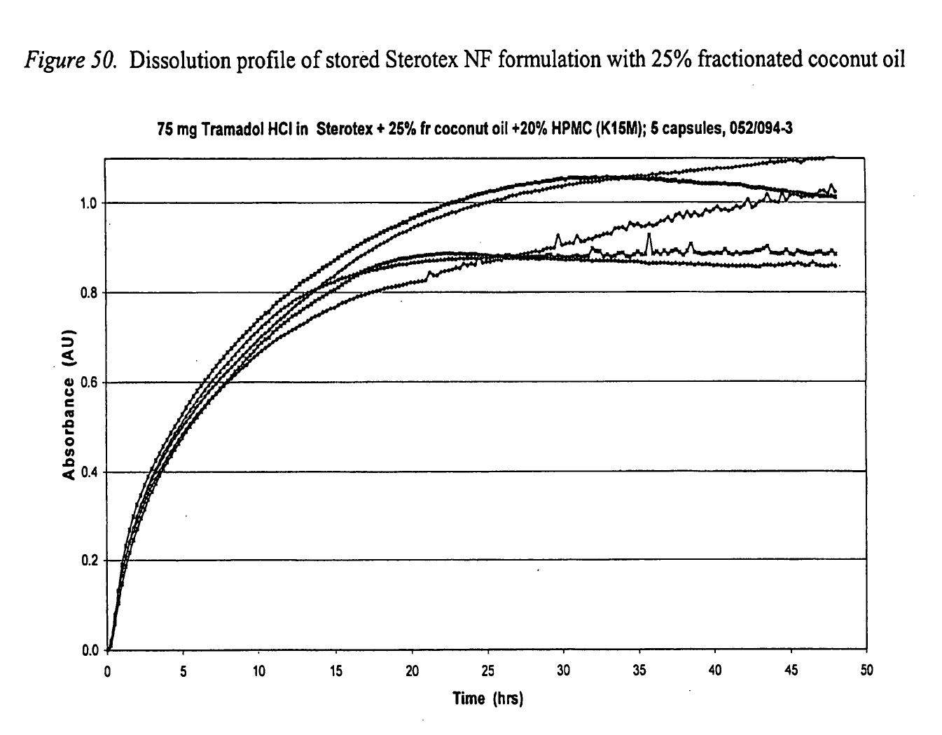

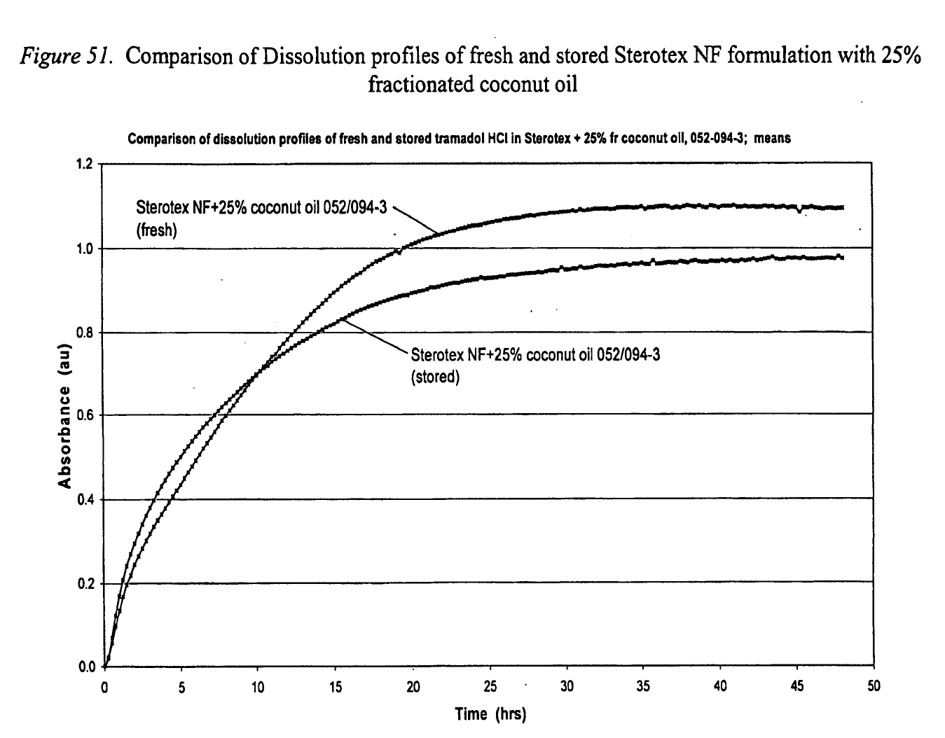

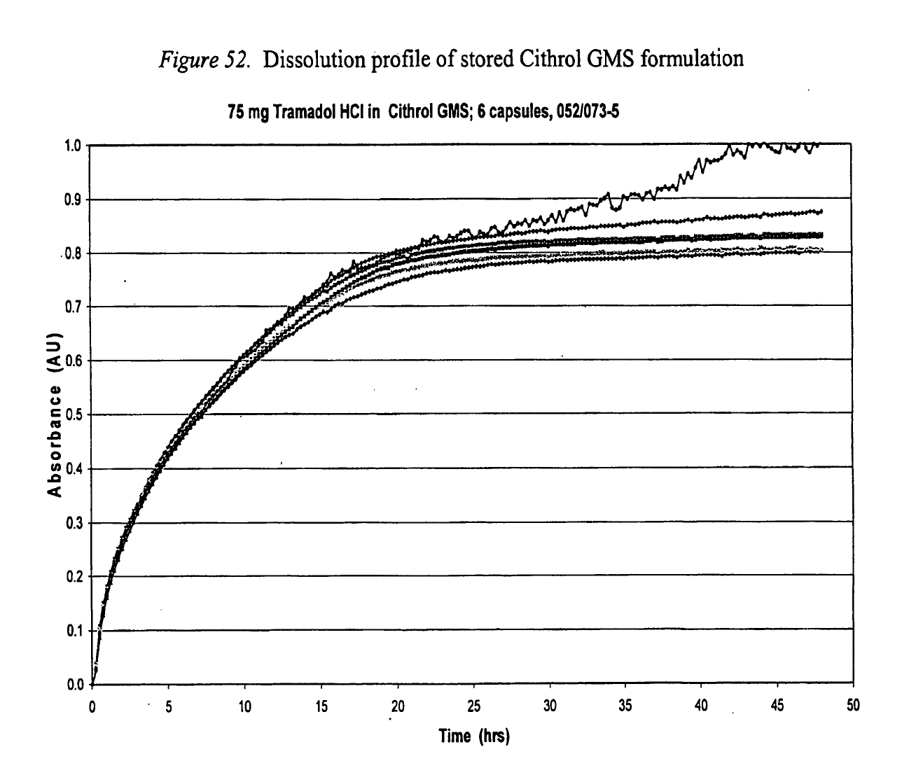

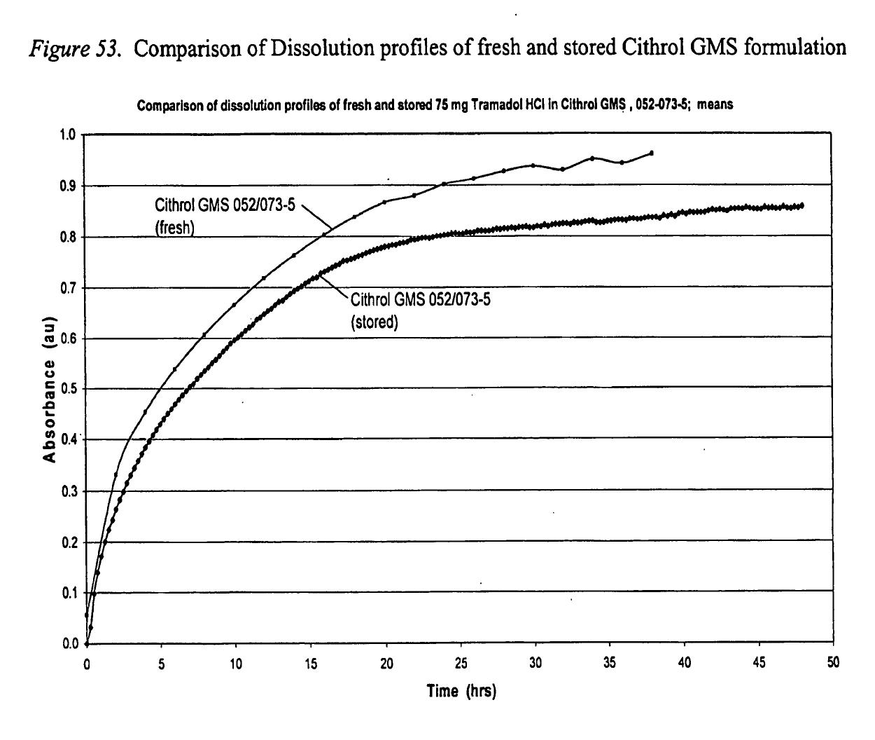

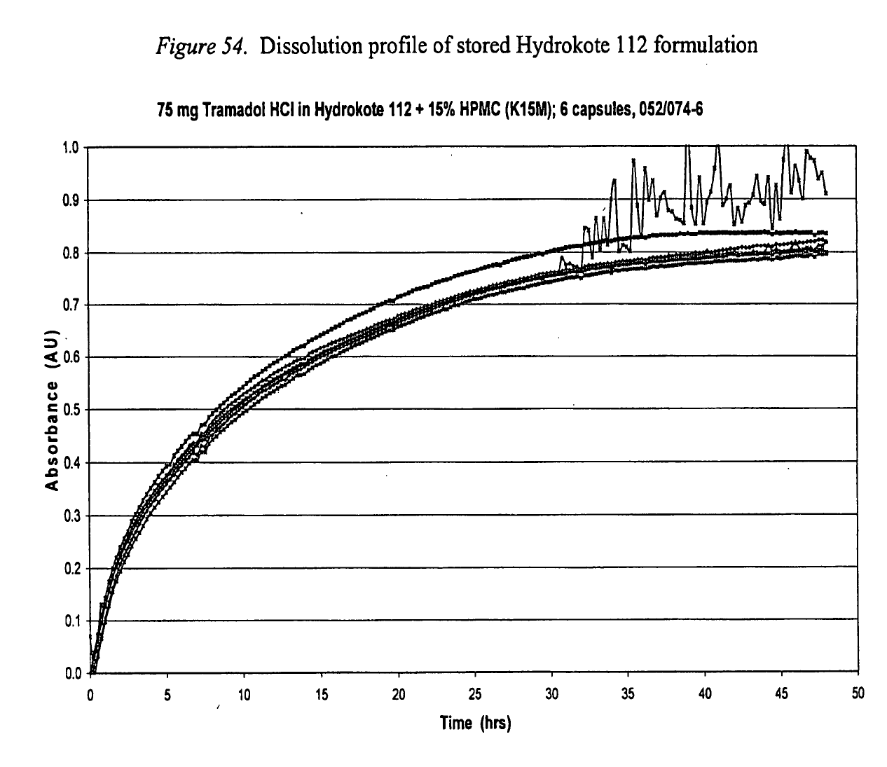

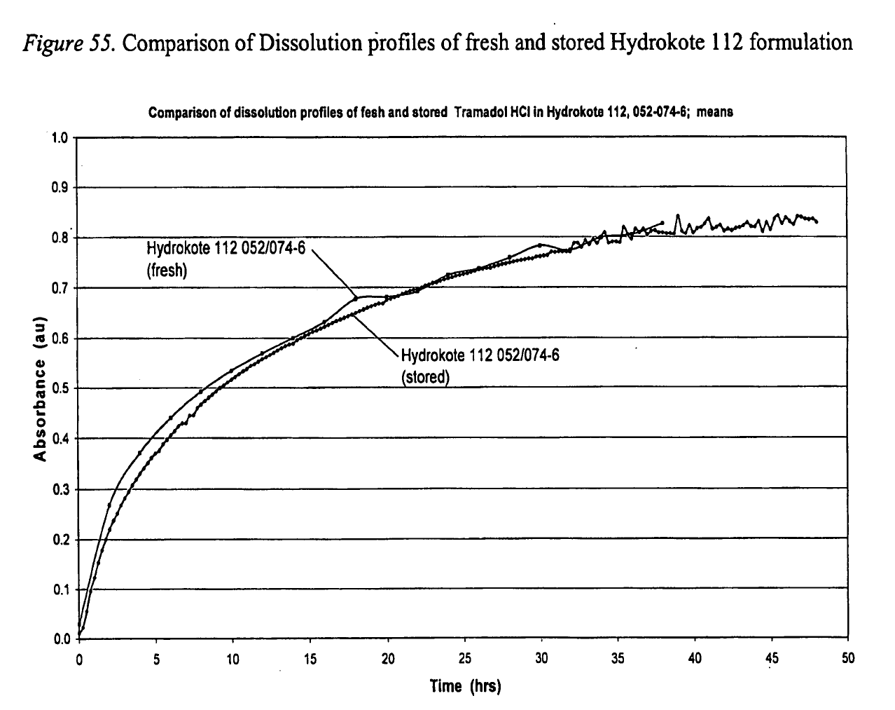

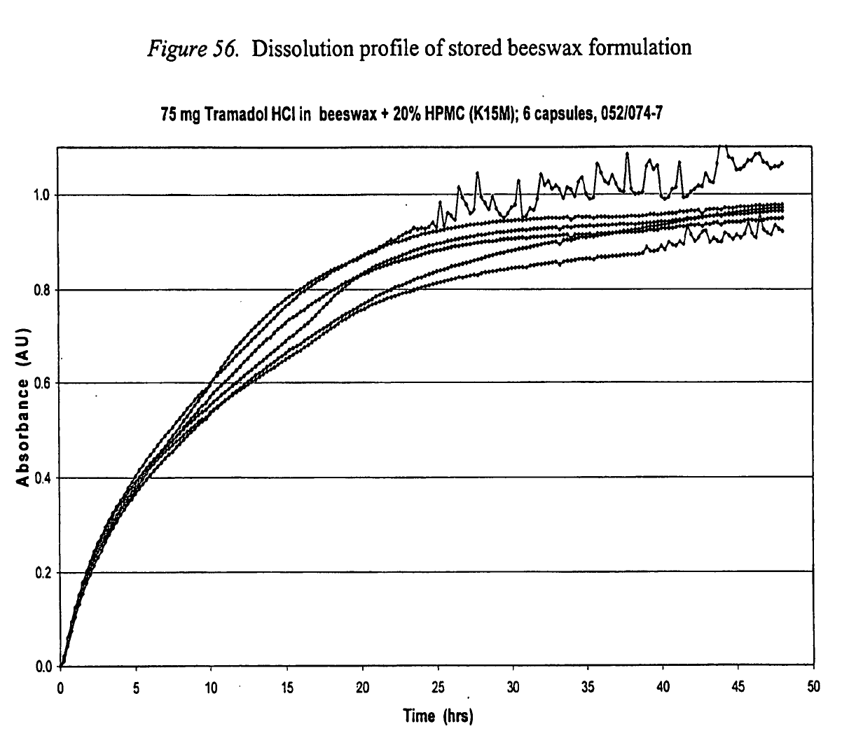

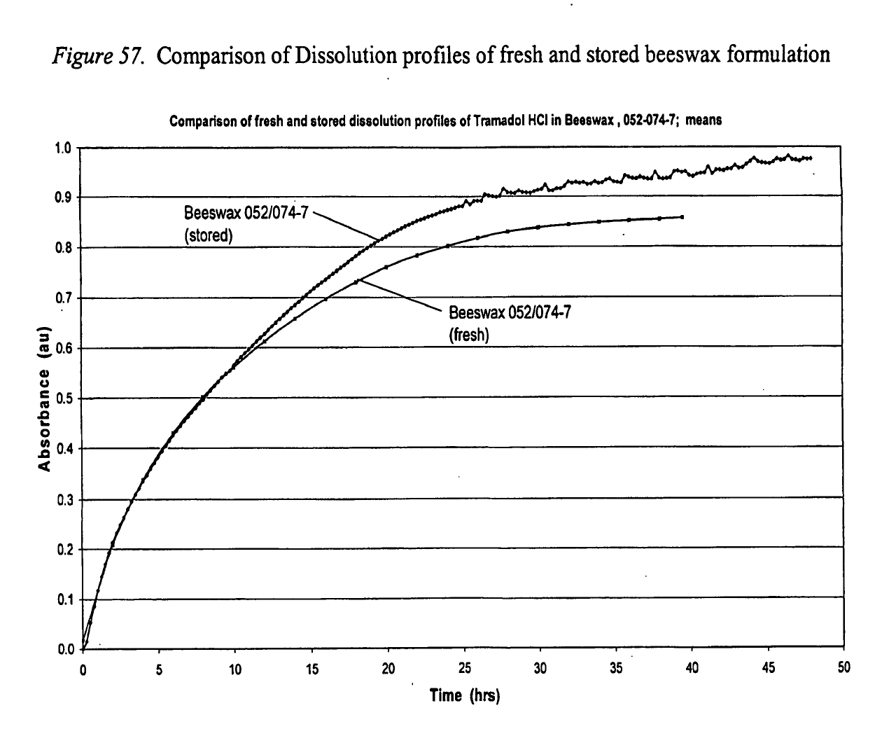

Samples of the above formulations were stored for a period of at least four weeks at room temperature (in some cases much longer) after which their dissolution release profile was redetermined. This was carried out to find out if there were any short term changes in the release rate. The tested formulations are shown in Table 13 and FIGS. 48 to 57.

TABLE 13
Formulations used for dissolution testing after a minimum of
4 weeks storage.

| | | Storage | |
|---|---|---|---|
| | | period | |
| Base Excipient | Formulation | days | Comment |
| Sterotex ® N | 052/087-1 | 75 | 20% HPMC |
| Sterotex ® NF with | 052/094-3 | 71 | |
| 25% fr. coconut oil | | | |
| Cithrol ® GMS | 052/073-5 | 95 | |
| Hydrokote 112 | 052/074-6 | 98 | |
| Beeswax | 052/074-7 | 83 | |

Manufacturing methods described above are utilized for the preparation of other opioids. Compositions and methods of the present invention provide (i) abuse deterrence; (ii) extended release; and (iii) simultaneous abuse deterrence and extended release, prepared using compounds selected from the group consisting of: (a) hydrogenated Type I or Type II vegetable oils (e.g., Hydrokote® 112); (b) polyoxyethylene stearates and distearates; (c) glycerol monostearate (e.g., Cithrol® GMS); (d) poorly water soluble, high melting point (mp=40 to 100° C.) waxes, and mixtures thereof, said compounds hereinafter referred to as “abuse deterrent, extended release” or “ADER”.

As shown in further examples below, any opioid agonist of the invention may be prepared to provide (i) abuse deterrence; (ii) extended release; and (iii) simultaneous abuse deterrence and extended release, prepared using compounds selected from the group consisting of: (a) hydrogenated Type I or Type II vegetable oils; (b) polyoxyethylene stearates and distearates; (c) glycerol monostearate; (d) poorly water soluble, high melting point (mp=40 to 100° C.) waxes, and mixtures thereof, said group of compounds hereinafter referred to as “abuse deterrent, extended release” or “ADER”.

### Example 31

| Ingredients | Quantity (mg)/Dose |
|---|---|
| Sterotex ® NF | 200 |
| Fractionated coconut oil | 70 |
| Methocel ® K 15M | 81 |
| Aerosil ® COK 84 | 4 |
| Hydromorphone HCl | 20 |
| Capsule fill weight | 375 |

### Example 32

| Ingredients | Quantity (mg)/Dose |
|---|---|
| Sterotex ® NF | 135 |
| Fractionated coconut oil | 50 |
| Methocel ® K 15M | 60 |
| Aerosil ® COK 84 | 3 |
| Fentanyl HCl | 2 |
| Capsule fill weight | 250 |

### Example 33

| Ingredients | Quantity (mg)/Dose | |
|---|---|---|
| Sterotex ® NF | 170 | |
| Fractionated coconut oil | 100 | |
| Methocel ® K 15M | 70 | |
| Aerosil ® COK 84 | 4.5 | |
| Levorphanol | 5.5 | mg |
| Capsule fill weight | 350 | |

### Example 34

| Ingredients | Quantity (mg)/Dose | |
|---|---|---|
| Sterotex ® NF | 200 | |
| Fractionated coconut oil | 90 | |
| Methocel ® K 15M | 80 | |
| Aerosil ® COK 84 | 5 | |
| Hydrocodone | 25 | |
| Capsule fill weight | 400 | mg |

### Example 35

| Ingredients | Quantity (mg)/Dose |
|---|---|
| Beeswax | 200 |
| HPMC, K15M | 80 |
| Aerosil COK 84 | 8 |
| Levorphanol Tartrate | 12 |
| Capsule fill weight | 300 |

### Example 36

| Ingredients | Quantity (mg)/Dose |
|---|---|
| Sterotex NF | 150 |
| HPMC, K15M | 75 |
| Coconut oil | 75 |
| Aerosil COK 84 | 5 |
| Oxymorphone | 20 |
| Capsule fill weight | 325 |

### Example 37

| Ingredients | Quantity (mg)/Dose |
|---|---|
| Cithrol GMS | 275 |
| HPMC, K100M | 40 |
| Aerosil COK 84 | 10 |
| Methadone | 25 |
| Capsule fill weight | 350 |

### Example 38

| Ingredients | Quantity (mg)/Dose |
|---|---|
| Hydrokote 112 | 250 |
| HPMC, K15M | 60 |
| Aerosil COK 84 | 10 |
| Morphine | 30 |
| Capsule fill weight | 350 |

### Example 39

| Ingredients | Quantity (mg)/Dose |
|---|---|
| Beeswax | 200 |
| HPMC, Pharmacoat 606 | 62.5 |
| Aerosil COK 84 | 7.5 |
| Hydrocodone | 30 |
| Capsule fill weight | 300 |

### Example 40

| Ingredients | Quantity (mg)/Dose |
|---|---|
| Gelucire 50/02 | 190 |
| Methocel K 100M | 35 |
| Aerosil COK 84 | 10 |
| Hydromorphone HCl | 15 |
| Capsule fill weight | 250 |

### Example 41

| Ingredients | Quantity (mg)/Dose |
|---|---|
| Cetyl alcohol | 280 |
| Methocel K 100M | 50 |
| Aerosil COK 84 | 10 |
| Levorphanol | 10 |
| Capsule fill weight | 350 |

### Example 42

| Ingredients | Quantity (mg)/Dose |
|---|---|
| Sterotex NF | 320 |
| Methocel K 15M | 60 |
| Aerosil COK 84 | 10 |
| Oxycodone | 10 |
| Capsule fill weight | 400 |

### Example 43

| Ingredients | Quantity (mg)/Dose |
|---|---|
| Cithrol GMS | 320 |
| Methocel K 100M | 55 |
| Aerosil COK 84 | 15 |
| Oxymorphone | 10 |
| Capsule fill weight | 400 |

### Example 44

| Ingredients | Quantity (mg)/Dose |
|---|---|
| Hydrokote 112 | 225 |
| Methocel K 15M | 50 |
| Aerosil COK 84 | 10 |
| Hydrocodone | 15 |
| Capsule fill weight | 300 |

### Example 45

| Ingredients | Quantity (mg)/Dose |
|---|---|
| Beeswax | 225 |
| Methocel K 15M | 75 |
| Aerosil COK 84 | 10 |
| Dihydrocodeine | 15 |
| Capsule fill weight | 325 |

### Example 46

| Ingredients | Quantity (mg)/Dose |
|---|---|
| Beeswax | 210 |
| HPMC, K15M | 80 |
| Aerosil COK 84 | 8 |
| Remifentanil | 2 |
| Capsule fill weight | 300 |

### Example 47

| Ingredients | Quantity (mg)/Dose |
|---|---|
| Sterotex NF | 166 |
| HPMC, K15M | 75 |
| Coconut oil | 75 |
| Aerosil COK 84 | 5 |
| Sufentanil | 4 |
| Capsule fill weight | 325 |

### Example 48

| Ingredients | Quantity (mg)/Dose |
|---|---|
| Cithrol GMS | 285 |
| HPMC, K100M | 49 |
| Aerosil COK 84 | 10 |
| Alfentanil | 6 |
| Capsule fill weight | 350 |

### Example 49

| Ingredients | Quantity (mg)/Dose |
|---|---|
| Hydrokote 112 | 240 |
| HPMC, K15M | 50 |
| Aerosil COK 84 | 10 |
| Propiram HCl | 100 |
| Capsule fill weight | 400 |

### Example 50

| Ingredients | Quantity (mg)/Dose |
|---|---|
| Beeswax | 195 |
| HPMC, Pharmacoat 606 | 45 |
| Aerosil COK 84 | 10 |
| Propiram | 150 |
| Capsule fill weight | 400 |

### Example 51

| Ingredients | Quantity (mg)/Dose |
|---|---|
| Gelucire 50/02 | 190 |
| Methocel K 100M | 30 |
| Aerosil COK 84 | 10 |
| Hydromorphone HCl | 20 |
| Capsule fill weight | 250 |

### Example 52

| Ingredients | Quantity (mg)/Dose |
|---|---|
| Cetyl alcohol | 290 |
| Methocel K 100M | 50 |
| Aerosil COK 84 | 10 |
| Hydrocodone | 50 |
| Capsule fill weight | 400 |

### Example 53

| Ingredients | Quantity (mg)/Dose |
|---|---|
| Sterotex NF | 320 |
| Methocel K 15M | 60 |
| Aerosil COK 84 | 10 |
| Oxymorphone | 40 |
| Capsule fill weight | 430 |

### Example 54

| Ingredients | Quantity (mg)/Dose |
|---|---|
| Cithrol GMS | 320 |
| Methocel K 100M | 68 |
| Aerosil COK 84 | 12 |
| Oxycodone | 60 |
| Capsule fill weight | 460 |

### Example 55

| Ingredients | Quantity (mg)/Dose |
|---|---|
| Hydrokote 112 | 225 |
| Methocel K 15M | 50 |
| Aerosil COK 84 | 10 |
| Methadone | 40 |
| Capsule fill weight | 325 |

### Example 56

| Ingredients | Quantity (mg)/Dose |
|---|---|
| Beeswax | 235 |
| Methocel K 15M | 75 |
| Aerosil COK 84 | 14 |
| Codeine S0₄ | 150 |
| Capsule fill weight | 474 |

### Example 57

| Ingredients | Quantity (mg)/Dose |
|---|---|
| Beeswax | 200 |
| HPMC, K15M | 90 |
| Aerosil COK 84 | 10 |
| Pentazocine | 100 |
| Capsule fill weight | 40 |

### Example 58

| Ingredients | Quantity (mg)/Dose |
|---|---|
| Sterotex NF | 150 |
| HPMC, K15M | 75 |
| Coconut oil | 80 |
| Aerosil COK 84 | 10 |
| Anleridine | 100 |
| Capsule fill weight | 415 |

### Example 59

| Ingredients | Quantity (mg)/Dose |
|---|---|
| Cithrol GMS | 290 |
| HPMC, K100M | 48 |
| Aerosil COK 84 | 12 |
| Lofentanil | 0.1 |
| Capsule fill weight | 350.1 |

### Example 60

| Ingredients | Quantity (mg)/Dose |
|---|---|
| Hydrokote 112 | 270 |
| HPMC, K15M | 65 |
| Aerosil COK 84 | 15 |
| Carfentanil | 0.2 |
| Capsule fill weight | 350.2 |

### Example 61

| Ingredients | Quantity (mg)/Dose |
|---|---|
| Beeswax | 177 |
| HPMC, Pharmacoat | 60 |
| 606 | |
| Aerosil COK 84 | 10 |
| Fentanyl | 3 |
| Capsule fill weight | 250 |

### Example 62

| Ingredients | Quantity (mg)/Dose |
|---|---|
| Gelucire 50/02 | 190 |
| Methocel K 100M | 40 |
| Aerosil COK 84 | 10 |
| Alfentanil | 10 |
| Capsule fill weight | 250 |

### Example 63

| Ingredients | Quantity (mg)/Dose |
|---|---|
| Cetyl alcohol | 270 |
| Methocel K 100M | 50 |
| Aerosil COK 84 | 10 |
| Buprenorphine | 20 |
| Capsule fill weight | 350 |

### Example 64

| Ingredients | Quantity (mg)/Dose |
|---|---|
| Sterotex NF | 293 |
| Methocel K 15M | 45 |
| Aerosil COK 84 | 10 |
| Sufentanil | 2 |
| Capsule fill weight | 350 |

### Example 65

| Ingredients | Quantity (mg)/Dose |
|---|---|
| Cithrol GMS | 325 |
| Methocel K 100M | 55 |
| Aerosil COK 84 | 15 |
| Fentanyl | 5 |
| Capsule fill weight | 400 |

### Example 66

| Ingredients | Quantity (mg)/Dose |
|---|---|
| Hydrokote 112 | 225 |
| Methocel K 15M | 50 |
| Aerosil COK 84 | 10 |
| Hydrocodone | 15 |
| Capsule fill weight | 300 |

### Example 67

| Ingredients | Quantity (mg)/Dose |
|---|---|
| Beeswax | 225 |
| Methocel K 15M | 75 |
| Aerosil COK 84 | 10 |
| Racemorphan | 20 |
| Capsule fill weight | 330 |

The present invention can also optionally include other ingredients in addition to the opioid and ADER to enhance dosage form and/or alter the release profile of a dosage form.

Some embodiments of the present invention include one or more pharmaceutically acceptable fillers, diluents, glidants and lubricants of various particle sizes and molecular weights.

The dosage form according to the invention may also comprise a coating which is resistant to gastric juices and dissolves as a function of the pH value of the release environment.

By means of this coating, it is possible to ensure that, when correctly administered, the dosage form according to the invention passes through the stomach undissolved and the active ingredient is only released in the intestines.

In some preferred embodiments, the dosage form may include a surfactant ingredient to impart suitable formulation characteristics to the composition. Surfactants may be hydrophilic preferably selected from the group consisting of non-ionic hydrophilic surfactants and anionic hydrophilic surfactants or the surfactant may have hydrophobic properties. Examples of non-ionic hydrophilic surfactants are polyoxyethylene sorbitan esters, cremophores and poloxamers. Examples of anionic surfactants are sodium lauryl sarcosinate, docusate and pharmaceutically acceptable docusate salts. Also a mixture of these surfactants can be used.

The formulation optionally comprises auxiliary materials. Examples of these auxiliary materials (or pharmaceutically acceptable excipients) are (i) Binders such as acacia, alginic acid and salts thereof, cellulose derivatives, methylcellulose, hydroxyethyl cellulose, hydroxypropyl cellulose, magnesium aluminum silicate, polyethylene glycol, gums, polysaccharide acids, bentonites, hydroxypropyl methylcellulose, gelatin, polyvinylpyrrolidone, polyvinylpyrrolidone/vinyl acetate copolymer, crospovidone, povidone, polymethacrylates, hydroxypropylmethylcellulose, hydroxypropylcellulose, starch, pregelatinized starch, ethylcellulose, tragacanth, dextrin, microcrystalline cellulose, sucrose, or glucose, and the like; (ii) Disintegrants such as starches, pregelatinized corn starch, pregelatinized starch, celluloses, cross-linked carboxymethylcellulose, crospovidone, cross-linked polyvinylpyrrolidone, a calcium or a sodium alginate complex, clays, alginates, gums, or sodium starch glycolate, and any disintegration agents used in tablet preparations; (iii) Filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like; (iv) Stabilizers such as any antioxidation agents, buffers, or acids, and the like; (v) Lubricants such as magnesium stearate, calcium hydroxide, talc, colloidal silicon dioxide, sodium stearyl fumarate, hydrogenated vegetable oil, stearic acid, glyceryl behenate, magnesium, calcium and sodium stearates, stearic acid, talc, waxes, Stearowet, boric acid, sodium benzoate, sodium acetate, sodium chloride, DL-leucine, polyethylene glycols, sodium oleate, or sodium lauryl sulfate, and the like; (vi) Wetting agents such as oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, or sodium lauryl sulfate, and the like; (vii) Diluents such lactose, starch, mannitol, sorbitol, dextrose, microcrystalline cellulose, dibasic calcium phosphate, sucrose-based diluents, confectioner's sugar, monobasic calcium sulfate monohydrate, calcium sulfate dihydrate, calcium lactate trihydrate, dextrates, inositol, hydrolyzed cereal solids, amylose, powdered cellulose, calcium carbonate, glycine, or bentonite, and the like; (viii) Anti-adherents or glidants such as talc, corn starch, DL-leucine, sodium lauryl sulfate, and magnesium, calcium, or sodium stearates, and the like (ix) Pharmaceutically compatible carriers such as acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, or pregelatinized starch, and the like.

These and other embodiments of the present invention will readily occur to those of ordinary skill in the art in view of the disclosure herein.

A wide variety of materials can be used for preparing the dosage form according to this invention. This invention therefore contemplates the use of materials other than those specifically disclosed herein, including those which may hereafter become known to the art to be capable of performing the necessary functions.

1-160. (canceled)

161. An abuse deterrent monolithic solidified oral dosage form prepared by a thermal process comprising the following material: (a) a therapeutically effective amount of opioid agonist or a pharmaceutically acceptable salt thereof or a mixture thereof; (b) one or more compounds selected from the group consisting of: (i) hydrogenated Type I or Type II vegetable oils; (ii) polyoxyethylene stearates and distearates; (iii) glycerol monostearate; (iv) poorly water soluble, high melting point (mp=45 to 100° C.) waxes; and (c) one or more additional pharmaceutically acceptable excipients, wherein the material are substantially uniformly dispersed, wherein abuse-deterrent and extended-release properties are simultaneously provided, wherein, upon physical tampering of the dosage form, the in-vitro release rate by weight of the opioid agonist from the dosage form over the first hour when measured by the USP paddle method at 100 rpm in 900 mL aqueous buffer at a pH of between 1.6 and 7.2 at 37° C. is less than for an immediate release form of the opioid agonist, and wherein, upon physical tampering of the dosage form, the in-vivo dose-normalized Cₘₐₓ upon first administration of the opioid agonist from the dosage form is less than for an immediate release form of the opioid agonist.

162. An abuse deterrent monolithic solidified oral dosage form prepared by a thermal process comprising the following material: (a) a therapeutically effective amount of opioid agonist or a pharmaceutically acceptable salt thereof or a mixture thereof; (b) one or more compounds selected from the group consisting of: (i) hydrogenated Type I or Type II vegetable oils; (ii) polyoxyethylene stearates and distearates; (iii) glycerol monostearate; (iv) poorly water soluble, high melting point (mp=45 to 100° C.) waxes; and (c) at least one pharmaceutically acceptable excipient selected from the group comprising a thixotrope and a release rate modifier, wherein the thixotrope and release rate modifier provide further abuse deterrence, wherein the material are substantially uniformly dispersed, wherein abuse-deterrent and extended-release properties are simultaneously provided, wherein, upon physical tampering of the dosage form, the in-vitro release rate by weight of the opioid agonist from the dosage form over the first hour when measured by the USP paddle method at 100 rpm in 900 mL aqueous buffer at a pH of between 1.6 and 7.2 at 37° C. is less than for an immediate release form of the opioid agonist, and wherein, upon physical tampering of the dosage form, the in-vivo dose-normalized Cₘₐₓ upon first administration of the opioid agonist from the dosage form is less than for an immediate release form of the opioid agonist.

163. A claim according to claim 162, wherein the dosage form consists of (a) a therapeutically effective amount of opioid agonist or a pharmaceutically acceptable salt thereof or a mixture thereof; (b) one or more compounds selected from the group consisting of: (i) hydrogenated Type I or Type II vegetable oils; (ii) polyoxyethylene stearates and distearates; (iii) glycerol monostearate; (iv) poorly water soluble, high melting point (mp=45 to 100° C.) waxes; and (c) a thixotrope.

164. A claim according to claim 162, wherein the dosage form consists of (a) a therapeutically effective amount of opioid agonist or a pharmaceutically acceptable salt thereof or a mixture thereof; (b) one or more compounds selected from the group consisting of: (i) hydrogenated Type I or Type II vegetable oils; (ii) polyoxyethylene stearates and distearates; (iii) glycerol monostearate; (iv) poorly water soluble, high melting point (mp=45 to 100° C.) waxes; and (c) a release rate modifier.

165. A claim according to claim 162, wherein the dosage form consists of (a) a therapeutically effective amount of opioid agonist or a pharmaceutically acceptable salt thereof or a mixture thereof; (b) one or more compounds selected from the group consisting of: (i) hydrogenated Type I or Type II vegetable oils; (ii) polyoxyethylene stearates and distearates; (iii) glycerol monostearate; (iv) poorly water soluble, high melting point (mp=45 to 100° C.) waxes; (c) a thixotrope; and (d) a release rate modifier.

166. A claim according to claim 162, wherein the thixotrope is silicon dioxide or a mixture of silicon dioxide and aluminum oxide and the release rate modifier is hydroxypropyl methylcellulose.

167. A claim according to claim 161 and claim 162, wherein the dosage form is prepared by liquid filling the material into capsules.

168. A claim according to claim 161 and claim 162, wherein the dosage form deters surreptitious adulteration of a beverage and surreptitious intoxication.

169. A claim according to claim 161 and claim 162, wherein the dosage form reduces or prevents the toxicity of the opioid agonist due to dose dumping when the dosage form is co-ingested with alcohol, wherein the ratio of the mean opioid agonist Cₘₐₓ upon first administration of the intact dosage form concurrently with 240 mL of 40% ethanol to the mean Cₘₐₓ after first administration of the intact dosage form without 240 mL of 40% ethanol, each administered orally to fasted subjects, is not more than about 4:1.

170. A claim according to claim 161 and claim 162, wherein the opioid agonist is the most water soluble commercially available pharmaceutically acceptable salt.

171. A claim according to claim 161 and claim 162, wherein the dosage form has diameter of more than 5 mm.

172. A claim according to claim 161 and claim 162, wherein the molar amount of the compound is at least 15-fold greater than the molar amount of the opioid agonist or its pharmaceutically acceptable salt, inclusive of it water of hydration (e.g., anhydrous, monohydrate, dihydrate) in the dosage form.

173. A claim according to claim 161 and claim 162, wherein the material substantially forms a single phase in the dosage form and/or wherein the dosage form is not in the form of an aggregate or composite of individual solid particulates.

174. A claim according to claim 161 and claim 162, wherein the material comprising the opioid agonist, the compound and pharmaceutically acceptable excipients are prepared or manufactured in situ (i.e., in a single reaction and mixing vessel) prior to transfer to a filling machine for final encapsulation.

175. A claim according to claim 161 and claim 162, providing an in-vitro release rate by weight of an opioid agonist of (i) less than about 35% after 30 minutes of agitation of the intact dosage form on an orbital shaker at 240 cycles/min in 18 mL of a 0.1 N HCl solution in a 60 mL amber bottle, said release less than about 60% following the addition of 12 mL of 90% to 95% ethanol and further agitation for 180 minutes on an orbital shaker at 240 cycles/min, each at 25° C.; (ii) less than about 50% after 30 minutes of agitation of the tampered dosage form on an orbital shaker at 240 cycles/min in 18 mL of a 0.1 N HCl solution in a 60 mL amber bottle, said release less than about 70% following the addition of 12 mL of ethanol 90% to 95% and further agitation for 180 minutes on an orbital shaker at 240 cycles/min, each at 25° C.; and (iii) between 0% and about 60% at 1 hour, between about 0% and about 80% at 2 hours, between about 1% and about 95% at 4 hours and between about 10% and about 100% at 8 hours, when measured by the USP paddle method at 100 rpm in 900 mL aqueous buffer at a pH of between 1.6 and 7.2 at 37° C.

176. A claim according to claim 161 and claim 162, wherein (i) the ratio of the mean opioid agonist Cₘₐₓ upon first administration after tampering to the mean Cₘₐₓ after first administration of an intact dosage form is not more than about 6:1; (ii) the ratio of the mean opioid agonist AUC₀₋₁ upon first administration after tampering to the mean AUC₀₋₁ after first administration of an intact dosage form is not more than about 6:1; (iii) the ratio of the mean opioid agonist Tₘₐₓ upon first administration of an intact dosage form to the mean Tₘₐₓ after first administration of a tampered dosage form is not more than about 6:1; and (iv) the ratio of the mean opioid agonist Cₘₐₓ upon first administration of the intact dosage form concurrently with 240 mL of 40% ethanol to the mean Cₘₐₓ after first administration of the intact dosage form without 240 mL of 40% ethanol, each to fasted subjects, is not more than about 4:1.

177. A claim according to claim 161 and claim 162, wherein: (i) the mean ratio of the time to meaningful pain relief after administration of the intact dosage form to a human patient to the time to meaningful pain relief after administration of the tampered dosage form is less than 6:1; (ii) mean ratio of the peak pain intensity difference score after administration of the tampered dosage form to a human patient to the peak pain intensity difference score after administration of the intact dosage form is less than 6:1; (iii) the mean ratio of the number needed to harm (NNH) due to moderate or severe nausea after administration of the tampered dosage form to opioid naïve healthy subjects to the NNH due to moderate or severe nausea after administration of the intact dosage form of less than 6:1; (iv) the mean ratio of the drug liking score in drug abusers and recreational drug users without pain after administration of the tampered dosage form to the drug liking score after administration of the of the intact dosage form is less than 6:1; and (v) the mean ratio of the drug effect score in drug abusers and recreational drug users without pain after administration of the tampered dosage form to the drug effect score after administration of the intact dosage form is less than 6:1.

178. A claim according to claim 161 and claim 162, wherein the compounds are further selected form the group comprising: glyceryl behenate, hydrogenated vegetable oil, hydrogenated cottonseed oil, hydrogenated palm oil, hydrogenated soybean oil, hydrogenated coconut oil, their respective esters and derivatives.

179. A claim according to claim 161 and claim 162, wherein the opioid agonist is selected from the group comprising (i) alfentanil, anileridine, buprenorphine, brifentanil, butorphanol, carfentanil, codeine, dextromoramide, dezocine, dihydrocodeine, fentanyl, heroin, hydrocodone, hydromorphone, ketobemidone, levorphanol, levomethadone, lofentanil, meperidine, meptazinol, methadone, 4-methoxymethylfentanyl, 3-methylfentanil, mirfentanil, morphine, morphine-6-glucuronide, nalbuphine, ohmefentanyl, O-desmethyl tramadol, opium, oxycodone, oxymorphone, pentazocine, phenazocine, propiram, propoxyphene, racemorphan, remifentanil, sulfentanil, tapentadol, trefentanil, tramadol, tilidine; and (ii) any opioid agonist belonging to the phenanthrene, morphinan, benzomorphan, methadone, phenylpiperidine, propionanilide 4-anilidopiperidine, 4-aryl piperidines and 4-heteroarylpiperidines class, and their pharmaceutically acceptable salts, prodrugs, esters, polymorphs, hydrates and metabolites, as racemates or an individual diastereoisomers or enantiomeric isomers thereof or mixtures thereof.

180. A method of preparing an abuse deterrent monolithic solidified oral dosage form by a thermal process comprising the material in claim 161 and claim 162, wherein the material are substantially uniformly dispersed, wherein abuse-deterrent and extended-release properties are simultaneously provided, wherein, upon physical tampering of the dosage form, the in-vitro release rate by weight of the opioid agonist from the dosage form over the first hour when measured by the USP paddle method at 100 rpm in 900 mL aqueous buffer at a pH of between 1.6 and 7.2 at 37° C. is less than for an immediate release form of the opioid agonist, wherein, upon physical tampering of the dosage form, the in-vivo dose-normalized Cₘₐₓ upon first administration of the opioid agonist from the dosage form is less than for an immediate release form of the opioid agonist and wherein, in the case of material from claim 162, the thixotrope and release rate modifier provide further abuse deterrence.
